(19) United States
(12) Patent Application Publication
(10) Pub. No.: US 2020/0327962 A1
(43) Pub. Date: Oct. 15, 2020

(54) STATISTICAL AI FOR ADVANCED DEEP LEARNING AND PROBABILISTIC PROGRAMING IN THE BIOSCIENCES

(71) Applicant: Wuxi Nextcode Genomics USA, Inc., Cambridge, MA (US)

(72) Inventors: Thomas W. Chittenden, Medford, MA (US); Nicholas A. Cilfone, Boston, MA (US); Pengwei Yang, Belmont, MA (US)

(21) Appl. No.: 16/851,949

(22) Filed: Apr. 17, 2020

Related U.S. Application Data
(63) Continuation of application No. PCT/US2018/056586, filed on Oct. 18, 2018.
(60) Provisional application No. 62/573,996, filed on Oct. 18, 2017.
(60) Provisional application No. 62/580,263, filed on Nov. 1, 2017.

(51) Int. Cl. G16B 40/20; G16B 20/00; G16B 5/20; G16B 40/30; G16B 45/00; G16B 25/00; G16H 50/80

(52) U.S. Cl. CPC G16B 40/20; G16B 20/00; G16B 5/20; G16H 50/80; G16B 45/00; G16B 25/00; G16B 40/30

(57) ABSTRACT

Statistical artificial intelligence for advanced deep learning and probabilistic programming in the biosciences is provided. In various embodiments, biological data of a population is read. The biological data include molecular features of the population. A plurality of features of the population is extracted from the biological data. The plurality of features is provided to a first trained classifier to determine a subset of the plurality of features distinguishing the population. A plurality of genes associated with the subset of the plurality of features is determined. The plurality of genes is provided to a second trained classifier to determine a subset of the plurality of genes distinguishing the population. A dependence model is applied to the subset of the plurality of genes to determine one or more drug target.

### CROSS-REFERENCE TO RELATED APPLICATIONS

This application is a continuation of International Application No. PCT/US2018/056586, filed Oct. 18, 2018, which claims the benefit of U.S. Provisional Application No. 62/573,996, filed Oct. 18, 2017 and U.S. Provisional Application No. 62/580,263, filed Nov. 1, 2017, each of which are hereby incorporated by reference herein in its entirety.

### BACKGROUND

Embodiments of the present disclosure relate to analysis of multi-omic data, and more specifically, to statistical artificial intelligence for advanced deep learning and probabilistic programming in the biosciences.

### BRIEF SUMMARY

According to embodiments of the present disclosure, methods of and computer program products for identifying drug targets are provided. Biological data of a population is read. The biological data include molecular features of the population. A plurality of features of the population is extracted from the biological data. The plurality of features is provided to a first trained classifier to determine a subset of the plurality of features distinguishing the population. A plurality of genes associated with the subset of the plurality of features is determined. The plurality of genes is provided to a second trained classifier to determine a subset of the plurality of genes distinguishing the population. A dependence model is applied to the subset of the plurality of genes to determine one or more drug target.

### BRIEF DESCRIPTION OF THE SEVERAL VIEWS OF THE DRAWINGS

FIG. 1 illustrates a method of genomic analysis according to embodiments of the present disclosure.

FIG. 2 is a schematic guide to cancer types, acronyms, and sample numbers from The Cancer Genome Atlas (TCGA).

FIG. 3A-FIG. 3I illustrate methods of genomic analysis according to embodiments of the present disclosure.

FIG. 4A-FIG. 4E depict binomial model comparisons at both the module and gene level specifically highlighting kidney renal papillary cell carcinoma (KIRP) versus kidney renal clear cell carcinoma (KIRC).

FIG. 5A-FIG. 5E depict multinomial models at the module and gene level comparing 22 cancer types from the TCGA database.

FIG. 6A-FIG. 6D show survival models at the module and gene level comparing 20 cancer types from the TCGA database.

FIG. 7A-FIG. 7F depict the analysis of the most informative survival genes.

FIG. 8 depicts a computing node according to an embodiment of the present invention.

FIG. 9A-FIG. 9D depict binomial model comparisons at both the module and gene level specifically highlighting breast cancer (BRCA) versus normal tissue.

FIG. 10A-FIG. 10D depict binomial model comparisons at both the module and gene level specifically highlighting LUAD versus LUSC lung cancer subtypes.

FIG. 11A-FIG. 11D depict binomial model comparisons at both the module and gene level specifically highlighting ER+ versus ER− breast cancer subtypes.

FIG. 12A-FIG. 12D depict binomial model comparisons at both the module and gene level specifically highlighting Luminal A versus Luminal B breast cancer subtypes.

FIG. 13A and FIG. 13B depict the top 20 most informative MEGENA genes at the gene level for Lung Adenocarcinoma (LUAD) versus Lung Squamous Cell (LUSC) lung cancer subtypes (for both training (FIG. 13B) and testing data sets (13A)).

FIG. 14A and FIG. 14B depict the top 20 most informative nGOseq genes at the gene level for Lung Adenocarcinoma (LUAD) versus Lung Squamous Cell (LUSC) lung cancer subtypes (for both training (FIG. 14B) and testing data sets (14A)).

FIG. 15A and FIG. 15B depicts the top 20 most informative MEGENA genes at the gene level for ER+ versus ER− breast cancer subtypes (for both training (FIG. 15B) and testing data sets (15A)).

FIG. 16A and FIG. 16B depicts the top 20 most informative nGOseq genes at the gene level for ER+ versus ER− breast cancer subtypes (for both training (FIG. 16B) and testing data sets (16A)).

FIG. 17A and FIG. 17B depicts the top 20 most informative MEGENA genes at the gene level for Luminal A versus Luminal B breast cancer subtypes (for both training (FIG. 17B) and testing data sets (17A)).

FIG. 18A and FIG. 18B depicts the top 20 most informative nGOseq genes at the gene level for Luminal A versus Luminal B breast cancer subtypes (for both training (FIG. 18A) and testing data sets (18B)).

FIG. 19A and FIG. 19B depicts the top 20 most informative MEGENA genes at the gene level for breast cancer (BRCA) versus normal tissue (for both training (FIG. 19B) and testing data sets (19A)).

FIG. 20A and FIG. 20B depicts the top 20 most informative nGOseq genes at the gene level for breast cancer (BRCA) versus normal tissue (for both training (FIG. 20B) and testing data sets (20A)).

FIG. 21A and FIG. 21B depicts the top 20 most informative MEGENA genes at the gene level for kidney renal papillary cell carcinoma (KIRP) versus kidney renal clear cell carcinoma (KIRC) (for both training (FIG. 21B) and testing data sets (21A)).

FIG. 22A and FIG. 22B depicts the top 20 most informative nGOseq genes at the gene level for kidney renal papillary cell carcinoma (KIRP) versus kidney renal clear cell carcinoma (KIRC) (for both training (FIG. 22B) and testing data sets (22A)).

FIG. 23A and FIG. 23B depicts the top 20 most informative MEGENA genes at the gene level for the pan 22 cancer comparison (for both training (FIG. 23B) and testing data sets (23A))

FIG. 24A and FIG. 24B depicts survival models at the nGOseq module level comparing 20 cancer types from the TCGA database.

FIG. 25A and FIG. 25B depicts survival models at the MEGENA gene level comparing 20 cancer types from the TCGA database.

FIG. 26A and FIG. 26B depicts survival models at the nGOseq gene level comparing 20 cancer types from the TCGA database.

### DETAILED DESCRIPTION

Improved sequencing technology has increased the breadth of data available for addressing questions in biology. Statistical methods may be applied to identify biologically relevant sets of genes whose collective state correlates with a given phenotype. However, placing these gene sets into a biologically relevant framework remains a significant challenge.

Gene expression profiling of DNA microarray and RNA-seq data provides wealth of data for diagnosing and predicting outcome of many human cancers. High-throughput technologies, such as DNA microarrays and next-generation sequencing (NGS), provide the means to examine how organisms respond, on a genome-wide scale, to experimental or natural perturbations and to the development of pathological conditions. However, widespread use of high-throughput gene expression profiling in clinical medicine has not been fully realized, due in part to precision and interoperability of available prediction models. Moreover, gene redundancy is a significant confounding factor in high-throughput expression profiling schemes and often leads to reduced information content of analytical outcomes. The large number of genes unrelated to a given state can serve to decrease prediction accuracy of classification strategies.

To address this and other challenges, the present disclosure provides for various feature learning methods that enhance quantitative assessment of annotated tissues of the Cancer Genome Atlas. These methods allow integrated molecular signals to be collapsed onto highly-informative gene sets across 22 cancer types. These network-based strategies improve performance and interoperability of two deep neural network strategies by identifying genes underlying cancer type specific biology and pan-cancer patient survival. The results described herein indicate the efficacy of these approaches to statistical issues associated with the analysis of a wide array of high-dimensional data.

In various embodiments, an ensemble computational intelligence platform is applied to single or multi-omic data on patient and/or control groups to determine the molecular differences between any 2 or more groups. The number of molecular features is reduced using a gene correlation methods. In various exemplary embodiments described below, two feature reduction methods are applied. First, a data-driven approach is applied that uses correlations among genes using the measured molecular data within these patient and/or control datasets to cluster genes into smaller number of features. Second, the nGOseq algorithm is applied to cluster genes based on previous biological annotations (for example, GOseq terms or other known gene ontologies). The systems and methods provided herein enable perfect and near perfect classifications of multiple human tumor type designations, independent of tissue-specific annotation, to identify known and previously undescribed integrated molecular signatures of pan-cancer etiology and patient survival, thus creating a new archetype for biological and therapeutic discovery.

According to various embodiments, deep learning methods such as DANN or DBNN are applied in parallel to the molecular data from the comparison sets of patients and/or controls to discover the most important gene clusters that distinguish the patient/control groups. The top gene clusters (e.g., 100) for each deep learning method are compared and again ranked to define the top gene clusters.

These top gene clusters are opened into the underlying genes and the deep learning methods are repeated in parallel to define the genes to the molecular data from the comparison sets of patients and/or controls to discover the most important individual gees that distinguish the patient/control groups. The top genes (e.g., 100) for each deep learning method are compared and again ranked to define the top genes. These genes are used to define the classification (and potential diagnostic) to define patients with certain tumor type, tumor subtype, or future survival prediction.

To define the most important driver genes within the top genes defined above, a Bayesian Belief Network is applied to the top genes. These driver genes represent drug targets that may be used for treatment of tumor types, tumor subtypes or most of all tumors.

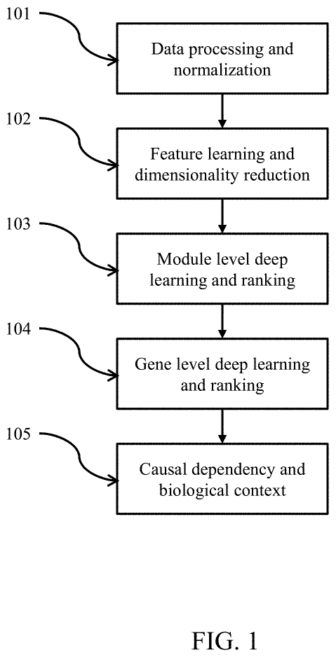

Referring now to FIG. 1, a schematic diagram of genomic analysis according to embodiments of the present disclosure is provided. It will be appreciated that although various examples herein are described with regard to The Cancer Genome Atlas (TCGA) data, the systems and methods described herein are generally applicable to disease condition having a genetic component.

As described further below, ensemble computational intelligence is applied to single or multi-omic data on patient and/or control groups to determine the molecular differences between any 2 or more groups. In various embodiments, multi-omic data includes omes such as genome, proteome, transcriptome, epigenome, and microbiome data.

At 101, input data are processed and normalized. In some embodiments, input data include messenger RNAs (mRNAs), somatic tumor variants (STVs), copy number variations (CNVs), micro RNAs (miRNAs), and DNA methylation (METH). In various embodiments, processing includes normalization and concatenation into a data matrix.

At 102, one or more feature learning algorithm is applied to generate a reduced feature space from the input data. It will be appreciated that a variety of feature learning and dimensional reduction techniques are suitable for use according to the present disclosure.

In various embodiments, the feature space is generated by clustering the biological data. In various embodiments clustering includes hierarchical clustering, k-means clustering, distribution-based clustering, Gaussian mixture models, density-based clustering, or highly connected subgraphs clustering.

In various embodiments, the number of molecular features is reduced using a gene correlation method. In exemplary embodiments discussed further below, two feature reduction methods are applied: 1) a data-driven approach that uses correlations among genes using the measured molecular data within these patient and/or control datasets to cluster genes into smaller number of features, and 2) nGOseq which clusters genes based on previous biological annotations in the public domain (for example, GOseq terms or other known gene ontologies).

In some embodiments, a plurality of feature learning techniques are applied. For example, in some embodiments, a data driven clustering approach (such as MEGENA) or an a priori biological knowledge based approach (such as nGOseq) is applied in addition to principal component analysis (PCA). In some embodiments, module-level data matrices are generated as a result of the feature learning step.

At 103, the module data are provided to one or more trained classifiers to determine the most informative modules. In some embodiments, multiple classifiers are applied to the data in an ensemble approach.

For example, in some embodiments, a Deep Artificial Neural Network (DANN) and a Deep Bayesian Neural Network (DBNN) are applied in parallel to the molecular data from the comparison sets of patients and/or controls to discover the most important gene clusters that distinguish the patient/control groups. A saliency map (or sensitivity map) may be used to determine the most informative input modules. The top gene clusters for each deep learning method may be compared and again ranked to define the top gene clusters. In some embodiments, a predetermined number of the top gene clusters are obtained, e.g., the top 100.

At 104, the genes from each of the important modules are broken out into gene level data matrices corresponding to the underlying genes. The gene level data are provided to one or more trained classifiers to determine the most informative genes. In some embodiments, multiple classifiers are applied to the data in an ensemble approach.

For example, in some embodiments, a Deep Artificial Neural Network (DANN) and a Deep Bayesian Neural Network (DBNN) are applied in parallel. The DANN or DBNN deep learning methods are repeated in parallel define the genes to the molecular data from the comparison sets of patients and/or controls to discover the most important individual genes that distinguish the patient/control groups. A saliency map may be used to determine the most informative genes.

The top genes for each deep learning method may be compared and again ranked to define the top genes. In some embodiments, a predetermined number of the top gene clusters are obtained, e.g., the top 100. These genes are used to define the classification (and potential diagnostic) to define patients with certain tumor type, tumor subtype, or future survival prediction.

At 105, the most informative genes are provided to a probabilistic model to determine causal genetic drivers. These driver genes represent potential drug targets that may be used for treatment of tumor types, tumor subtypes or most of all tumors. In some embodiments, the number of genes provided is limited to the most informative determined from prior steps (e.g., 100-200). In some embodiments, the probabilistic model is a Bayesian belief network. However, it will be appreciated that a variety of probabilistic models are suitable for use according to the present disclosure. In some embodiments, biological relevance is queried with natural language processing.

As described above, various learning systems are applied according to embodiments of the present disclosure. Various exemplary embodiments are described with respect to artificial neural networks, but it will be appreciated that a variety of learning systems are otherwise suitable. In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Various supervised and unsupervised machine learning methods may be used in accordance with the present disclosure, such as LASSO, Support Vector Machines, K-nearest-neighbor, Multivariate Partial Least Squares and Discriminant Analysis, Principal Component Analysis, Correspondence Analysis, and K-Means/K-Medians and Hierarchical clustering.

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

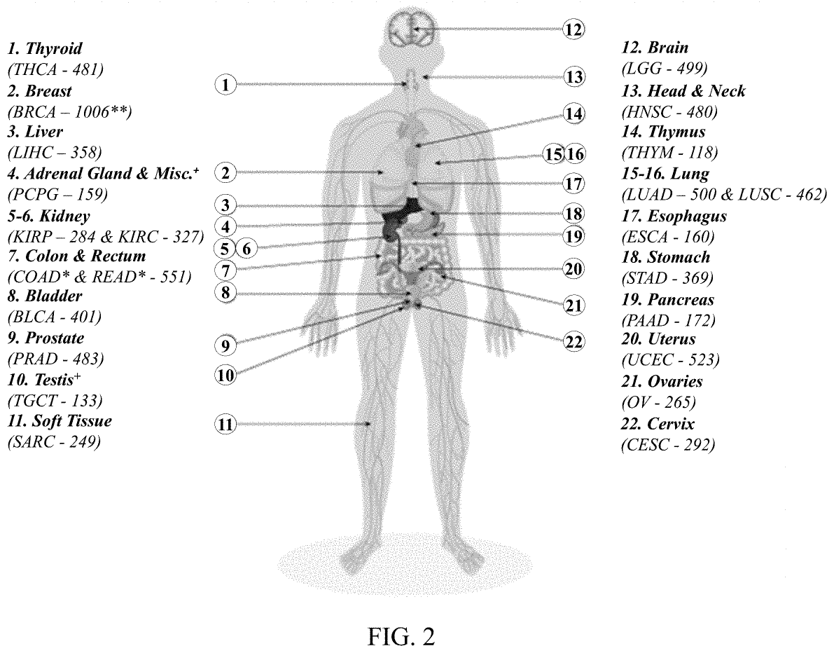

Referring to FIG. 2, a schematic guide to cancer types, acronyms, and sample numbers from The Cancer Genome Atlas (TCGA) is provided. As discussed further below, in an exemplary embodiment, 22 cancer types are studied. All available TCGA cancer types were filtered based on total sample number and availability of all five data types. Colon Adenocarcinoma (COAD) and Rectum Adenocarcinoma (READ) were merged into a single cancer type (CRAD) due to their similarity. Breast Invasive Carcinoma contains subtypes including ER status (+/−) and Luminal A/B used in subsequent binomial comparisons. Cancer of the Adrenal Gland (4) and Testis (10) were excluded from survival analysis. The total sample number for the below example is 8,272 for 22 cancers and 7,822 for 20 cancers.

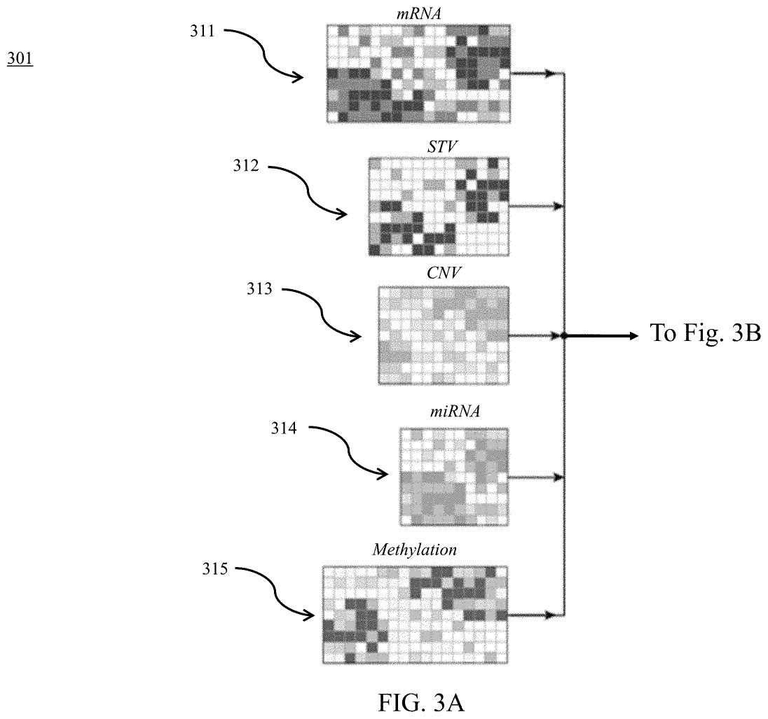

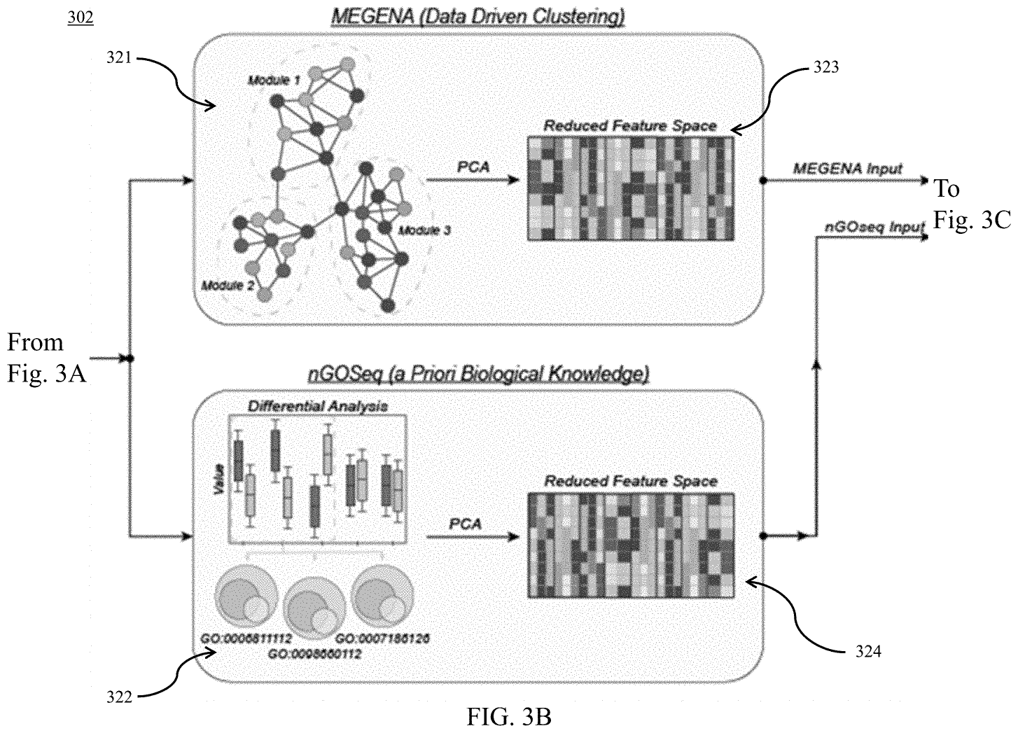

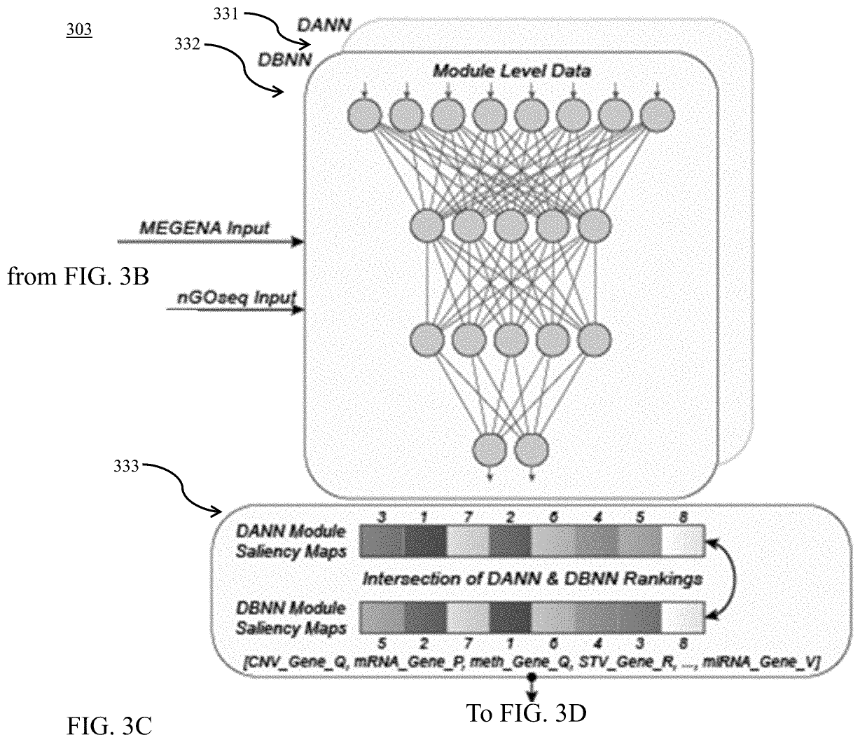

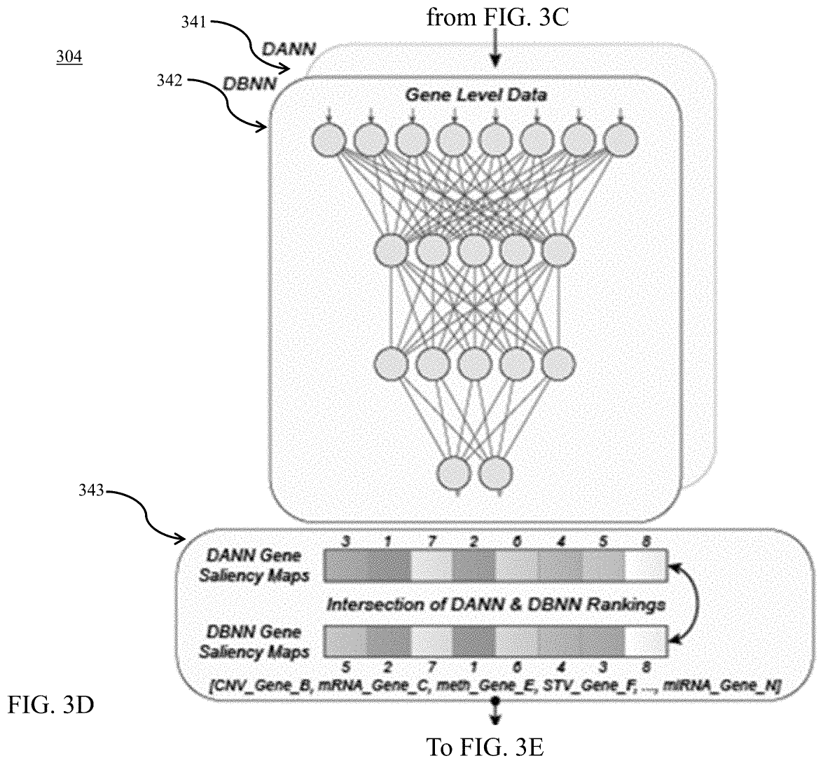

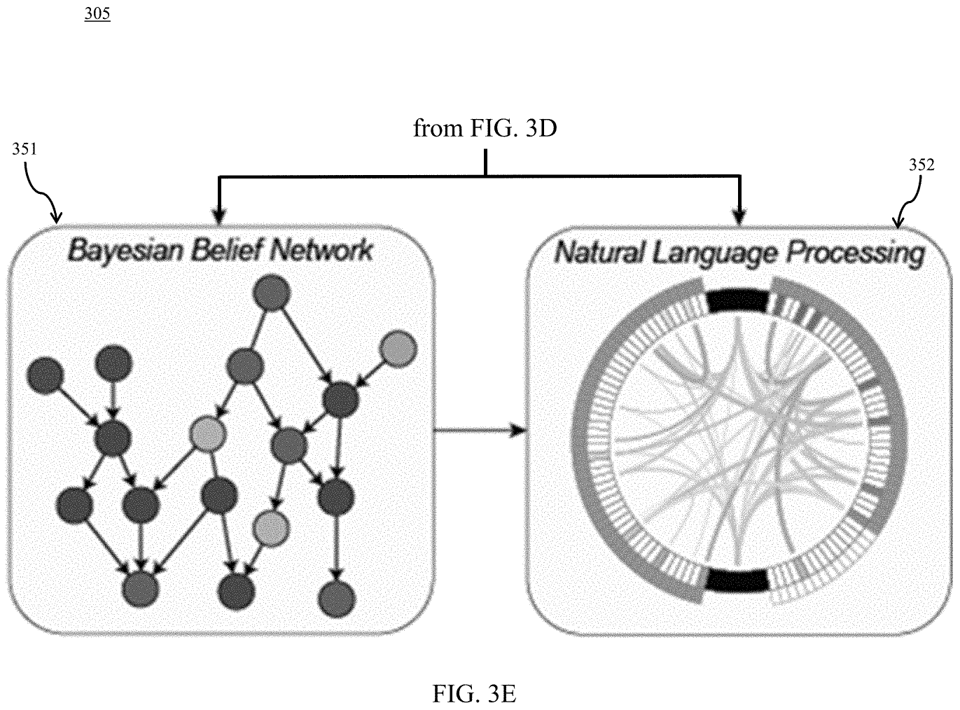

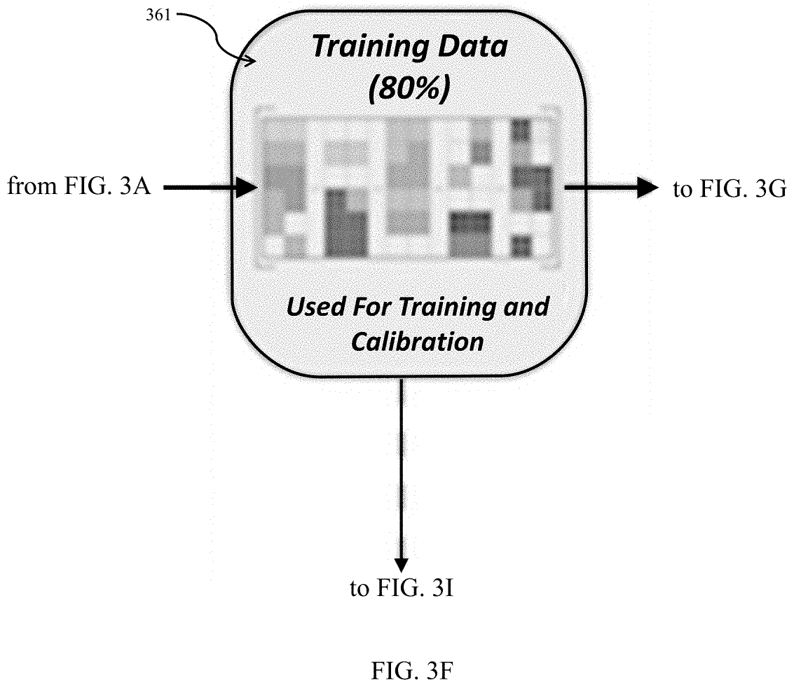

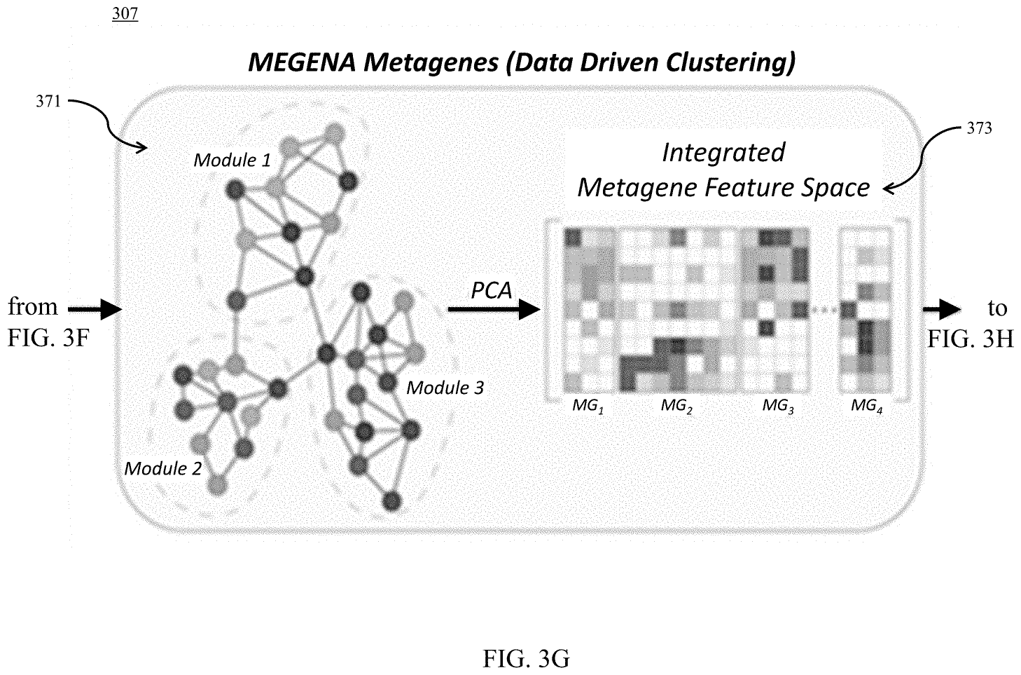

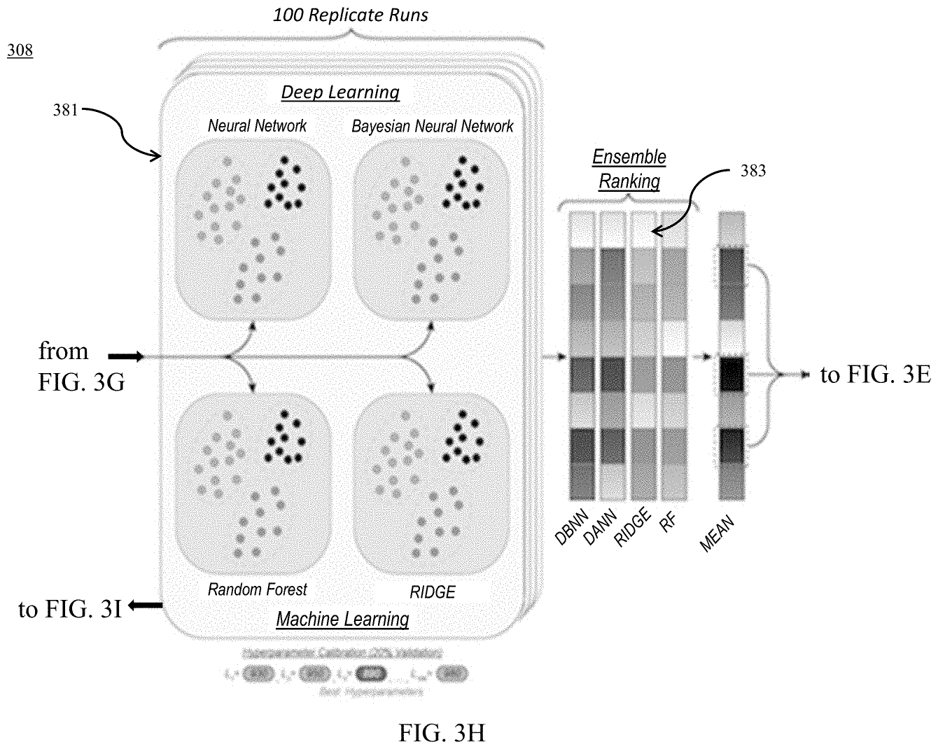

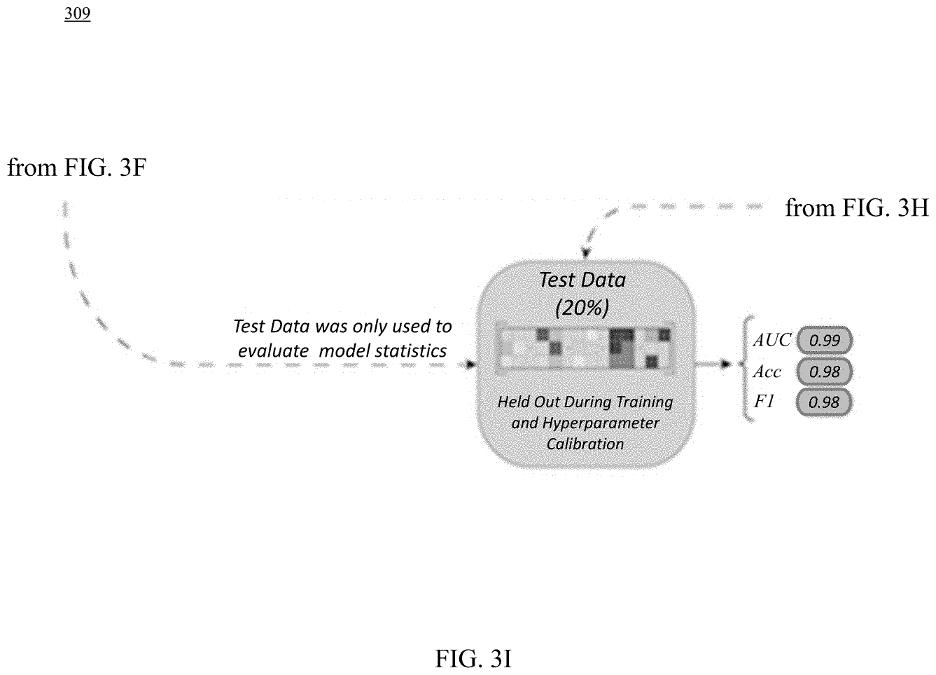

Referring now to FIGS. 3A-E, a schematic diagram of genomic analysis according to an exemplary embodiment of the present disclosure is provided. In this exemplary embodiment, the overall process steps of FIG. 1 are performed with particular data sets and algorithms by way of illustration and not limitation. In particular, as further described below, FIG. 3A corresponds to a data pre-processing and normalization step, FIG. 3B correspond to a feature learning and dimensionality reduction step; FIG. 3C corresponds to a module-level deep learning and ranking step, FIG. 3D corresponds to a gene-level deep learning and ranking step, and FIG. 3E corresponds to a causal dependency and biological context step.

In data pre-processing step 301, whole Exome Sequencing, RNA-Seq, miRNA-Seq, Methylation Array, and Genotyping Array data for 8272 samples, representing 22 cancer types were retrieved from either the Genome Data Commons (GDC) data portal (https://portal.gdc.cancer.gov/—Data Release 4.0) or cBioportal (http://www.cbioportal.org/). Whole exome sequencing data from VarScan2 and MuTect2 files annotated with Variant Effect Predictor (VEP) v84 and DeepCODE scores were used, subsequently filtered for quality and relevancy, mapped to genes, and all variants for a given gene added together. Raw read counts of mRNA from HT-Seq were normalized using trimmed mean of M-values (TMM), filtered (counts >1 per 10 reads in >10% of samples), and batch corrected using ComBat. Raw counts for known miRNAs were normalized in a similar fashion to mRNA. miRNA experimentally validated gene targets were downloaded from miRTarBase. GISTIC2 processed copy number variation (CNV) data were downloaded from cBioportal. Methylation beta values were filtered, converted to M values, and batch corrected using ComBat. Multiple probes were collapsed to a single gene by selecting the probe with the largest standard deviation.

All five input data types 311 . . . 315 were concatenated into a single data matrix and randomly split 80% (training data) and 20% (testing data) stratified by cancer and/or molecular subtype (survival analysis—also stratified by age, overall survival, and survival status). Each feature was standardized to zero mean and unit variance (z-score).

As noted above, in this exemplary embodiment, data for five experimental strategies—WXS, RNA-Seq, miRNA-Seq, Genotyping Array, Methylation Array-were retrieved from the GDC (Genome Data Commons) data portal (https://portal.gdc.cancer.gov/) and the cBioportal. Cancer types with fewer than 100 samples were excluded from analysis. In total, 8272 samples representing 22 cancer types were used for modeling as described further below.

For whole exome sequencing, GDC harmonized level 2 Variant Call Format (VCF) files from VarScan2 and MuTect2 annotated with the Variant Effect Predictor (VEP) v84 by the GDC somatic annotation workflow were used. VCF files were converted to Genomically Ordered Relational (GOR) database file format. DeepCODE scores were calculated for all variants. Variants with VCF ‘Filter’=‘Pass’ and VarScan2 p-value <=0.05 were kept. Variants with ‘Somatic’ status were also kept. Variants were further filtered on VEP annotation ‘impact’ and deepCODE score (described below) as follows: variants with a) ‘HIGH’ VEP impact, b) deepCODE score greater than 0.51 and ‘MODERATE’ VEP impact, or c) only ‘MODERATE’ VEP impact at the absence of deepCODE scores were kept. Call copies for each case, for each variant were retrieved from GOR tables after filtering. The variants were represented as a comma separated string. These were converted to a tab delimited table as one column for each case. The counts of call copies of all variants for a given gene were added together and presented as a single count value.

Variants for the breast cancer tumor vs. normal comparison were detected in aligned reads of GDC harmonized level 1 BAM files for tumor and normal samples using the Genome Analysis Toolkit (GATK) Haplotypecaller. Joint genotyping was performed on gVCF files produced by the HaplotypeCaller using GATK GenotypeGVCFs and hg38 as reference. VEP v85 annotations were obtained by mapping to chromosome position. Variant filtering and call-copy collapsing methods are described below.

For RNA-Seq, GDC harmonized level 3 mRNA quantification data was used. This data measures gene level expression as raw read counts from HT-Seq. Raw mapping counts were combined into a count matrix with genes as rows and samples as columns. Normalization was performed for all samples using the trimmed mean of M-values (TMM) method from the edgeR R package. Lowly expressed genes were filtered out by requiring read counts greater than 1 per million reads for more than 10% of samples. ComBat from the sva R package was used to assess possible batch effects in the normalized count data for all breast cancer samples using batch information extracted from TCGA barcodes (i.e., the plate number). There were no detectible batch effects as assessed by the Multi-Dimensional Scaling (MDS) either before or after batch correction.

For miRNA-Seq, GDC harmonized level 3 miRNA expression as raw counts for known miRNAs in the miRBase (http://www.mirbase.org/) reference was used. miRNA experimentally validated gene targets were downloaded from miRTarBase. The raw mapping counts were processed, normalized, and loaded into a count matrix similar to RNA-Seq data.

For the genotyping array, copy number variation (CNV) data from the cBioportal generated by the GISTIC2 algorithm were used. For the tumor comparison models, CNV data was compiled into a matrix with samples as rows and genes as columns. The copy-number value for each gene was an integer ranging from −2 to +2. All NA values were removed. For the breast cancer vs. normal comparison, GDC harmonized level-3 copy number data from Affymetrix SNP 6.0 arrays were used in the analysis. The segment means in the downloaded data were converted to linear copy numbers as 2*(2\hat{ }Segment_Mean), and mapped to gene symbols using ENSEMBLGRCh38 as reference. The CNV segments with less than 5 probes, and probe sets indicated to have frequent germline copy-number variation (using SNP6 array probe set file as reference) were discarded. A gene-level matrix was constructed across all samples for downstream analysis.

For methylation data, GDC harmonized level 3 methylation data with beta values from the Illumina Infinium Human Methylation273 (HM27) and HumanMethylation450 (HM450) arrays were used. In total, 24,889 probes, which map to 17,298 genes, were selected from these arrays based on the following criteria: probes were: i) shared between the two platforms, ii) mapped to genes or their promoters, and iii) not present in chromosome X, Y, and MT. In each subtype comparison, the sample beta values from methylation analysis were combined into a large matrix. Probes with NA values across all samples were removed. Remaining NA and zero beta values were replaced with the minimum beta value of non-zero beta values across all probes and all samples in each batch (defined by the TCGA plate barcode), as described in the REMPR package. Beta values of 1 were replaced with the maximum beta value less than 1 across all probes and all samples in each batch. All beta values were converted to M values using the formula M=log 2(beta/(1-beta)). ComBat from the sva R package was used to remove batch effects on plates within each cancer subtype. The samples were split randomly by 80:20 ratios into training and testing sets. Among multiple probes mapped to the same gene, the probe with the largest standard deviation across all training samples was selected to represent the gene level M value.

In data integration, the five molecular data types were combined into data matrices with samples represented in rows and genes presented in columns. For the binomial and multinomial comparisons, samples were randomly split into 80/20 training and testing datasets based on their cancer type (or molecular subtype). The clinical characteristics of the TCGA survival data for the pan-cancer survival analysis was equally distributed between the training and testing data sets. Therefore, stratification of training and testing sets was achieved on the following variables: i) age, ii) cancer type, iii) overall survival (in 2 month intervals), and iv) survival status. The data in the training matrix were converted to z-scores. Mean and variance from the training data were used to calculate z-scores for the test data.

In feature learning and dimensionality reduction step 302, two feature learning methods were used. It will be appreciated that various embodiments include a different selection of feature learning methods. In this exemplary embodiment, a data driven clustering approach, MEGENA 321, and an a priori biological knowledge based method, nGOseq 322, were applied.

MEGENA 321 uses a false-discovery controlled pairwise similarity metric to construct planar-filtered networks between features and subsequently calculates a directed acyclic graph of integrated cluster membership for all input data types.

For nGOseq 322, differential analysis was performed on each of the input data types (training data, two group—binomial class or survival status), filtered by false-discovery corrected p-value cutoff, and used in nested GOseq functional enrichment (nGOseq), a modified version of the nested Expression Analysis Systematic Explorer (nEASE) algorithm, to identify enriched nested GO terms.

The first principal component from principal component analysis (PCA) 323 . . . 324 was calculated for each gene-set/module, thus reducing the dimensionality of the learned feature space. The reduced feature space is aggregated into new data matrices for downstream modeling.

As noted above, in this exemplary embodiment, two feature engineering methods were used: a data-driven method (MEGENA) and an apriori knowledge based method (nGOseq) were applied to produce informative gene clusters. The first principal component of all members in each cluster was computed to serve as a summary statistic or “metagene” for the cluster to reduce the dimensionality of the engineered feature space.

Multiscale embedded gene co-expression network analysis (MEGENA) was used to carry out data-driven feature engineering for binomial and multinomial comparisons. MEGENA uses a quality controlled pairwise similarity metric (specifically false-discovery corrected Pearson correlation coefficients) to construct planar-filtered networks between features. Clusters in the network were identified with a multi-scaled approach, leading to a directed acyclic graph of cluster membership. The cluster membership was taken to create MEGENA modules. The MEGENA R package was used for the analysis. This package was not originally designed to deal with more than a single data type, therefore, the projective K means algorithm in the Weighted Gene Co-expression Network Analysis (WGNCA) R package was used to determine uncorrelated blocks of approximately 3000 features. This allowed for the use of significantly larger data matrices.

Differential analysis was performed for each of the five data types on the samples in the training set. The Wilcoxon Rank Sum test was used to find genes with differential copy number variation. The dmpFinder function from the minfi R package was used to find differentially methylated genes based on M values. The edgeR package was used to determine differentially expressed mRNAs and miRNAs. The Optimized Sequence Kernel Association Test (SKAT-O) was used to assess differential SNV patterns. The analysis was performed using default parameters, and the ‘optimal.adj’ method, after computing the SKAT_NULL_Model. Genes with differential patterns across the five data types were combined, and used in downstream functional enrichment analysis.

Functional enrichment analysis of differential genes was carried out with nGOseq as an a priori knowledge based feature engineering method for binomial comparisons. Initially, differential genes from the five data types were combined into a single gene set after removing gene redundancy. GOseq analysis was performed on the combined differential gene set to identify enriched gene ontology (GO) terms using all annotated genes as background. Nested GOseq (nGOseq), a modified version of the nested Expression Analysis Systematic Explorer (nEASE) algorithm, was then used to identify enriched nested GO terms driving the statistical enrichment of upper-level GOseq terms. Enriched non-redundant nGOseq gene sets were used as features for downstream modeling. Differentially expressed miRNA signals were incorporated into enriched nGOseq gene sets if their miRTarBase experimentally validated mRNA targets were also differentially expressed.

Principal component analysis (PCA) was applied to each nGOseq pathway and MEGENA module, which transformed the gene set data into a lower-dimensional coordinate system. Data matrices were then created for the downstream modeling with first principal component (PC1) values. The corresponding PC1 values served as “metagenes” for each nGOseq pathway and MEGENA module, further reducing dimensionality of the engineered feature space.

In module level deep learning and ranking step 303, Deep Artificial Neural Networks (DANNs) 331 and Deep Bayesian Neural Networks (DBNNs) 332 are trained and applied to the reduced feature space.

Lasagna and nolearn, and Theano python packages were used to construct Deep Artificial Neural Netowrks (DANNs). DANNs were initialized with an input layer, three hidden layers using Rectify non-linear activation functions (RELUs), and a softmax output layer. Weights were learned with stochastic gradient descent (with Nesterov momentum and dropout) using the categorical cross-entropy loss function.

Deep Bayesian Neural Networks (DBNNs) are an extension of DANNs that prescribe a prior distribution to the weights (W) of the neural network. The Edward and TensorFlow python packages were used to construct DBNNs with Gaussian priors, hidden layers used hyperbolic tangent activation functions (tan h), and a softmax output layer. Weights were learned with variational inference using the Kullback Leibler divergence (using mini-batches and ADAM for back-propagation) and sampled 500 times from the posterior distributions for final predictions.

The PyTorch python package was used to create Deep Hazard Neural Networks (DHNNs). DHNNs were formulated as deep versions of cox-proportional hazards model with hidden layers using tan h activation functions and a loss layer defined by the cox-proportional hazard log-likelihood function. Model hyper-parameters for DANN, DBNN, and DHNN models (e.g., learning rate, dropout rate, layer-size, number of layers, etc.) were optimized by cross-validated grid-search or random search (with early stopping). Models were evaluated using multiple metrics assessing fit quality.

For each of the classifiers, the relative importance of input variables with respect to output classes is computed. In this example, saliency mapping, a gradient-based sensitivity analysis that evaluates the relative importance of input variables with respect to output classes, is used. The result is a saliency map 333 indicating the feature importance for each of the DANNs, DBNNs, and DHNNs. For binomial comparisons, saliency maps were calculated at the gene-set/module level and the intersection of genes from each model type (DANN and DBNN) for each feature learning methodology (nGOseq and MEGNEA) were concatenated into new training and testing data matrices for downstream modeling at the gene-level.

In this exemplary embodiment, all deep artificial neural network (DANN) models were trained with deep neural networks in CUDA-enabled GPU computing platforms. The lasagna and nolearn python modules were used to construct these deep learning models with the Theano compiler. The deep neural networks were initialized with an input layer, three hidden layers using the Rectify non-linear activation function for artificial neurons as in Equation 1 and an output layer using the Softmax activation function as in Equation 2 where K is the total number of neurons in the layer.

$$\begin{matrix}ϕ ( x ) = max ( 0 , x ) & Equation 1 \\ {ϕ ( x )}_{j} = \frac{{e}^{{x}_{j}}}{{\sum }_{k = 1}^{K} {e}^{{x}_{k}}} & Equation 2\end{matrix}$$

Stochastic Gradient Descent (SGD) was performed for parameter updates with Nesterov momentum and the categorical cross-entropy loss function of Equation 3 where t is the target giving the correct class index per data point and p is the softmax output of the neural network with class probabilities.

$${L}_{i} = - {\sum }_{j} {t}_{i , j} log ( {p}_{i , j} ) \qquad Equation 3$$

A dropout technique was applied to prevent the deep neural networks from overfitting. Model parameters such as update learning rate, number of units, dropout rate and max epoch number were optimized by the cross-validated grid-search method over the parameter grid.

A genomic missense DNA variant DANN model (deepCODE) model was built for predicting the pathogenicity of human missense single-nucleotide variants (SNVs) across the genome. The model was trained on 59 genomic features extracted as a subset from a published annotation resource, the Combined Annotation Dependent Depletion data set (CADD: http://cadd.gs.washington.edu/home) from University of Washington. CADD includes a table with 115 columns of annotations derived from public domain resources on all possible human genetic variants in the genome. The data sources for the CADD table (version 1.3) includes ENSEMBL (v.75), variant-effect predictor (VEP, v.76), regulatory data from Encode, and missense prediction scores from Polyphen and SIFT. CADD C-score for functional prediction were not used for training the deepCODE DANN model.

The model was built with non-synonymous missense variants derived from the intersection of two data sources: 1) whole genome variants obtained from CADD, and 2) exonic coordinate regions for hg19 obtained from the UCSC genome browser. This classification scheme was trained and tested with a total of 2100 missense variants: 1050 missense variants from ClinVar (annotated by multiple labs as pathogenic), and 1050 common missense variants with allelic frequencies of 5 to 10%, randomly selected from the Exome Sequencing Project, ESP6500. We assumed that the vast majority of the latter are neutral/benign as they are common. The Clinvar “pathogenic” missense variants submitted by multiple labs served as “true values” for functional missense variants in the deepCODE models. Similarly, the 1050 ESP6500 variants served as “true values” for neutral missense variants. For model training purposes, 80% of the 2100 total variants were used.

DeepCODE is based on a non-linear deep neural network model built on 310 predictors derived from 59 of the 115 annotation columns from the CADD table. The model was tested by predicting pathogenicity for the remaining 20% of the total 2100 variants. The deepCODE model was evaluated with ROC curves and AUC metrics; the model had AUCs greater than 0.99 for both the training set and the testing set of missense variants. After the deepCODE model was trained and tested, GRC38 genomic position coordinates were obtained through use of the “liftover” function of Sequence Miner software.

DBNNs allow for uncertainty in neural networks by prescribing a prior distribution to the weights (W) of a feed-forward neural network and learning the posterior distribution via inference. In this example, the Edward library in conjunction with a TensorFlow backend was utilized to build the DBNNs. Gaussian priors were used for the weights of each layer (W), variational inference was carried out with the Kullback Leibler divergence (using mini-batches and ADAM for back-propagation), used hyperbolic tangent activation functions at each layer, and utilized a softmax layer for predicting class probabilities. The following hyper-parameters were optimized with a random search strategy: layer-size (128-2048), number of layers (2-3), and learning rate. The number of training epochs for each hyper-parameter tuning was determined by early stopping, implemented by monitoring both the accuracy and loss on a validation data set (10% of the training data). Final model predictions were made by sampling 500 times from the posterior distributions of the weights and taking the mean of the softmax prediction probabilities.

The DANN and DBNN models were evaluated using ROC and precision-recall (PR) curves (for binomial models), F1-scores, overall accuracy, and balanced accuracy metrics (for both binomial and multinomial models).

The Deep Hazard Neural Networks (DHNNs) were formulated as a deep version of the traditional cox-proportional hazards model. A traditional feed-forward neural network structure with a loss layer defined as the cox-proportional hazard log-likelihood function of Equation 4 was used where Xᵢ are the covariate vectors, Yᵢ denote the observed time and θⱼ=exp(Xⱼ·β).

$$l ( β ) = {\sum }_{i : {C}_{i} = 1} ( {X}_{i} \cdot β - log {\sum }_{j : {Y}_{j} \geq {Y}_{i}} {θ}_{j} ) \qquad Equation 4$$

This allows learning deep features in the neural network layers which are then the input to the traditional cox-proportional hazards model at the final layer. The model was implemented using the python library PyTorch with a custom-defined loss layer. The backpropagation using mini-batches and stochastic gradient descent with nesterov momentum (set to 0.9) was carried out and hyperbolic tangent activation functions at each layer was used. The following hyper-parameters were optimized with a random search strategy: layer-size (128-2048), number of layers (2-3), dropout fraction (0.1-0.8), and learning rate. The number of training epochs for each hyper-parameter run was determined by early stopping, implemented by monitoring both the accuracy and loss on a validation data set (10% of the training data). Model accuracy was assessed using both Harrell's c-index and a temporal AUC metric.

The supervised machine learning method, Least Absolute Shrinkage and Selection Operator (LASSO), was also used as complementary classification model for the deep neural network strategies described above. LASSO is a Li-penalized linear regression model. More specifically, the glmnet R package was used to solve the following optimization problem for Li-penalized regression as in Equation 5 where λ>0 equals the regularization parameter.

$$\hat{β} ( λ ) = {min}_{β} [ - log \{ L ( y ; β \} \} + λ {‖ β ‖}_{1} ] \qquad Equation 5$$

The constraint placed on the sum of the absolute values of regression parameters caused coefficients of uninformative features to shrink to zero. With this shrinkage process, a simpler model that selects only a few important features was produced. The cv.glmnet function from the glmnet R package was used to train the LASSO model, applying α=1 for Li-penalization. The λ was optimized via 10-fold cross-validation, and the value that gave a minimum mean cross-validated error was used for the model.

Saliency maps were derived from the trained deep neural networks described above to evaluate the relative importance of input variables based on computing the gradient of the network's prediction with respect to the input, holding the weights fixed through a single back-propagation pass throughout the multiple layers of the network.

The deep neural network consists of multiple layers of neurons, activated as in Equation 6 with zᵢⱼ=αᵢ^{(l)}wᵢⱼ^{(l,l+1)}, where αⱼ^{(l+1)} is the activation of a neuron j in the layer l+1, and zᵢⱼ is the contribution of neuron i at the previous layer l to the activation of the neuron j at layer l+1.

$${a}_{j}^{( l + 1 )} = f ( {\sum }_{i} {z}_{ij} + {b}_{j}^{( l + 1 )} ) \qquad Equation 6$$

The function ƒ is the activation function at layer l+1, wᵢⱼ^{(l,l+1)} is the weights from the layer l to the layer l+1 and bⱼ^{(l+1)} is the bias term.

The back-propagation chain rule from one layer to another layer for computing partial derivatives as in Equation 7 where x^{(l)} and x^{(l+1)} are the neuron activities at two conservative layers (l+1, l).

$$\frac{\partial f}{\partial {x}^{( l )}} = \frac{\partial {x}^{( l + 1 )}}{\partial {x}^{( l )}} \frac{\partial f}{\partial {x}^{( l + 1 )}} \qquad Equation 7$$

In gene level deep learning and ranking step 304, this analysis was repeated using models (DANN 341 and DBNN 342) trained at gene level. The top intersecting genes (e.g., 100) were extracted as final gene lists. For the multinomial comparison, the intersection (DANN and DBNN) of the top informative MEGENA modules was taken for each cancer type. At the gene-level, the top (e.g., 100) most informative genes were calculated for each cancer, and the final 200 genes were obtained by sorting the union set by the number of occurrences (filtered by ≥4 cancers).

Significant hazard ratios (false discovery rate≤0.05) for DHNN models were calculated using univariate cox-proportional hazard models for each cancer and formulated into an undirected graph structure. Model predictions for all samples (from each DHNN) were stratified into 3 risk quantiles (low, moderate, and high) and p-values were calculated via log-rank tests for each pairwise comparison.

Based on the ranks from the saliency mappings of the DANN nGOseq and DBNN nGOseq models (training data only), genes from the top 50% of the most informative nGOseq terms from each model were extracted. The intersection of the genes from each model was then calculated and intersecting genes were concatenated into new training and testing data matrix for further modeling at the gene-level.

Similarly, rankings from the saliency mappings of the DANN MEGENA and DBNN MEGENA models (training data only), genes from the intersection of the top 10% of informative modules from each model were extracted. This cut-off is significantly more restrictive than that used for the nGOSeq models (described above), since the sizes of MEGENA modules are larger than nGOseq pathways. The individual genes from each of the intersecting modules were then concatenated into new training and testing data matrix for further modeling at the gene-level.

Saliency maps were calculated for both DANN and DBNN models at the gene level and the top 100 intersecting genes were extracted for final gene lists. Both of the binomial classes contributed to the ranking—the top 50 or more from each class were used.

The ranking procedure for the binomial comparisons was modified due to the increase in the number of classes (from 2 to 22) in the multinomial models. Based on the ranking from the saliency mappings of the DANN MEGENA and DBNN MEGENA models (training data only) the intersection of the top informative modules for each class (cancer type) from each model was taken. The individual genes from these modules were then concatenated into new training and testing data matrix for further modeling at the gene-level.

Saliency maps were calculated for both DANN and DBNN models at the gene level and the top 100 intersecting genes were extracted for each of the 22 cancer types. The union of these genes was then calculated along with the number of occurrences in the union set. The final ranking was obtained by sorting the union set by the number of occurrences and subsequently filtered the list by removing genes with an occurrence in less than 15% of tumor types.

In causal dependency and biological context determination step 305, conditional dependence is assessed between the most informative genes from the prior step. In this embodiment, Bayesian belief networks (BNNs) 351 were used to assess conditional dependence between the top 100 most informative genes for each feature learning methodology. BNNs were learned with the bnlearn R package using a heuristic search strategy and the Bayesian information criterion score. Consensus networks were generated from 100 random network seeds and statistical significance of edges was calculated via 10,000 random permutations of the data set (edges with a false discovery rate ≥0.05 were removed).

Natural language processing 352 is performed to evaluate existing literature. Chilibot Natural Language Processing was used to identify associations among the top 100 most informative genes and specific cancer types for each model comparison (binomial, multinomial, survival). Chilibot uses natural language processing to search MEDLINE/PubMed abstracts for relationships between genes of interest and query terms (MeSH vocabulary terms). Gene association with drug targets was determined by querying both DrugBank (https://www.drugbank.ca/) and Pharmacodia (http://en.pharmacodia.com/) and filtering based on clinical trials in any indication.

Bayesian Belief Networks (BNN) were used to assess conditional dependence and to explore the probabilistic relationships among the most informative genes of each deep neural network model. A BNN is a graphic model where nodes represent random variables and the directed edges represent conditional dependence between the nodes. The probability distribution of the variables in a BNN must satisfy the Markov property, that is, each variable is conditionally independent of all other variables except its parents and descendants, given its parent variable. Thus a DAG (directed acyclic graph) G=(V, E), where V is the node set and E is the edge set, encodes factorizations by a set of local probability distributions.

Bayesian network structures were learned with the bnlearn R package, from which the derivations and equation below are cited and summarized. The score-based, Hill-climbing algorithm was used for heuristic search on the space of the DAGs. During the hill-climbing process, assessment of each candidate BNN, which describes the data set D, was measured with a Bayesian information criterion score (BIC score) as in Equation 8, where X₁, . . . , Xᵥ is the node set, d is the number of free parameters of the multivariate Gaussian distribution, and n is the sample size of data set D.

$$BIC = log L ( {X}_{1 , … ,} {X}_{v} ) - \frac{d}{2} log n \qquad Equation 8$$

The penalty term was used to prevent overly complicated structures and overfitting. The algorithm returns a structure that maximizes the BIC score. BNN consensus networks were generated for each binomial and Pan-Cancer survival gene list with 100 random network seeds. To assess statistical significance of node edges within each imposed consensus network, 100 k random permutations were performed. Node edges with a false discovery rate of 1% or greater were removed from the final network.

Chilibot Natural Language Processing was used to identify associations among the top 100 statistically informative genes and specific cancer types for each binomial and multinomial comparison described above. Chilibot is a web-based application that uses natural language processing to search MEDLINE/PubMed abstracts for relationships between genes of interest and query terms. Each gene was compared with every other gene in the query group and assigned a relationship (stimulatory, inhibitory, neutral, parallel and abstract co-occurrence) based on data in the abstract. Cancer, cancer type, and patient survival U.S. National Library of Medicine Medical Subject Headings (MeSH) vocabulary terms were used as synonyms to refine each NLP search.

FIG. 3F-I illustrate an alternative ensemble computational method. In particular, in such embodiments, training data 361 obtained from preprocessing 301 step of FIG. 3A are provided to feature learning and dimensionality reduction step 307 of FIG. 3G and to model evaluation step 309 of FIG. 3. FIG. 3H corresponds to an ensemble module-level deep learning (ML/DL) and feature ranking step, the results of which are provided to the causal dependency and biological context step of FIG. 3E. In the example pictured, 80% of the data obtained from step

In the example pictured, 80% of the data obtained from preprocessing step 301 is used for training in step 307, while 20% is reserved for step 309. However, it will be appreciated that this ratio is merely exemplary.

A data driven clustering approach, MEGENA 371, is applied as described further above. Principal component analysis (PCA) is applied for each gene-set/module, thus reducing the dimensionality of the learned feature space. The reduced feature space 373 is aggregated into new data matrices for downstream modeling.

A plurality of deep learning and/or machine learning methods 381 are applied at step 308. For example, a neural network, a Bayesian neural network, a random forest, and/or a ridge regression model are applied. The results are provided back to step 309 for evaluation of each model applied. Ensemble ranking is applied to output saliency maps 383 for each model. In some embodiments, a composite salience map, for example based on a weighted mean of the ensemble. The result is provided to step 304, described further above.

The term “biological sample” includes, but not limited to, whole blood, plasma, serum, saliva, urine, stool (e.g., feces), tears, any other bodily fluid, a tissue sample (e.g., biopsy) such as a surgical resection tissue, cells, tissues, or organs. In certain instances, the method of the present invention further comprises obtaining the sample from the subject prior to detecting or determining the presence or level of at least one therapeutic or drug target in the sample.

The term “diagnosing cancer” includes the use of the methods, systems, algorithms, programs, and codes of the present invention to determine the presence or absence of a cancer or subtype thereof in subject. The term also includes methods, systems, algorithms, programs, and codes for assessing the level of disease activity in an individual.

The term “pan-cancer” includes, but not limited to, the cancers listed in Table A.

TABLE A
The Cancer Genome Atlas (TCGA) cancer samples

| count | TCGA_project | TCGA_disease_type |
|---|---|---|
| 401 | BLCA | Bladder Urothelial Carcinoma |
| 1006 | BRCA | Breast Invasive Carcinoma |
| 292 | CESC | Cervical Squamous Cell Carcinoma |
| | | and Endocervical Adenocarcinoma |
| 551 | COAD/READ | Colon Adenocarcinoma/Rectum Adenocarcinoma |
| 160 | ESCA | Esophageal Carcinoma |
| 480 | HNSC | Head and Neck Squamous Cell Carcinoma |
| 327 | KIRC | Kidney Renal Clear Cell Carcinoma |
| 284 | KIRP | Kidney Renal Papillary Cell Carcinoma |
| 499 | LGG | Brain Lower Grade Glioma |
| 358 | LIHC | Liver Hepatocellular Carcinoma |
| 500 | LUAD | Lung Adenocarcinoma |
| 462 | LUSC | Lung Squamous Cell Carcinoma |
| 265 | OV | Ovarian Serous Cystadenocarcinoma |
| 172 | PAAD | Pancreatic Adenocarcinoma |
| 159 | PCPG | Pheochromocytoma and Paraganglioma |
| 483 | PRAD | Prostate Adenocarcinoma |
| 249 | SARC | Sarcoma |
| 369 | STAD | Stomach Adenocarcinoma |
| 133 | TGCT | Testicular Germ Cell Tumors |
| 481 | THCA | Thyroid Carcinoma |
| 118 | THYM | Thymoma |
| 523 | UCEC | Uterine Corpus Endometrial Carcinoma |
| 740 | ER_Positive | |
| 219 | ER_Negative | |
| 199 | Luminal_A | |
| 112 | Luminal_B | |

For example, whole Exome Sequencing, RNA-Seq, miRNA-Seq, Methylation Array, and Genotyping Array data for 8272 samples, representing 22 cancer types (FIG. 1 and Table A), were retrieved from either the Genome Data Commons (GDC) data portal (https./portal.gdc.cancer.gov/—data release 4.0) or cBioportal (http://www.cbioportal.org/)69. Whole exome sequencing data from VarScan2 (Koboldt, D. C. et al. Genome Res 22, 568-576, (2012)) and MuTect2(Cibulskis, K. et al. Nat Biotechnol 31, 213-219 (2013)) files annotated with Variant Effect Predictor (VEP)(McLaren, W. et al. Genome Biol 17, 122 (2016)) v84 and DeepCODE scores were used, subsequently filtered for quality and relevancy, mapped to genes, and all variants for a given gene added together. Raw read counts of mRNA from HT-Seq(Anders, S. et al. Bioinformatics 31, 166-169 (2015) were normalized using trimmed mean of M-values (TMM) (Robinson, M. D. et al. Genome Biol 11, R25, (2010); Robinson, M. D. et al. Bioinformatics 26, 139-140, (2010)), filtered (counts >1 per 106 reads in >10% of samples), and batch corrected using ComBat (Johnson, W. E. et al. Biostatistics 8, 118-127 (2007); Johnson, W. E. et al. Biostatistics 8, 118-127 (2007)). Raw counts for known miRNAs were normalized in a similar fashion to mRNA. miRNA experimentally validated gene targets were downloaded from miRTarBase (Chou, C. H. et al. Nucleic Acids Res 44, D239-247, (2016)). GISTIC2 (Beroukhim, R. et al. Proc Natl Acad Sci USA 104, 20007-20012, (2007)) processed copy number variation (CNV) data were downloaded from cBioportal (Cerami, E. et al. Cancer Discov 2, 401-404 (2012); Gao, J. et al. Sci Signal 6, pl1, (2013)). Methylation beta values were filtered, converted to M values, and batch corrected using ComBat. Multiple probes were collapsed to a single gene by selecting the probe with the largest standard deviation. All 5 data types were concatenated into a single data matrix and randomly split 80% (training data) and 20% (testing data) stratified by cancer and/or molecular subtype (survival analysis—also stratified by age, overall survival, and survival status). Each feature was standardized to zero mean and unit variance (z-score).

Additional cancers may include, but not limited to, cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hypopharyngeal cancer, intraocular melanoma, islet cell tumors (endocrine pancreas), Kaposi sarcoma, Langerhans cell histiocytosis, laryngeal cancer, leukemia, lung cancer, non-small cell lung cancer, small cell lung cancer, Hodgkin lymphoma, lymphoma, medulloblastoma, medulloepithelioma, melanoma, mesothelioma, mouth cancer, multiple myeloma, nasopharyngeal cancer, neuroblastoma, non-Hodgkin lymphoma, oral cancer, oropharyngeal cancer, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm, pleuropulmonary blastoma, primary central nervous system lymphoma, prostate cancer, rectal cancer, renal cell (kidney) cancer, rhabdomyosarcoma, salivary gland cancer, sarcoma, Ewing sarcoma family of tumors, sarcoma, Sezary syndrome, skin cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, or Wilms tumor.

The pan-cancer model-derived driver therapeutic or drug targets or genes generated according to the methods, systems, algorithms, programs, and codes described above are set forth in Appendix K (full listing) and Tables L (top 51 genes) and M (top 200 genes).

TABLE L
Top 50 genes from pan-cancer from Table A (22 cancer types) MEGENA (see full listings in Appendix K and L)

| | | | | Number_Of- | |
|---|---|---|---|---|---|
| Full_Name | Data_Type | HUGO_GENE | GO_Annotated | GO_Annotations | Cancers_In_Rank |
| meth_KCNQ1 | meth | KCNQ1 | YES | 69 | BRCA, CRAD, ESCA, KIRC, |
| | | | | | KIRP, OV, PRAD, TGCT, UCEC |
| meth_PIK3CA | meth | PIK3CA | YES | 67 | BRCA, HNSC, LGG, LUSC, |
| | | | | | OV, PCPG, SARC, THCA, THYM |
| meth_IL20 | meth | IL20 | YES | 11 | BLCA, BRCA, CESC, CRAD, |
| | | | | | HNSC, KIRC, OV, STAD, UCEC |
| meth_STON2 | meth | STON2 | YES | 17 | BLCA, BRCA, CRAD, HNSC, |
| | | | | | LUAD, LUSC, PRAD, STAD |
| meth_RP11.540D14.8 | meth | RP11.540D14.8 | NO | 0 | BLCA, BRCA, CESC, CRAD, |
| | | | | | KIRC, KIRP, LGG, UCEC |
| meth_AGT | meth | AGT | YES | 111 | KIRP, LIHC, LUSC, PAAD, |
| | | | | | SARC, STAD, TGCT, THCA |
| mRNA_HAS2-AS1 | mRNA | HAS2-AS1 | NO | 0 | BLCA, CRAD, KIRC, LGG, |
| | | | | | OV, SARC, TGCT, UCEC |
| mRNA_XPR1 | mRNA | XPR1 | YES | 17 | CESC, ESCA, LIHC, LUAD, |
| | | | | | PRAD, THCA, UCEC |
| mRNA_NFIX | mRNA | NFIX | YES | 15 | BLCA, BRCA, KIRP, LUSC, |
| | | | | | PCPG, PRAD, SARC |
| meth_MGMT | meth | MGMT | YES | 31 | BRCA, CESC, LIHC, PCPG, |
| | | | | | PRAD, THCA, UCEC |
| meth_C16orf87 | meth | C16orf87 | YES | 1 | CRAD, ESCA, LIHC, PAAD, |
| | | | | | SARC, STAD, UCEC |
| meth_NPL | meth | NPL | YES | 10 | BLCA, BRCA, CRAD, KIRP, |
| | | | | | LGG, PAAD, PRAD |
| meth_CRAT | meth | CRAT | YES | 15 | CRAD, HNSC, LUAD, LUSC, |
| | | | | | OV, PAAD, THYM |
| mRNA_HOXD-AS2 | mRNA | HOXD-AS2 | NO | 0 | CESC, CRAD, HNSC, KIRP, |
| | | | | | LGG, LIHC, LUAD |
| meth_TLK1 | meth | TLK1 | YES | 16 | BLCA, KIRC, LUAD, PCPG, |
| | | | | | PRAD, THCA, THYM |
| meth_ALDH18A1 | meth | ALDH18A1 | YES | 26 | KIRC, LUAD, LUSC, PAAD, |
| | | | | | THCA, THYM, UCEC |
| mRNA_CACHD1 | mRNA | CACHD1 | YES | 2 | CRAD, KIRP, LUSC, OV, |
| | | | | | PAAD, PCPG, THCA |
| mRNA_PHACTR4 | mRNA | PHACTR4 | YES | 22 | CESC, CRAD, LIHC, OV, |
| | | | | | STAD, THYM, UCEC |
| meth_FLRT1 | meth | FLRT1 | YES | 32 | BRCA, KIRP, LUSC, PAAD, |
| | | | | | PCPG, UCEC |
| mRNA_HNRNPUL2-BSCL2 | mRNA | HNRNPUL2-BSCL2 | YES | 5 | ESCA, HNSC, LGG, OV, |
| | | | | | STAD, THCA |
| meth_ACSF2 | meth | ACSF2 | YES | 12 | BRCA, CRAD, HNSC, |
| | | | | | LGG, LIHC, SARC |
| meth_ARG1 | meth | ARG1 | YES | 53 | BLCA, CRAD, KIRP, LIHC, |
| | | | | | PRAD, THCA |
| meth_SYCP2 | meth | SYCP2 | YES | 16 | BRCA, CESC, CRAD, KIRP, |
| | | | | | LUAD, PCPG |
| meth_LIPC | meth | LIPC | YES | 28 | BLCA, BRCA, KIRC, KIRP, |
| | | | | | LGG, PRAD |
| mRNA_RAET1E-AS1 | mRNA | RAET1E-AS1 | NO | 0 | BLCA, CESC, CRAD, ESCA, |
| | | | | | SARC, STAD |
| mRNA_MKLN1-AS | mRNA | MKLN1-AS | NO | 0 | BLCA, KIRC, KIRP, LUSC, |
| | | | | | PAAD, PCPG |
| meth_SLC35F6 | meth | SLC35F6 | YES | 17 | BLCA, BRCA, TGCT, THCA, |
| | | | | | THYM, UCEC |
| meth_ALDH1B1 | meth | ALDH1B1 | YES | 12 | BLCA, LUAD, LUSC, OV, |
| | | | | | PAAD, STAD |
| mRNA_PAG1 | mRNA | PAG1 | YES | 20 | BLCA, CRAD, HNSC, KIRP, |
| | | | | | PRAD, THYM |
| mRNA_EPB41L2 | mRNA | EPB41L2 | YES | 31 | CRAD, HNSC, LUSC, PCPG, |
| | | | | | SARC, TGCT |
| mRNA_EIF4BP3 | mRNA | EIF4BP3 | NO | 0 | CESC, ESCA, HNSC, OV, |
| | | | | | STAD, THCA |
| mRNA_ZFYVE27 | mRNA | ZFYVE27 | YES | 23 | BRCA, KIRC, KIRP, LGG, |
| | | | | | PAAD, PCPG |
| meth_FAM131A | meth | FAM131A | YES | 1 | BRCA, HNSC, KIRC, LUAD, |
| | | | | | LUSC, STAD |
| mRNA_RP11-398K22.12 | mRNA | RP11-398K22.12 | NO | 0 | ESCA, HNSC, LGG, LUSC, |
| | | | | | THCA, THYM |
| meth_CIB3 | meth | CIB3 | YES | 4 | BRCA, CRAD, ESCA, PAAD, |
| | | | | | STAD, THYM |
| meth_C2CD2 | meth | C2CD2 | YES | 4 | BLCA, BRCA, CESC, LGG, |
| | | | | | LUSC, PRAD |
| mRNA_MKRN3 | mRNA | MKRN3 | YES | 6 | CRAD, HNSC, KIRP, LGG, |
| | | | | | STAD, THCA |
| meth_RIOK3 | meth | RIOK3 | YES | 28 | ESCA, PCPG, SARC, STAD, |
| | | | | | TGCT, UCEC |
| mRNA_AC004987.9 | mRNA | AC004987.9 | NO | 0 | BLCA, CESC, OV, PAAD, |
| | | | | | STAD, UCEC |
| meth_RABL6 | meth | RABL6 | YES | 8 | CESC, CRAD, HNSC, KIRP, |
| | | | | | LIHC, OV |
| mRNA_KCNS3 | mRNA | KCNS3 | YES | 21 | BLCA, HNSC, LUAD, LUSC, |
| | | | | | PRAD, UCEC |
| mRNA_MARCKS | mRNA | MARCKS | YES | 20 | BRCA, LIHC, PAAD, SARC, |
| | | | | | THCA, UCEC |
| meth_FABP7 | meth | FABP7 | YES | 20 | CRAD, HNSC, KIRC, LGG, |
| | | | | | LIHC, OV |
| meth_LDHD | meth | LDHD | YES | 10 | KIRC, KIRP, LGG, LIHC, |
| | | | | | LUAD, UCEC |
| meth_SIDT1 | meth | SIDT1 | YES | 4 | BLCA, BRCA, HNSC, |
| | | | | | LIHC, PRAD, THYM |
| meth_SCGB3A2 | meth | SCGB3A2 | YES | 3 | ESCA, HNSC, KIRC, LGG, |
| | | | | | PRAD, THCA |
| mRNA_RPS6KA6 | mRNA | RPS6KA6 | YES | 24 | CESC, CRAD, LUAD, |
| | | | | | PRAD, TGCT, THYM |
| mRNA_POT1-AS1 | mRNA | POT1-AS1 | NO | 0 | CESC, CRAD, LUSC, |
| | | | | | PRAD, SARC, THYM |
| meth_NDUFAF4 | meth | NDUFAF4 | YES | 8 | CESC, CRAD, LUAD, |
| | | | | | LUSC, THCA, UCEC |

TABLE M
Top 200 genes from pan-cancer from Table A (22 cancer types) MEGENA (no need to include Appendix L as same as Table M)

| | | | | Number_Of- | |
|---|---|---|---|---|---|
| Full_Name | Data_Type | HUGO_GENE | GO_Annotated | GO_Annotations | Cancers_In_Rank |
| meth_KCNQ1 | meth | KCNQ1 | YES | 69 | BRCA, CRAD, ESCA, KIRC, |
| | | | | | KIRP, OV, PRAD, TGCT, UCEC |
| meth_PIK3CA | meth | PIK3CA | YES | 67 | BRCA, HNSC, LGG, LUSC, |
| | | | | | OV, PCPG, SARC, THCA, THYM |
| meth_IL20 | meth | IL20 | YES | 11 | BLCA, BRCA, CESC, CRAD, |
| | | | | | HNSC, KIRC, OV, STAD, UCEC |
| meth_STON2 | meth | STON2 | YES | 17 | BLCA,BRCA, CRAD, HNSC, |
| | | | | | LUAD, LUSC, PRAD, STAD |
| meth_RP11.540D14.8 | meth | RP11.540D14.8 | NO | 0 | BLCA, BRCA, CESC, CRAD, |
| | | | | | KIRC, KIRP, LGG, UCEC |
| meth_AGT | meth | AGT | YES | 111 | KIRP, LIHC, LUSC, PAAD, |
| | | | | | SARC, STAD, TGCT, THCA |
| mRNA_HAS2-AS1 | mRNA | HAS2-AS1 | NO | 0 | BLCA, CRAD, KIRC, LGG, |
| | | | | | OV, SARC, TGCT, UCEC |
| mRNA_XPR1 | mRNA | XPR1 | YES | 17 | CESC, ESCA, LIHC, LUAD, |
| | | | | | PRAD, THCA, UCEC |
| mRNA_NFIX | mRNA | NFIX | YES | 15 | BLCA, BRCA, KIRP, LUSC, |
| | | | | | PCPG, PRAD, SARC |
| meth_MGMT | meth | MGMT | YES | 31 | BRCA, CESC, LIHC, PCPG, |
| | | | | | PRAD, THCA, UCEC |
| meth_C16orf87 | meth | C16orf87 | YES | 1 | CRAD, ESCA, LIHC, PAAD, |
| | | | | | SARC, STAD, UCEC |
| meth_NPL | meth | NPL | YES | 10 | BLCA, BRCA, CRAD, KIRP, |
| | | | | | LGG, PAAD, PRAD |
| meth_CRAT | meth | CRAT | YES | 15 | CRAD, HNSC, LUAD, LUSC, |
| | | | | | OV, PAAD, THYM |
| mRNA_HOXD-AS2 | mRNA | HOXD-AS2 | NO | 0 | CESC, CRAD, HNSC, KIRP, |
| | | | | | LGG, LIHC, LUAD |
| meth_TLK1 | meth | TLK1 | YES | 16 | BLCA, KIRC, LUAD, PCPG, |
| | | | | | PRAD, THCA, THYM |
| meth_ALDH18A1 | meth | ALDH18A1 | YES | 26 | KIRC, LUAD, LUSC, PAAD, |
| | | | | | THCA, THYM, UCEC |
| mRNA_CACHD1 | mRNA | CACHD1 | YES | 2 | CRAD, KIRP, LUSC, OV, |
| | | | | | PAAD, PCPG, THCA |
| mRNA_PHACTR4 | mRNA | PHACTR4 | YES | 22 | CESC, CRAD, LIHC, OV, |
| | | | | | STAD, THYM, UCEC |
| meth_FLRT1 | meth | FLRT1 | YES | 32 | BRCA, KIRP, LUSC, PAAD, |
| | | | | | PCPG, UCEC |
| mRNA_HNRNPUL2-BSCL2 | mRNA | HNRNPUL2-BSCL2 | YES | 5 | ESCA, HNSC, LGG, OV, |
| | | | | | STAD, THCA |
| meth_ACSF2 | meth | ACSF2 | YES | 12 | BRCA, CRAD, HNSC, |
| | | | | | LGG, LIHC, SARC |
| meth_ARG1 | meth | ARG1 | YES | 53 | BLCA, CRAD, KIRP, LIHC, |
| | | | | | PRAD, THCA |
| meth_SYCP2 | meth | SYCP2 | YES | 16 | BRCA, CESC, CRAD, KIRP, |
| | | | | | LUAD, PCPG |
| meth_LIPC | meth | LIPC | YES | 28 | BLCA, BRCA, KIRC, KIRP, |
| | | | | | LGG, PRAD |
| mRNA_RAET1E-AS1 | mRNA | RAET1E-AS1 | NO | 0 | BLCA, CESC, CRAD, ESCA, |
| | | | | | SARC, STAD |
| mRNA_MKLN1-AS | mRNA | MKLN1-AS | NO | 0 | BLCA, KIRC, KIRP, LUSC, |
| | | | | | PAAD, PCPG |
| meth_SLC35F6 | meth | SLC35F6 | YES | 17 | BLCA, BRCA, TGCT, THCA, |
| | | | | | THYM, UCEC |
| meth_ALDH1B1 | meth | ALDH1B1 | YES | 12 | BLCA, LUAD, LUSC, OV, |
| | | | | | PAAD, STAD |
| mRNA_PAG1 | mRNA | PAG1 | YES | 20 | BLCA, CRAD, HNSC, KIRP, |
| | | | | | PRAD, THYM |
| mRNA_EPB41L2 | mRNA | EPB41L2 | YES | 31 | CRAD, HNSC, LUSC, PCPG, |
| | | | | | SARC, TGCT |
| mRNA_EIF4BP3 | mRNA | EIF4BP3 | NO | 0 | CESC, ESCA, HNSC, OV, |
| | | | | | STAD, THCA |
| mRNA_ZFYVE27 | mRNA | ZFYVE27 | YES | 23 | BRCA, KIRC, KIRP, LGG, |
| | | | | | PAAD, PCPG |
| meth_FAM131A | meth | FAM131A | YES | 1 | BRCA, HNSC, KIRC,LUAD, |
| | | | | | LUSC,STAD |
| mRNA_RP11-398K22.12 | mRNA | RP11-398K22.12 | NO | 0 | ESCA, HNSC, LGG, LUSC, |
| | | | | | THCA, THYM |
| meth_CIB3 | meth | CIB3 | YES | 4 | BRCA, CRAD, ESCA, PAAD, |
| | | | | | STAD, THYM |
| meth_C2CD2 | meth | C2CD2 | YES | 4 | BLCA, BRCA, CESC, LGG, |
| | | | | | LUSC, PRAD |
| mRNA_MKRN3 | mRNA | MKRN3 | YES | 6 | CRAD, HNSC, KIRP, LGG, |
| | | | | | STAD, THCA |
| meth_RIOK3 | meth | RIOK3 | YES | 28 | ESCA, PCPG, SARC, STAD, |
| | | | | | TGCT, UCEC |
| mRNA_AC004987.9 | mRNA | AC004987.9 | NO | 0 | BLCA, CESC, OV, PAAD, |
| | | | | | STAD, UCEC |
| meth_RABL6 | meth | RABL6 | YES | 8 | CESC, CRAD, HNSC, KIRP, |
| | | | | | LIHC, OV |
| mRNA_KCNS3 | mRNA | KCNS3 | YES | 21 | BLCA, HNSC, LUAD, LUSC, |
| | | | | | PRAD, UCEC |
| mRNA_MARCKS | mRNA | MARCKS | YES | 20 | BRCA, LIHC, PAAD, SARC, |
| | | | | | THCA, UCEC |
| meth_FABP7 | meth | FABP7 | YES | 20 | CRAD, hnsc, KIRC, |
| | | | | | LGG, LIHC, OV |
| meth_LDHD | meth | LDHD | YES | 10 | KIRC, KIRP, LGG, LIHC, |
| | | | | | LUAD, UCEC |
| meth_SIDT1 | meth | SIDT1 | YES | 4 | BLCA, BRCA, HNSC, |
| | | | | | LIHC, PRAD, THYM |
| meth_SCGB3A2 | meth | SCGB3A2 | YES | 3 | ESCA, HNSC, KIRC, LGG, |
| | | | | | PRAD, THCA |
| mRNA_RPS6KA6 | mRNA | RPS6KA6 | YES | 24 | CESC, CRAD, LUAD, |
| | | | | | PRAD, TGCT, THYM |
| mRNA_POT1-AS1 | mRNA | POT1-AS1 | NO | 0 | CESC, CRAD, LUSC, |
| | | | | | PRAD, SARC, THYM |
| meth_NDUFAF4 | meth | NDUFAF4 | YES | 8 | CESC, CRAD, LUAD, |
| | | | | | LUSC, THCA, UCEC |
| meth_ABHD14A.ACY1 | meth | ABHD14A.ACY1 | NO | 0 | CRAD, KIRC, KIRP, LIHC, |
| | | | | | PAAD, UCEC |
| meth_THRSP | meth | THRSP | YES | 12 | ESCA, KIRC, LUAD, PAAD, |
| | | | | | PRAD, THCA |
| meth_PI4KA | meth | PI4KA | YES | 25 | BLCA, CESC, KIRC, LIHC, OV |
| mRNA_VDAC2 | mRNA | VDAC2 | YES | 23 | BRCA, ESCA, HNSC, STAD, UCEC |
| meth_PSPN | meth | PSPN | YES | 10 | BLCA, BRCA, KIRC, PRAD, UCEC |
| mRNA_RP11-8L2.1 | mRNA | RP11-8L2.1 | NO | 0 | BLCA, LUSC, OV, SARC, UCEC |
| meth_SLC01C1 | meth | SLCO1C1 | YES | 15 | BLCA, HNSC, LUSC, TGCT, THCA |
| meth_NNMT | meth | NNMT | YES | 11 | CRAD, KIRC, KIRP, PRAD, SARC |
| mRNA_VLDLR | mRNA | VLDLR | YES | 37 | BLCA, CRAD, KIRC, KIRP, UCEC |
| meth_PKLR | meth | PKLR | YES | 29 | CESC, CRAD, KIRC, LIHC, UCEC |
| meth_TRAPPC10 | meth | TRAPPC10 | YES | 19 | CESC, CRAD, ESCA, HNSC, KIRC |
| meth_ITIH1 | meth | ITIH1 | YES | 9 | BLCA, KIRC, LIHC, SARC, THYM |
| mRNA_ZFPM1 | mRNA | ZFPM1 | YES | 46 | BLCA, CRAD, PRAD, STAD, UCEC |
| meth_CAP1P2 | meth | CAP1P2 | NO | 0 | BLCA, BRCA, STAD, THCA, UCEC |
| meth_PPL | meth | PPL | YES | 17 | BLCA, CESC, PAAD, SARC, UCEC |
| mRNA_RFXAP | mRNA | RFXAP | YES | 6 | CRAD, ESCA, HNSC, KIRC, STAD |
| meth_JDP2 | meth | JDP2 | YES | 16 | BRCA,KIRP,PRAD,STAD,UCEC |
| meth_SLC27A5 | meth | SLC27A5 | YES | 29 | CRAD, KIRP, LGG, LIHC, UCEC |
| mRNA_ARHGEF3 | mRNA | ARHGEF3 | YES | 12 | BLCA, LIHC, SARC, THYM, UCEC |
| mRNA_TUSC3 | mRNA | TUSC3 | YES | 18 | CRAD, LUAD, LUSC, PAAD, THYM |
| mRNA_KCNC4 | mRNA | KCNC4 | YES | 19 | BLCA, CRAD, TGCT, THCA, THYM |
| meth_ANKRD46 | meth | ANKRD46 | YES | 2 | BLCA,HNSC,KIRC,OV,TGCT |
| meth_HA02 | meth | HAO2 | YES | 17 | KIRC, KIRP, LUAD, PCPG, SARC |
| meth_HINT3 | meth | HINT3 | YES | 6 | CRAD, LUAD, LUSC, OV, STAD |
| mRNA_HMGN2P5 | mRNA | HMGN2P5 | NO | 0 | CRAD, HNSC, LGG, LUSC, STAD |
| meth_MYOZ3 | meth | MYOZ3 | YES | 8 | CESC, CRAD, HNSC, PRAD, THYM |
| mRNA_GRAMD2 | mRNA | GRAMD2 | YES | 1 | KIRP, LIHC, LUAD, LUSC, PCPG |
| meth_ARIDlB | meth | ARID1B | YES | 19 | CRAD, HNSC, LUAD, OV, UCEC |
| meth_ZNF776 | meth | ZNF776 | YES | 7 | BRCA, CESC, KIRC, LUAD, THCA |
| meth_HSD17B11 | meth | HSD17B11 | YES | 12 | HNSC, KIRC, LIHC, THCA, THYM |
| meth_KCTD15 | meth | KCTD15 | YES | 4 | BLCA, ESCA, KIRC, LGG, THYM |
| mRNA_DOCK4 | mRNA | DOCK4 | YES | 22 | BLCA, CESC, KIRP, PAAD, PRAD |
| mRNA_SNRNP27 | mRNA | SNRNP27 | YES | 9 | CESC, PAAD, PCPG, STAD, TGCT |
| mRNA_ADAM28 | mRNA | ADAM28 | YES | 12 | BLCA, KIRP, PAAD, PRAD, TGCT |
| mRNA_PLCH2 | mRNA | PLCH2 | YES | 20 | HNSC, LUSC, PAAD, PRAD, SARC |
| meth_CLCNKB | meth | CLCNKB | YES | 20 | BRCA, CRAD, ESCA, LUAD, THCA |
| meth_PTPN1 | meth | PTPN1 | YES | 54 | CRAD, LUSC, OV, TGCT, THYM |
| meth_SETD6 | meth | SETD6 | YES | 15 | BLCA, LUSC, PCPG, SARC, THCA |
| meth_RNF41 | meth | RNF41 | YES | 36 | KIRC, OV, SARC, THYM, UCEC |
| meth_ZFAND5 | meth | ZFAND5 | YES | 16 | BLCA, OV, PAAD, STAD, TGCT |
| meth_UQCRC2 | meth | UQCRC2 | YES | 21 | CESC,ESCA,LIHC,LUSC,OV |
| meth_VASP | meth | VASP | YES | 27 | CESC,ESCA,OV,PAAD,THYM |
| meth_CLPTM1L | meth | CLPTM1L | YES | 3 | BLCA,ESCA,PAAD,SARC,UCEC |
| mRNA_SNRPGP10 | mRNA | SNRPGP10 | NO | 0 | BLCA,BRCA,ESCA,LGG,PRAD |
| mRNA_CALM2 | mRNA | CALM2 | YES | 61 | BRCA, PAAD, PCPG, TGCT, THCA |
| mirna_MIR378A | miRNA | MIR378A | YES | 2 | HNSC,LIHC,LUAD,PCPG,THYM |
| meth_CUTA | meth | CUTA | YES | 8 | ESCA, SARC, STAD, TGCT, THYM |
| mRNA_ERF | mRNA | ERF | YES | 14 | BRCA, KIRP, LIHC, PRAD, THYM |
| meth_NHLRC3 | meth | NHLRC3 | YES | 4 | BRCA, LUSC,OV, STAD, THCA |
| mRNA_RCHY1 | mRNA | RCHY1 | YES | 19 | BLCA, CRAD, LUAD, PAAD, PCPG |
| meth_ANGPTL3 | meth | ANGPTL3 | YES | 37 | HNSC, LGG, OV, SARC, THCA |
| mRNA_STRADA | mRNA | STRADA | YES | 20 | CRAD, LGG, LUSC, PRAD |
| mRNA_HNRNPH3 | mRNA | HNRNPH3 | YES | 13 | CESC, HNSC, THYM, UCEC |
| mRNA_BTN2A1 | mRNA | BTN2A1 | YES | 7 | HNSC, PAAD, PRAD, STAD |
| meth_EMCN | meth | EMCN | YES | 9 | PRAD, THCA, THYM, UCEC |
| mRNA_ZHX3 | mRNA | ZHX3 | YES | 17 | KIRC, KIRP, LGG, LIHC |
| meth_F2 | meth | F2 | YES | 58 | BRCA, LIHC, LUAD, TGCT |
| meth_OSGIN1 | meth | OSGIN1 | YES | 10 | HNSC, LUAD, LUSC, THCA |
| meth_KBTBD8 | meth | KBTBD8 | YES | 14 | BLCA, KIRC, LGG, PAAD |
| meth_NADK2 | meth | NADK2 | YES | 12 | BRCA, KIRP, LIHC, STAD |
| meth_PIEZO1 | meth | PIEZO1 | YES | 20 | BRCA, CRAD, TGCT, UCEC |
| meth_ZNF267 | meth | ZNF267 | YES | 9 | BLCA, KIRC, PRAD, UCEC |
| mRNA_ST8SIAl | mRNA | ST8SIA1 | YES | 16 | BRCA, HNSC, LGG, PAAD |
| meth_CLDN16 | meth | CLDN16 | YES | 15 | CRAD, KIRP, PAAD, UCEC |
| mRNA_RPL5P34 | mRNA | RPL5P34 | NO | 0 | BRCA, ESCA, PRAD, STAD |
| mRNA_RNF141 | mRNA | RNF141 | YES | 6 | ESCA, HNSC, LGG, PRAD |
| meth_RP11.299J3.8 | meth | RP11.299J3.8 | NO | 0 | BRCA, CRAD, ESCA, LUAD |
| meth_COG6 | meth | COG6 | YES | 11 | HNSC, SARC, THCA, THYM |
| mRNA_GNA12 | mRNA | GNA12 | YES | 33 | BLCA, HNSC, LUSC, TGCT |
| meth_ATP6AP1L | meth | ATP6AP1L | YES | 6 | LUAD, LUSC, PCPG, STAD |
| meth_DIO2 | meth | DIO2 | YES | 16 | CESC, ESCA, PRAD, UCEC |
| mRNA_HOXC9 | mRNA | HOXC9 | YES | 12 | BRCA, CRAD, KIRC, thca |
| meth_CTD.2544N14.3 | meth | CTD.2544N14.3 | NO | 0 | BRCA, CESC, KIRP, THCA |
| meth_CYP17Al | meth | CYP17A1 | YES | 54 | BLCA, CRAD, LGG, THCA |
| mRNA_RPL5P4 | mRNA | RPL5P4 | NO | 0 | ESCA, KIRP, STAD, UCEC |
| mirna_MIR708 | miRNA | MIR708 | NO | 0 | HNSC, LGG, LUSC, THYM |
| mRNA_MEF2BNB-MEF2B | mRNA | MEF2BNB-MEF2B | YES | 10 | LGG, LUSC, STAD, UCEC |
| meth_FAM84B | meth | FAM84B | YES | 3 | BRCA, OV, PAAD, THYM |
| meth_GOLT1A | meth | GOLT1A | YES | 7 | BLCA, BRCA, HNSC, LIHC |
| meth_MLXIP | meth | MLXIP | YES | 16 | CESC, HNSC, KIRC, PCPG |
| mRNA_DCP1B | mRNA | DCP1B | YES | 16 | HNSC, LUSC, OV, TGCT |
| meth_DDR2 | meth | DDR2 | YES | 41 | CESC, PRAD, SARC, TGCT |
| meth_FGF1 | meth | FGF1 | YES | 57 | BLCA, BRCA, LUAD, LUSC |
| meth_TOR1A | meth | TOR1A | YES | 50 | BRCA, KIRC, STAD, THCA |
| mRNA_GPR63 | mRNA | GPR63 | YES | 12 | CRAD, LUAD, PRAD, SARC |
| meth_ADCY7 | meth | ADCY7 | YES | 29 | HNSC, OV, PRAD, UCEC |
| mRNA_CCSER1 | mRNA | CCSER1 | NO | 0 | BLCA, KIRP, LGG, SARC |
| meth_CTC.492K19.7 | meth | CTC.492K19.7 | NO | 0 | HNSC, LUAD, OV, THYM |
| mRNA_GUCY1A2 | mRNA | GUCY1A2 | YES | 15 | KIRC, KIRP, LGG, SARC |
| meth_HOXB6 | meth | HOXB6 | YES | 13 | LUAD, LUSC, THCA, UCEC |
| meth_TAL2 | meth | TAL2 | YES | 13 | BLCA, BRCA, CRAD, PRAD |
| mRNA_SPAG9 | mRNA | SPAG9 | YES | 26 | KIRP, LGG, OV, SARC |
| meth_DYNLL2 | meth | DYNLL2 | YES | 34 | BRCA, SARC, THCA, THYM |
| mRNA_STRIP1 | mRNA | STRIP1 | YES | 8 | KIRC, LIHC, TGCT, THYM |
| meth_FAM47E | meth | FAM47E | YES | 3 | BRCA, LUSC, OV, PRAD |
| meth_ELP3 | meth | ELP3 | YES | 30 | CESC, LUSC, OV, THYM |
| mRNA_PAM | mRNA | PAM | YES | 53 | LUAD, LUSC, PCPG, THCA |
| meth_UFM1 | meth | UFM1 | YES | 10 | BRCA, LUAD, LUSC, THCA |
| mRNA_FEZ1 | mRNA | FEZ1 | YES | 25 | HNSC, LGG, LUSC, PCPG |
| meth_Clorf43 | meth | Clorf43 | YES | 4 | HNSC, PAAD, PCPG, STAD |
| meth_EGF | meth | EGF | YES | 67 | BRCA, KIRC, SARC, THYM |
| meth_AP000692.10 | meth | AP000692.10 | NO | 0 | BRCA, KIRC, LUAD, TGCT |
| meth_FKBP14 | meth | FKBP14 | YES | 11 | BLCA, LUAD, THCA, UCEC |
| mRNA_MAZ | mRNA | MAZ | YES | 15 | KIRP, PRAD, STAD, THCA |
| mRNA_CTD-2314G24.2 | mRNA | CTD-2314G24.2 | NO | 0 | BLCA, LUSC, PRAD, THYM |
| mRNA_COX7A1 | mRNA | COX7A1 | YES | 9 | BLCA, KIRC, OV, UCEC |
| mRNA_CNN3 | mRNA | CNN3 | YES | 16 | KIRP, LGG, SARC, THYM |
| meth_DBF4 | meth | DBF4 | YES | 11 | HNSC, KIRP, LGG, SARC |
| meth_APOM | meth | APOM | YES | 25 | BLCA, KIRC, LIHC, PRAD |
| meth_GJA1 | meth | GJA1 | YES | 88 | PAAD, PRAD, THCA, THYM |
| meth_RP11.482M8.1 | meth | RP11.482M8.1 | NO | 0 | KIRP, LGG, LUSC, PAAD |
| meth_MOK | meth | MOK | YES | 19 | PAAD, PCPG, SARC, THCA |
| meth_FKBP1A | meth | FKBP1A | YES | 60 | CESC, CRAD, KIRC, UCEC |
| meth_GGTLC1 | meth | GGTLC1 | YES | 7 | KIRC, LUAD, TGCT, THCA |
| mRNA_SOX2 | mRNA | SOX2 | YES | 70 | LGG, LIHC, LUSC, PAAD |
| meth_HABP4 | meth | HABP4 | YES | 13 | BRCA, ESCA, PCPG, THCA |
| mRNA_ADAMTS20 | mRNA | ADAMTS20 | YES | 17 | LUAD, PRAD, THCA, UCEC |
| meth_TARS2 | meth | TARS2 | YES | 18 | BLCA, BRCA, OV, PCPG |
| meth_LRRC8D | meth | LRRC8D | YES | 16 | CESC, KIRP, SARC, TGCT |
| meth_CUL2 | meth | CUL2 | YES | 21 | LGG, LIHC, SARC, THYM |
| meth_WDYHV1 | meth | WDYHV1 | YES | 8 | HNSC, KIRP, LUSC, OV |
| mRNA_ZNF275 | mRNA | ZNF275 | YES | 7 | CRAD, OV, STAD, TGCT |
| meth_SGMS1 | meth | SGMS1 | YES | 26 | HNSC, KIRC, STAD, THCA |
| meth_ISLR | meth | ISLR | YES | 6 | CESC, KIRP, SARC, THYM |
| meth_FAM195A | meth | FAM195A | YES | 1 | BRCA, CESC, PRAD, TGCT |
| meth_CALU | meth | CALU | YES | 15 | BRCA, CESC, LIHC, TGCT |
| meth_RNU6.510P | meth | RNU6.510P | NO | 0 | ESCA, KIRC, THCA, UCEC |
| mRNA_WIZ | mRNA | WIZ | YES | 11 | BLCA, KIRC, OV, SARC |
| mRNA_FEV | mRNA | FEV | YES | 18 | BLCA, CESC, CRAD, LIHC |
| meth_RAPGEF3 | meth | RAPGEF3 | YES | 35 | CESC, LUAD, SARC, THYM |
| meth_CLDN15 | meth | CLDN15 | YES | 11 | CESC, LUSC, PAAD, PRAD |
| meth_LMO1 | meth | LMO1 | YES | 8 | CRAD, ESCA, KIRC, LUAD |
| mRNA_FIBIN | mRNA | FIBIN | YES | 3 | ESCA, KIRC, KIRP, LUAD |
| mRNA_CHD3 | mRNA | CHD3 | YES | 30 | LIHC, PRAD, STAD, UCEC |
| meth_ROPN1L | meth | ROPN1L | YES | 4 | KIRP, THCA, THYM, UCEC |
| meth_ATP6V1H | meth | ATP6V1H | YES | 24 | BRCA, LIHC, STAD, TGCT |
| meth_PPCDC | meth | PPCDC | YES | 9 | CRAD, LUAD, PCPG, THCA |
| mRNA_SRSF12 | mRNA | SRSF12 | YES | 12 | CRAD, PAAD, PRAD, UCEC |
| meth_MCM3 | meth | MCM3 | YES | 22 | BLCA, LGG, LUAD, THCA |
| mRNA_SIMC1 | mRNA | SIMC1 | YES | 1 | BLCA, CRAD, LGG, SARC |
| meth_TAB2 | meth | TAB2 | YES | 32 | ESCA, HNSC, KIRC, OV |
| meth_RNF19A | meth | RNF19A | YES | 19 | BLCA, LUAD, OV, THCA |
| meth TMEM81 | meth | TMEM81 | YES | 2 | CRAD, KIRP, LIHC, TGCT |
| meth_PSMC3 | meth | PSMC3 | YES | 55 | ESCA, PAAD, SARC, STAD |
| mRNA_BRMS1L | mRNA | BRMS1L | YES | 10 | ESCA, KIRC, PAAD,THYM |
| mRNA_PHLDA1 | mRNA | PHLDA1 | YES | 9 | OV, PRAD, TGCT, UCEC |
| meth_NEDD9 | meth | NEDD9 | YES | 23 | KIRP, LIHC, LUAD, SARC |
| mRNA_NAV1 | mRNA | NAVI | YES | 10 | BLCA, HNSC, KIRP, PCPG |
| meth_ZNF764 | meth | ZNF764 | YES | 8 | HNSC, LUAD, PAAD, THYM |
| mirna_MIR500B | miRNA | MIR500B | NO | 0 | KIRC, KIRP, PCPG, SARC |
| mRNA_LRRC37B | mRNA | LRRC37B | YES | 3 | CRAD, OV, PCPG, THYM |

The pan-cancer survival model-derived driver therapeutic or drug targets or genes generated according to the methods, systems, algorithms, programs, and codes described above are set forth in Appendices M and N (full listings) and Tables N (top 51 genes) and O (top 51 genes).

TABLE N
Top 51 genes from pan-cancer from Table A (20 cancer types) (survival) MEGENA (from Appendix M)

| Rank | Full_Name | Data_Type | HUGO_GENE | GO_Annotated | Number_Of_GO_Annotations |
|---|---|---|---|---|---|
| 1 | Age | Age | | NO | 0 |
| 2 | mRNA_FCGR2A | mRNA | FCGR2A | YES | 10 |
| 3 | mRNA_SLFN11 | mRNA | SLFN11 | YES | 11 |
| 4 | mRNA_RGS19 | mRNA | RGS19 | YES | 16 |
| 5 | mRNA_FAM227B | mRNA | FAM227B | NO | 0 |
| 6 | METH_AKNAD1 | METH | AKNAD1 | YES | 1 |
| 7 | mRNA_SHC1 | mRNA | SHC1 | YES | 49 |
| 8 | mRNA_TADA2B | mRNA | TADA2B | YES | 20 |
| 9 | mRNA_PAX5 | mRNA | PAX5 | YES | 29 |
| 11 | METH_MAP2K2 | METH | MAP2K2 | YES | 60 |
| 11 | mRNA_ARL4C | mRNA | ARL4C | YES | 16 |
| 12 | STV_CDK4 | STV | CDK4 | YES | 63 |
| 13 | METH_TERC | METH | TERC | NO | 0 |
| 15 | METH_NFATC3 | METH | NFATC3 | YES | 24 |
| 16 | METH_SLC10A1 | METH | SLC10A1 | YES | 16 |
| 16 | mRNA_GCNT4 | mRNA | GCNT4 | YES | 18 |
| 17 | METH_HADHA | METH | HADHA | YES | 30 |
| 18 | METH_HOXA10.HOXA9 | METH | HOXA10.HOXA9 | NO | 0 |
| 19 | mRNA_CLDN1 | mRNA | CLDN1 | YES | 39 |
| 20 | mRNA_RP11-1055B8.1 | mRNA | RP11-1055B8.1 | NO | 0 |
| 21 | mRNA_RP11-403A3.3 | mRNA | RP11-403A3.3 | NO | 0 |
| 22 | mirna_MIR146A | miRNA | MIR146A | YES | 1 |
| 24 | mRNA_INHBA | mRNA | INHBA | YES | 66 |
| 24 | mRNA_TMEM189 | mRNA | TMEM189 | YES | 8 |
| 26 | STV_FGFRL1 | STV | FGFRL1 | YES | 17 |
| 27 | METH_GPR22 | METH | GPR22 | YES | 11 |
| 27 | mRNA_FOSL1 | mRNA | FOSL1 | YES | 41 |
| 29 | mRNA_DACT2 | mRNA | DACT2 | YES | 14 |
| 29 | STV_CAMK2N2 | STV | CAMK2N2 | YES | 8 |
| 31 | mRNA_LRMP | mRNA | LRMP | YES | 19 |
| 32 | METH_MAPK13 | METH | MAPK13 | YES | 26 |
| 33 | mRNA_SMIM14 | mRNA | SMIM14 | YES | 5 |
| 34 | mRNA_GALNT16 | mRNA | GALNT16 | YES | 11 |
| 35 | mRNA_TNC | mRNA | TNC | YES | 34 |
| 36 | METH_IL1R1 | METH | IL1R1 | YES | 24 |
| 36 | mRNA_IFITM2 | mRNA | IFITM2 | YES | 15 |
| 37 | mRNA_SFPQ | mRNA | SFPQ | YES | 40 |
| 39 | mRNA_SLC25A35 | mRNA | SLC25A35 | YES | 8 |
| 39 | mRNA_TUBB2B | mRNA | TUBB2B | YES | 16 |
| 40 | mRNA_PLEKHA8P1 | mRNA | PLEKHA8P1 | NO | 0 |
| 41 | mRNA_TRPV4 | mRNA | TRPV4 | YES | 84 |
| 42 | mRNA_NR2E1 | mRNA | NR2E1 | YES | 53 |
| 44 | METH_TBC1D8 | METH | TBC1D8 | YES | 10 |
| 44 | mRNA_FOXP3 | mRNA | FOXP3 | YES | 85 |
| 45 | mirna_MIR6503 | miRNA | MIR6503 | NO | 0 |
| 46 | mRNA_AP000439.3 | mRNA | AP000439.3 | NO | 0 |
| 47 | mRNA_MSL3P1 | mRNA | MSL3P1 | NO | 0 |
| 48 | mRNA_PHYHD1 | mRNA | PHYHD1 | YES | 5 |
| 49 | mRNA_AC098820.3 | mRNA | AC098820.3 | NO | 0 |
| 51 | METH_ALDOA | METH | ALDOA | YES | 39 |
| 51 | METH_CCL28 | METH | CCL28 | YES | 14 |

TABLE O
Top 51 genes from pan-cancer from Table A (20 cancer types) (survival) nGOseq (from Appendix N)

| Rank | Full_Name | Data_Type | HUGO_GENE | GO_Annotated | Number_Of_GO_Annotations |
|---|---|---|---|---|---|
| 1 | Age | Age | | NO | 0 |
| 2 | METH_CACNB2 | METH | CACNB2 | YES | 32 |
| 3 | CNV_PALM | CNV | PALM | YES | 32 |
| 4 | METH_DDR2 | METH | DDR2 | YES | 41 |
| 5 | mRNA_SLC22A5 | mRNA | SLC22A5 | YES | 33 |
| 6 | mRNA_TBC1D10C | mRNA | TBC1D10C | YES | 18 |
| 8 | METH_TP63 | METH | TP63 | YES | 103 |
| 8 | STV_ATP6V0A1 | STV | ATP6V0A1 | YES | 38 |
| 9 | STV_ARL4C | STV | ARL4C | YES | 16 |
| 10 | METH_CACNG4 | METH | CACNG4 | YES | 20 |
| 12 | CNV_FAM49B | CNV | FAM49B | YES | 12 |
| 12 | METH_ATRAID | METH | ATRAID | YES | 15 |
| 13 | CNV_GNA15 | CNV | GNA15 | YES | 20 |
| 14 | mRNA_PDLIM5 | mRNA | PDLIM5 | YES | 22 |
| 16 | mRNA_LRRK2 | mRNA | LRRK2 | YES | 157 |
| 16 | mRNA_MICALL1 | mRNA | MICALL1 | YES | 26 |
| 19 | METH_MIP | METH | MIP | YES | 21 |
| 19 | STV_RPL32 | STV | RPL32 | YES | 18 |
| 20 | CNV_HCK | CNV | HCK | YES | 61 |
| 20 | mRNA_PIK3R3 | mRNA | PIK3R3 | YES | 12 |
| 22 | METH_RAB15 | METH | RAB15 | YES | 16 |
| 22 | mRNA_PIM1 | mRNA | PIM1 | YES | 32 |
| 23 | METH_C2 | METH | C2 | YES | 17 |
| 24 | METH_PAM | METH | PAM | YES | 53 |
| 27 | CNV_SORBS2 | CNV | SORBS2 | YES | 23 |
| 28 | mRNA_TSHR | mRNA | TSHR | YES | 38 |
| 29 | METH_CD80 | METH | CD80 | YES | 30 |
| 29 | METH_EPPIN | METH | EPPIN | YES | 15 |
| 30 | METH_KLHL10 | METH | KLHL10 | YES | 12 |
| 30 | METH_SLURP1 | METH | SLURP1 | YES | 14 |
| 32 | STV_MYH7 | STV | MYH7 | YES | 31 |
| 34 | mRNA_CUZD1 | mRNA | CUZD1 | YES | 11 |
| 35 | METH_SNX4 | METH | SNX4 | YES | 22 |
| 35 | mRNA_PPIA | mRNA | PPIA | YES | 36 |
| 36 | CNV_HYAL3 | CNV | HYAL3 | YES | 19 |
| 37 | mRNA_SEMA3A | mRNA | SEMA3A | YES | 47 |
| 38 | CNV_HTR3D | CNV | HTR3D | YES | 12 |
| 38 | METH_ADAM2 | METH | ADAM2 | YES | 18 |
| 40 | CNV_NPRL2 | CNV | NPRL2 | YES | 15 |
| 41 | CNV_EFNA2 | CNV | EFNA2 | YES | 13 |
| 41 | STV_EHD2 | STV | EHD2 | YES | 32 |
| 43 | CNV_AHSG | CNV | AHSG | YES | 28 |
| 43 | mRNA_INHBA | mRNA | INHBA | YES | 66 |
| 45 | mRNA_SNAI2 | mRNA | SNAI2 | YES | 57 |
| 46 | STV_STRAP | STV | STRAP | YES | 19 |
| 47 | mRNA_SEMA7A | mRNA | SEMA7A | YES | 23 |
| 47 | STV_PPP2R1A | STV | PPP2R1A | YES | 49 |
| 48 | mRNA_EPHA2 | mRNA | EPHA2 | YES | 77 |
| 49 | mRNA_ASPH | mRNA | ASPH | YES | 45 |
| 51 | CNV_POLR2H | CNV | POLR2H | YES | 35 |

In some embodiments, pan-cancer enriched genes with no association with cancer or other genes in published literature are set forth in Table AAJ.

In some embodiments, the pan-cancer 22 cancer types (e.g., cancers set forth in Table A) enriched genes with no association with cancer or other genes in published literature are set forth in Table AAJ. In some embodiments, pan-cancer enriched genes with no associated functional annotations are set forth in Table AAK.

TABLE AAJ
pan-cancer22 enriched genes (MEGENA) with
no association with cancer or other genes in published literature
genes

ABHD14A.ACY1
AC004987.9
AP000692.10
CAP1P2
CTC.492K19.7
CTD-2314G24.2
CTD.2544N14.3
EIF4BP3
HMGN2P5
MIR500B
MIR708
MKLN1-AS
POT1-AS1
RAET1E-AS1
RNU6.510P
RP11-398K22.12
RP11-8L2.1
RP11.299J3.8
RP11.482M8.1
RP11.540D14.8
RPL5P34
RPL5P4
SNRPGP10
ATP6AP1L
C16orf87
C1orf43
CACHD1
CIB3
FAM131A
FAM195A
FAM47E
FLRT1
GRAMD2
GUCY1A2
HNRNPH3
HNRNPUL2-BSCL2
KBTBD8
LRRC37B
MEF2BNB-MEF2B
MY0Z3
NHLRC3
SNRNP27
TMEM81
ZNF275
ZNF764
ZNF776

TABLE AAK
pan-cancer22 enriched genes (MEGENA)
with no associated functional annotations
genes

ABHD14A.ACY1
AC004987.9
AP000692.10
CAP1P2
CCSER1
CTC.492K19.7
CTD-2314G24.2
CTD.2544N14.3
EIF4BP3
HAS2-AS1
HMGN2P5
HOXD-AS2
MIR500B
MIR708
MKLN1-AS
POT1-AS1
RAET1E-AS1
RNU6.510P
RP11.299J3.8
RP11-398K22.12
RP11.482M8.1
RP11.540D14.8
RP11-8L2.1
RPL5P34
RPL5P4
SNRPGP10

In some embodiments, pan-cancer survival enriched genes with no association with cancer or other genes in published literature are set forth in Table AAL and Table AAN. In some embodiments, pan-cancer survival enriched genes with no associated functional annotations are set forth in Table AAM and AAO.

TABLE AAL
pan-cancer survival enriched genes (MEGENA) with
no association with cancer or other genes in published literature
genes

C19orf35
CAMK2N2
GPR22
AC092667.2
AC098820.3
AP000439.3
C9orf173
CH17-360D5.2
FAM227B
HOXA10.HOXA9
IPO5P1
MIR629
MIR6503
MSL3P1
PAXIP1-AS1
RP11-1055B8.1
RP11-212121.2
RP11-403A3.3
RP11-774O3.3
RP11.387A1.5
RP5-943J3.2
MIR374A
PHYHD1
SLC25A35
TMEM189
UBXN6
ZMYM6NB

TABLE AAM
pan-cancer survival enriched genes (MEGENA)
with no associated functional annotations
genes

AC092667.2
AC098820.3
AP000439.3
C9orf173
CH17-360D5.2
CTD-2357A8.3
FAM227B
HOXA10.HOXA9
IPO5P1
LINC00941
MIR629
MIR6503
MSL3P1
NA
PAXIP1-AS1
PLEKHA8P1
RP11-1055B8.1
RP11-212121.2
RP11.387A1.5
RP11-403A3.3
RP11-774O3.3
RP5-943J3.2
TERC

TABLE AAN
pan-cancer survival enriched genes (nGOseq) with
no association with cancer or other genes in published literature
genes

KLHL10
OR2A4
TMPRSS15

TABLE AAO
pan-cancer survival enriched genes (nGOseq)
with no associated functional annotations
genes

NA

The term “subject” refers in one embodiment to an animal or mammal in need of therapy for, or susceptible to, a condition or its sequelae. The subject can include dogs, cats, pigs, cows, sheep, goats, horses, rats, mice, monkeys, and humans.

As used herein, the term “therapeutic or drug target” or “drug target” includes diagnostic and prognostic genes, described herein which are useful in the diagnosis, prognosis, or treatment of cancer, e.g., over- or under-activity, emergence, expression, growth, remission, recurrence or resistance of tumors before, during or after therapy. The levels of the therapeutic or drug targets may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression (e.g., by ISH, Northern Blot, or qPCR), increased or decreased protein level (e.g., by IHC), or increased or decreased; (2) its presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its presence or absence in clinical subset of subjects who have not been diagnosed with cancer or who have cancer, including subjects responding to a particular therapy or those developing resistance.

In some embodiments, the therapeutic or drug targets for BRCA as used herein are set forth in Appendices A and B (full listing) and Tables B (top 50 genes), C (top 52 genes), AP (28 genes), AQ (22 genes), AR (3 genes), AS (1 gene), or combinations thereof.

TABLE B
Top 50 genes from BRCA vs. Normal MEGENA (see full listing in Appendix A)

| Rank | Full_Name | Data_Type | HUGO_GENE | GO_Annotated | Number_OLGO_Annotations |
|---|---|---|---|---|---|
| 1 | cnv_MT1H | cnv | MT1H | YES | 9 |
| 1 | cnv_ZPLD1 | cnv | ZPLD1 | YES | 2 |
| 2 | mrna_C6orf203 | mrna | C6orf203 | YES | 1 |
| 2 | stv_LINC00996 | stv | LINC00996 | NO | 0 |
| 3 | mrna_PSMD11 | mrna | PSMD11 | YES | 43 |
| 3 | mrna_ACLY | mrna | ACLY | YES | 31 |
| 4 | cnv_MTVR2 | cnv | MTVR2 | NO | 0 |
| 4 | mrna_FBXO3 | mrna | FBXO3 | YES | 7 |
| 5 | meth_AKAP12 | meth | AKAP12 | YES | 16 |
| 5 | mrna_SLC4A8 | mrna | SLC4A8 | YES | 22 |
| 6 | cnv_GLYAT | cnv | GLYAT | YES | 13 |
| 6 | mrna_MAMDC2 | mrna | MAMDC2 | YES | 6 |
| 7 | cnv_ABHD10 | cnv | ABHD10 | YES | 8 |
| 7 | mrna_PRIMA1 | mrna | PRIMA1 | YES | 8 |
| 8 | cnv_ZC3H12A | cnv | ZC3H12A | YES | 92 |
| 8 | meth_DUSP26 | meth | DUSP26 | YES | 22 |
| 9 | cnv_TOX3 | cnv | TOX3 | YES | 13 |
| 9 | stv_EXOC3L1 | stv | EXOC3L1 | YES | 9 |
| 10 | mrna_PPAT | mrna | PPAT | YES | 26 |
| 10 | mrna_SGOL1 | mrna | SGOL1 | YES | 17 |
| 11 | cnv_PLXND1 | cnv | PLXND1 | YES | 27 |
| 11 | cnv_TMEM184C | cnv | TMEM184C | YES | 4 |
| 12 | mrna_FAM35A | mrna | FAM35A | NO | 0 |
| 12 | mrna_CACHD1 | mrna | CACHD1 | YES | 2 |
| 13 | cnv_CXCL8 | cnv | CXCL8 | YES | 38 |
| 13 | cnv_SLC16A6 | cnv | SLC16A6 | YES | 9 |
| 14 | mrna_METTL17 | mrna | METTL17 | YES | 8 |
| 14 | mrna_RP5-1065J22.8 | mrna | RP5-1065J22.8 | NO | 0 |
| 15 | meth_CUL1 | meth | CUL1 | YES | 36 |
| 15 | mrna_MYOM2 | mrna | MYOM2 | YES | 18 |
| 16 | meth_FOXC1 | meth | FOXC1 | YES | 77 |
| 16 | mrna_CTCF | mrna | CTCF | YES | 41 |
| 17 | meth_HK1 | meth | HK1 | YES | 31 |
| 18 | meth_AATK | meth | AATK | YES | 20 |
| 18 | mrna_TOB1-AS1 | mrna | TOB1-AS1 | NO | 0 |
| 19 | cnv_HMGN1 | cnv | HMGN1 | YES | 18 |
| 19 | mrna_MAFG | mrna | MAFG | YES | 19 |
| 20 | mirna _MIR4738 | mirna | MIR4738 | NO | 0 |
| 20 | stv_KIF13A | stv | KIF13A | YES | 35 |
| 21 | mrna_PRR11 | mrna | PRR11 | YES | 5 |
| 21 | mrna_GSTT2B | mrna | GSTT2B | YES | 9 |
| 22 | meth_CCL18 | meth | CCL18 | YES | 23 |
| 22 | stv_BRD9 | stv | BRD9 | YES | 8 |
| 23 | meth_RASSF4 | meth | RASSF4 | YES | 3 |
| 23 | mrna_SPRED2 | mrna | SPRED2 | YES | 17 |
| 24 | mrna_EFR3B | mrna | EFR3B | YES | 7 |
| 24 | stv_TLR8 | stv | TLR8 | YES | 38 |
| 25 | mrna_ANKMY2 | mrna | ANKMY2 | YES | 6 |
| 25 | mrna_GFM1 | mrna | GFM1 | YES | 12 |
| 26 | cnv_SGSM1 | cnv | SGSM1 | YES | 12 |
| 26 | cnv_TMCO5B | cnv | TMCO5B | NO | 0 |
| 27 | mrna_TBC1D8 | mrna | TBC1D8 | YES | 10 |
| 27 | mrna_GS1-124K5.11 | mrna | GS1-124K5.11 | NO | 0 |
| 28 | cnv_CES5A | cnv | CES5A | YES | 5 |
| 28 | mrna_EZH2 | mrna | EZH2 | YES | 69 |
| 29 | cnv_PSMG1 | cnv | PSMG1 | YES | 11 |
| 29 | mrna_LRRIQ1 | mrna | LRRIQ1 | YES | 1 |
| 30 | mirna_MIR676 | mirna | MIR676 | NO | 0 |
| 30 | stv_NQO1 | stv | NQO1 | YES | 28 |
| 31 | meth_C19orf70 | meth | C19orf70 | YES | 8 |
| 31 | mrna_ABCG1 | mrna | ABCG1 | YES | 56 |
| 32 | mirna _MIR3940 | mirna | MIR3940 | NO | 0 |
| 32 | mrna_PTS | mrna | PTS | YES | 14 |
| 33 | cnv_LOC101929268 | cnv | LOC101929268 | NO | 0 |
| 33 | mrna_B4GALT1 | mrna | B4GALT1 | YES | 59 |
| 34 | mrna_MAP3K14-AS1 | mrna | MAP3K14-AS1 | NO | 0 |
| 34 | stv_AQP3 | stv | AQP3 | YES | 25 |
| 35 | mrna_SAMD11 | mrna | SAMD11 | YES | 6 |
| 35 | mrna_ZDHHC11B | mrna | ZDHHC11B | YES | 5 |
| 36 | meth_ACADS | meth | ACADS | YES | 19 |
| 36 | stv_RNF141 | stv | RNF141 | YES | 6 |
| 37 | meth_RPS24 | meth | RPS24 | YES | 28 |
| 37 | stv_ZNF3 | stv | ZNF3 | YES | 14 |
| 38 | cnv_EEF1E1 | cnv | EEF1E1 | YES | 18 |
| 38 | cnv_LRBA | cnv | LRBA | YES | 11 |
| 39 | cnv_CASC3 | cnv | CASC3 | YES | 27 |
| 39 | stv_DDX39B | stv | DDX39B | YES | 45 |
| 40 | meth_ADAMTS15 | meth | ADAMTS15 | YES | 14 |
| 40 | mrna_OSR1 | mrna | OSR1 | YES | 63 |
| 41 | mrna_OSCP1 | mrna | OSCP1 | YES | 5 |
| 41 | stv_PCDH7 | stv | PCDH7 | YES | 9 |
| 42 | cnv_LOC101928580 | cnv | LOC101928580 | NO | 0 |
| 42 | meth_PLIN2 | meth | PLIN2 | YES | 13 |
| 43 | mrna_SNF8 | mrna | SNF8 | YES | 40 |
| 43 | mrna_CFAP36 | mrna | CFAP36 | YES | 3 |
| 44 | cnv_ZC4H2 | cnv | ZC4H2 | YES | 13 |
| 44 | stv_FXR2 | stv | FXR2 | YES | 15 |
| 45 | mrna_PEX10 | mrna | PEX10 | YES | 11 |
| 45 | stv_AVPI1 | stv | AVPI1 | YES | 3 |
| 46 | cnv_SH3BGR | cnv | SH3BGR | YES | 5 |
| 46 | meth_CCKBR | meth | CCKBR | YES | 27 |
| 47 | cnv_LIPI | cnv | LIPI | YES | 10 |
| 47 | stv_SEPP1 | stv | SEPP1 | YES | 10 |
| 48 | meth_SP100 | meth | SP100 | YES | 43 |
| 48 | mrna_PP14571 | mrna | PP14571 | NO | 0 |
| 49 | mrna_TBRG4 | mrna | TBRG4 | YES | 8 |
| 49 | mrna_SLC25A32 | mrna | SLC25A32 | YES | 14 |
| 50 | meth_FBLN1 | meth | FBLN1 | YES | 27 |
| 50 | mrna_ZSCAN21 | mrna | ZSCAN21 | YES | 13 |

TABLE C
Top 52 genes from BRCA vs. Normal nGOseq (see full listing in Appendix B)

| Rank | Full_Name | Data_Type | HUGO_GENE | GO_Annotated | Number_OLGO_Annotations |
|---|---|---|---|---|---|
| 1 | mrna_PAPPA2 | mrna | PAPPA2 | YES | 13 |
| 1 | mrna_DRD2 | mrna | DRD2 | YES | 128 |
| 2 | cnv_BLZF1 | cnv | BLZF1 | YES | 18 |
| 2 | mrna_TMED2 | mrna | TMED2 | YES | 42 |
| 3 | meth_PHOX2A | meth | PHOX2A | YES | 19 |
| 3 | mrna_CHST3 | mrna | CHST3 | YES | 12 |
| 4 | meth _SYNGR2 | meth | SYNGR2 | YES | 8 |
| 4 | meth_TRIM38 | meth | TRIM38 | YES | 16 |
| 5 | cnv_PBXIP1 | cnv | PBXIP1 | YES | 10 |
| 5 | meth_ITK | meth | ITK | YES | 33 |
| 6 | meth_MAP2K2 | meth | MAP2K2 | YES | 60 |
| 6 | mrna_CORO2B | mrna | CORO2B | YES | 8 |
| 7 | cnv_LAMTOR2 | cnv | LAMTOR2 | YES | 25 |
| 7 | meth_TNFRSF10D | meth | TNFRSF10D | YES | 20 |
| 8 | meth_CTNNAL1 | meth | CTNNAL1 | YES | 11 |
| 8 | meth_SLC5A7 | meth | SLC5A7 | YES | 27 |
| 9 | meth_AGAP2 | meth | AGAP2 | YES | 27 |
| 9 | mrna_BCL9 | mrna | BCL9 | YES | 15 |
| 10 | cnv_RGS1 | cnv | RGS1 | YES | 16 |
| 10 | mrna_E2F8 | mrna | E2F8 | YES | 29 |
| 11 | cnv_MARC2 | cnv | MARC2 | YES | 17 |
| 11 | mrna_SIRPA | mrna | SIRPA | YES | 10 |
| 12 | mrna_ESM1 | mrna | ESM1 | YES | 9 |
| 13 | cnv_PDC | cnv | PDC | YES | 15 |
| 13 | meth_DDR2 | meth | DDR2 | YES | 41 |
| 14 | cnv_ATF6 | cnv | ATF6 | YES | 41 |
| 14 | meth_GPR142 | meth | GPR142 | YES | 9 |
| 15 | meth_ACKR1 | meth | ACKR1 | YES | 18 |
| 15 | meth_GIPR | meth | GIPR | YES | 25 |
| 16 | meth_GUCY2D | meth | GUCY2D | YES | 23 |
| 16 | meth_TGFBI | meth | TGFBI | YES | 21 |
| 17 | meth_NMBR | meth | NMBR | YES | 13 |
| 17 | mrna_LYVE1 | mrna | LYVE1 | YES | 19 |
| 18 | meth_OR7C2 | meth | OR7C2 | YES | 11 |
| 18 | stv_KIFC3 | stv | KIFC3 | YES | 28 |
| 19 | cnv_HLX | cnv | HLX | YES | 19 |
| 19 | cnv_OR10J1 | cnv | OR10J1 | YES | 16 |
| 20 | meth_CD1C | meth | CD1C | YES | 18 |
| 21 | meth_HYAL2 | meth | HYAL2 | YES | 67 |
| 21 | meth_RECK | meth | RECK | YES | 17 |
| 22 | meth_CEMIP | meth | CEMIP | YES | 25 |
| 22 | mrna_LRRC59 | mrna | LRRC59 | YES | 11 |
| 23 | mrna_RAD51 | mrna | RAD51 | YES | 72 |
| 23 | mrna_TIMELESS | mrna | TIMELESS | YES | 28 |
| 24 | mrna_SFXN1 | mrna | SFXN1 | YES | 13 |
| 24 | mrna_H2AFX | mrna | H2AFX | YES | 32 |
| 25 | meth_GDA | meth | GDA | YES | 13 |
| 25 | meth_SPRR2A | meth | SPRR2A | YES | 10 |
| 26 | cnv_CD247 | cnv | CD247 | YES | 20 |
| 26 | meth_ZIC1 | meth | ZIC1 | YES | 26 |
| 27 | cnv_RAB3GAP2 | cnv | RAB3GAP2 | YES | 21 |
| 27 | mrna_PDE2A | mrna | PDE2A | YES | 49 |
| 28 | cnv_STX6 | cnv | STX6 | YES | 33 |
| 29 | cnv_CRTC2 | cnv | CRTC2 | YES | 17 |
| 29 | meth_FXYD1 | meth | FXYD1 | YES | 27 |
| 30 | meth_NDUFAF6 | meth | NDUFAF6 | YES | 8 |
| 30 | mirna_MIR100 | mirna | MIR100 | YES | 2 |
| 31 | cnv_ARL8A | cnv | ARL8A | YES | 24 |
| 31 | mrna_FOXM1 | mrna | FOXM1 | YES | 38 |
| 32 | cnv_CREB3L4 | cnv | CREB3L4 | YES | 22 |
| 32 | cnv_TGFB2 | cnv | TGFB2 | YES | 119 |
| 33 | meth_KCNIP1 | meth | KCNIP1 | YES | 21 |
| 33 | mrna_AURKB | mrna | AURKB | YES | 61 |
| 34 | mrna_CXCL2 | mrna | CXCL2 | YES | 17 |
| 34 | mrna_KIF15 | mrna | KIF15 | YES | 21 |
| 35 | meth_C6 | meth | C6 | YES | 15 |
| 35 | mrna_DEPDC1B | mrna | DEPDC1B | YES | 8 |
| 36 | mirna_MIR96 | mirna | MIR96 | YES | 2 |
| 36 | mrna_SYT13 | mrna | SYT13 | YES | 15 |
| 37 | mrna_ACADL | mrna | ACADL | YES | 26 |
| 37 | mrna_KLB | mrna | KLB | YES | 24 |
| 38 | cnv_GCSAML | cnv | GCSAML | YES | 2 |
| 38 | cnv_HNRNPU | cnv | HNRNPU | YES | 37 |
| 39 | mrna_CAV1 | mrna | CAV1 | YES | 141 |
| 39 | mrna_B4GALT3 | mrna | B4GALT3 | YES | 17 |
| 40 | cnv_ASH1L | cnv | ASH1L | YES | 40 |
| 40 | meth_GPLD1 | meth | GPLD1 | YES | 43 |
| 41 | cnv_SPRR2G | cnv | SPRR2G | YES | 7 |
| 41 | mrna_LMOD1 | mrna | LMOD1 | YES | 15 |
| 42 | meth_PNOC | meth | PNOC | YES | 13 |
| 42 | mrna_NSF | mrna | NSF | YES | 39 |
| 43 | meth_FMO2 | meth | FMO2 | YES | 19 |
| 43 | mrna_GPIHBP1 | mrna | GPIHBP1 | YES | 35 |
| 44 | cnv_LPGAT1 | cnv | LPGAT1 | YES | 16 |
| 44 | meth_HAMP | meth | HAMP | YES | 30 |
| 45 | cnv_QSOX1 | cnv | QSOX1 | YES | 26 |
| 45 | mrna_COPA | mrna | COPA | YES | 24 |
| 46 | cnv_SMG7 | cnv | SMG7 | YES | 17 |
| 46 | mrna_PRCD | mrna | PRCD | YES | 7 |
| 47 | meth_MAML1 | meth | MAML1 | YES | 21 |
| 47 | mrna _SYNGR3 | mrna | SYNGR3 | YES | 12 |
| 48 | cnv_WNT3A | cnv | WNT3A | YES | 101 |
| 48 | mrna_DIAPH3 | mrna | DIAPH3 | YES | 9 |
| 49 | meth_MRGPRF | meth | MRGPRF | YES | 10 |
| 50 | meth_CTNNA2 | meth | CTNNA2 | YES | 32 |
| 50 | mrna_MAMDC2 | mrna | MAMDC2 | YES | 6 |
| 51 | cnv_ZBTB18 | cnv | ZBTB18 | YES | 18 |
| 51 | meth_STXBP6 | meth | STXBP6 | YES | 15 |
| 52 | cnv_DENND1B | cnv | DENND1B | YES | 16 |
| 52 | meth_SLC7A2 | meth | SLC7A2 | YES | 32 |

In some embodiments, the therapeutic or drug targets for ER positive and ER generated according to the methods, systems, algorithms, programs, and codes described above are set forth in Appendices C and D(full listings) and Tables D(top 52 genes), E(top 52 genes), AX (32 genes), AY (17 genes), AZ (1 gene), AAA (2 genes), or combinations thereof.

TABLE D
Top 52 genes from ER+vs. ER- MEGENA (see full listing in Appendix C)

| Rank | Full_Name | Data_Type | HUGO_GENE | GO_Annotated | Number_Of_GO_Annotations |
|---|---|---|---|---|---|
| 1 | mrna_ANXA3 | mrna | ANXA3 | YES | 27 |
| 1 | mrna_WDR43 | mrna | WDR43 | YES | 12 |
| 2 | meth_CHAC1 | meth | CHAC1 | YES | 19 |
| 2 | mrna_RP11-1081L13.4 | mrna | RP11-1081L13.4 | NO | 0 |
| 3 | meth_DCAF12 | meth | DCAF12 | YES | 6 |
| 3 | meth_NOSIP | meth | NOSIP | YES | 14 |
| 4 | cnv_RPRML | cnv | RPRML | YES | 2 |
| 4 | mrna_PLEKHG1 | mrna | PLEKHG1 | YES | 6 |
| 5 | mrna_IL12RB1 | mrna | IL12RB1 | YES | 26 |
| 5 | mrna_ILF3-AS1 | mrna | ILF3-AS1 | NO | 0 |
| 6 | meth_SNORD116-1 | meth | SNORD116-1 | NO | 0 |
| 6 | mrna_CPNE8 | mrna | CPNE8 | YES | 3 |
| 7 | mrna_CX3CL1 | mrna | CX3CL1 | YES | 42 |
| 7 | mrna_STX7 | mrna | STX7 | YES | 33 |
| 8 | meth_C6orf48 | meth | C6orf48 | NO | 0 |
| 8 | mrna_IGHV3-21 | mrna | IGHV3-21 | YES | 17 |
| 9 | meth_DPM1 | meth | DPM1 | YES | 21 |
| 9 | meth_RCVRN | meth | RCVRN | YES | 10 |
| 10 | meth_CPA3 | meth | CPA3 | YES | 15 |
| 10 | mrna_ESYT3 | mrna | ESYT3 | YES | 15 |
| 11 | mrna_SLC37A3 | mrna | SLC37A3 | YES | 10 |
| 11 | stv_HMX3 | stv | HMX3 | YES | 15 |
| 12 | mrna_AFAP1 | mrna | AFAP1 | YES | 8 |
| 12 | mrna_RPS7P1 | mrna | RPS7P1 | NO | 0 |
| 13 | cnv_WNT9B | cnv | WNT9B | YES | 43 |
| 13 | mrna_IGKV1-16 | mrna | IGKV1-16 | YES | 16 |
| 14 | meth_ZMYND10 | meth | ZMYND10 | YES | 9 |
| 14 | mrna_TIA1 | mrna | TIA1 | YES | 19 |
| 15 | meth_C1QTNF7 | meth | C1QTNF7 | YES | 4 |
| 15 | meth_PLA2G4E-AS1 | meth | PLA2G4E-AS1 | NO | 0 |
| 16 | meth_CSN1S1 | meth | CSN1S1 | YES | 4 |
| 16 | mrna_LYN | mrna | LYN | YES | 134 |
| 17 | cnv_DLG3 | cnv | DLG3 | YES | 37 |
| 17 | stv_ANGPTL1 | stv | ANGPTL1 | YES | 5 |
| 18 | cnv_CLECL1 | cnv | CLECL1 | YES | 4 |
| 18 | stv_CTSD | stv | CTSD | YES | 23 |
| 19 | meth_AL021807.1 | meth | AL021807.1 | NO | 0 |
| 19 | mrna_BIRC2 | mrna | BIRC2 | YES | 64 |
| 20 | meth_CYP2D6 | meth | CYP2D6 | YES | 34 |
| 20 | mrna_AGBL5 | mrna | AGBL5 | YES | 19 |
| 21 | mrna_ARID5B | mrna | ARID5B | YES | 34 |
| 21 | stv_STAM2 | stv | STAM2 | YES | 18 |
| 22 | mrna_FNDC3B | mrna | FNDC3B | YES | 4 |
| 22 | mrna_C9orf43 | mrna | C9orf43 | YES | 1 |
| 23 | meth_CUL9 | meth | CUL9 | YES | 13 |
| 23 | meth_FGF22 | meth | FGF22 | YES | 22 |
| 24 | meth_IQCK | meth | IQCK | NO | 0 |
| 24 | mrna _PDE10A | mrna | PDE10A | YES | 24 |
| 25 | mrna_AP000344.4 | mrna | AP000344.4 | NO | 0 |
| 25 | mrna_IQCJ-SCHIP1 | mrna | IQCJ-SCHIP1 | YES | 4 |
| 26 | mrna_OPN1SW | mrna | OPN1SW | YES | 18 |
| 26 | mrna_EXTL2 | mrna | EXTL2 | YES | 18 |
| 27 | mrna_FERMT1 | mrna | FERMT1 | YES | 25 |
| 27 | mrna_CTNNB1 | mrna | CTNNB1 | YES | 260 |
| 28 | meth_DHRS4-AS1 | meth | DHRS4-AS1 | NO | 0 |
| 28 | meth_MGP | meth | MGP | YES | 14 |
| 29 | meth_SSRP1 | meth | SSRP1 | YES | 16 |
| 29 | mrna_ZNF454 | mrna | ZNF454 | YES | 8 |
| 30 | meth_SGCG | meth | SGCG | YES | 15 |
| 30 | mrna_MLX | mrna | MLX | YES | 19 |
| 31 | mrna_SLC16A1 | mrna | SLC16A1 | YES | 30 |
| 32 | meth_TMCO5A | meth | TMCO5A | YES | 2 |
| 33 | meth_HLA-DQB1 | meth | HLA-DQB1 | YES | 31 |
| 33 | mrna_ID4 | mrna | ID4 | YES | 33 |
| 34 | meth_C22orf39 | meth | C22orf39 | YES | 1 |
| 34 | mrna_AMOTL1 | mrna | AMOTL1 | YES | 14 |
| 35 | meth_MAN2B1 | meth | MAN2B1 | YES | 19 |
| 35 | mrna_UGT2B7 | mrna | UGT2B7 | YES | 16 |
| 36 | meth_AC002451.3 | meth | AC002451.3 | NO | 0 |
| 36 | mrna_PLEKHG4B | mrna | PLEKHG4B | YES | 4 |
| 37 | meth_AC126407.1 | meth | AC126407.1 | NO | 0 |
| 37 | meth_WFDC10B | meth | WFDC1OB | YES | 3 |
| 38 | mrna_SH3BP5 | mrna | SH3BP5 | YES | 10 |
| 39 | mrna_CD40 | mrna | CD40 | YES | 63 |
| 39 | mrna_AC072062.1 | mrna | AC072062.1 | NO | 0 |
| 40 | meth_C8orf4 | meth | C8orf4 | YES | 21 |
| 40 | mrna_STK32A | mrna | STK32A | YES | 14 |
| 41 | meth_ARTN | meth | ARTN | YES | 15 |
| 41 | meth_GLYAT | meth | GLYAT | YES | 13 |
| 42 | mrna_SLC25A5 | mrna | SLC25A5 | YES | 29 |
| 42 | mrna_AKAP2 | mrna | AKAP2 | YES | 3 |
| 43 | cnv_SLC25A39 | cnv | SLC25A39 | YES | 9 |
| 43 | meth_AC087651.1 | meth | AC087651.1 | NO | 0 |
| 44 | meth_TDRD3 | meth | TDRD3 | YES | 10 |
| 45 | mrna_MRAP2 | mrna | MRAP2 | YES | 17 |
| 45 | mrna_NCK1-AS1 | mrna | NCK1-AS1 | NO | 0 |
| 46 | meth_FAM206A | meth | FAM206A | YES | 4 |
| 46 | meth_RNF186 | meth | RNF186 | YES | 3 |
| 47 | mirna_MIR455 | mirna | MIR455 | NO | 0 |
| 47 | mrna_TIGD5 | mrna | TIGD5 | YES | 6 |
| 48 | cnv_DEFB110 | cnv | DEFB110 | YES | 5 |
| 48 | mrna_WNK3 | mrna | WNK3 | YES | 29 |
| 49 | cnv_AMD1 | cnv | AMD1 | YES | 11 |
| 49 | meth_CSRP2BP | meth | CSRP2BP | YES | 12 |
| 50 | meth_PRKCE | meth | PRKCE | YES | 71 |
| 50 | mrna_MFHAS1 | mrna | MFHAS1 | YES | 5 |
| 51 | meth_C2orf57 | meth | C2orf57 | NO | 0 |
| 51 | mrna_TNFRSF11B | mrna | TNFRSF11B | YES | 27 |
| 52 | meth_GTSF1L | meth | GTSF1L | YES | 2 |
| 52 | mrna_MUC13 | mrna | MUC13 | YES | 13 |

TABLE E
Top 52 genes from ER+ vs. ER− nGOseq (see full listing in Appendix D)

| Rank | Full_Name | Data_Type | HUGO_GENE | GO_Annotated | Number_Of_GO_Annotations |
|---|---|---|---|---|---|
| 1 | meth_MYO1A | meth | MYO1A | YES | 21 |
| 1 | meth_PCSK4 | meth | PCSK4 | YES | 17 |
| 2 | mrna_MMP9 | mrna | MMP9 | YES | 45 |
| 2 | mrna_LIMK1 | mrna | LIMK1 | YES | 30 |
| 3 | mrna_DNAJC2 | mrna | DNAJC2 | YES | 21 |
| 3 | mrna_GCNT2 | mrna | GCNT2 | YES | 22 |
| 4 | meth_ADIPOQ | meth | ADIPOQ | YES | 92 |
| 4 | stv_ACVR2A | stv | ACVR2A | YES | 53 |
| 5 | mrna_TFDP1 | mrna | TFDP1 | YES | 31 |
| 5 | stv_RNF207 | stv | RNF207 | YES | 16 |
| 6 | mrna_GARS | mrna | GARS | YES | 22 |
| 6 | mrna_MAL | mrna | MAL | YES | 19 |
| 7 | cnv_DEPDC1B | cnv | DEPDC1B | YES | 8 |
| 7 | mrna_ENPP3 | mrna | ENPP3 | YES | 22 |
| 8 | mrna_NMU | mrna | NMU | YES | 20 |
| 8 | stv_TRERF1 | stv | TRERF1 | YES | 24 |
| 9 | meth_COL11A1 | meth | COL11A1 | YES | 29 |
| 9 | meth_DCDC2 | meth | DCDC2 | YES | 20 |
| 10 | meth_IL1RN | meth | !URN | YES | 37 |
| 10 | mrna_DACH1 | mrna | DACH1 | YES | 26 |
| 11 | stv_GRK7 | stv | GRK7 | YES | 17 |
| 11 | stv_PREX1 | stv | PREX1 | YES | 33 |
| 12 | mrna_MYO10 | mrna | MYO10 | YES | 34 |
| 12 | mrna_SHC4 | mrna | SHC4 | YES | 15 |
| 13 | meth_ALDH1A3 | meth | ALDH1A3 | YES | 32 |
| 13 | stv_PLCG2 | stv | PLCG2 | YES | 36 |
| 14 | stv_ANO6 | stv | ANO6 | YES | 52 |
| 14 | stv_CRY1 | stv | CRY1 | YES | 43 |
| 15 | mrna_FTCD | mrna | FTCD | YES | 28 |
| 15 | mrna_SOX11 | mrna | SOX11 | YES | 66 |
| 16 | mrna_DNMT3A | mrna | DNMT3A | YES | 48 |
| 16 | stv_PTPRJ | stv | PTPRJ | YES | 44 |
| 17 | mirna_MIR182 | mirna | MIR182 | YES | 1 |
| 17 | mrna_MSL3 | mrna | MSL3 | YES | 15 |
| 18 | meth_CDX2 | meth | CDX2 | YES | 36 |
| 18 | mrna_RHCG | mrna | RHCG | YES | 20 |
| 19 | mrna_AKR1E2 | mrna | AKR1E2 | YES | 8 |
| 19 | stv_PTTG2 | stv | PTTG2 | YES | 10 |
| 20 | meth_SOSTDC1 | meth | SOSTDC1 | YES | 16 |
| 20 | meth_STOM | meth | STOM | YES | 31 |
| 21 | meth_DDAH2 | meth | DDAH2 | YES | 18 |
| 21 | stv_FRAS1 | stv | FRAS1 | YES | 14 |
| 22 | meth_SEPP1 | meth | SEPP1 | YES | 10 |
| 22 | mrna_VAV3 | mrna | VAV3 | YES | 40 |
| 23 | meth_KAT6B | meth | KAT6B | YES | 24 |
| 23 | mrna_ETV6 | mrna | ETV6 | YES | 25 |
| 24 | cnv_PLB1 | cnv | PLB1 | YES | 16 |
| 24 | stv_MAPK14 | stv | MAPK14 | YES | 92 |
| 25 | meth_PRTN3 | meth | PRTN3 | YES | 20 |
| 25 | stv_NR1H3 | stv | NR1H3 | YES | 58 |
| 26 | meth_ALK | meth | ALK | YES | 37 |
| 26 | mrna_PLOD1 | mrna | PLOD1 | YES | 19 |
| 27 | cnv_RGMB | cnv | RGMB | YES | 12 |
| 27 | mirna_MIR29C | mirna | MIR29C | YES | 17 |
| 28 | meth_KLHL10 | meth | KLHL10 | YES | 12 |
| 28 | mrna_NFE2L3 | mrna | NFE2L3 | YES | 15 |
| 29 | stv_TIMM8A | stv | TIMM8A | YES | 10 |
| 30 | mrna_UGT8 | mrna | UGT8 | YES | 21 |
| 30 | mrna_ABAT | mrna | ABAT | YES | 42 |
| 31 | mrna_BCL11A | mrna | BCL11A | YES | 23 |
| 31 | stv_JAK2 | stv | JAK2 | YES | 123 |
| 32 | cnv_CDK7 | cnv | CDK7 | YES | 47 |
| 32 | meth_MEST | meth | MEST | YES | 8 |
| 33 | mrna_RSU1 | mrna | RSU1 | YES | 7 |
| 33 | stv_LSR | stv | LSR | YES | 13 |
| 34 | cnv_PDGFRB | cnv | PDGFRB | YES | 108 |
| 34 | stv_PLAU | stv | PLAU | YES | 30 |
| 35 | meth_NCKAP1L | meth | NCKAP1L | YES | 49 |
| 35 | mrna_MRPS5 | mrna | MRPS5 | YES | 10 |
| 36 | meth_RNF103 | meth | RNF103 | YES | 14 |
| 36 | mrna_UNC13D | mrna | UNC13D | YES | 25 |
| 37 | meth_LUC7L | meth | LUC7L | YES | 9 |
| 37 | mrna_DKC1 | mrna | DKC1 | YES | 38 |
| 38 | mrna_TMEM25 | mrna | TMEM25 | YES | 5 |
| 38 | stv_RIMS1 | stv | RIMS1 | YES | 37 |
| 39 | meth_CAV1 | meth | CAV1 | YES | 141 |
| 39 | stv_MMP15 | stv | MMP15 | YES | 21 |
| 40 | meth_RNH1 | meth | RNH1 | YES | 10 |
| 41 | mirna_LET7B | mirna | LET7B | NO | 0 |
| 41 | stv_PGF | stv | PGF | YES | 29 |
| 42 | cnv_RAB3C | cnv | RAB3C | YES | 17 |
| 42 | stv_SUPV3L1 | stv | SUPV3L1 | YES | 31 |
| 43 | stv_GRM8 | stv | GRM8 | YES | 16 |
| 43 | stv_TNFAIP3 | stv | TNFAIP3 | YES | 78 |
| 44 | stv_LIN9 | stv | LIN9 | YES | 8 |
| 45 | meth_NEK6 | meth | NEK6 | YES | 44 |
| 45 | stv_ALOX15 | stv | ALOX15 | YES | 43 |
| 46 | mrna_SRPK1 | mrna | SRPK1 | YES | 31 |
| 46 | mrna _RDH10 | mrna | RDH10 | YES | 30 |
| 47 | stv_CA2 | stv | CA2 | YES | 39 |
| 47 | stv_SDHAF2 | stv | SDHAF2 | YES | 12 |
| 48 | cnv_COMMD1 | cnv | COMMD1 | YES | 37 |
| 48 | mrna_GLIPR2 | mrna | GLIPR2 | YES | 9 |
| 49 | cnv_H2AFY | cnv | H2AFY | YES | 49 |
| 49 | mrna_CDC42EP1 | mrna | CDC42EP1 | YES | 17 |
| 50 | mrna_ADORA2B | mrna | ADORA2B | YES | 28 |
| 51 | meth_NR1I2 | meth | NR1I2 | YES | 32 |
| 52 | meth_FSCN1 | meth | FSCN1 | YES | 43 |
| 52 | meth_GPR55 | meth | GPR55 | YES | 16 |

In some embodiments, the therapeutic or drug targets for KTRP and KIRC generated according to the methods, systems, algorithms, programs, and codes described above are set forth in Appendices E and F(full listings) and Tables F(top 57 genes), G(top 53 genes), Table AP (28 genes), AQ (22 genes), AR (3 genes), AS (1 gene), or combinations thereof.

TABLE F
Top 57 genes from MRP vs. KIRC MEGENA (see full listing in Appendix E)

| Rank | Full_Name | Data_Type | HUGO_GENE | GO_Annotated | Number_Of_GO_Annotations |
|---|---|---|---|---|---|
| 1 | meth_CTD-2371O3.3 | meth | CTD-2371O3.3 | NO | 0 |
| 1 | mrna_RP11-59C5.3 | mrna | RP11-59C5.3 | NO | 0 |
| 2 | meth_CDCA4 | meth | CDCA4 | YES | 6 |
| 3 | meth_ACAT1 | meth | ACAT1 | YES | 35 |
| 3 | meth_HK1 | meth | HK1 | YES | 31 |
| 4 | meth_EI24 | meth | EI24 | YES | 13 |
| 5 | meth_FAM84B | meth | FAM84B | YES | 3 |
| 5 | meth_PDC | meth | PDC | YES | 15 |
| 6 | meth_GPATCH3 | meth | GPATCH3 | YES | 2 |
| 6 | meth_RP11-517H2.6 | meth | RP11-517H2.6 | NO | 0 |
| 7 | meth_CCDC141 | meth | CCDC141 | YES | 2 |
| 7 | meth_CCT8 | meth | CCT8 | YES | 37 |
| 8 | meth_METAP1 | meth | METAP1 | YES | 13 |
| 8 | mrna_SLC6A3 | mrna | SLC6A3 | YES | 52 |
| 9 | meth_CCR1 | meth | CCR1 | YES | 38 |
| 9 | meth_SNF8 | meth | SNF8 | YES | 40 |
| 10 | meth_CLCC1 | meth | CLCC1 | YES | 12 |
| 10 | meth_NUP93 | meth | NUP93 | YES | 31 |
| 11 | meth_DENND1B | meth | DENND1B | YES | 16 |
| 11 | mrna_CDON | mrna | CDON | YES | 29 |
| 12 | meth_SETD1A | meth | SETD1A | YES | 32 |
| 12 | meth_USF1 | meth | USF1 | YES | 37 |
| 13 | meth_CCDC79 | meth | CCDC79 | YES | 3 |
| 14 | mrna_SLC5A12 | mrna | SLC5A12 | YES | 15 |
| 15 | meth_ALDH18A1 | meth | ALDH18A1 | YES | 26 |
| 15 | meth_RP11-38C17.1 | meth | RP11-38C17.1 | NO | 0 |
| 16 | meth_NME8 | meth | NME8 | YES | 17 |
| 17 | meth_RACGAP1 | meth | RACGAP1 | YES | 50 |
| 17 | meth_TMEM81 | meth | TMEM81 | YES | 2 |
| 18 | meth_RP11-299J3.8 | meth | RP11-299J3.8 | NO | 0 |
| 19 | meth_BHLHA15 | meth | BHLHA15 | YES | 21 |
| 19 | mirna_MIR124 | mirna | MIR124 | NO | 0 |
| 20 | meth_DNMBP | meth | DNMBP | YES | 13 |
| 20 | mirna_MIR4473 | mirna | MIR4473 | NO | 0 |
| 21 | mrna_HCG4P7 | mrna | HCG4P7 | NO | 0 |
| 21 | mrna_ENPP7P8 | mrna | ENPP7P8 | NO | 0 |
| 22 | meth_FOXJ3 | meth | FOXJ3 | YES | 12 |
| 22 | meth_OPN1SW | meth | OPN1SW | YES | 18 |
| 23 | meth_SNORD38 | meth | SNORD38 | NO | 0 |
| 24 | meth_ACTL7A | meth | ACTL7A | YES | 10 |
| 24 | mrna_RP11-302L19.3 | mrna | RP11-302L19.3 | NO | 0 |
| 25 | meth_CMTM8 | meth | CMTM8 | YES | 13 |
| 25 | meth_SLC19A1 | meth | SLC19A1 | YES | 15 |
| 26 | meth_HAUS3 | meth | HAUS3 | YES | 20 |
| 26 | meth_LCK | meth | LCK | YES | 65 |
| 27 | mrna_CEBPB-AS1 | mrna | CEBPB-AS1 | NO | 0 |
| 28 | cnv_RNA55P349 | cnv | RNA55P349 | NO | 0 |
| 28 | meth_SYCP3 | meth | SYCP3 | YES | 11 |
| 29 | meth_OXT | meth | OXT | YES | 57 |
| 29 | mrna_GABRB3 | mrna | GABRB3 | YES | 34 |
| 30 | meth_PDHA2 | meth | PDHA2 | YES | 17 |
| 30 | meth_TIGD3 | meth | TIGD3 | YES | 3 |
| 31 | mrna_RP11-236L14.2 | mrna | RP11-236L14.2 | NO | 0 |
| 32 | meth_POMP | meth | POMP | YES | 10 |
| 32 | mrna_FBXO17 | mrna | FBXO17 | YES | 6 |
| 33 | meth_IFNA4 | meth | IFNA4 | YES | 22 |
| 33 | mrna_HNRNPD | mrna | HNRNPD | YES | 51 |
| 34 | mrna_NFIC | mrna | NFIC | YES | 17 |
| 35 | meth_RP11-888D10.3 | meth | RP11-888D10.3 | NO | 0 |
| 35 | mrna_TNFRSF10D | mrna | TNFRSF10D | YES | 20 |
| 36 | mrna_SCTR | mrna | SCTR | YES | 14 |
| 36 | mrna_MAPK11 | mrna | MAPK11 | YES | 41 |
| 37 | meth_AF127936.9 | meth | AF127936.9 | NO | 0 |
| 37 | mrna_UPB1 | mrna | UPB1 | YES | 12 |
| 38 | mrna_POLN | mrna | POLN | YES | 17 |
| 38 | stv_SUCO | stv | SUCO | YES | 10 |
| 39 | meth_PCMTD1 | meth | PCMTD1 | YES | 6 |
| 39 | stv_WNT10A | stv | WNT10A | YES | 20 |
| 40 | meth_EIF4G1 | meth | EIF4G1 | YES | 47 |
| 40 | mrna_ZNF395 | mrna | ZNF395 | YES | 11 |
| 41 | meth_FAM126A | meth | FAM126A | YES | 11 |
| 41 | mrna_RP11-348J24.2 | mrna | RP11-348J24.2 | NO | 0 |
| 42 | mrna_RP11-394O4.5 | mrna | RP11-394O4.5 | NO | 0 |
| 43 | cnv_C2orf70 | cnv | C2orf70 | YES | 1 |
| 43 | mrna_SLC16A12 | mrna | SLC16A12 | YES | 4 |
| 44 | meth_QTRT1 | meth | QTRT1 | YES | 16 |
| 44 | meth_TGM3 | meth | TGM3 | YES | 18 |
| 45 | meth_GALNT3 | meth | GALNT3 | YES | 20 |
| 45 | meth_SLC7A6 | meth | SLC7A6 | YES | 17 |
| 46 | meth_ETS1 | meth | ETS1 | YES | 49 |
| 46 | meth_HIVEP1 | meth | HIVEP1 | YES | 19 |
| 47 | meth_ATP2C1 | meth | ATP2C1 | YES | 27 |
| 47 | mrna_MLEC | mrna | MLEC | YES | 11 |
| 48 | meth_FAM217B | meth | FAM217B | YES | 2 |
| 48 | meth_TNFSF13B | meth | TNFSF13B | YES | 25 |
| 49 | mrna_SLC6A19 | mrna | SLC6A19 | YES | 17 |
| 49 | stv_COPS2 | stv | COPS2 | YES | 21 |
| 50 | meth_SLC39A3 | meth | SLC39A3 | YES | 16 |
| 51 | mrna_MUC4 | mrna | MUC4 | YES | 17 |
| 52 | mrna_EFNA1 | mrna | EFNA1 | YES | 40 |
| 53 | meth_MTPN | meth | MTPN | YES | 23 |
| 54 | meth_LINC00311 | meth | LINC00311 | NO | 0 |
| 54 | mrna_SDAD1P1 | mrna | SDAD1P1 | NO | 0 |
| 55 | cnv_U3\|ENSG00000251800.1 | cnv | U3\|ENSG00000251800.1 | NO | 0 |
| 55 | mrna_CTD-2034I21.1 | mrna | CTD-2034I21.1 | NO | 0 |
| 56 | meth_MPG | meth | MPG | YES | 23 |
| 56 | mrna_SEPT5 | mrna | SEP15 | YES | 19 |
| 57 | meth_MZT2A | meth | MZT2A | YES | 8 |
| 57 | meth_RAB1A | meth | RAB1A | YES | 40 |

TABLE G
Top 53 genes from MRP vs. KIRC nGOseq (see full listing in Appendix F)

| Rank | Full_Name | Data_Type | HUGO_GENE | GO_Annotated | Number_OLGO_Annotations |
|---|---|---|---|---|---|
| 1 | meth_BBX | meth | BBX | YES | 7 |
| 1 | meth_CCNT2 | meth | CCNT2 | YES | 30 |
| 2 | meth_CCNE2 | meth | CCNE2 | YES | 19 |
| 2 | meth_NEDD9 | meth | NEDD9 | YES | 23 |
| 3 | meth_ACAD9 | meth | ACAD9 | YES | 12 |
| 3 | meth_TEP1 | meth | TEP1 | YES | 20 |
| 4 | mirna_MIR10B | mirna | MIR10B | YES | 2 |
| 4 | mirna_MIR21 | mirna | MIR21 | YES | 84 |
| 5 | meth_CNGA4 | meth | CNGA4 | YES | 18 |
| 5 | meth_FOXJ3 | meth | FOXJ3 | YES | 12 |
| 6 | mrna_NFATC2 | mrna | NFATC2 | YES | 38 |
| 6 | stv_NRXN3 | stv | NRXN3 | YES | 27 |
| 7 | meth_UBE2Q1 | meth | UBE2Q1 | YES | 17 |
| 7 | mrna_STEAP4 | mrna | STEAP4 | YES | 19 |
| 8 | meth_PPP2R5B | meth | PPP2R5B | YES | 21 |
| 8 | mrna_HRC | mrna | HRC | YES | 27 |
| 9 | meth_B9D2 | meth | B9D2 | YES | 17 |
| 9 | mrna_GMDS | mrna | GMDS | YES | 12 |
| 10 | cnv_TADA3 | cnv | TADA3 | YES | 30 |
| 10 | meth_ANXA2 | meth | ANXA2 | YES | 77 |
| 11 | meth_LMNB1 | meth | LMNB1 | YES | 13 |
| 11 | meth_TOR3A | meth | TOR3A | YES | 7 |
| 12 | meth_ING2 | meth | ING2 | YES | 35 |
| 12 | meth_SCAP | meth | SCAP | YES | 26 |
| 13 | meth_PCBP2 | meth | PCBP2 | YES | 25 |
| 13 | meth_PPIF | meth | PPIF | YES | 33 |
| 14 | meth_NOP56 | meth | NOP56 | YES | 19 |
| 14 | meth_TBCA | meth | TBCA | YES | 13 |
| 15 | cnv_IL17RD | cnv | IL17RD | YES | 12 |
| 15 | meth_FAM134C | meth | FAM134C | YES | 1 |
| 16 | cnv_MBTD1 | cnv | MBTD1 | YES | 8 |
| 16 | meth_SVIL | meth | SVIL | YES | 22 |
| 17 | meth_ANKRA2 | meth | ANKRA2 | YES | 16 |
| 17 | mrna_CD34 | mrna | CD34 | YES | 57 |
| 18 | meth_ABCC2 | meth | ABCC2 | YES | 46 |
| 19 | stv_ARFGEF3 | stv | ARFGEF3 | YES | 11 |
| 19 | stv_TESK1 | stv | TESK1 | YES | 18 |
| 20 | meth_AGFG1 | meth | AGFG1 | YES | 23 |
| 21 | meth_MRPS10 | meth | MRPS10 | YES | 9 |
| 21 | meth_PFKFB4 | meth | PFKFB4 | YES | 16 |
| 22 | meth_CFL2 | meth | CFL2 | YES | 20 |
| 22 | meth_RIC8B | meth | RIC8B | YES | 10 |
| 23 | meth_MYOG | meth | MYOG | YES | 60 |
| 23 | meth_PRKCA | meth | PRKCA | YES | 84 |
| 24 | meth_MANBA | meth | MANBA | YES | 15 |
| 25 | meth_JUN | meth | JUN | YES | 102 |
| 25 | stv_KLHL21 | stv | KLHL21 | YES | 13 |
| 26 | meth_MAP3K7 | meth | MAP3K7 | YES | 56 |
| 26 | stv_FNBP1L | stv | FNBP1L | YES | 23 |
| 27 | meth_MKRN2 | meth | MKRN2 | YES | 8 |
| 27 | stv_MMP16 | stv | MMP16 | YES | 29 |
| 28 | mrna_HILPDA | mrna | HILPDA | YES | 17 |
| 28 | stv_FAM83G | stv | FAM83G | YES | 5 |
| 29 | meth_CREM | meth | CREM | YES | 23 |
| 29 | meth_RAC1 | meth | RAC1 | YES | 87 |
| 30 | meth_GNB3 | meth | GNB3 | YES | 16 |
| 30 | meth_IRX3 | meth | IRX3 | YES | 14 |
| 31 | mrna_ENG | mrna | ENG | YES | 64 |
| 31 | mrna_KCNAB1 | mrna | KCNAB1 | YES | 40 |
| 32 | meth_PAK4 | meth | PAK4 | YES | 34 |
| 32 | mrna_PYGM | mrna | PYGM | YES | 16 |
| 33 | cnv_APOH | cnv | APOH | YES | 31 |
| 33 | mrna_GBP1 | mrna | GBP1 | YES | 31 |
| 34 | meth_DOK2 | meth | DOK2 | YES | 11 |
| 34 | meth_KPNB1 | meth | KPNB1 | YES | 46 |
| 35 | meth_SUCLG1 | meth | SUCLG1 | YES | 21 |
| 36 | meth_TRIM63 | meth | TRIM63 | YES | 22 |
| 36 | mrna_GABPA | mrna | GABPA | YES | 27 |
| 37 | cnv_GNL3 | cnv | GNL3 | YES | 21 |
| 37 | meth_LIN54 | meth | LIN54 | YES | 8 |
| 38 | meth_NME8 | meth | NME8 | YES | 17 |
| 38 | mrna_SEPT4 | mrna | SEPT4 | YES | 32 |
| 39 | mirna_MIR211 | mirna | MIR211 | NO | 0 |
| 40 | mrna_SARAF | mrna | SARAF | YES | 10 |
| 41 | mrna_ST8SIA4 | mrna | ST8SIA4 | YES | 16 |
| 41 | mrna_IFIT3 | mrna | IFIT3 | YES | 14 |
| 42 | meth_IL25 | meth | IL25 | YES | 14 |
| 42 | mrna_RLF | mrna | RLF | YES | 14 |
| 43 | meth_NDUFAB1 | meth | NDUFAB1 | YES | 25 |
| 43 | mrna_TSGA10 | mrna | TSGA10 | YES | 11 |
| 44 | cnv_XYLB | cnv | XYLB | YES | 17 |
| 44 | stv_MET | stv | MET | YES | 50 |
| 45 | meth_NEO1 | meth | NEO1 | YES | 15 |
| 45 | meth_TRIM24 | meth | TRIM24 | YES | 42 |
| 46 | meth_ATM | meth | ATM | YES | 98 |
| 47 | meth_ANXA4 | meth | ANXA4 | YES | 24 |
| 47 | meth_GLOD4 | meth | GLOD4 | YES | 3 |
| 48 | cnv_KCNH8 | cnv | KCNH8 | YES | 19 |
| 48 | stv_PVR | stv | PVR | YES | 32 |
| 49 | cnv_CIDEC | cnv | CIDEC | YES | 14 |
| 49 | meth_ZDHHC8 | meth | ZDHHC8 | YES | 16 |
| 50 | meth_DAND5 | meth | DAND5 | YES | 16 |
| 50 | meth_PADI4 | meth | PADI4 | YES | 27 |
| 51 | meth_CDK5 | meth | CDK5 | YES | 121 |
| 51 | mirna_MIR185 | mirna | MIR185 | YES | 1 |
| 52 | cnv_UBE2Z | cnv | UBE22 | YES | 15 |
| 52 | mrna_NRARP | mrna | NRARP | YES | 13 |
| 53 | mrna_SLC1A4 | mrna | SLC1A4 | YES | 37 |
| 53 | mrna_MIEF2 | mrna | MIEF2 | YES | 9 |

In some embodiments, the therapeutic or drug targets for LUAD and LUSC generated according to the methods, systems, algorithms, programs, and codes described above are set forth in Appendices G and H(full listings) and Tables H (top 50 genes), I (top 50 genes), AAB (25 genes), AAC (14 genes), AAD (3 genes), AAE, or combinations thereof.

TABLE H
Top 50 genes from LUAD vs. LUSC MEGENA (see full listing in Appendix G)

| Rank | Full_Name | Data_Type | HUGO_GENE | GO_Annotated | Number_Of_GO_Annotations |
|---|---|---|---|---|---|
| 1 | meth_NPTX1 | meth | NPTX1 | YES | 14 |
| 1 | mirna_MIR1292 | mirna | MIR1292 | NO | 0 |
| 2 | meth_CTB-129P6.4 | meth | CTB-129P6.4 | NO | 0 |
| 2 | meth_IGFBP4 | meth | IGFBP4 | YES | 23 |
| 3 | meth_CNOT3 | meth | CNOT3 | YES | 18 |
| 3 | meth_KIAA0232 | meth | KIAA0232 | YES | 2 |
| 4 | meth_SETDB1 | meth | SETDB1 | YES | 24 |
| 4 | meth_ZBTB26 | meth | ZBTB26 | YES | 11 |
| 5 | meth_FAIM2 | meth | FAIM2 | YES | 20 |
| 5 | meth_MIR6850 | meth | MIR6850 | NO | 0 |
| 6 | meth_BOD1 | meth | BOD1 | YES | 9 |
| 6 | meth_TCERG1 | meth | TCERG1 | YES | 12 |
| 7 | meth_SLC25A4 | meth | SLC25A4 | YES | 23 |
| 7 | meth_TRMT61B | meth | TRMT61B | YES | 14 |
| 8 | meth_AKIRIN1 | meth | AKIRIN1 | YES | 4 |
| 8 | meth_PPDX | meth | PPDX | YES | 16 |
| 9 | meth_DYNLL1 | meth | DYNLL1 | YES | 52 |
| 9 | meth_TIMELESS | meth | TIMELESS | YES | 28 |
| 10 | meth_ANG | meth | ANG | YES | 49 |
| 10 | meth_FGF9 | meth | FGF9 | YES | 53 |
| 11 | meth_IRF2BP2 | meth | IRF2BP2 | YES | 6 |
| 11 | meth_JUN | meth | JUN | YES | 102 |
| 12 | meth_AC006946.15 | meth | AC006946.15 | NO | 0 |
| 12 | meth_ASRGL1 | meth | ASRGL1 | YES | 10 |
| 13 | meth_UTP18 | meth | UTP18 | YES | 11 |
| 13 | meth_VAMP3 | meth | VAMP3 | YES | 44 |
| 14 | meth_CABIN1 | meth | CABIN1 | YES | 10 |
| 14 | meth_KCNC1 | meth | KCNC1 | YES | 41 |
| 15 | meth_ZFP69B | meth | ZFP69B | YES | 9 |
| 15 | mrna_CLEC17A | mrna | CLEC17A | YES | 7 |
| 16 | meth_SLC44A1 | meth | SLC44A1 | YES | 13 |
| 16 | meth_VAMP1 | meth | VAMP1 | YES | 24 |
| 17 | meth_ETFA | meth | ETFA | YES | 10 |
| 17 | mrna_ZNF695 | mrna | ZNF695 | YES | 6 |
| 18 | meth_CPNE7 | meth | CPNE7 | YES | 11 |
| 18 | meth_TMED9 | meth | TMED9 | YES | 20 |
| 19 | meth_AC140481.8 | meth | AC140481.8 | NO | 0 |
| 19 | meth_CAV1 | meth | CAV1 | YES | 141 |
| 20 | meth_ABALON | meth | ABALON | NO | 0 |
| 20 | meth_CACNG2 | meth | CACNG2 | YES | 32 |
| 21 | meth_C21orf59 | meth | C21orf59 | YES | 4 |
| 21 | meth_MAGEF1 | meth | MAGEF1 | YES | 2 |
| 22 | meth_IDE | meth | IDE | YES | 52 |
| 22 | mrna_RABAC1 | mrna | RABAC1 | YES | 13 |
| 23 | meth_AC015849.12 | meth | AC015849.12 | NO | 0 |
| 23 | meth_SPG11 | meth | SPG11 | YES | 20 |
| 24 | meth_TROVE2 | meth | TROVE2 | YES | 14 |
| 24 | mrna_MECR | mrna | MECR | YES | 11 |
| 25 | meth_PPIL2 | meth | PPIL2 | YES | 18 |
| 25 | meth_RTF1 | meth | RTF1 | YES | 25 |
| 26 | meth_PDCD5 | meth | PDCD5 | YES | 18 |
| 26 | meth_SERTAD3 | meth | SERTAD3 | YES | 7 |
| 27 | meth_ARRDC2 | meth | ARRDC2 | YES | 3 |
| 27 | meth_ZNF414 | meth | ZNF414 | YES | 7 |
| 28 | meth_CLK2 | meth | CLK2 | YES | 23 |
| 28 | meth_EIF4A1 | meth | EIF4A1 | YES | 25 |
| 29 | meth_ITGB4 | meth | ITGB4 | YES | 31 |
| 29 | meth_RNF39 | meth | RNF39 | YES | 6 |
| 30 | meth_AC002310.14 | meth | AC002310.14 | NO | 0 |
| 30 | meth_EIF2AK2 | meth | EIF2AK2 | YES | 53 |
| 31 | meth_PPM1E | meth | PPM1E | YES | 17 |
| 31 | meth_USP31 | meth | USP31 | YES | 9 |
| 32 | meth_ADAT1 | meth | ADAT1 | YES | 7 |
| 32 | meth_CYB5R4 | meth | CYB5R4 | YES | 20 |
| 33 | meth_INTS6 | meth | INTS6 | YES | 9 |
| 33 | mrna_RP11-184M15.1 | mrna | RP11-184M15.1 | NO | 0 |
| 34 | meth_FKBP1A | meth | FKBP1A | YES | 60 |
| 34 | mirna_MIR222 | mirna | MIR222 | YES | 27 |
| 35 | meth_ATG5 | meth | ATG5 | YES | 49 |
| 35 | meth_RTN1 | meth | RTN1 | YES | 7 |
| 36 | meth_KPNA4 | meth | KPNA4 | YES | 17 |
| 36 | mrna_RP11-132F7.2 | mrna | RP11-132F7.2 | NO | 0 |
| 37 | cnv_OR4B1 | cnv | OR4B1 | YES | 13 |
| 37 | meth_MPZL1 | meth | MPZL1 | YES | 10 |
| 38 | meth_CTSC | meth | CTSC | YES | 39 |
| 38 | meth_HIST1H2AE | meth | HIST1H2AE | YES | 9 |
| 39 | meth_ARL4C | meth | ARL4C | YES | 16 |
| 39 | meth_EFCAB7 | meth | EFCAB7 | YES | 9 |
| 40 | meth_CNDP2 | meth | CNDP2 | YES | 16 |
| 40 | mrna_RP4-758J18.2 | mrna | RP4-758J18.2 | NO | 0 |
| 41 | meth_HAX1 | meth | HAX1 | YES | 33 |
| 41 | meth_HIBADH | meth | HIBADH | YES | 13 |
| 42 | meth_CTC-425F1.4 | meth | CTC-425F1.4 | NO | 0 |
| 42 | mirna_MIR151B | mirna | MIR151B | YES | 1 |
| 43 | meth_C5orf30 | meth | C5orf30 | YES | 11 |
| 43 | mrna_C1orf233 | mrna | C1orf233 | YES | 1 |
| 44 | meth_ABI2 | meth | ABI2 | YES | 26 |
| 44 | meth_GPRC5C | meth | GPRC5C | YES | 13 |
| 45 | meth_BYSL | meth | BYSL | YES | 19 |
| 45 | meth_CD164 | meth | CD164 | YES | 19 |
| 46 | meth_RSRC1 | meth | RSRC1 | YES | 11 |
| 46 | meth_TRPS1 | meth | TRPS1 | YES | 28 |
| 47 | meth_LA16c-358B7.4 | meth | LA16c-358B7.4 | NO | 0 |
| 47 | meth_RP11-643M14.1 | meth | RP11-643M14.1 | NO | 0 |
| 48 | meth_EGR4 | meth | EGR4 | YES | 12 |
| 48 | meth_WTAP | meth | VVTAP | YES | 12 |
| 49 | meth_CALCA | meth | CALCA | YES | 64 |
| 49 | meth_EIF2B4 | meth | EIF2B4 | YES | 23 |
| 50 | meth_BOLA1 | meth | BOLA1 | YES | 2 |
| 50 | meth_KCNIP1 | meth | KCNIP1 | YES | 21 |

TABLE I
Top 50 genes from LUAD vs. LUSC nGOseq (see full listing in Appendix H)

| Rank | Full_Name | Data_Type | HUGO_GENE | GO_Annotated | Number_OLGO_Annotations |
|---|---|---|---|---|---|
| 1 | meth_AKTIP | meth | AKTIP | YES | 19 |
| 1 | meth_BFAR | meth | BFAR | YES | 20 |
| 2 | meth_CCAR1 | meth | CCAR1 | YES | 17 |
| 2 | meth_NR2C1 | meth | NR2C1 | YES | 22 |
| 3 | cnv_NCK1 | cnv | NCK1 | YES | 49 |
| 3 | mrna_B4GALT4 | mrna | B4GALT4 | YES | 16 |
| 4 | cnv_ACOX2 | cnv | ACOX2 | YES | 24 |
| 4 | cnv_GHSR | cnv | GHSR | YES | 60 |
| 5 | meth_BLM | meth | BLM | YES | 71 |
| 5 | meth_SGK3 | meth | SGK3 | YES | 29 |
| 6 | cnv_ACTRT3 | cnv | ACTRT3 | YES | 4 |
| 6 | cnv_PLSCR1 | cnv | PLSCR1 | YES | 35 |
| 7 | meth_ITM2B | meth | ITM2B | YES | 19 |
| 7 | mrna_MAGI3 | mrna | MAGI3 | YES | 20 |
| 8 | meth_SDC1 | meth | SDC1 | YES | 39 |
| 8 | meth_TRMT61B | meth | TRMT61B | YES | 14 |
| 9 | meth_SIVA1 | meth | SIVA1 | YES | 16 |
| 9 | meth_TBRG1 | meth | TBRG1 | YES | 8 |
| 10 | cnv_MAP3K13 | cnv | MAP3K13 | YES | 22 |
| 10 | mrna_TBPL1 | mrna | TBPL1 | YES | 17 |
| 11 | meth_MARCH8 | meth | MARCH8 | YES | 16 |
| 11 | meth_TOMM7 | meth | TOMM7 | YES | 18 |
| 12 | cnv_BCHE | cnv | BCHE | YES | 28 |
| 12 | meth_PPIA | meth | PPIA | YES | 36 |
| 13 | cnv_DPPA4 | cnv | DPPA4 | YES | 8 |
| 13 | cnv_SLITRK3 | cnv | SLITRK3 | YES | 5 |
| 14 | cnv_GRM2 | cnv | GRM2 | YES | 26 |
| 14 | meth_TMEM115 | meth | TMEM115 | YES | 18 |
| 15 | cnv_PPP4R2 | cnv | PPP4R2 | YES | 15 |
| 15 | meth _MCM6 | meth | MCM6 | YES | 19 |
| 16 | meth_DCP1A | meth | DCP1A | YES | 19 |
| 16 | meth_MRPL38 | meth | MRPL38 | YES | 7 |
| 17 | cnv_ATP11B | cnv | ATP11B | YES | 27 |
| 17 | mrna_MRPS22 | mrna | MRPS22 | YES | 10 |
| 18 | cnv_SHQ1 | cnv | SHQ1 | YES | 11 |
| 18 | meth_PIGG | meth | PIGG | YES | 14 |
| 19 | meth_H3F3A | meth | H3F3A | YES | 47 |
| 19 | meth_PRKAR2A | meth | PRKAR2A | YES | 31 |
| 20 | meth_GSTK1 | meth | GSTK1 | YES | 18 |
| 20 | meth_JTB | meth | JTB | YES | 19 |
| 21 | meth_PSMC4 | meth | PSMC4 | YES | 49 |
| 21 | meth_TAF5 | meth | TAF5 | YES | 22 |
| 22 | cnv_NDUFB5 | cnv | NDUFB5 | YES | 11 |
| 22 | meth_CDC23 | meth | CDC23 | YES | 22 |
| 23 | meth_CPSF2 | meth | CPSF2 | YES | 15 |
| 23 | meth_RPLP1 | meth | RPLP1 | YES | 21 |
| 24 | meth_EIF4A1 | meth | EIF4A1 | YES | 25 |
| 24 | meth_NAB2 | meth | NAB2 | YES | 16 |
| 25 | cnv_P2RY13 | cnv | P2RY13 | YES | 14 |
| 25 | meth_CLTC | meth | CLTC | YES | 61 |
| 26 | meth_BBC3 | meth | BBC3 | YES | 32 |
| 26 | mirna_MIR139 | mirna | MIR139 | YES | 2 |
| 27 | cnv_PLD1 | cnv | PLD1 | YES | 30 |
| 27 | meth_PARP1 | meth | PARP1 | YES | 87 |
| 28 | meth_BCL6 | meth | BCL6 | YES | 61 |
| 28 | meth_RNF19B | meth | RNF19B | YES | 17 |
| 29 | cnv_MST1R | cnv | MST1R | YES | 33 |
| 29 | meth_STIL | meth | STIL | YES | 24 |
| 30 | meth_PRKCI | meth | PRKCI | YES | 57 |
| 30 | stv_RNF8 | stv | RNF8 | YES | 41 |
| 31 | cnv_CADPS | cnv | CADPS | YES | 20 |
| 31 | cnv_GYG1 | cnv | GYG1 | YES | 16 |
| 32 | cnv_ADPRH | cnv | ADPRH | YES | 8 |
| 33 | cnv_UQCRC1 | cnv | UQCRC1 | YES | 23 |
| 33 | meth_ATP5E | meth | ATP5E | YES | 19 |
| 34 | cnv_CHST2 | cnv | CHST2 | YES | 15 |
| 34 | meth_PDLIM7 | meth | PDLIM7 | YES | 18 |
| 35 | stv_DHX36 | stv | DHX36 | YES | 38 |
| 35 | stv_DTX3L | stv | DTX3L | YES | 17 |
| 36 | meth_E2F8 | meth | E2F8 | YES | 29 |
| 36 | mrna_DVL3 | mrna | DVL3 | YES | 27 |
| 37 | meth_USP5 | meth | USPS | YES | 18 |
| 37 | mrna_CSTA | mrna | CSTA | YES | 21 |
| 38 | meth_EIF3M | meth | EIF3M | YES | 10 |
| 38 | meth_PSME1 | meth | PSME1 | YES | 36 |
| 39 | cnv_PRKCD | cnv | PRKCD | YES | 91 |
| 39 | meth_NSUN4 | meth | NSUN4 | YES | 16 |
| 40 | cnv_RASA2 | cnv | RASA2 | YES | 14 |
| 40 | meth_PTBP1 | meth | PTBP1 | YES | 20 |
| 41 | meth_DAGLB | meth | DAGLB | YES | 14 |
| 41 | meth_USP1 | meth | USP1 | YES | 20 |
| 42 | meth_COG1 | meth | COG1 | YES | 11 |
| 42 | meth_MYDGF | meth | MYDGF | YES | 17 |
| 43 | meth_CD63 | meth | CD63 | YES | 38 |
| 43 | meth_RABIF | meth | RABIF | YES | 12 |
| 44 | meth_NFIL3 | meth | NFIL3 | YES | 17 |
| 44 | meth_PSMA5 | meth | PSMA5 | YES | 44 |
| 45 | meth_CHMP4B | meth | CHMP4B | YES | 46 |
| 45 | meth_RBPJ | meth | RBPJ | YES | 85 |
| 46 | cnv_RAP2B | cnv | RAP2B | YES | 26 |
| 46 | stv_RAC1 | stv | RAC1 | YES | 87 |
| 47 | cnv_MUC4 | cnv | MUC4 | YES | 17 |
| 47 | meth_HRSP12 | meth | HRSP12 | YES | 6 |
| 48 | cnv_POLR2H | cnv | POLR2H | YES | 35 |
| 48 | meth_TAF1B | meth | TAF1B | YES | 22 |
| 49 | cnv_SIAH2 | cnv | SIAH2 | YES | 32 |
| 49 | meth_SPTLC2 | meth | SPTLC2 | YES | 21 |
| 50 | meth_CREBL2 | meth | CREBL2 | YES | 15 |
| 50 | meth_MTIF2 | meth | MTIF2 | YES | 15 |

In some embodiments, the therapeutic or drug targets for Luminal A and Luminal B generated according to the methods, systems, algorithms, programs, and codes described above are set forth in Appendices I and J (full listings) and Tables J (top 51 genes), K (top 51 genes), AAF (32 genes), AAG (17 genes), AAH (3 genes), AAI, or combinations thereof.

TABLE J
Top 51 genes from Luminal A vs. Luminal B MEGENA (see full listing in Appendix I)

| Rank | Full_Name | Data_Type | HUGO_GENE | GO_Annotated | Number_Of_GO_Annotations |
|---|---|---|---|---|---|
| 1 | meth_AC091729.9 | meth | AC091729.9 | NO | 0 |
| 1 | mrna_DPY19L3 | mrna | DPY19L3 | YES | 7 |
| 2 | cnv_C10orf55 | cnv | C10orf55 | NO | 0 |
| 2 | mrna_ANXA8L1 | mrna | ANXA8L1 | YES | 4 |
| 3 | cnv_ZNF91 | cnv | ZNF91 | YES | 11 |
| 3 | meth_POT1 | meth | POT1 | YES | 33 |
| 4 | cnv_LGALS16 | cnv | LGALS16 | YES | 7 |
| 4 | mrna_LAD1 | mrna | LAD1 | YES | 6 |
| 5 | meth_DUS2 | meth | DUS2 | YES | 17 |
| 5 | meth_SAMD12 | meth | SAMD12 | YES | 3 |
| 6 | cnv_EPS8L3 | cnv | EPS8L3 | YES | 2 |
| 6 | cnv_MRPS12 | cnv | MRPS12 | YES | 15 |
| 7 | mrna_GYLTL1B | mrna | GYLTL1B | YES | 4 |
| 7 | mrna_RGMA | mrna | RGMA | YES | 24 |
| 8 | cnv_ZNF644 | cnv | ZNF644 | YES | 6 |
| 8 | mrna_HBP1 | mrna | HBP1 | YES | 10 |
| 9 | cnv_LINC00845 | cnv | LINC00845 | NO | 0 |
| 9 | mrna_DLG1 | mrna | DLG1 | YES | 105 |
| 10 | cnv_DNAJC9 | cnv | DNAJC9 | YES | 10 |
| 10 | cnv_NPFFR1 | cnv | NPFFR1 | YES | 14 |
| 11 | mrna_CCNA2 | mrna | CCNA2 | YES | 37 |
| 11 | mrna_TCF7L1 | mrna | TCF7L1 | YES | 26 |
| 12 | cnv_FAM86HP | cnv | FAM86HP | NO | 0 |
| 12 | meth_THEM4 | meth | THEM4 | YES | 20 |
| 13 | meth_SUCLA2 | meth | SUCLA2 | YES | 17 |
| 13 | mrna_TMEM209 | mrna | TMEM209 | YES | 2 |
| 14 | cnv_MYBPHL | cnv | MYBPHL | YES | 15 |
| 14 | cnv_RNA5SP470 | cnv | RNA5SP470 | NO | 0 |
| 15 | mrna_NEURL3 | mrna | NEURL3 | YES | 4 |
| 15 | mrna_ARMCX2 | mrna | ARMCX2 | YES | 3 |
| 16 | meth_AF235103.1 | meth | AF235103.1 | NO | 0 |
| 16 | mrna_SLC7A10 | mrna | SLC7A10 | YES | 19 |
| 17 | cnv_SARS2 | cnv | SARS2 | YES | 13 |
| 17 | meth_PAEP | meth | PAEP | YES | 11 |
| 18 | mrna_LEPR | mrna | LEPR | YES | 29 |
| 18 | mrna_FABP5 | mrna | FABP5 | YES | 20 |
| 19 | mrna_URI1 | mrna | URI1 | YES | 24 |
| 19 | mrna_ZNF724P | mrna | ZNF724P | YES | 7 |
| 20 | cnv_TGFBR3 | cnv | TGFBR3 | YES | 63 |
| 20 | mrna_COL25A1 | mrna | COL25A1 | YES | 12 |
| 21 | mrna_ACO1 | mrna | ACO1 | YES | 24 |
| 21 | mrna_KTI12 | mrna | KTI12 | YES | 3 |
| 22 | cnv_SLC44A3 | cnv | SLC44A3 | YES | 8 |
| 22 | mrna_PSME4 | mrna | PSME4 | YES | 43 |
| 23 | meth_CCNE2 | meth | CCNE2 | YES | 19 |
| 23 | mrna_ZNF285 | mrna | ZNF285 | YES | 7 |
| 24 | cnv_RBM42 | cnv | RBM42 | YES | 6 |
| 24 | mrna_UBE2M | mrna | UBE2M | YES | 18 |
| 25 | mrna_ELF5 | mrna | ELF5 | YES | 20 |
| 25 | mrna_RP11-58E21.3 | mrna | RP11-58E21.3 | NO | 0 |
| 26 | cnv_SHKBP1 | cnv | SHKBP1 | YES | 4 |
| 26 | mrna_SMO | mrna | SMO | YES | 101 |
| 27 | cnv_LRRC39 | cnv | LRRC39 | YES | 1 |
| 27 | stv_OR1L4 | stv | OR1L4 | YES | 11 |
| 28 | cnv_WDR62 | cnv | WDR62 | YES | 18 |
| 28 | mrna_FAM60A | mrna | FAM60A | YES | 4 |
| 29 | cnv_SNORD74\| | cnv | SNORD74\| | NO | 0 |
| | ENSG00000200897.1 | | ENSG00000200897.1 | | |
| 29 | mrna_ITIH5 | mrna | ITIH5 | YES | 3 |
| 30 | mrna_CRYBG3 | mrna | CRYBG3 | YES | 1 |
| 30 | mrna_SERPINB5 | mrna | SERPINB5 | YES | 12 |
| 31 | mrna_DEPDC4 | mrna | DEPDC4 | YES | 3 |
| 32 | cnv_RAB31 | cnv | RAB31 | YES | 24 |
| 32 | cnv_ZNF260 | cnv | ZNF260 | YES | 11 |
| 33 | mrna_ESF1 | mrna | ESF1 | YES | 7 |
| 33 | mrna_MLXIP | mrna | MLXIP | YES | 16 |
| 34 | cnv_MSS51 | cnv | MSS51 | YES | 2 |
| 34 | mrna_SSBP3 | mrna | SSBP3 | YES | 20 |
| 35 | meth_GPR22 | meth | GPR22 | YES | 11 |
| 35 | mrna_RP11-266K4.9 | mrna | RP11-266K4.9 | NO | 0 |
| 36 | cnv_KIAA1257 | cnv | KIAA1257 | NO | 0 |
| 36 | cnv_ZNF566 | cnv | ZNF566 | YES | 9 |
| 37 | cnv_LYPD4 | cnv | LYPD4 | YES | 5 |
| 37 | mrna_KLF11 | mrna | KLF11 | YES | 22 |
| 38 | cnv_LRFN3 | cnv | LRFN3 | YES | 15 |
| 38 | meth_AGO2 | meth | AGO2 | YES | 65 |
| 39 | cnv_SART3 | cnv | SART3 | YES | 27 |
| 39 | mrna_MON2 | mrna | MON2 | YES | 8 |
| 40 | cnv_SNORA48\| | cnv | SNORA48\| | NO | 0 |
| | ENSG00000212626.1 | | ENSG00000212626.1 | | |
| 40 | meth_CMBL | meth | CMBL | YES | 5 |
| 41 | cnv_UOX | cnv | UOX | NO | 0 |
| 41 | mrna_TMEM123 | mrna | TMEM123 | YES | 7 |
| 42 | cnv_HAMP | cnv | HAMP | YES | 30 |
| 42 | cnv_PBLD | cnv | PBLD | YES | 15 |
| 43 | cnv_CEACAM21 | cnv | CEACAM21 | YES | 2 |
| 44 | cnv_snoU13\| | cnv | snoU13\| | NO | 0 |
| | ENSG00000238983.1 | | ENSG00000238983.1 | | |
| 44 | mrna_GYG2 | mrna | GYG2 | YES | 8 |
| 45 | cnv_LINC00662 | cnv | LINC00662 | NO | 0 |
| 45 | meth_MXRA7 | meth | MXRA7 | YES | 2 |
| 46 | cnv_EFCAB12 | cnv | EFCAB12 | YES | 3 |
| 46 | cnv_RPL32P3 | cnv | RPL32P3 | NO | 0 |
| 47 | cnv_RNA5SP53 | cnv | RNA5SP53 | NO | 0 |
| 47 | mrna_CTC-459F4.1 | mrna | CTC-459F4.1 | NO | 0 |
| 48 | cnv_HPN | cnv | HPN | YES | 36 |
| 48 | cnv_MTF2 | cnv | MTF2 | YES | 18 |
| 49 | mrna_AMER1 | mrna | AMER1 | YES | 26 |
| 49 | stv_RPL28 | stv | RPL28 | YES | 21 |
| 50 | mrna_PISD | mrna | PISD | YES | 13 |
| 51 | mrna_GLCE | mrna | GLCE | YES | 12 |
| 51 | stv_TRIM6 | stv | TRIM6 | YES | 32 |

TABLE K
Top 51 genes from Luminal A vs. Luminal B nGOseq (see full listing in Appendix J)

| Rank | Full_Name | Data_Type | HUGO_GENE | GO_Annotated | Number_OLGO_Annotations |
|---|---|---|---|---|---|
| 1 | mrna_CX3CR1 | mrna | CX3CR1 | YES | 37 |
| 1 | stv_CERCAM | stv | CERCAM | YES | 6 |
| 2 | mrna_CENPL | mrna | CENPL | YES | 8 |
| 2 | mrna_KIF15 | mrna | KIF15 | YES | 21 |
| 3 | cnv_FREM1 | cnv | FREM1 | YES | 11 |
| 3 | mrna_LIM52 | mrna | LIM52 | YES | 15 |
| 4 | cnv_KCNH6 | cnv | KCNH6 | YES | 16 |
| 4 | mrna_CEP131 | mrna | CEP131 | YES | 32 |
| 5 | meth_HYOU1 | meth | HYOU1 | YES | 21 |
| 5 | meth_UTS2 | meth | UTS2 | YES | 30 |
| 6 | cnv_C1QTNF1 | cnv | C1QTNF1 | YES | 18 |
| 6 | mrna_CASC5 | mrna | CASC5 | YES | 15 |
| 7 | meth_HPDL | meth | HPDL | YES | 6 |
| 7 | meth_KCNK9 | meth | KCNK9 | YES | 16 |
| 8 | cnv_MPZL3 | cnv | MPZL3 | YES | 6 |
| 8 | meth_LEP | meth | LEP | YES | 112 |
| 9 | mirna_MIR191 | mirna | MIR191 | YES | 2 |
| 9 | stv_GAP43 | stv | GAP43 | YES | 29 |
| 10 | meth_GPX7 | meth | GPX7 | YES | 12 |
| 10 | mrna_PTPN21 | mrna | PTPN21 | YES | 10 |
| 11 | meth_DAND5 | meth | DAND5 | YES | 16 |
| 11 | mrna_INSIG1 | mrna | INSIG1 | YES | 24 |
| 12 | mrna_TXNRD1 | mrna | TXNRD1 | YES | 44 |
| 12 | stv_NUFIP1 | stv | NUFIP1 | YES | 23 |
| 13 | mrna_ORC6 | mrna | ORC6 | YES | 13 |
| 13 | mrna_GRIN2A | mrna | GRIN2A | YES | 66 |
| 14 | mrna_LARP1 | mrna | LARP1 | YES | 27 |
| 14 | mrna_HTR1F | mrna | HTR1F | YES | 15 |
| 15 | cnv_ORAOV1 | cnv | ORAOV1 | YES | 10 |
| 15 | stv_PRICKLE2 | stv | PRICKLE2 | YES | 8 |
| 16 | mrna_TP63 | mrna | TP63 | YES | 103 |
| 16 | mrna_KIF18B | mrna | KIF18B | YES | 25 |
| 17 | meth_EREG | meth | EREG | YES | 59 |
| 17 | mrna_DPP3 | mrna | DPP3 | YES | 14 |
| 18 | meth_PLG | meth | PLG | YES | 48 |
| 18 | meth_STX1B | meth | STX1B | YES | 42 |
| 19 | cnv_ASPSCR1 | cnv | ASPSCR1 | YES | 21 |
| 19 | mrna_PCNA | mrna | PCNA | YES | 65 |
| 20 | cnv_NUP85 | cnv | NUP85 | YES | 36 |
| 20 | meth_FCRL4 | meth | FCRL4 | YES | 7 |
| 21 | cnv_APC2 | cnv | APC2 | YES | 20 |
| 21 | mrna_STRBP | mrna | STRBP | YES | 13 |
| 22 | meth_FAM20A | meth | FAM20A | YES | 14 |
| 22 | meth_TSC1 | meth | TSC1 | YES | 63 |
| 23 | cnv_POLRMT | cnv | POLRMT | YES | 15 |
| 23 | meth_ATM | meth | ATM | YES | 98 |
| 24 | cnv_SGTA | cnv | SGTA | YES | 13 |
| 24 | mrna_WDHD1 | mrna | WDHD1 | YES | 12 |
| 25 | meth_KLK4 | meth | KLK4 | YES | 12 |
| 25 | meth_KRT16 | meth | KRT16 | YES | 23 |
| 26 | mrna_MKI67 | mrna | MKI67 | YES | 25 |
| 26 | stv_PLK4 | stv | PLK4 | YES | 27 |
| 27 | mrna_LMNB1 | mrna | LMNB1 | YES | 13 |
| 27 | mrna_PIWIL2 | mrna | PIWIL2 | YES | 35 |
| 28 | mrna_DIAPH3 | mrna | DIAPH3 | YES | 9 |
| 28 | mrna_HPGD | mrna | HPGD | YES | 32 |
| 29 | cnv_JMJD6 | cnv | JMJD6 | YES | 44 |
| 29 | mrna_DMBX1 | mrna | DMBX1 | YES | 19 |
| 30 | cnv_RGS9 | cnv | RGS9 | YES | 22 |
| 30 | stv_C5AR1 | stv | C5AR1 | YES | 46 |
| 31 | cnv_ADRM1 | cnv | ADRM1 | YES | 19 |
| 31 | meth_PADI4 | meth | PADI4 | YES | 27 |
| 32 | mrna_CENPN | mrna | CENPN | YES | 12 |
| 32 | stv_SRRM4 | stv | SRRM4 | YES | 10 |
| 33 | meth_NPR3 | meth | NPR3 | YES | 26 |
| 33 | meth_ZFP41 | meth | ZFP41 | YES | 8 |
| 34 | mrna_HELLS | mrna | HELLS | YES | 24 |
| 35 | cnv_KDM4C | cnv | KDM4C | YES | 29 |
| 35 | mrna_DACT3 | mrna | DACT3 | YES | 12 |
| 36 | meth_TNFRSF18 | meth | TNFRSF18 | YES | 22 |
| 36 | mrna_CENPK | mrna | CENPK | YES | 10 |
| 37 | cnv_EOGT | cnv | EOGT | YES | 9 |
| 37 | mrna_BLM | mrna | BLM | YES | 71 |
| 38 | cnv_ARCN1 | cnv | ARCN1 | YES | 23 |
| 38 | stv_ADGRL2 | stv | ADGRL2 | YES | 10 |
| 39 | cnv_PPP6R3 | cnv | PPP6R3 | YES | 11 |
| 39 | meth_ACTR5 | meth | ACTR5 | YES | 13 |
| 40 | meth_SEC61A2 | meth | SEC61A2 | YES | 11 |
| 40 | mrna_GBGT1 | mrna | GBGT1 | YES | 14 |
| 41 | cnv_IL33 | cnv | IL33 | YES | 34 |
| 41 | meth_XCR1 | meth | XCR1 | YES | 14 |
| 42 | cnv_TAF1D | cnv | TAF1D | YES | 12 |
| 42 | meth_DZIP1 | meth | DZIP1 | YES | 23 |
| 43 | meth_MNX1 | meth | MNX1 | YES | 23 |
| 43 | stv_GPC3 | stv | GPC3 | YES | 51 |
| 44 | mrna_KIF14 | mrna | KIF14 | YES | 51 |
| 44 | stv_GTF3C4 | stv | GTF3C4 | YES | 17 |
| 45 | meth_NT5C1A | meth | NT5C1A | YES | 15 |
| 45 | mrna_NME1 | mrna | NME1 | YES | 59 |
| 46 | cnv_IFNA14 | cnv | IFNA14 | YES | 20 |
| 46 | stv_NFKBIZ | stv | NFKBIZ | YES | 9 |
| 47 | cnv_LPAR3 | cnv | LPAR3 | YES | 22 |
| 47 | cnv_TBRG1 | cnv | TBRG1 | YES | 8 |
| 48 | mrna_LGR6 | mrna | LGR6 | YES | 17 |
| 48 | stv_SORCS1 | stv | SORCS1 | YES | 5 |
| 49 | meth_AVPR1B | meth | AVPR1B | YES | 18 |
| 49 | meth_B3GNT5 | meth | B3GNT5 | YES | 17 |
| 50 | cnv_BIRC5 | cnv | BIRC5 | YES | 50 |
| 51 | cnv_RYBP | cnv | RYBP | YES | 14 |
| 51 | mrna_RASL11A | mrna | RASL11A | YES | 12 |

In some embodiments, the KIRC vs. KIRP enriched genes with no association with cancer or other genes in published literature are set forth in Table AP and Table AR. In some embodiments, the KIRC vs. KTRP enriched genes with no associated functional annotations are set forth in Table AQ and Table AS.

TABLE AP
KIRC vs. MRP enriched genes
(MEGENA) with no association
with cancer or other genes in
published literature

Genes
C2orf70
CCDC79
FAM217B
AF127936.9
CEBPB-AS1
CTD-2034I21.1
CTD-2371O3.3
ENPP7P8
HCG4P7
LINC00311
MIR124-3
MIR4473
RNA5SP349
RP11-236L14.2
RP11-299J3.8
RP11-302L19.3
RP11-348J24.2
RP11-38C17.1
RP11-394O4.5
RP11-517H2.6
RP11-59C5.3
RP11-888D10.3
SDAD1P1
SNORD38
MZT2A
QTRT1
TIGD3
TMEM81

TABLE AQ
KIRC vs. KIRP enriched genes
(MEGENA) with no associated
functional annotations

Genes
AF127936.9
CEBPB-AS1
CTD-2034I21.1
CTD-2371O3.3
ENPP7P8
HCG4P7
LINC00311
MIR124-3
MIR4473
RNA5SP349
RP11-236L14.2
RP11-299J3.8
RP11-302L19.3
RP11-348J24.2
RP11-38C17.1
RP11-394O4.5
RP11-517H2.6
RP11-59C5.3
RP11-888D10.3
SDAD1P1
SNORD38
U3|ENSG00000251800.1

TABLE AR
KIRC vs. MRP enriched
genes (nGOseq) with no
association with cancer
orother genes in published
literature

Genes
ACAD9
B9D2
FAM134C

TABLE AS
KIRC vs. KIRP enriched genes
(nGOseq) with no associated
functional annotations

Genes
MIR211

In some embodiments, the BRCA vs. normal enriched genes with no association with cancer or other genes in published literature are set forth in Table AT and Table AV. In some embodiments, the BRCA vs. normal enriched genes with no associated functional annotations are set forth in Table AU.

TABLE AT
BRCA vs. Normal enriched genes
(MEGENA) with no association
with cancer or other genes in
published literature

Genes
ABHD10
ANKMY2
AVPI1
C19orf70
C6orf203
CACHD1
EFR3B
EXOC3L1
FAM35A
GS1-124K5.11
LINC00996
LOC101928580
LOC101929268
MAP3K14-AS1
MIR3940
MIR4738
MIR676
PP14571
RP5-1065J22.8
TMCO5B
TOB1-AS1
ZC4H2
ZPLD1

TABLE AU
BRCA vs. Normal enriched genes
(MEGENA) with no associated
functional annotations

Genes
FAM35A
GS1-124K5.11
LINC00996
LOC101928580
LOC101929268
MAP3K14-AS1
MIR3940
MIR4738
MIR676
MTVR2
PP14571
RP5-1065J22.8
TMCO5B
TOB1-AS1

TABLE AV
BRCA vs. Normal enriched genes
(nGOseq) with no association with
cancer or other genes in published
literature

genes
ARL8A
GCSAML
OR10J1
OR7C2
TMED2

In some embodiments, the ER+vs ER− enriched genes with no association with cancer or other genes in published literature are set forth in Table AX and Table AZ. In some embodiments, the ER+vs ER− enriched genes with no associated functional annotations are set forth in Table AY and Table AAA.

TABLE AX
ER+ vs. ER− enriched genes
(MEGENA) with no association
with cancer or other genes in
published literature

genes
C22orf39
C8orf4
C9orf43
CLECL1
CSRP2BP
AC002451.3
AC072062.1
AC087651.1
AC126407.1
AL021807.1
AP000344.4
C2orf57
C6orf48
DHRS4-AS1
ILF3-AS1
IQCK
MIR455
NCK1-AS1
PLA2G4E-AS1
RP11-1081L13.4
RPS7P1
SNORD116-1
FAM206A
GTSF1L
IGKV1-16
IQCJ-SCHIP1
NOSIP
PLEKHG4B
RNF186
SLC25A39
SLC37A3
WFDC1OB

TABLE AY
ER+ vs. ER− enriched genes
(MEGENA) with no associated
functional annotations

genes
AC002451.3
AC072062.1
AC087651.1
AC126407.1
AL021807.1
AP000344.4
C2orf57
C6orf48
DHRS4-AS1
ILF3-AS1
IQCK
MIR455
NCK1-AS1
PLA2G4E-AS1
RP11-1081L13.4
RPS7P1
SNORD116-1

TABLE AZ
ER+ vs. ER− enriched genes
(nGOseq) with no association
with cancer or other genes in
published literature

genes
KLHL 1 0

TABLE AAA
ER+ vs. ER− enriched genes
(nGOseq) with no associated
functional annotations

genes
LET7B
MIRLET7B

In some embodiments, the LUAD vs. LUSC enriched genes with no association with cancer or other genes in published literature are set forth in Table AAB and Table AAD. In some embodiments, the LUAD vs. LUSC enriched genes with no associated functional annotations are set forth in Table AAC.

TABLE AAB
LUAD vs. LUSC enriched genes
(MEGENA) with no association
withcancer or other genes in
published literature

genes
ADAT1
ARRDC2
BOLA1
C1orf233
C21orf59
C5orf30
CYB5R4
EFCAB7
AC002310.14
AC006946.15
AC015849.12
AC140481.8
CTB-129P6.4
CTC-425F1.4
LA16c-358B7.4
MIR1292
RP11-132F7.2
RP11-184M15.1
RP11-643M14.1
RP4-758J18.2
KIAA0232
MIR151B
OR4B1
RNF39
ZFP69B

TABLE AAC
LUAD vs. LUSC enriched genes
(MEGENA) with no associated
functional annotations

genes
ABALON
AC002310.14
AC006946.15
AC015849.12
AC140481.8
CTB-129P6.4
CTC-425F1.4
LA16c-358B7.4
MIR1292
MIR6850
RP11-132F7.2
RP11-184M15.1
RP11-643M14.1
RP4-758J18.2

TABLE AAD
LUAD vs. LUSC enriched genes (nGOseq) with no
association with cancer or other genes in published literature
genes

HRSP12
MIR139
MTIF2

In some embodiments, the Luminal A vs. Luminal B enriched genes with no association with cancer or other genes in published literature are set forth in Table AAF and Table AAH. In some embodiments, the Luminal A vs. Luminal B enriched genes with no associated functional annotations are set forth in Table AAG.

TABLE AAF
Luminal A vs. Luminal B enriched genes (MEGENA) with no
association with cancer or other genes in published literature
genes

CRYBG3
DEPDC4
EFCAB12
ESF1
GYG2
KTI12
AC091729.9
AF235103.1
C10orf55
CTC-459F4.1
FAM86HP
KIAA1257
LINC00662
LINC00845
RNA5SP470
RNA5SP53
RP11-266K4.9
RP11-58E21.3
RPL32P3
SNORA48|ENSG00000212626.1
snoU13|ENSG00000238983.1
LGALS16
LRRC39
LYPD4
MXRA7
MYBPHL
NEURL3
OR1L4
RBM42
TRIM6
ZNF285
ZNF724P

TABLE AAG
Luminal A vs. Luminal B enriched genes (MEGENA)
with no associated functional annotations
genes

AC091729.9
AF235103.1
C10orf55
CTC-459F4.1
FAM86HP
KIAA1257
LINC00662
LINC00845
RNA5SP470
RNA5SP53
RP11-266K4.9
RP11-58E21.3
RPL32P3
SNORA48|ENSG00000212626.1
SNORD74|ENSG00000200897.1
snoU13|ENSG00000238983.1
UOX

TABLE AAH
Luminal A vs. Luminal B enriched genes (nGOseq) with
no association with cancer or other genes in published literature
genes

CERCAM
MPZL3
ZFP41

As used herein “therapeutic agent” refers to a drug or therapeutic composition or compound identified from, but not limited to, DrugBank and Pharmacodia as associated with the therapeutic or drug targets or genes set forth in Tables B-O and Appendices A-N. In some embodiments, the therapeutic agents for BRCA as used herein are set forth in Tables P, Q, AC, AD, or combinations thereof. In some embodiments, the therapeutic agents for ER positive or ER negative as used herein are set forth in Tables R, S, AE, AF, or combinations thereof. In some embodiments, the therapeutic agents for KIRP or KIRC as used herein are set forth in Tables T, U, AG, AH, or combinations thereof. In some embodiments, the therapeutic agents for LUAD or LUSC as used herein are set forth in Tables V, W, A, AJ, or combinations thereof. In some embodiments, the therapeutic agents for Luminal A or Luminal B as used herein are set forth in Tables X, Y, AK, AL, or combinations thereof. In some embodiments, the therapeutic agents for pan-cancer (e.g., the cancers listed in Table A) as used herein are set forth in Tables Z, AA, AB, AM, AN, AO, or combinations thereof.

TABLE P
DrugBank drug targets for BRCA vs Normal using MEGENA

| Gene | Drug Name | Groups |
|---|---|---|
| ACADS | Flavin adenine dinucleotide | Approved |
| CXCL8 | ABT-510 | Investigational |
| NQO1 | Cisplatin | Approved |
| NQO1 | Oxaliplatin | Approved, Investigational |
| NQO1 | Carboplatin | Approved |
| NQO1 | Doxorubicin | Approved, Investigational |
| NQO1 | Flavin adenine dinucleotide | Approved |
| PPAT | Fluorouracil | Approved |
| PPAT | Mercaptopurine | Approved |
| TLR8 | Imiquimod | Approved, Investigational |

TABLE Q
DrugBank drug targets for BRCA vs Normal using nGOseq

| Gene | Drug Name | Groups |
|---|---|---|
| ATF6 | Pseudoephedrine | Approved |
| AURKB | HESPERIDIN | Experimental |
| AURKB | AT9283 | Investigational |
| CD247 | Muromonab | Approved, Investigational |
| DDR2 | Regorafenib | Approved |
| DRD2 | Amphetamine | Approved, Illicit |
| DRD2 | Ziprasidone | Approved |
| DRD2 | Cabergoline | Approved |
| DRD2 | Ropinirole | Approved, Investigational |
| DRD2 | Olanzapine | Approved, Investigational |
| DRD2 | Clozapine | Approved |
| DRD2 | Mirtazapine | Approved |
| DRD2 | Sulpiride | Approved |
| DRD2 | Loxapine | Approved |
| DRD2 | Pramipexole | Approved, Investigational |
| DRD2 | Prochlorperazine | Approved, Vet Approved |
| DRD2 | Droperidol | Approved, Vet Approved |
| DRD2 | Imipramine | Approved |
| DRD2 | Chlorpromazine | Approved, Vet Approved |
| DRD2 | Buspirone | Approved, Investigational |
| DRD2 | Haloperidol | Approved |
| DRD2 | Nortriptyline | Approved |
| DRD2 | Cinnarizine | Approved |
| DRD2 | Lisuride | Approved |
| DRD2 | Fluphenazine | Approved |
| DRD2 | Thioridazine | Withdrawn |
| DRD2 | Ergotamine | Approved |
| DRD2 | Apomorphine | Approved, Investigational |
| DRD2 | Trimipramine | Approved |
| DRD2 | Risperidone | Approved, Investigational |
| DRD2 | Trifluoperazine | Approved |
| DRD2 | Perphenazine | Approved |
| DRD2 | Flupentixol | Approved, Withdrawn |
| DRD2 | Amantadine | Approved |
| DRD2 | Mesoridazine | Approved |
| DRD2 | Maprotiline | Approved |
| DRD2 | Dopamine | Approved |
| DRD2 | Memantine | Approved, Investigational |
| DRD2 | Ergoloid mesylate | Approved |
| DRD2 | Promethazine | Approved |
| DRD2 | Pimozide | Approved |
| DRD2 | Doxepin | Approved |
| DRD2 | Desipramine | Approved |
| DRD2 | Domperidone | Approved, Investigational, Vet |
| | | Approved |
| DRD2 | Pergolide | Approved, Vet Approved, Withdrawn |
| DRD2 | Bromocriptine | Approved, Investigational |
| DRD2 | Ketamine | Approved, Vet Approved |
| DRD2 | Quetiapine | Approved |
| DRD2 | Metoclopramide | Approved, Investigational |
| DRD2 | Levodopa | Approved |
| DRD2 | Aripiprazole | Approved, Investigational |
| DRD2 | Chlorprothixene | Approved, Withdrawn |
| DRD2 | Paliperidone | Approved |
| DRD2 | Yohimbine | Approved, Vet Approved |
| DRD2 | Methotrimeprazine | Approved |
| DRD2 | Molindone | Approved |
| DRD2 | Pipotiazine | Approved |
| DRD2 | Thioproperazine | Approved |
| DRD2 | Thiothixene | Approved |
| DRD2 | Zuclopenthixol | Approved, Investigational |
| DRD2 | Fluspirilene | Approved |
| DRD2 | Tetrabenazine | Approved |
| DRD2 | Bifeprunox | Investigational |
| DRD2 | Bicifadine | Investigational |
| DRD2 | Itopride | Investigational |
| DRD2 | Iloperidone | Approved |
| DRD2 | Rotigotine | Approved |
| DRD2 | Pimavanserin | Investigational |
| DRD2 | BL-1020 | Investigational |
| DRD2 | ACP-104 | Investigational |
| DRD2 | Cariprazine | Approved |
| DRD2 | Lumateperone | Investigational |
| DRD2 | Sertindole | Approved, Withdrawn |
| DRD2 | Mianserin | Approved |
| DRD2 | Asenapine | Approved |
| DRD2 | Amisulpride | Approved, Investigational |
| DRD2 | Lurasidone | Approved |
| DRD2 | Bromopride | Approved |
| DRD2 | Brexpiprazole | Approved |
| DRD2 | Tiapride | Approved, Investigational |
| ITK | Pazopanib | Approved |
| MAP2K2 | Bosutinib | Approved |
| MAP2K2 | Trametinib | Approved |

TABLE R
DrugBank drug targets for ER+ vs. ER− using MEGENA

| Gene | Drug Name | Groups |
|---|---|---|
| CYP2D6 | Peginterferon | Approved |
| | alfa-2b | |
| CYP2D6 | Cyclosporine | Approved, Investigational, Vet |
| | | Approved |
| CYP2D6 | Pravastatin | Approved |
| CYP2D6 | Fluvoxamine | Approved, Investigational |
| CYP2D6 | Amphetamine | Approved, Illicit |
| CYP2D6 | Nicotine | Approved |
| CYP2D6 | Cevimeline | Approved |
| CYP2D6 | Bortezomib | Approved, Investigational |
| CYP2D6 | Phentermine | Approved, Illicit |
| CYP2D6 | Tramadol | Approved, Investigational |
| CYP2D6 | Betaxolol | Approved |
| CYP2D6 | Sildenafil | Approved, Investigational |
| CYP2D6 | Pyrimethamine | Approved, Vet Approved |
| CYP2D6 | Ticlopidine | Approved |
| CYP2D6 | Trospium | Approved |
| CYP2D6 | Midodrine | Approved |
| CYP2D6 | Citalopram | Approved |
| CYP2D6 | Eletriptan | Approved, Investigational |
| CYP2D6 | Nelfinavir | Approved |
| CYP2D6 | Indinavir | Approved |
| CYP2D6 | Lovastatin | Approved, Investigational |
| CYP2D6 | Reboxetine | Approved, Investigational |
| CYP2D6 | Nevirapine | Approved |
| CYP2D6 | Ranolazine | Approved, Investigational |
| CYP2D6 | Benzatropine | Approved |
| CYP2D6 | Ziprasidone | Approved |
| CYP2D6 | Clotrimazole | Approved, Vet Approved |
| CYP2D6 | Sulfanilamide | Approved |
| CYP2D6 | Metoprolol | Approved, Investigational |
| CYP2D6 | Ropinirole | Approved, Investigational |
| CYP2D6 | Amsacrine | Approved |
| CYP2D6 | Theophylline | Approved |
| CYP2D6 | Lidocaine | Approved, Vet Approved |
| CYP2D6 | Clemastine | Approved |
| CYP2D6 | Venlafaxine | Approved |
| CYP2D6 | Atomoxetine | Approved |
| CYP2D6 | Morphine | Approved, Investigational |
| CYP2D6 | Ropivacaine | Approved |
| CYP2D6 | Bupivacaine | Approved, Investigational |
| LYN | Bosutinib | Approved |
| LYN | Ponatinib | Approved |
| LYN | Nintedanib | Approved |
| PDE10A | Dipyridamole | Approved |
| PDE10A | Papaverine | Approved |
| PDE10A | Triflusal | Approved |
| PRKCE | Tamoxifen | Approved |
| SLC16A1 | Pravastatin | Approved |
| SLC16A1 | Valproic Acid | Approved, Investigational |
| SLC16A1 | Aminohippuric | Approved |
| | acid | |
| SLC16A1 | Ampicillin | Approved, Vet Approved |
| SLC16A1 | Foscarnet | Approved |
| SLC16A1 | Methotrexate | Approved |
| SLC16A1 | Nateglinide | Approved, Investigational |
| SLC16A1 | Salicylic acid | Approved, Vet Approved |
| SLC16A1 | Probenecid | Approved |
| SLC16A1 | Gamma Hydroxy- | Approved, Illicit |
| | butyric Acid | |
| SLC16A1 | Acetic acid | Approved |
| SLC16A1 | Benzoic Acid | Approved |
| SLC16A1 | Quercetin | Experimental |
| SLC16A1 | Lactic Acid | Approved, Vet Approved |
| SLC16A1 | Arbaclofen | Investigational |
| | Placarbil | |
| SLC25A5 | Clodronic Acid | Approved, Investigational, Vet |
| | | Approved |
| UGT2B7 | Troglitazone | Withdrawn |
| UGT2B7 | Lovastatin | Approved, Investigational |
| UGT2B7 | Morphine | Approved, Investigational |
| UGT2B7 | Valproic Acid | Approved, Investigational |
| UGT2B7 | Codeine | Approved, Illicit |
| UGT2B7 | Indomethacin | Approved, Investigational |
| UGT2B7 | Epirubicin | Approved |
| UGT2B7 | Zidovudine | Approved |
| UGT2B7 | Carbamazepine | Approved, Investigational |
| UGT2B7 | Diclofenac | Approved, Vet Approved |
| UGT2B7 | Simvastatin | Approved |
| UGT2B7 | Losartan | Approved |
| UGT2B7 | Mycophenolate | Approved, Investigational |
| | mofetil | |
| UGT2B7 | Flurbiprofen | Approved, Investigational |
| UGT2B7 | Etodolac | Approved, Investigational, Vet |
| | | Approved |
| UGT2B7 | Naproxen | Approved, Vet Approved |
| UGT2B7 | Oxazepam | Approved |
| UGT2B7 | Ezetimibe | Approved |
| UGT2B7 | Mycophenolic | Approved |
| | acid | |
| UGT2B7 | Ibuprofen | Approved |
| UGT2B7 | Atorvastatin | Approved |

TABLE S
DrugBank drug targets for ER+ vs. ER− using nGOseq

| Gene | Drug Name | Groups |
|---|---|---|
| ABAT | Valproic Acid | Approved, Investigational |
| ABAT | Phenelzine | Approved |
| ABAT | Vigabatrin | Approved |
| ADORA2B | Theophylline | Approved |
| ADORA2B | Adenosine | Approved, Investigational |
| ADORA2B | Enprofylline | Approved |
| ADORA2B | Defibrotide | Approved, Investigational |
| CA2 | Topiramate | Approved |
| CA2 | Bendroflumethiazide | Approved |
| CA2 | Furosemide | Approved, Vet Approved |
| CA2 | Methazolamide | Approved |
| CA2 | Hydroflumethiazide | Approved |
| CA2 | Acetazolamide | Approved, Vet Approved |
| CA2 | Dorzolamide | Approved |
| CA2 | Chlorothiazide | Approved, Vet Approved |
| CA2 | Zonisamide | Approved, Investigational |
| CA2 | Hydrochlorothiazide | Approved, Vet Approved |
| CA2 | Diazoxide | Approved |
| CA2 | Diclofenamide | Approved |
| CA2 | Brinzolamide | Approved |
| CA2 | Ellagic Acid | Investigational |
| CDK7 | Alvocidib | Experimental, Investigational |
| IL1RN | Rilonacept | Approved |
| JAK2 | XL019 | Investigational |
| JAK2 | Ruxolitinib | Approved |
| JAK2 | Tofacitinib | Approved, Investigational |
| LIMK1 | Dabrafenib | Approved |
| MAPK14 | 1-(5-Tert-Butyl-2-P- | Experimental |
| | Tolyl-2h-Pyrazol-3- | |
| | Yl)-3-[4-(2-Morpholin- | |
| | 4-Yl-Ethoxy)-Naphthalen- | |
| | 1-Yl]-Urea | |
| MAPK14 | KC706 | Investigational |
| MAPK14 | Talmapimod | Investigational |
| MAPK14 | VX-702 | Investigational |
| MMP15 | Marimastat | Approved, Investigational |
| MMP9 | Marimastat | Approved, Investigational |
| MMP9 | Minocycline | Approved, Investigational |
| MMP9 | Captopril | Approved |
| MMP9 | Glucosamine | Approved |
| MMP9 | AE-941 | Investigational |
| MMP9 | PG-530742 | Investigational |
| NR1I2 | Erlotinib | Approved, Investigational |
| NR1I2 | Estradiol | Approved, Investigational, |
| | | Vet Approved |
| NR1I2 | Ethinyl Estradiol | Approved |
| NR1I2 | Rifampicin | Approved |
| NR1I2 | Rifaximin | Approved, Investigational |
| NR1I2 | Paclitaxel | Approved, Vet Approved |
| NR1I2 | Docetaxel | Approved, Investigational |
| NR1I2 | Rilpivirine | Approved |
| PDGFRB | Becaplermin | Approved, Investigational |
| PDGFRB | Sorafenib | Approved, Investigational |
| PDGFRB | Imatinib | Approved |
| PDGFRB | Dasatinib | Approved, Investigational |
| PDGFRB | Sunitinib | Approved, Investigational |
| PDGFRB | XL999 | Investigational |
| PDGFRB | XL820 | Investigational |
| PDGFRB | Pazopanib | Approved |
| PDGFRB | Regorafenib | Approved |
| PGF | Aflibercept | Approved |
| PLAU | Urokinase | Approved, Investigational, |
| | | Withdrawn |
| PLAU | Amiloride | Approved |
| PLAU | Fibrinolysin | Investigational |

TABLE T
DrugBank drug targets for KIRP vs. KIRC using MEGENA

| Gene | Drug Name | Groups |
|---|---|---|
| ACAT1 | Ezetimibe | Approved |
| GABRB3 | Lorazepam | Approved |
| GABRB3 | Temazepam | Approved |
| GABRB3 | Butalbital | Approved, Illicit |
| GABRB3 | Topiramate | Approved |
| GABRB3 | Olanzapine | Approved, Investigational |
| GABRB3 | Clobazam | Approved, Illicit |
| GABRB3 | Eszopiclone | Approved |
| GABRB3 | Alprazolam | Approved, Illicit, |
| | | Investigational |
| GABRB3 | Chlordiazepoxide | Approved, Illicit |
| GABRB3 | Ivermectin | Approved, Vet Approved |
| GABRB3 | Clorazepate | Approved, Illicit |
| GABRB3 | Acamprosate | Approved, Investigational |
| GABRB3 | Midazolam | Approved, Illicit |
| GABRB3 | Flurazepam | Approved, Illicit |
| GABRB3 | Primidone | Approved, Vet Approved |
| GABRB3 | Diazepam | Approved, Illicit, Vet |
| | | Approved |
| GABRB3 | Oxazepam | Approved |
| GABRB3 | Triazolam | Approved |
| GABRB3 | Ergoloid mesylate | Approved |
| GABRB3 | Clonazepam | Approved, Illicit |
| GABRB3 | Flumazenil | Approved |
| GABRB3 | Estazolam | Approved, Illicit |
| GABRB3 | Bromazepam | Approved, Illicit |
| GABRB3 | Nitrazepam | Approved |
| GABRB3 | Thiocolchicoside | Approved |
| LCK | Dasatinib | Approved, Investigational |
| LCK | Ponatinib | Approved |
| LCK | Nintedanib | Approved |
| MAPK11 | KC706 | Investigational |
| MAPK11 | Regorafenib | Approved |
| OXT | Oxytocin | Approved, Vet Approved |
| SCTR | Secretin | Approved, Investigational |
| SLC19A1 | Methotrexate | Approved |
| SLC19A1 | Pralatrexate | Approved |
| SLC6A3 | Amphetamine | Approved, Illicit |
| SLC6A3 | Phentermine | Approved, Illicit |
| SLC6A3 | Citalopram | Approved |
| SLC6A3 | Benzatropine | Approved |
| SLC6A3 | Venlafaxine | Approved |
| SLC6A3 | Atomoxetine | Approved |
| SLC6A3 | Mirtazapine | Approved |
| SLC6A3 | Loxapine | Approved |
| SLC6A3 | Methylphenidate | Approved, Investigational |
| SLC6A3 | Pethidine | Approved |
| SLC6A3 | Imipramine | Approved |
| SLC6A3 | Duloxetine | Approved |
| SLC6A3 | Mazindol | Approved |
| SLC6A3 | Procaine | Approved, Investigational, |
| | | Vet Approved |
| SLC6A3 | Trimipramine | Approved |
| SLC6A3 | Modafinil | Approved, Investigational |
| SLC6A3 | Pseudoephedrine | Approved |
| SLC6A3 | Cocaine | Approved, Illicit |
| SLC6A3 | Diethylpropion | Approved, Illicit |
| SLC6A3 | Dopamine | Approved |
| SLC6A3 | Sertraline | Approved |
| SLC6A3 | Sibutramine | Approved, Illicit, |
| | | Investigational, Withdrawn |
| SLC6A3 | Chlorphenamine | Approved |
| SLC6A3 | Diphenylpyraline | Approved |
| SLC6A3 | Nefazodone | Approved, Withdrawn |
| SLC6A3 | Bupropion | Approved |
| SLC6A3 | Chloroprocaine | Approved |
| SLC6A3 | Escitalopram | Approved, Investigational |
| SLC6A3 | Lisdexamfetamine | Approved, Investigational |
| SLC6A3 | Dextroamphetamine | Approved, Illicit |
| SLC6A3 | Methamphetamine | Approved, Illicit |
| SLC6A3 | Altropane | Investigational |
| SLC6A3 | Mianserin | Approved |
| SLC6A3 | Armodafinil | Approved, Investigational |
| SLC6A3 | Dexmethylphenidate | Approved |
| SLC6A3 | Ioflupane I-123 | Approved |
| SLC6A3 | Methyl salicylate | Approved, Vet Approved |
| TNFSF13B | Belimumab | Approved |

TABLE U
DrugBank drug targets for KIRP vs. KIRC using nGOseq

| Gene | Drug Name | Groups |
|---|---|---|
| ABCC2 | Vasopressin | Approved |
| ABCC2 | Cyclosporine | Approved, Investigational, |
| | | Vet Approved |
| ABCC2 | Pravastatin | Approved |
| ABCC2 | Reserpine | Approved |
| ABCC2 | Indinavir | Approved |
| ABCC2 | Lovastatin | Approved, Investigational |
| ABCC2 | Phenytoin | Approved, Vet Approved |
| ABCC2 | Clotrimazole | Approved, Vet Approved |
| ABCC2 | Olmesartan | Approved, Investigational |
| ABCC2 | Conjugated estrogens | Approved |
| ABCC2 | Tenofovir disoproxil | Approved, Investigational |
| ABCC2 | Indomethacin | Approved, Investigational |
| ABCC2 | Aminohippuric acid | Approved |
| ABCC2 | Grepafloxacin | Withdrawn |
| ABCC2 | Sorafenib | Approved, Investigational |
| ABCC2 | Spironolactone | Approved |
| ABCC2 | Ritonavir | Approved, Investigational |
| ABCC2 | Cisplatin | Approved |
| ABCC2 | Oxaliplatin | Approved, Investigational |
| ABCC2 | Vincristine | Approved, Investigational |
| ABCC2 | Methotrexate | Approved |
| ABCC2 | Carbamazepine | Approved, Investigational |
| ABCC2 | Vinblastine | Approved |
| ABCC2 | Ivermectin | Approved, Vet Approved |
| ABCC2 | Simvastatin | Approved |
| ABCC2 | Verapamil | Approved |
| ABCC2 | Tamoxifen | Approved |
| ABCC2 | Mycophenolate mofetil | Approved, Investigational |
| ABCC2 | Daunorubicin | Approved |
| ABCC2 | Furosemide | Approved, Vet Approved |
| ABCC2 | Lamivudine | Approved, Investigational |
| ABCC2 | Irinotecan | Approved, Investigational |
| ABCC2 | Etoposide | Approved |
| ABCC2 | Sulfasalazine | Approved |
| ABCC2 | Eprosartan | Approved |
| ABCC2 | Quinidine | Approved |
| ABCC2 | Norgestimate | Approved |
| ABCC2 | Carboplatin | Approved |
| ABCC2 | Telmisartan | Approved, Investigational |
| ABCC2 | Ezetimibe | Approved |
| ABCC2 | Ethinyl Estradiol | Approved |
| ABCC2 | Lomefloxacin | Approved |
| ABCC2 | Doxorubicin | Approved, Investigational |
| ABCC2 | Glyburide | Approved |
| ABCC2 | Probenecid | Approved |
| ABCC2 | Rifampicin | Approved |
| ABCC2 | Atorvastatin | Approved |
| ABCC2 | Nifedipine | Approved |
| ABCC2 | Ofloxacin | Approved |
| ABCC2 | Arsenic trioxide | Approved, Investigational |
| ABCC2 | Phenobarbital | Approved |
| ABCC2 | Levetiracetam | Approved, Investigational |
| ABCC2 | Sparfloxacin | Approved |
| ABCC2 | Paclitaxel | Approved, Vet Approved |
| ABCC2 | Saquinavir | Approved, Investigational |
| ABCC2 | Dexamethasone | Approved, Investigational, |
| | | Vet Approved |
| ABCC2 | Docetaxel | Approved, Investigational |
| ABCC2 | Sunitinib | Approved, Investigational |
| ABCC2 | Pranlukast | Approved |
| ABCC2 | Ursodeoxycholic acid | Approved, Investigational |
| ABCC2 | Cholic Acid | Approved |
| ABCC2 | Fusidic Acid | Approved |
| ABCC2 | Quercetin | Experimental |
| ABCC2 | Pitavastatin | Approved |
| ABCC2 | Gadoxetic acid | Approved |
| ABCC2 | Canagliflozin | Approved |
| ABCC2 | Avibactam | Approved |
| ABCC2 | Eluxadoline | Approved |
| ABCC2 | Indocyanine green | Approved |
| ABCC2 | Levomefolic acid | Approved |
| ANXA2 | Tenecteplase | Approved |
| CDK5 | Alvocidib | Experimental, Investigational |
| JUN | Vinblastine | Approved |
| JUN | Pseudoephedrine | Approved |
| JUN | Irbesartan | Approved, Investigational |
| JUN | Arsenic trioxide | Approved, Investigational |
| MMP16 | Marimastat | Approved, Investigational |
| PADI4 | Azithromycin | Approved |
| PADI4 | Doxycycline | Approved, Investigational, |
| | | Vet Approved |
| PADI4 | Tetracycline | Approved, Vet Approved |
| PADI4 | Streptomycin | Approved, Vet Approved |
| PPIF | Cyclosporine | Approved, Investigational, |
| | | Vet Approved |
| PRKCA | Tamoxifen | Approved |
| PRKCA | Ingenol Mebutate | Approved |
| PRKCA | Ellagic Acid | Investigational |
| PYGM | Alvocidib | Experimental, Investigational |
| RAC1 | Dextromethorphan | Approved |

TABLE V
DrugBank drug targets for LUAD vs. LUSC using MEGENA

| Gene | Drug Name | Groups |
|---|---|---|
| FKBP1A | Pimecrolimus | Approved, Investigational |
| FKBP1A | Tacrolimus | Approved, Investigational |
| FKBP1A | Sirolimus | Approved, Investigational |
| FKBP1A | GPI-1485 | Investigational |
| IDE | Bacitracin | Approved, Vet Approved |
| JUN | Vinblastine | Approved |
| JUN | Pseudoephedrine | Approved |
| JUN | Irbesartan | Approved, Investigational |
| JUN | Arsenic trioxide | Approved, Investigational |
| KCNC1 | Dalfampridine | Approved |
| PPOX | Pidolic Acid | Experimental |
| SLC25A4 | Clodronic Acid | Approved, Investigational, |
| | | Vet Approved |
| VAMP1 | Botulinum Toxin Type B | Approved |

TABLE W
DrugBank drug targets for LUAD vs. LUSC using nGOseq

| Gene | Drug Name | Groups |
|---|---|---|
| BCHE | Pegvisomant | Approved |
| BCHE | Ramipril | Approved |
| BCHE | Succinylcholine | Approved |
| BCHE | Mefloquine | Approved |
| BCHE | Tacrine | Withdrawn |
| BCHE | Sulpiride | Approved |
| BCHE | Ethopropazine | Approved |
| BCHE | Dipivefrin | Approved |
| BCHE | Chlorpromazine | Approved, Vet Approved |
| BCHE | Cisplatin | Approved |
| BCHE | Pyridostigmine | Approved |
| BCHE | Nizatidine | Approved |
| BCHE | Triamcinolone | Approved, Vet Approved |
| BCHE | Galantamine | Approved |
| BCHE | Isoflurophate | Approved, Withdrawn |
| BCHE | Diethylcarbamazine | Approved, Vet Approved |
| BCHE | Procaine | Approved, Investigational, |
| | | Vet Approved |
| BCHE | Pralidoxime | Approved, Vet Approved |
| BCHE | Irinotecan | Approved, Investigational |
| BCHE | Malathion | Approved, Investigational |
| BCHE | Perindopril | Approved |
| BCHE | Terbutaline | Approved |
| BCHE | Oxybuprocaine | Approved |
| BCHE | Cyclopentolate | Approved |
| BCHE | Rivastigmine | Approved, Investigational |
| BCHE | Procainamide | Approved |
| BCHE | Echothiophate | Approved |
| BCHE | Trimethaphan | Approved |
| BCHE | Chloroprocaine | Approved |
| BCHE | Mivacurium | Approved |
| BCHE | Ephedrine | Approved |
| BCHE | Drospirenone | Approved |
| BCHE | Neostigmine | Approved, Vet Approved |
| BCHE | Bambuterol | Approved |
| BCHE | Butyric Acid | Experimental |
| BCHE | Clevidipine | Approved |
| BCHE | recombinant human | Investigational |
| | GM-CSF | |
| BCHE | substance P | Investigational |
| BCHE | Capsaicin | Approved |
| BCHE | Mirabegron | Approved |
| BCHE | Aclidinium | Approved |
| GRM2 | LY2140023 | Investigational |
| HRSP12 | Benzoic Acid | Approved |
| PARP1 | Nicotinamide | Approved |
| PARP1 | Veliparib | Investigational |
| PARP1 | Olaparib | Approved |
| PARP1 | Rucaparib | Approved, Investigational |
| PLD1 | LAX-101 | Investigational |
| PLD1 | Miltefosine | Approved |
| PPIA | Cyclosporine | Approved, Investigational, |
| | | Vet Approved |
| PRKCD | Tamoxifen | Approved |
| PRKCD | Ingenol Mebutate | Approved |
| PRKCI | Tamoxifen | Approved |
| RAC1 | Dextremethorphan | Approved |

TABLE X
DrugBank drug targets for Luminal
A vs. Luminal B using MEGENA

| Gene | Drug Name | Groups |
|---|---|---|
| FABP5 | Palmitic Acid | Experimental |
| HPN | Coagulation factor Vila Recombinant Human | Approved |
| HPN | Bentiromide | Withdrawn |

TABLE Y
DrugBank drug targets for Luminal A vs. Luminal B using nGOseq

| Gene | Drug Name | Groups |
|---|---|---|
| AVPR1B | Desmopressin | Approved |
| AVPR1B | Vasopressin | Approved |
| AVPR1B | Terlipressin | Approved, Investigational |
| BIRC5 | LY2181308 | Investigational |
| GRIN2A | Atomoxetine | Approved |
| GRIN2A | Pentobarbital | Approved, Vet Approved |
| GRIN2A | Pethidine | Approved |
| GRIN2A | Acamprosate | Approved, Investigational |
| GRIN2A | Felbamate | Approved |
| GRIN2A | Gabapentin | Approved, Investigational |
| GRIN2A | Memantine | Approved, Investigational |
| GRIN2A | Phenobarbital | Approved |
| GRIN2A | Tenocyclidine | Experimental, Illicit |
| GRIN2A | Milnacipran | Approved |
| GRIN2A | Acetylcysteine | Approved, Investigational |
| GRIN2A | Ketobemidone | Approved |
| HTR1F | Eletriptan | Approved, Investigational |
| HTR1F | Zolmitriptan | Approved, Investigational |
| HTR1F | Sumatriptan | Approved, Investigational |
| HTR1F | Ergotamine | Approved |
| HTR1F | Naratriptan | Approved, Investigational |
| HTR1F | Rizatriptan | Approved |
| HTR1F | Ergoloid mesylate | Approved |
| HTR1F | Ketamine | Approved, Vet Approved |
| HTR1F | Mianserin | Approved |
| HTR1F | Tiapride | Approved, Investigational |
| KCNH6 | Ibutilide | Approved |
| KCNH6 | Prazosin | Approved |
| KCNH6 | Doxazosin | Approved |
| KCNH6 | Miconazole | Approved, Investigational, |
| | | Vet Approved |
| KCNH6 | Terazosin | Approved |
| KCNK9 | Doxapram | Approved, Vet Approved |
| KCNK9 | Halothane | Approved, Vet Approved |
| NME1 | Tenofovir disoproxil | Approved, Investigational |
| NME1 | Lamivudine | Approved, Investigational |
| NME1 | Adefovir Dipivoxil | Approved, Investigational |
| NPR3 | Nesiritide | Approved, Investigational |
| PADI4 | Azithromycin | Approved |
| PADI4 | Doxycycline | Approved, Investigational, |
| | | Vet Approved |
| PADI4 | Tetracycline | Approved, Vet Approved |
| PADI4 | Streptomycin | Approved, Vet Approved |
| PLG | Alteplase | Approved |
| PLG | Urokinase | Approved, Investigational, |
| | | Withdrawn |
| PLG | Reteplase | Approved |
| PLG | Tenecteplase | Approved |
| PLG | Streptokinase | Approved |
| PLG | Tranexamic Acid | Approved |
| PLG | Aminocaproic Acid | Approved, Investigational |
| PLG | Desmoteplase | Investigational |
| PLG | Aprotinin | Approved, Withdrawn |
| TXNRD1 | Arsenic trioxide | Approved, Investigational |
| TXNRD1 | Flavin adenine dinucleotide | Approved |
| TXNRD1 | Fotemustine | Experimental |
| TXNRD1 | motexafin gadolinium | Investigational |
| TXNRD1 | PX-12 | Investigational |

TABLE Z
DrugBank drug targets for pan-22 cancer
multinomial modeling using MEGENA

| Gene | Drug Name | Groups |
|---|---|---|
| ADAM28 | Pidolic Acid | Experimental |
| COX7A1 | Cholic Acid | Approved |
| CRAT | L-Carnitine | Approved |
| CYP17A1 | Progesterone | Approved, Vet Approved |
| CYP17A1 | Metoclopramide | Approved, Investigational |
| CYP17A1 | Dexamethasone | Approved, Investigational, |
| | | Vet Approved |
| CYP17A1 | Aldosterone | Experimental |
| CYP17A1 | Abiraterone | Approved |
| DDR2 | Regorafenib | Approved |
| EGF | Sucralfate | Approved |
| EGF | Tesevatinib | Investigational |
| F2 | Lepirudin | Approved |
| F2 | Bivalirudin | Approved, Investigational |
| F2 | Antihemophilic factor, | Approved, Investigational |
| | human recombinant | |
| F2 | Drotrecogin alfa | Approved, Investigational, |
| | | Withdrawn |
| F2 | Coagulation Factor | Approved |
| | IX (Recombinant) | |
| F2 | Argatroban | Approved, Investigational |
| F2 | Proflavine | Approved |
| F2 | Suramin | Approved |
| F2 | Ximelagatran | Approved, Investigational, |
| | | Withdrawn |
| F2 | Thrombomodulin Alfa | Approved, Investigational |
| F2 | Human Cl-esterase | Approved |
| | inhibitor | |
| F2 | Dabigatran etexilate | Approved |
| F2 | Conestat alfa | Approved |
| FGF1 | Pentosan Poly sulfate | Approved |
| FGF1 | Amlexanox | Approved, Investigational |
| FGF1 | Formic Acid | Experimental |
| FGF1 | Pazopanib | Approved |
| FKBP1A | Pimecrolimus | Approved, Investigational |
| FKBP1A | Tacrolimus | Approved, Investigational |
| FKBP1A | Sirolimus | Approved, Investigational |
| FKBP1A | GPI-1485 | Investigational |
| GJA1 | Carvedilol | Approved, Investigational |
| GUCY1A2 | Isosorbide Mononitrate | Approved |
| GUCY1A2 | Riociguat | Approved |
| GUCY1A2 | Methylene blue | Investigational |
| GUCY1A2 | Plecanatide | Approved |
| HABP4 | Hyaluronic acid | Approved, Vet Approved |
| JDP2 | Pseudoephedrine | Approved |
| KCNQ1 | Indapamide | Approved |
| KCNQ1 | Azimilide | Investigational |
| KCNQ1 | ICA-105665 | Investigational |
| PIK3CA | XL765 | Investigational |
| PTPN1 | Tiludronic acid | Approved, Vet Approved |
| PTPN1 | ISIS 113715 | Investigational |
| SLCO1C1 | Phenytoin | Approved, Vet Approved |
| SLCO1C1 | Liothyronine | Approved, Vet Approved |
| SLCO1C1 | Conjugated estrogens | Approved |
| SLCO1C1 | Digoxin | Approved |
| SLCO1C1 | Levothyroxine | Approved |
| SLCO1C1 | Dextrothyroxine | Approved |
| SLCO1C1 | Methotrexate | Approved |
| SLCO1C1 | Diclofenac | Approved, Vet Approved |
| SLCO1C1 | Estradiol | Approved, Investigational, |
| | | Vet Approved |
| SLCO1C1 | Dinoprostone | Approved |
| SLCO1C1 | Meclofenamic acid | Approved, Vet Approved |
| SLCO1C1 | Probenecid | Approved |
| VDAC2 | PRLX 93936 | Investigational |

TABLE AA
DrugBank drug targets for pan-20 cancer survival using MEGENA

| Gene | Drug Name | Groups |
|---|---|---|
| CDK4 | Alvocidib | Experimental, Investigational |
| CDK4 | Palbociclib | Approved |
| CDK4 | Ribociclib | Approved |
| FCGR2A | Cetuximab | Approved |
| FCGR2A | Etanercept | Approved, Investigational |
| FCGR2A | Immune Globulin Human | Approved, Investigational |
| FCGR2A | Adalimumab | Approved |
| FCGR2A | Abciximab | Approved |
| FCGR2A | Gemtuzumab ozogamicin | Approved |
| FCGR2A | Trastuzumab | Approved, Investigational |
| FCGR2A | Rituximab | Approved |
| FCGR2A | Basiliximab | Approved, Investigational |
| FCGR2A | Muromonab | Approved, Investigational |
| FCGR2A | Ibritumomab tiuxetan | Approved |
| FCGR2A | Tositumomab | Approved |
| FCGR2A | Alemtuzumab | Approved, Investigational |
| FCGR2A | Alefacept | Approved, Withdrawn |
| FCGR2A | Efalizumab | Approved, Investigational |
| FCGR2A | Natalizumab | Approved, Investigational |
| FCGR2A | Palivizumab | Approved, Investigational |
| FCGR2A | Daclizumab | Approved, Investigational |
| FCGR2A | Bevacizumab | Approved, Investigational |
| IL1R1 | Anakinra | Approved |
| MAP2K2 | Bosutinib | Approved |
| MAP2K2 | Trametinib | Approved |
| MAPK13 | KC706 | Investigational |
| PRKAG2 | Acetylsalicylic acid | Approved, Vet Approved |
| SLC10A1 | Cyclosporine | Approved, Investigational, Vet Approved |
| SLC10A1 | Liothyronine | Approved, Vet Approved |
| SLC10A1 | Conjugated estrogens | Approved |
| SLC10A1 | Indomethacin | Approved, Investigational |
| SLC10A1 | Progesterone | Approved, Vet Approved |
| SLC10A1 | Testosterone | Approved, Investigational |
| SLC10A1 | Bumetanide | Approved |
| SLC10A1 | Ethinyl Estradiol | Approved |
| SLC10A1 | Probenecid | Approved |
| SLC10A1 | Ursodeoxycholic acid | Approved, Investigational |
| SLC10A1 | Cholic Acid | Approved |
| SLC10A1 | Deoxycholic Acid | Approved |
| SLC10A1 | Pitavastatin | Approved |
| TGFB1 | Hyaluronidase | Approved, Investigational |
| TGFB1 | Hyaluronidase (Human Recombinant) | Approved |
| TUBB2B | CYT997 | Investigational |

TABLE AB
DrugBank drug targets for pan-22 cancer multinomial modeling using nGOseq

| Gene | Drug Name | Groups |
|---|---|---|
| ACOX1 | Flavin adenine dinucleotide | Approved |
| ACPP | Sipuleucel-T | Approved |
| CACNB2 | Isradipine | Approved |
| CACNB2 | Amlodipine | Approved |
| CACNB2 | Nimodipine | Approved |
| CACNB2 | Nisoldipine | Approved |
| CACNB2 | Spironolactone | Approved |
| CACNB2 | Nicardipine | Approved |
| CACNB2 | Magnesium Sulfate | Approved, Vet Approved |
| CACNB2 | Verapamil | Approved |
| CACNB2 | Felodipine | Approved, Investigational |
| CACNB2 | Nitrendipine | Approved |
| CACNB2 | Nifedipine | Approved |
| CACNB2 | Mibefradil | Withdrawn |
| CACNB2 | Dronedarone | Approved |
| CACNB2 | Nilvadipine | Approved |
| CD80 | Abatacept | Approved |
| CD80 | Galiximab | Investigational |
| CD80 | Belatacept | Approved |
| CYP4F12 | Fingolimod | Approved, Investigational |
| DDR2 | Regorafenib | Approved |
| EPHA2 | Dasatinib | Approved, Investigational |
| EPHA2 | Regorafenib | Approved |
| HCK | Quercetin | Experimental |
| HCK | Bosutinib | Approved |
| HTR1F | Eletriptan | Approved, Investigational |
| HTR1F | Zolmitriptan | Approved, Investigational |
| HTR1F | Sumatriptan | Approved, Investigational |
| HTR1F | Ergotamine | Approved |
| HTR1F | Naratriptan | Approved, Investigational |
| HTR1F | Rizatriptan | Approved |
| HTR1F | Ergoloid mesylate | Approved |
| HTR1F | Ketamine | Approved, Vet Approved |
| HTR1F | Mianserin | Approved |
| HTR1F | Tiapride | Approved, Investigational |
| HTR3D | Ergoloid mesylate | Approved |
| HTR3D | Tiapride | Approved, Investigational |
| HTR7 | Eletriptan | Approved, Investigational |
| HTR7 | Ziprasidone | Approved |
| HTR7 | Cabergoline | Approved |
| HTR7 | Amitriptyline | Approved |
| HTR7 | Olanzapine | Approved, Investigational |
| HTR7 | Clozapine | Approved |
| HTR7 | Mirtazapine | Approved |
| HTR7 | Loxapine | Approved |
| HTR7 | Imipramine | Approved |
| HTR7 | Chlorpromazine | Approved, Vet Approved |
| HTR7 | Epinastine | Approved, Investigational |
| HTR7 | Maprotiline | Approved |
| HTR7 | Dopamine | Approved |
| HTR7 | Ergoloid mesylate | Approved |
| HTR7 | Bromocriptine | Approved, Investigational |
| HTR7 | Quetiapine | Approved |
| HTR7 | Aripiprazole | Approved, Investigational |
| HTR7 | Iloperidone | Approved |
| HTR7 | Mianserin | Approved |
| HTR7 | Asenapine | Approved |
| HTR7 | Amisulpride | Approved, Investigational |
| HTR7 | Lurasidone | Approved |
| HTR7 | Vortioxetine | Approved |
| HTR7 | Tiapride | Approved, Investigational |
| IL13RA2 | AER001 | Investigational |
| IL23A | Briakinumab | Investigational |
| IL23A | Ustekinumab | Approved, Investigational |
| KLK3 | Ecallantide | Approved |
| KLK3 | Human Cl-esterase inhibitor | Approved |
| KLK3 | Conestat alfa | Approved |
| PIK3R3 | Isoprenaline | Approved |
| PIK3R3 | SF1126 | Investigational |
| PIM1 | Quercetin | Experimental |
| PPIA | Cyclosporine | Approved, Investigational, Vet Approved |
| SLC22A5 | Amphetamine | Approved, Illicit |
| SLC22A5 | Nicotine | Approved |
| SLC22A5 | Lidocaine | Approved, Vet Approved |
| TSHR | Thyrotropin Alfa | Approved, Vet Approved |
| TUBA1B | Epothilone D | Experimental, Investigational |
| TUBA1B | Patupilone | Experimental, Investigational |
| TUBA1B | CYT997 | Investigational |
| TUBA3D | Epothilone D | Experimental, Investigational |
| TUBA3D | Patupilone | Experimental, Investigational |
| TUBA3D | CYT997 | Investigational |

TABLE AC
Pharmacodia drug targets for BRCA vs Normal using MEGENA

| Gene | Drug Name | Description | Clinical Trials |
|---|---|---|---|
| EZH2 | Tazemetostat | An enhancer Of zeste homolog 2 (EZH2) inhibitor | Phase II |
| | | potentially potentially for the treatment of non- | |
| | | Hodgkin's lymphoma (NHL). | |
| | CPI-1205 | An enhancer of zeste homolog 2 (EZH2) inhibitor | Phase I |
| | | potentially for the treatment of B-cell lymphoma. | |
| | GSK-2816126 | An enhancer of zeste homolog 2 (EZH2) inhibitor | Phase I |
| | | potentially for the treatment of diffuse large B cell | |
| | | lymphoma and follicular lymphoma. | |
| PTS | Nepicastat | A dopamine beta-hydroxylase (DBH) inhibitor | Phase II |
| | Hydrochloride | potentially for the treatment of post-traumatic stress | |
| | | disorder (PTSD) and substance abuse and dependence. | |
| TLR8 | Motolimod | A toll-like receptor 8 (TLR8) agonist potentially for the | Phase II |
| | | treatment of ovarian cancer, peritoneum cancers and | |
| | | head and neck cancer. | |
| | MEDI-9197 | A dual agonist of toll-like receptor 7 (TLR7) and toll- | Phase I |
| | | like receptor 8 (TLR8) potentially for the treatment of | |
| | | solid tumors. | |
| | IMO-8400 | A TLR7, TLR8 and TLR9 antagonist potentially for the | Phase II |
| | | treatment of dermatomyositis, Waldenstrom's | |
| | | macroglobulinemia, diffuse large B-cell lymphoma. | |
| | VTX-1463 | A toll-like receptor 8 (TLR8) agonist potentially for the | Phase I |
| | | treatment of allergic rhinitis. | |
| | Resiquimod | A toll-like receptor 7 (TLR7) and toll-like receptor 8 | Phase II |
| | | (TLR8) agonist potentially for treatment of cutaneous | |
| | | T-cell lymphoma and actinic keratosis. | |

TABLE AD
Pharmacodia drug targets for BRCA vs Normal using nGOseq

| Gene | Drug Name | Description | Clinical Trials |
|---|---|---|---|
| C6 | Citarinostat | A histone deacetylase 6 (HDAC6) inhibitor potentially for the treatment of | Phase II |
| | | multiple myeloma (MM). | |
| DRD2 | Lu-AF-35700 | A dopamine D2 receptor (DRD2) modulator potentially for the treatment of | Phase III |
| | | schizophrenia. | |
| | Cariprazine | A dopamine receptor D2 (DRD2)/serotonin 5-HT1A receptor agonist and | Approved |
| | Hydrochloride | serotonin 5-HT2A receptor antagonist used to treat schizophrenia and | |
| | | bipolar I disorder. | |
| | Aplindore | A dopamine D2 receptor (DRD2) agonist potentially for the treatment of | Phase II |
| | Fumarate | Parkinson's disease and restless legs syndrome. | |
| | DSP-1200 | An alpha 2a adrenergic receptor (ADRA2A) antagonist, a dopamine D2 | Phase I |
| | | receptor (DRD2) antagonist and a serotonin 2A receptor antagonist | |
| | | potentially for the treatment of depressive disorders. | |
| | PF-217830 | A dopamine D2 receptor (DRD2) agonist, serotonin 5-HT1A receptor | Phase II |
| | | agonist and serotonin 5-HT2A receptor antagonist potentially for the | |
| | | treatment of schizophrenia. | |
| | ATC-1906 | A dopamine D2 receptor (DRD2) antagonist and dopamine D3 receptor | Phase I |
| | | (DRD3) antagonist potentially for the treatment of gastroparesis. | |
| | Perospirone | An antagonist of dopamine D2 receptor (DRD2) and serotonin 5-HT2A | Approved |
| | Hydrochloride | receptor used to treat schizophrenia and bipolar mania. | |
| | Hydrate | | |
| | Ocaperidone | A 5-hydroxytryptamine receptor 2A (5-HT2A receptor) antagonist and | Phase II |
| | | dopamine D2 receptor (DRD2) antagonist potentially for the treatment of | |
| | | schizophrenia. | |
| | JNJ-37822681 | A dopamine D2 receptor (DRD2) antagonist potentially for the treatment of | Phase II |
| | | schizophrenia. | |
| | Ziprasidone | A dopamine D2 receptor (DRD2) and serotonin 5-HT2 receptor antagonist | Approved |
| | | used to treat schizophrenia and bipolar I disorder. | |
| | Roxindole | A dopamine D2 receptor (DRD2) agonist, serotonin 5-HT1A receptor | Phase |
| | | agonist and serotonin uptake inhibitor potentially for the treatment of | III |
| | | psychotic disorders. | |
| | Pergolide | A D(2) dopamine receptor (DRD2) agonist and D(1) dopamine receptor | Approved |
| | Mesilate | (DRD1) agonist used to treat Parkinson's disease. | |
| | Prochlorperazine | A dopamine D2 receptor (DRD2) antagonist used to treat schizophrenia | Approved |
| | edisylate | and anxiety disorder. | |
| | JNJ-37822681 | A dopamine D2 receptor (DRD2) antagonist potentially for the treatment of | Phase II |
| | | schizophrenia. | |
| ITK | JTE-051 | An IL2 inducible T-cell kinase (ITK) inhibitor potentially for the treatment | Phase II |
| | | of autoimmune diseases, hypersensitivity and rheumatoid arthritis (RA). | |
| KLB | RG-7992 | A bispecific antibody targeting KLB and FGFR1 potentially for the | Phase I |
| | | treatment of type 2 diabetes. | |
| PDC | CPI-613 | An oxoglutarate dehydrogenase complex (OGDC) and pyruvate | Phase II |
| | | dehydrogenase complex (PDC) inhibitor potentially for the treatment of | |
| | | small cell lung cancer (SCLC), myelodysplastic syndrome (MDS) and | |
| | | metastatic pancreatic cancer. | |
| PDE2A | OSI-461 | A Phosphodiesterase 2A/5A (PDE2A/5A) inhibitor potentially for the | Phase II |
| | | treatment of renal cell carcinoma, prostate cancer, Crohn's disease, and | |
| | | chronic lymphocytic leukemia (CLL). | |
| | TAK-915 | A phosphodiesterase 2A (PDE2A) inhibitor potentially for the treatment of | Phase I |
| | | schizophrenia. | |
| | PF-05180999 | A phosphodiesterase PDE2A inhibitor potentially for the treatment of | Phase I |
| | | migraine and schizophrenia. | |
| | ND-7001 | A phosphodiesterase PDE2A inhibitor potentially for the treatment of | Phase I |
| | | anxiety and depression. | |
| | Fluticasone | A phosphodiesterase 2A (PDE2A) agonist and glucocorticoid receptor (GR) | Approved |
| | Propionate | agonist used for the relief of the inflammatory and pruritic manifestations of | |
| | | corticosteroid-responsive dermatoses. | |
| TGFB2 | ISTH-0036 | A TGFB2 inhibitor potentially for the treatment of glaucoma. | Phase I |

TABLE AE
Pharmacodia drug targets for ER+ vs. ER− using MEGENA

| Gene | Drug Name | Description | Clinical Trials |
|---|---|---|---|
| CD40 | ADC-1013 | An agonistic CD40 antibody potentially for the treatment of | Phase I |
| | | solid tumours. | |
| | Bleselumab | A CD40 targeted antibody potentially for the treatment of renal | Phase II |
| | | transplant rejection and other transplant rejection. | |
| | SEA-CD40 | A CD40 targeted antibody potentially for the treatment of | Phase I |
| | | haematological malignancies and solid tumours. | |
| | Lucatumumab | A CD40 targeted antibody potentially for the treatment of | Phase II |
| | | chronic lymphocytic leukaemia, follicular lymphoma and | |
| | | multiple myeloma. | |
| | CP-870893 | An agonistic CD40 antibody potentially for the treatment of | Phase I |
| | | malignant melanoma. | |
| | BI-655064 | A CD40 targeted monoclonal antibody potentially for the | Phase II |
| | | treatment of immune thrombocytopenic purpura, lupus nephritis | |
| | | and rheumatoid arthritis. | |
| | RG-7876 | A CD40 agonist potentially for the treatment of pancreatic | Phase I |
| | | cancer and some other solid tumours. | |
| | Dacetuzumab | A CD40 targeted antibody potentially for the treatment of | Phase II |
| | | diffuse large B cell lymphoma. | |
| | BMS-986090 | An anti-CD40 antibody potentially for the treatment of | Phase I |
| | | immunological disorders. | |
| | FFP-104 | A CD40 targeted antibody potentially for the treatment of | Phase II |
| | | Crohn's disease and primary biliary cirrhosis. | |
| | APX-005M | A CD40 agonistic antibody potentially for the treatment of solid | Phase I |
| | | tumors. | |
| | BIIB-063 | A CD40 ligand (CD40L) inhibitor potentially for the treatment | Phase I |
| | | of Sjoegren's syndrome. | |
| | MEDI-4920 | An anti-CD40L-Tn3 fusion protein potentially for the treatment | Phase I |
| | | of primary Sjogren's syndrome and rheumatoid arthritis. | |
| | Letolizumab | A CD40 ligand inhibitor potentially for the treatment of immune | Phase II |
| | | thrombocytopenic purpura. | |
| | Dapirolizumab pegol | A CD40 ligand (CD40L) inhibitor potentially for the treatment | Phase II |
| | | of systemic lupus erythematosus (SLE). | |
| CX3CL1 | E-6011 | A fractalkine (CX3CL1) inhibitor potentially for the treatment | Phase II |
| | | of Crohn's disease, rheumatoid arthritis. | |
| | AB-001 | An anti-fractalkine (CX3CL1; FKN) for the treatment of chronic | Phase II |
| | | low back pain, musculoskeletal pain and arthritis. | |
| CYP2D6 | Bupropion | A CYP2D6 inhibitor used to treat depression. | Approved |
| | Hydrochloride; | | |
| | Amfebutamone | | |
| | hydrochloride | | |
| | Halofantrine | A CYP2D6 inhibitor used to treat plasmodium falciparum | Approved |
| | Hydrochloride | malaria and plasmodium vivax malaria. | |
| | Hydralazine | A CYP2D6 inhibitor used to treat hypertension. | Approved |
| | hydrochloride | | |
| PDE10A | TAK-063 | A phosphodiesterase 10A (PDE10A) inhibitor potentially for the | Phase II |
| | | treatment of schizophrenia. | |
| | PBF-999 | An adenosine A2A receptor antagonist and PDE10A inhibitor | Phase I |
| | | potentially for the treatment of Huntington's disease. | |
| | TAK-063 | A phosphodiesterase 10A (PDE10A) inhibitor potentially for the | Phase II |
| | | treatment of schizophrenia. | |
| | OMS-643762 | A phosphodiesterase 10A (PDE10A) inhibitor potentially for the | Phase II |
| | | treatment of schizophrenia and Huntington's disease. | |
| | PF-02545920 | A phosphodiesterase 10A (PDE10A) inhibitor potentially for the | Phase II |
| | | treatment of Huntington's Disease. | |
| | AMG-579 | A phosphodiesterase PDE10A inhibitor potentially for the | Phase I |
| | | treatment of schizoaffective disorder and schizophrenia. | |

TABLE AF
Pharmacodia drug targets for ER+ vs. ER− using MEGENA nGOseq

| Gene | Drug Name | Description | Clinical Trials |
|---|---|---|---|
| ADORA2B | ATL-844 | An adenosine A2b receptor (ADORA2B) antagonist potentially for the | Phase II |
| | | treatment of asthma and type-2 diabetes. | |
| | GS-6201 | An adenosine A2B receptor (ADORA2B) antagonist potentially for the | Phase I |
| | | treatment of pulmonary diseases. | |
| | LAS-101057 | An adenosine A2B receptor (ADORA2B) antagonist potentially for the | Phase I |
| | | treatment of asthma. | |
| ALK | ZL-2302 | An anaplastic lymphoma kinase (ALK) inhibitor potentially for the | IND |
| | | treatment of anaplastic lymphoma kinase (ALK)-positive NSCLC. | Filing |
| | Foritinib | An anaplastic lymphoma kinase (ALK) inhibitor potentially for the | Phase I |
| | Succinate | treatment of lung cancer. | |
| | Lorlatinib | An ALK inhibitor and ROS1 inhibitor potentially for the treatment of | Phase III |
| | | non-small cell lung cancer. | |
| | Ceritinib | A kinase inhibitor used to treat ALK-positive metastatic non-small cell | Approved |
| | | lung cancer (NSCLC) following treatment with crizotinib. | |
| | TSR-011 | A TrKA/ALK inhibitor potentially for the treatment of solid tumours and | Phase II |
| | | lymphoma. | |
| | Ensartinib | An anaplastic lymphoma kinase (ALK) inhibitor potentially for the | Phase III |
| | | treatment of central nervous system tumors and non small cell lung | |
| | | cancer. | |
| | EBI-215 | An anaplastic lymphoma kinase (ALK) inhibitor for the treatment of non | Phase I |
| | | small cell lung cancer (NSCLC). | |
| | TQ-B3101 | A anaplastic lymphoma kinase (ALK) inhibitor potentially for the | Phase I |
| | | treatment of non small cell lung cancer (NSCLC), gastric cancer and | |
| | | lymphoma. | |
| | CEP-37440 | An ALK and FAK inhibitor potentially for the treatment of solid tumors. | Phase I |
| | PLB-1003 | An nnaplastic lymphoma kinase (ALK) inhibitor potentially for the | Phase I |
| | | treatment of ALK positive non small cell lung cancer (NSCLC). | |
| | Entrectinib | A multi-kinase (ALK, TrkB, TrkC, TrkA, ROS1) inhibitor potentially for | Phase II |
| | | the treatment of non small cell lung cancer (NSCLC) and colorectal | |
| | | cancer. | |
| | TPX-0005 | A multi-target ALK/ROS1/TRK/SRC inhibitor potentially for the | Phase II |
| | | treatment of non small cell lung cancer (NSCLC) and solid tumours. | |
| | ASP-3026 | An ALK inhibitor potentially for the treatment of solid tumors and B-cell | Phase I |
| | | lymphoma. | |
| | Alectinib | A tyrosine kinase (ALK and RET) inhibitor used to treat non small cell | Approved |
| | Hydrochloride | lung cancer. | |
| | Frizotinib | An anaplastic lymphoma kinase (ALK) inhibitor potentially for the | Phase I |
| | | treatment of non small cell lung cancer (NSCLC). | |
| | Brigatinib | A multi-target inhibitor used for the treament of ALK+ non-small cell | Approved |
| | | lung cancer (NSCLC). | |
| CA2 | Brinzolamide | A carbonic anhydrase 2 (CA2) inhibitor used to treat ocular hypertension | Approved |
| | | and open-angle glaucoma. | |
| CDK7 | SY-1365 | A cyclin-dependent kinase 7 (CDK7) inhibitor potentially for the | Phase I |
| | | treatment of solid tumours. | |
| ENPP3 | AGS-16C3F | A ENPP3 targeted antibody conjugated to MMAF potentially for the | Phase II |
| | | treatment of renal cell carcinoma. | |
| JAK2 | Gandotinib | A Janus kinase 2 (JAK2) inhibitor potentially for the treatment of | Phase II |
| | | myeloproliferative disorders (MPD). | |
| | Ruxolitinib | An inhibitor of Janus kinase 1 (JAK1) and Janus kinase 2 (JAK2) used to | Approved |
| | Phosphate | treat bone marrow cancer. | |
| | BMS-911543 | A Janus kinase 2 (JAK2) inhibitor potentially for the treatment of | Phase II |
| | | myelofibrosis. | |
| | Fedratinib | A JAK2/FLT3 inhibitor potentially for the treatment of myelofibrosis, | Phase III |
| | | essential thrombocythaemia (ET) and solid tumours. | |
| | Lestaurtinib | An Fms-like tyrosine kinase 3 (FLT-3) inhibitor and a janus kinase 2 | Phase III |
| | | (JAK2) inhibitor potentially for the treatment of acute lymphoblastic | |
| | | leukaemia (ALL). | |
| | BMS-911543 | A Janus kinase 2 (JAK2) inhibitor potentially for the treatment of | Phase II |
| | | myelofibrosis. | |
| | Baricitinib | An inhibitor of Janus kinase 1(JAK1) and Janus kinase 2(JAK2) | Approved |
| | | potentially for the treatment of rheumatoid arthritis. | |
| | Itacitinib | A Janus kinase (JAK1, JAK2) inhibitor potentially for the treatment of | Phase II |
| | | non-small cell lung cancer and pancreatic cancer. | |
| | AC-410 | A janus kinase 2 (JAK2) inhibitor potentially for the treatment of cancer, | Phase I |
| | | autoimmune and inflammatory diseases. | |
| PGF | Aflibercept | A vascular endothelial growth factor A (VEGFA) and placental growth | Approved |
| | | factor (PGF) inhibitor used to treat neovascular (Wet) age-related | |
| | | macular degeneration, macular edema following retinal vein occlusion | |
| | | and diabetic macularedema. | |
| | Anti-placental | A placental growth factor (PGF) inhibitor potentially for the treatment of | Phase II |
| | growth factor | diabetic macular oedema and medulloblastoma. | |
| | monoclonal | | |
| | antibody | | |
| | Ziv-aflibercept | A vascular endothelial growth factor A (VEGFA) and placental growth | Approved |
| | | factor (PGF) inhibitor used to treat metastatic colorectal cancer. | |
| | Latanoprostene | A nitric oxide-donating prostaglandin F2-alpha (PGF2-α) analogue | NDA |
| | Bunod | potentially for the treatment of glaucoma in patients with open angle | Filing |
| | | glaucoma and ocular hypertension. | |
| PLAU | BAY-1129980 | A Ly6/PLAUR domain-containing protein 3 (LYPD3/C4.4a) targeted | Phase I |
| | | antibody conjugated to auristatin potentially for the treatment of cancer. | |

TABLE AG
Pharmacodia drug targets for KIRP vs. KIRC using MEGENA

| Gene | Drug Name | Description | Clinical Trials |
|---|---|---|---|
| CCR1 | BX-471 | A C-C motif chemokine receptor 1 (CCR1) antagonist potentially for the treatment of | Phase II |
| | | multiple myeloma, multiple sclerosis, endometriosis, psoriasis and Alzheimer's disease | |
| | | (AD). | |
| | MLN3701 | A CCR1 receptor antagonist potentially for the treatment of inflammation and | Phase I |
| | | rheumatoid arthritis (RA). | |
| | CCX-354 | A C-C motif chemokine receptor 1 (CCR1) antagonist potentially for the treatment of | Phase II |
| | | rheumatoid arthritis. | |
| | MLN3897 | A chemokine CCR1 antagonist potentially for the treatment of multiple sclerosis and | Phase I |
| | | rheumatoid arthritis. | |
| PDC | CPI-613 | An oxoglutarate dehydrogenase complex (OGDC) and pyruvate dehydrogenase | Phase II |
| | | complex (PDC) inhibitor potentially for the treatment of small cell lung cancer | |
| | | (SCLC), myelodysplastic syndrome (MDS) and metastatic pancreatic cancer. | |

TABLE AH
Pharmacodia drug targets for KIRP vs. KIRC using nGOseq

| Gene | Drug Name | Description | Clinical Trials |
|---|---|---|---|
| ATM | AZD-0156 | An ataxia telangiectasia mutated kinase (ATM) inhibitor potentially for the | Phase I |
| | | treatment of solid tumors. | |
| MET | Onartuzumab | A MET blocker used to treat metastatic non-small cell lung cancer and gastric | Phase III |
| | | cancer. | |
| | LY-3164530 | An epidermal growth factor receptor (EGFR) and mesenchymal-epithelial | Phase I |
| | | transition factor (MET) antagonist potentially for the treatment of cancer. | |
| | SGX-523 | A HGFR (MET; c-Met) inhibitor potentially for the treatment of patients with | Phase I |
| | | solid tumours. | |
| MIR21 | RG-012 | A microRNA 21 (MIR21) inhibitor potentially for the treatment of nephritis. | Phase II |
| PAK4 | KPT-9274 | A nicotinamide phosphoribosyltransferase (NAMPT) inhibitor and p21- | Phase I |
| | | activated kinase 4 (PAK4) inhibitor potentially for the treatment of non-Hodgkin | |
| | | B-cell lymphomas and solid tumours. | |
| | PF-3758309 | A serine/threonine-protein kinase PAK4 inhibitor potentially for the treatment of | Phase I |
| | | solid tumours. | |

TABLE AI
Pharmacodia drug targets for LUAD vs. LUSC using MEGENA

| Gene | Drug Name | Description | Clinical Trials |
|---|---|---|---|
| CTSC | AZD-7986 | A Cathepsin C (CTSC) modulator potentially for the treatment of chronic | Phase I |
| | | obstructive pulmonary disease. | |
| KCNC1 | AUT-00063 | A voltage-gated potassium channel subunitKv3.1 (KCNC1) modulator potentially | Phase II |
| | | for the treatment of hearing loss and tinnitus. | |

TABLE AJ
Pharmacodia drug targets for LUAD vs. LUSC using nGOseq

| Gene | Drug Name | Description | Clinical Trials |
|---|---|---|---|
| GHSR | Relamorelin | A growth hormone secretagogue receptor (GHSR) agonist potentially for the | Phase II |
| | | treatment of gastroparesis diabeticomm, anorexia nervosa and constipation. | |
| | GTP-200 | A growth hormone releasing factor (GHSR) agonist potentially for the treatment | Phase II |
| | | of cachexia. | |
| MST1R | ASLAN-002 | A macrophage stimulating 1 receptor (MST1R) and hepatocyte growth factor | Phase II |
| | | receptor (c-Met/HGFR) inhibitor potentially for the treatment of gastric and | |
| | | breast cancer. | |
| | MK-8033 | A c-MET and MST1R inhibitor potentially for the treatment of solid tumors. | Phase I |
| USP1 | VLX-600 | An UCHL5 and USP14 protein inhibitor potentially for the treatment of solid | Phase I |
| | | tumours. | |

TABLE AK
Pharmacodia drug targets for Luminal A vs. Luminal B using MEGENA

| | | | Clinical |
|---|---|---|---|
| Gene | Drug Name | Description | Trials |
| SMO | Glasdegib | A smoothened (SMO) receptor antagonist potentially for treatment of | Phase II |
| | | myelodysplastic syndrome (MDS), chronic myeloid leukemia (CML) and | |
| | | acute myeloid leukemia(AML). | |
| | BMS-833923 | A smoothened (SMO) receptor antagonist potentially for the treatment of basal | Phase II |
| | | cell nevus syndrome. | |
| | LEQ-506 | A SMO receptor antagonist potentially for the treatment of advanced solid | Phase I |
| | | tumors. | |
| | BMS-833923 | A smoothened (SMO) receptor antagonist potentially for the treatment of basal | Phase II |
| | | cell nevus syndrome. | |
| | Cipromedegib | A smoothened receptor (SMO) inhibitor potentially for the treatment of gastric | Phase I |
| | | cancer, lung cancer, medulloblastoma and basal cell carcinoma (BCC). | |
| | CUR-61414 | A smoothened (SMO) receptor antagonist potentially for the treatment of basal | Phase I |
| | | cell carcinoma (BCC). | |
| | Vismodegib | A smoothened receptor (SMO) antagonist used to treat basal cell carcinoma | Approved |
| | | (BCC). | |
| | Taladegib | A smoothened (SMO) receptor antagonist potentially for the treatment of | Phase II |
| | Hydrochloride | esophageal cancer and small cell lung cancer (SCLC). | |
| | TAK-441 | A smoothened receptor (SMO) antagonist potentially for the treatment of Solid | Phase I |
| | | tumours. | |
| | Sonidegib | A smoothened receptor (SMO) antagonist used to treat advanced basal cell | Approved |
| | Phosphate | carcinoma (BCC). | |

TABLE AL
Pharmacodia drug targets for Luminal A vs. Luminal B using nGOseq

| | Drug | | Clinical |
|---|---|---|---|
| Gene | Name | Description | Trials |
| ATM | AZD-0156 | An ataxia telangiectasia mutated kinase (ATM) inhibitor potentially for the | Phase I |
| | | treatment of solid tumors. | |
| AVPR1B | Nelivaptan | A vasopressin 1B receptor (AVPR1B) antagonist potentially for the | Phase II |
| | | treatment of generalised anxiety disorder and major depressive disorder. | |
| | ABT-436 | A vasopressin 1B receptor (AVPR1B) antagonist potentially for the | Phase II |
| | | treatment of alcohol dependence. | |
| BIRC5 | EZN-3042 | A BIRC5 protein inhibitor potentially for the treatment of acute | Phase I |
| | | lymphoblastic leukaemia, lymphoma and solid tumours. | |
| | SVN53-67/M57-KLH | A peptide mimic vaccine targeting survivin (BIRC5) for the treatment of | Phase II |
| | peptide | glioblastoma. | |
| | vaccine | | |
| | Terameprocol | A baculoviral inhibitor of apoptosis repeat-containing 5 (BIRC5) inhibitor | Phase II |
| | | potentially for the treatment of cervical intraepithelial neoplasia, glioma | |
| | | and human papillomavirus infections. | |
| | Sepantronium | A baculoviral inhibitor of apoptosis repeat-containing 5 (BIRC5) inhibitor | Phase II |
| | Bromide | potentially for the treatment of cancer. | |
| C5AR1 | PMX-53 | A complement component 5a receptor 1 (C5AR1) antagonist potentially | Phase II |
| | | for the treatment of osteoarthritis (OA), rheumatoid arthritis and psoriasis. | |
| CX3CR1 | BI-655088 | A nanobody targeting C-X3-C motif chemokine receptor 1 (CX3CR1) | Phase I |
| | | potentially for the treatment of kidney disorders. | |
| GPC3 | ERY-974 | A bispecific antibody targeting glypican3 (GPC3) and CD3 potentially for | Phase I |
| | | the treatment of solid tumors. | |
| | Codrituzumab | A glypican 3 (GPC3) targeted antibody potentially for the treatment of | Phase II |
| | | metastatic hepatocellular carcinoma. | |
| LPAR3 | SAR-100842 | A lysophosphatidic acid receptor (LPAR1, LPAR3) antagonist potentially | Phase II |
| | | for the treatment of systemic scleroderma. | |
| NPR3 | Linaclotide | A natriuretic peptide receptor 3 (NPR3) agonist used to treat irritable | Approved |
| | | bowel syndrome with constipation (IBS-C) and chronic idiopathic | |
| | | constipation (CIC). | |
| TNFRSF18 | MEDI-1873 | An antibody targeting tumour necrosis factor receptor superfamily member | Phase I |
| | | 18 (TNFRSF18, GITR) potentially for the treatment of solid tumour. | |
| XCR1 | Reparixin | A inhibitor of C-X-C motif chemokine receptor 1/2 (CXCR1/2) potentially | Phase III |
| | | for the treatment of delayed graft function. | |
| | Navarixin | A C-X-C motif chemokine receptor 1 (CXCR1) antagonist and C-X-C | Phase II |
| | | motif chemokine receptor 2 (CXCR2) antagonist potentially for the | |
| | | treatment of chronic obstructive pulmonary disease (COPD), asthma and | |
| | | psoriasis. | |
| | Ladarixin | A C-X-C motif chemokine receptor (CXCR1, CXCR2) antagonist | Phase II |
| | Sodium | potentially for the treatment of type I diabetes. | |
| | CXCR1/2 | A CXCR1/2 ligands inhibitor potentially for the treatment of | Phase I |
| | ligands | immunological disorders. | |
| | antibody | | |

TABLE AM
Pharmacodia drug targets for pan-22 cancer multinomial modeling using MEGENA.

| | | | Clinical |
|---|---|---|---|
| Gene | Drug Name | Description | Trials |
| AGT | Lomeguatrib | An O6-alkylguanine-DNA alkyltransferase | Phase II |
| | | (AGT/MGMT/AGAT) inhibitor potentially for the treatment of | |
| | | metastatic melanoma and metastatic colorectal cancer. | |
| ANGPTL3 | Evinacumab | An angiopoietin like 3 (ANGPTL3) targeted antibody potentially | Phase II |
| | | for the treatment of hypertriglyceridemia and | |
| | | hypercholesterolemia. | |
| | IONIS- | An angiopoietin like 3 (ANGPTL3) protein inhibitor potentially | Phase II |
| | ANGPTL3Rx | for the treatment of hyperlipoproteinaemia type IIa. | |
| CYP17A1 | ODM-204 | An androgen receptor (AR) antagonist and steroid 17-alpha- | Phase II |
| | | hydroxylase (CYP17A1) inhibitor potentially for the treatment of | |
| | | prostate cancer. | |
| | Abiraterone Acetate | A prodrug of abiraterone with CYP17A1 enzyme inhibition used | Approved |
| | | to treat prostate cancer. | |
| | Orteronel | A steroid 17-alpha-hydroxylase (CYP17A1) inhibitor potentially | Phase III |
| | | for the treatment of prostate cancer. | |
| | Orteronel | A steroid 17-alpha-hydroxylase (CYP17A1) inhibitor potentially | Phase III |
| | | for the treatment of prostate cancer. | |
| | ASN-001 | A steroid 17-alpha-hydroxylase (CYP17A1) inhibitor | Phase II |
| | | potentially for the treatment of prostate cancer. | |
| EGF | Panitumumab | An epidermal growth factor receptor (EGFR) antagonist used to | Approved |
| | | treat wild-type KRAS (exon 2) metastatic colorectal cancer | |
| | | (mCRC). | |
| | Recombinant | An epidermal growth factor receptor (EGFR) agonist used to | Approved |
| | epidermal | treat bums, diabetic foot ulcer and wounds. | |
| | growth factor | | |
| | (Bharat Biotech) | | |
| | KHK-2866 | A heparin binding EGF like growth factor (HB-EGF) inhibitor | Phase I |
| | | for the treatment of ovarian cancer and some other solid tumour. | |
| | Recombinant | An epidermal growth factor receptor (EGFR) agonist used to | Approved |
| | epidermal growth | treat bums, diabetic foot ulcer and wounds. | |
| | factor (Bharat | | |
| | Biotech) | | |
| | Lapatinib Ditosylate | A dual epidermal growth factor receptor (EGFR) and human | Approved |
| | Hydrate | epidermal growth factor receptor 2 (ErbB2/HER2) inhibitor used | |
| | | to treat breast cancer and other solid tumours. | |
| | Tarloxotinib | A EGFR/ErbB2/ErbB4 inhibitor potentially for the treatment of | Phase II |
| | Bromide | squamous cell carcinoma of head and neck and non-small cell | |
| | | lung cancer. | |
| | Cetuximab biosimilar | An epidermal growth factor receptor (EGFR) antagonist | Phase III |
| | (Shanghai Zhangjiang | potentially for the treatment of colorectal cancer. | |
| | Biotechnology) | | |
| | Epitinib Succinate | An EGFR inhibitor potentially for the treatment of solid tumours | Phase II |
| | | and non small cell lung cancer (NSCLC). | |
| | RM-1929 | An EGFR targeted antibody conjugated to IR-700 potentially for | Phase I |
| | | the treatment of head and neck cancer. | |
| | Allitinib Tosylate | An EGFR and ErbB2 inhibitor potentially for the treatment of | Phase II |
| | | lung cancer and breast cancer. | |
| | Cetuximab | An epidermal growth factor receptor (EGFR) antagonist used to | Approved |
| | | treat colorectal cancer, head and neck cancer. | |
| | Theliatinib | An epidermal growth factor receptor (EGFR) inhibitor potentially | Phase I |
| | | for the treatment of esophagus cancer and other advanced solid | |
| | | tumours. | |
| FGF1 | Sprifermin | A recombinant human fibroblast growth factor 18 (FGF18) | Phase II |
| | | potentially for the treatment of osteoarthritis. | |
| GJA1 | CODA-001 | A gap junction alpha-1 protein (GJA1) inhibitor potentially for | Phase II |
| | | the treatment of diabetic foot ulcer, leg ulcer and wounds. | |
| MGMT | Lomeguatrib | An O6-alkylguanine-DNA alkyltransferase | Phase II |
| | | (AGT/MGMT/AGAT) inhibitor potentially for the treatment of | |
| | | metastatic melanoma and metastatic colorectal cancer. | |
| | O6-Benzylguanine | A O6-alkylguanine-DNA alkyltransferase (MGMT) potentially | Phase II |
| | | for the treatment of glioblastoma multiforme. | |
| PTPN1 | KQ-791 | A protein tyrosine phosphatase non receptor type 1 (PTPN1) | Phase I |
| | | antagonist potentially for the treatment of type 2 diabetes and | |
| | | insulin resistance. | |

TABLE AN
Pharmacodia drug targets for pan-20 cancer survival using MEGENA

| | Drug | | Clinical |
|---|---|---|---|
| Gene | Name | Description | Trials |
| CDK4 | Trilaciclib | A cyclin-dependent kinase 4 (CDK4) inhibitor and cyclin-dependent kinase 6 | Phase II |
| | Hydrochloride | (CDK6) inhibitor potentially for the treatment of small cell lung cancer. | |
| | Palbociclib | A cyclin-dependent kinase (CDK4/6) inhibitor potentially for the treatment of | Phase I |
| | Isethionate | central nervous system tumors. | |
| | G1T-38 | A cyclin-dependent kinase 4 (CDK4) inhibitor and a cyclin-dependent kinase | Phase II |
| | | 6 (CDK6) inhibitor potentially for the treatment of cancer. | |
| | SHR-6390 | A CDK4/6 inhibitor potentially for the treatment of melanoma and | Phase I |
| | | malignancies. | |
| | Palbociclib | A cyclin-dependent kinase (CDK4/6) inhibitor used to treat advanced breast | Approved |
| | | cancer. | |
| | Birociclib | A CDK4/6 inhibitor potentially for the treatment of breast cancer and | Phase I |
| | | malignant brain tumor. | |
| | MM-D37K | A cyclin-dependent kinase 4/6 (CDK4/6) inhibitor ptentially for the treatment | Phase II |
| | | of bladder cancer, gastrointestinal cancer, glioblastoma and malignant | |
| | | melanoma. | |
| | Riviciclib | A CDK4 and CDK9 inhibitor potentially for the treatment of breast cancer | Phase III |
| | | and radiation induced mucositis in head and neck cancer. | |
| | Abemaciclib | A CDK4/6 inhibitor used for the treatment of HR-positive, HER2-negative | Approved |
| | | advanced or metastatic breast cancer. | |
| | Ribociclib | A cyclin-dependent kinase 4/6 (CDK4/6) inhibitor used for the treatment of | Approved |
| | Succinate | postmenopausal women with hormone receptor (HR)-positive, human | |
| | | epidermal growth factor receptor 2 (HER2)-negative advanced or metastatic | |
| | | breast cancer. | |
| OLR1 | EC-1456 | A folate receptor 1 inhibitor (FOLR1) potentially for the treatment of solid | Phase I |
| | | tumours and non small cell lung cancer (NSCLC). | |
| | Mirvetuximab | A FOLR1 targeted antibody conjugated to maytansinoid DM4 potentially for | Phase II |
| | soravtansine | the treatment of fallopian tube cancer, ovarian cancer, peritoneal cancer and | |
| | | endometrial cancer. | |
| TRPV4 | GSK-2798745 | A transient receptor potential cation channel subfamily V member 4 (TRPV4) | Phase II |
| | | antagonist potentially for the treatment of heart failure and pulmonary edema. | |

TABLE AO
Pharmacodia drug targets for pan-20 cancer survival using nGOseq

| | | | Clinical |
|---|---|---|---|
| Gene | Drug Name | Description | Trials |
| C2 | Vistusertib | A mammalian target of rapamycin complex 1 (mTORC1) inhibitor and | Phase II |
| | | mammalian target of rapamycin complex 2 (mTORC2) inhibitor | |
| | | potentially for the treatment of solid tumours. | |
| CD80 | Galiximab | A CD80 targeted antibody potentially for the treatment of autoimmune | Phase II |
| | | disorders, non-Hodgkin's lymphoma and psoriasis. | |
| | AV-1142742 | A cluster of differentiation 80 (CD80) inhibitor potentially for the | Phase II |
| | | treatment of autoimmune disease (AID). | |
| MIP | Macrophage | A (MIP)-1α analogue potentially for the treatment of breast cancer | Phase II |
| | inflammatory | chemo/radiotherapy-induced myelosuppression, HIV infections and | |
| | protein-1α | myeloid leukaemia. | |
| | analogue | | |
| | ECI-301 | A derivative of human chemokine MIP-1α potentially for the treatment | Phase I |
| | | of hepatocellular carcinoma and cancer. | |
| SCARB1 | ITX-5061 | A scavenger receptor B1 antagonist (SCARB1) potentially for the | Phase II |
| | | treatment of HCV infection. | |

As used herein, “plurality” means two or more and includes a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or more or any range inclusive.

Methods

Methods of Identifying Therapeutic or Drug Targets

Methods of the invention include identifying at least one therapeutic or drug target for at least one cancer type (e.g., any of the cancers listed in Table A). The methods also include binomial comparisons to classify cancers of the same tissue of origin or between molecular subtypes. Such binomial comparisons include, LUAD vs. LUSC, KIRC vs. KIRP, ER+vs. ER− BRCA subtypes, and Luminal A vs. Luminal B BRCA subtypes.

The methods can identify at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, or more therapeutic or drug targets. The methods can comprise receiving or obtaining at least one, two, three, four, or more data sets from at least one cancer type (e.g., any of the cancers listed in Table A). The data sets can comprise whole genome sequencing data, whole exome sequencing data, RNA-Seq data, miRNA-SEQ data, cDNA sequencing data, and Methylation Array data from a company, hospital, researcher, and the like, who is interested in identifying biologically relevant sets of gens whose collective state correlates with a given phenotype. Once received, downloaded, or obtained, the data sets are processed according to the methods, systems, algorithms, programs, and codes set forth above to identify therapeutic or drug targets or genes. The methods, systems, algorithms, programs, and codes enable perfect and near perfect classifications of multiple human tumor type designations, independent of tissue-specific annotation, to identify known and previously undescribed integrated molecular signatures of pan-cancer etiology and patient survival, thus creating a new archetype for biological and therapeutic discovery identify at least one therapeutic or drug target.

In some embodiments, the therapeutic or drug targets or genes are set forth in Table B, Table C, Table D, Table E, Table F, Table G, Table H, Table I, Table J, Table K, Table L, Table M, Table N, Table O, Table AP, Table AQ, Table AR, Table AS, Table AT, Table AU, Table AV, Table AX, Table AY, Table AZ, Table AAA, Table AAB, Table AAC, Table AAD, Table AAF, Table AAG, Table AAH, Table AAJ, Table AAK, Table AAL, Table AAM, Table AAN, Table AAO, or combinations thereof.

In certain embodiments, the therapeutic or drug targets or genes for BRCA are set forth in Appendix A, Appendix B, Table B, Table C, Table AT, Table AU, Table AV, or combinations thereof. In some embodiments, the at least one therapeutic or drug target for BRCA is at least fifty therapeutic or drug targets, wherein said at least fifty therapeutic or drug targets correspond to the fifty genes listed in Table B. In some embodiments, the at least one therapeutic or drug target for BRCA is at least fifty-two therapeutic or drug targets, wherein said at least fifty-two therapeutic or drug targets correspond to the fifty-two genes listed in Table C. In some embodiments, the at least one therapeutic or drug target for BRCA is at least twenty-three therapeutic or drug targets, wherein said at least twenty-three therapeutic or drug targets correspond to the twenty-three genes listed in Table AT. In some embodiments, the at least one therapeutic or drug target for BRCA is at least fourteen therapeutic or drug targets, wherein said at least fourteen therapeutic or drug targets correspond to the fourteen genes listed in Table AU. In some embodiments, the at least one therapeutic or drug target for BRCA is at least five therapeutic or drug targets, wherein said at least five therapeutic or drug targets correspond to the at least genes listed in Table AV.

In certain embodiments, the therapeutic or drug targets of genes for LUAD or LUSC are set forth in Appendix G, Appendix H, Table H, Table I, Table AAB, Table AAC, Table AAD, or combinations thereof. In some embodiments, the at least one therapeutic or drug target for LUAD or LUSC is at least fifty therapeutic or drug targets, wherein said therapeutic or drug targets correspond to the fifty genes listed Table H. In some embodiments, the at least one therapeutic or drug target for LUAD or LUSC is at least fifty therapeutic or drug targets, wherein said therapeutic or drug targets correspond to the fifty genes listed Table E. In some embodiments, the at least one therapeutic or drug target for LUAD or LUSC is at least twenty-five therapeutic or drug targets, wherein said at least twenty-five therapeutic or drug targets correspond to the twenty-five genes listed in Table AAB. In some embodiments, the at least one therapeutic or drug target for LUAD or LUSC is at least fourteen therapeutic or drug targets, wherein said at least fourteen therapeutic or drug targets correspond to the fourteen genes listed in Table AAC. In some embodiments, the at least one therapeutic or drug target for LUAD or LUSC is at least three therapeutic or drug targets, wherein said at least three therapeutic or drug targets correspond to the three genes listed in Table AAD.

In certain embodiments, the therapeutic or drug targets or genes for ER positive or ER negative are set forth in Appendix C, Appendix D, Table D, Table E, Table AX, Table AY, Table AZ, Table AAA, or combinations thereof. In some embodiments, the at least one therapeutic or drug target for ER positive or ER negative is at least fifty-two therapeutic or drug targets, wherein said therapeutic or drug targets correspond to the fifty-two genes listed Table D. In some embodiments, the at least one therapeutic or drug target for ER positive or ER negative is at least fifty-two therapeutic or drug targets, wherein said therapeutic or drug targets correspond to the fifty-two genes listed Table E. In some embodiments, the at least one therapeutic or drug target for ER positive or ER negative is at least thirty-two therapeutic or drug targets, wherein said at least thirty-two therapeutic or drug targets correspond to the thirty-two genes listed in Table AX. In some embodiments, the at least one therapeutic or drug target for ER positive or ER negative is at least seventeen therapeutic or drug targets, wherein said at least seventeen therapeutic or drug targets correspond to the seventeen genes listed in Table AY. In some embodiments, the at least one therapeutic or drug target for ER positive or ER negative corresponds to the one gene listed in Table AZ. In some embodiments, the at least one therapeutic or drug target for ER positive or ER negative is at least two therapeutic or drug targets, wherein said at least two therapeutic or drug targets correspond to the two genes listed in Table AAA.

In certain embodiments, the therapeutic or drug targets or genes for Luminal A or Luminal B are set forth in Appendix I, Appendix J, Table J, Table K, Table AAF, Table AAG, Table AAH, or combinations thereof. In some embodiments, the at least one therapeutic or drug target for Luminal A or Luminal B is at least fifty-one therapeutic or drug targets, wherein said therapeutic or drug targets correspond to the fifty-one genes listed Table J. In some embodiments, the at least one therapeutic or drug target for Luminal A or Luminal B is at least fifty-one therapeutic or drug targets, wherein said therapeutic or drug targets correspond to the fifty-one genes listed Table K. In some embodiments, the at least one therapeutic or drug target for Luminal A or Luminal B is at least thirty-two therapeutic or drug targets, wherein said at least thirty-two therapeutic or drug targets correspond to the thirty-two genes listed in Table AAF. In some embodiments, the at least one therapeutic or drug target for Luminal A or Luminal B is at least seventeen therapeutic or drug targets, wherein said at least seventeen therapeutic or drug targets correspond to the seventeen genes listed in Table AAG. In some embodiments, the at least one therapeutic or drug target for Luminal A or Luminal B is at least three therapeutic or drug targets, wherein said at least therapeutic or drug targets correspond to the three genes listed in Table AAH.

In certain embodiments, the therapeutic or drug targets or genes for KIRP or KIRC are set forth in Appendix E, Appendix F, Table F, Table G, Table AP, Table AQ, Table AR, Table AS, or combinations thereof. In some embodiments, the at least one therapeutic or drug target for KIRP or KIRC is at least fifty-seven therapeutic or drug targets, wherein said therapeutic or drug targets correspond to the fifty-seven genes listed Table F. In some embodiments, the at least one therapeutic or drug target for KIRP or KIRC is at least fifty-three therapeutic or drug targets, wherein said therapeutic or drug targets correspond to the fifty-three genes listed Table G. In some embodiments, the at least one therapeutic or drug target for KIRP or KIRC is at least twenty-eight therapeutic or drug targets, wherein said at least twenty-eight therapeutic or drug targets correspond to the twenty-eight genes listed in Table AP. In some embodiments, the at least one therapeutic or drug target for KIRP or KIRC is at least twenty-two therapeutic or drug targets, wherein said at least twenty-two therapeutic or drug targets correspond to the twenty-two genes listed in Table AQ. In some embodiments, the at least one therapeutic or drug target for KIRP or KIRC is at least three therapeutic or drug targets, wherein said at least three therapeutic or drug targets correspond to the three genes listed in Table AR. In some embodiments, the at least one therapeutic or drug target for KIRP or KIRC corresponds to the one gene listed in Table AS.

In certain embodiments, the therapeutic or drug targets or genes shared between multiple cancer types (e.g. any of the cancers in Table A) are set forth in Appendix K, Appendix, L, Table L, Table M, Table AAJ, Table AAK, or combinations thereof. In some embodiments, the at least one therapeutic or drug target for pan-cancer is at least two hundred therapeutic or drug targets, wherein said therapeutic or drug targets correspond to the two hundred genes listed in Table M. In some embodiments, the at least one therapeutic or drug target for pan-cancer is at least fifty-one therapeutic or drug targets, wherein said therapeutic or drug targets correspond to the fifty-one genes listed in Table L. In some embodiments, the at least one therapeutic or drug target for pan-cancer is at least forty-six therapeutic or drug targets, wherein said at least forty-six therapeutic or drug targets correspond to the forty-six genes listed in Table AAJ. In some embodiments, the at least one therapeutic or drug target for pan-cancer is at least twenty-six therapeutic or drug targets, wherein said at least twenty-six therapeutic or drug targets correspond to the twenty-six genes listed in Table AAK.

In certain embodiments, the therapeutic or drug targets or genes shared between multiple cancer types (e.g. any of the cancers in Table A) that are indicative of survival are set forth in Appendix M, Appendix N, Table N, Table O, Table AAL, Table AAM, Table AAN, Table AAO, or combinations thereof. In some embodiments, the at least one therapeutic or drug target shared between multiple cancer types that are indicative of survival is at least fifty-one therapeutic or drug targets, wherein said therapeutic or drug targets correspond to the fifty-one genes listed in Table N. In some embodiments, the at least one therapeutic or drug target shared between multiple cancer types that are indicative of survival is at least fifty-one therapeutic or drug targets, wherein said therapeutic or drug targets correspond to the fifty-one genes listed in Table O. In some embodiments, the at least one therapeutic or drug target shared between multiple cancer types that are indicative of survival is at least twenty-seven therapeutic or drug targets, wherein said at least twenty-seven therapeutic or drug targets correspond to the twenty-seven genes listed in Table AAL. In some embodiments, the at least one therapeutic or drug target shared between multiple cancer types that are indicative of survival is at least twenty-three therapeutic or drug targets, wherein said at least twenty-three therapeutic or drug targets correspond to the twenty-three genes listed in Table AAM. In some embodiments, the at least one therapeutic or drug target shared between multiple cancer types that are indicative of survival is at least three therapeutic or drug targets, wherein said at least three therapeutic or drug targets correspond to the three genes listed in Table AAN.

Methods of Detecting and/or Diagnosing Cancers

Methods of the invention include detecting and/or diagnosing a cancer in a subject having or suspected of having a cancer (e.g., any of the cancers listed in Table A). The method can include determining the expression levels of a plurality of therapeutic or drug targets or genes (e.g., RNA transcripts or expression products thereof of) at pre-selected number or plurality of therapeutic or drug targets or genes in a biological sample from a subject having or suspected of having a cancer such as a cancer.

The methods generally begin by collecting, obtaining, or receiving a biological sample from a subject having or suspected of having a cancer (e.g., any of the cancers listed in Table A). The biological sample can comprise any collection of cells, tissues, organs or bodily fluids in which expression of a therapeutic or drug target or gene can be detected. Examples of such samples include, but are not limited to, biopsy specimens of cells, tissues or organs, bodily fluids and smears.

When the sample is a biopsy specimen, it can include, but is not limited to, cells from a biopsy, such as a tumor tissue sample. Biopsy specimens can be obtained by a variety of techniques including, but not limited to, scraping or swabbing an area, using a needle to aspirate cells or bodily fluids, or removing a tissue sample. Methods for collecting various body samples/biopsy specimens are well known in the art, and may include, for example, fine needle aspiration biopsy, core needle biopsy, or excisional biopsy.

Fixative and staining solutions can be applied to, for example, cells or tissues for preserving them and for facilitating examination. Body samples, particularly tissue samples, can be transferred to a glass slide for viewing under magnification. The body sample can be a formalin-fixed, paraffin-embedded tissue sample, particularly a primary tumor sample.

When the sample is a bodily fluid, it can include, but is not limited to, blood, lymph, urine, saliva, aspirates or any other bodily secretion or derivative thereof. When the sample is blood, it can include whole blood, plasma, serum or any derivative of blood.

After collecting and preparing the specimen from the subject having or suspected of having cancer (e.g., any of the cancers listed in Table A), the methods then include detecting expression of the therapeutic or drug targets or genes. One can use any method available for detecting expression of polynucleotides and polypeptides. As used herein, “detecting expression” means determining the quantity or presence of a therapeutic or drug target or gene polynucleotide or its expression product. As such, detecting expression encompasses instances where a therapeutic or drug target or gene is determined not to be expressed, not to be detectably expressed, expressed at a low level, expressed at a normal level, or overexpressed.

Methods of Determining Expression Levels

Expression of a therapeutic or drug target or gene can be determined by normalizing the level of a reference marker/control, which can be all measured transcripts (or their products) in the sample or a particular reference set of RNA transcripts (or their products). Normalization can be performed to correct for or normalize away both differences in the amount of therapeutic or drug target or gene assayed and variability in the quality of the therapeutic or drug target or gene type used. Therefore, an assay typically measures and incorporates the expression of certain normalizing polynucleotides or polypeptides, including well known housekeeping genes, such as, for example, GAPDH and/or actin. Alternatively, normalization can be based on the mean or median signal of all of the assayed therapeutic or drug targets or genes or a large subset thereof (global normalization approach).

To determine overexpression, the sample can be compared with a corresponding sample that originates from a healthy individual. That is, the “normal” level of expression is the level of expression of the therapeutic or drug target or gene in, for example, a tissue sample from an individual not afflicted with cancer. Such a sample can be present in standardized form. Sometimes, determining therapeutic or drug target or gene overexpression requires no comparison between the sample and a corresponding sample that originated from a healthy individual. For example, detecting overexpression of a therapeutic or drug target or gene indicative of a poor prognosis in a tumor sample may preclude the need for comparison to a corresponding tissue sample that originates from a healthy individual. Moreover, no expression, underexpression or normal expression (i.e., the absence of overexpression) of a therapeutic or drug target or gene or combination of therapeutic or drug targets or genes of interest provides useful information regarding the prognosis of a cancer patient.

Methods of detecting and quantifying polynucleotide therapeutic or drug target or genes in a sample are well known in the art. Such methods include, but are not limited to gene expression profiling, which are based on hybridization analysis of polynucleotides, and sequencing of polynucleotides. The most commonly used methods art for detecting and quantifying polynucleotide expression in include northern blotting and in situ hybridization (Parker & Barnes (1999) Methods Mol. Biol. 106:247-283), RNAse protection assays (Hod (1992) Biotechniques 13:852-854), PCR-based methods, such as RT-PCR (Weis et al. (1992) TIG 8:263-264), and array-based methods (Schena et al. (1995) Science 270:467-470). Alternatively, antibodies may be employed that can recognize specific duplexes, including DNA duplexes, RNA duplexes, and DNA-RNA hybrid duplexes, or DNA-protein duplexes in, for example, an oligonucleotide-linked immunosorbent assay (“OLISA”). See, Lee et al. (1985) FEBS Lett. 190:120-124; Han et al. (2010) Bioconjug. Chem. 21:2190-2196; Miura et al. (1987) Biochem. Biophys. Res. Commun. 144:930-935; and Tanha & Lee (1997) Nucleic Acids Res. 25:1442-1449. Representative methods for sequencing-based gene expression analysis include Serial Analysis of Gene Expression (“SAGE”) and gene expression analysis by massively parallel signature sequencing. See, Velculescu et al. (1995) Science 270: 484-487.

Isolated RNA can be used to determine the level of therapeutic or drug target or gene transcripts (i.e., mRNA) in a sample, as many expression detection methods use isolated RNA. The starting material typically is total RNA isolated from a body sample, such as a tumor or tumor cell line, and corresponding normal tissue or cell line, respectively. Thus, RNA can be isolated from a variety of primary tumors, including breast, lung, colon, prostate, brain, liver, kidney, pancreas, spleen, thymus, testis, ovary, uterus, and the like, or tumor cell lines. If the source of mRNA is a primary tumor, mRNA can be extracted, for example, from frozen or archived paraffin-embedded and fixed (e.g., formalin-fixed) tissue samples.

Methods of isolating polynucleotides such as RNA from a sample are well known in the art. See, e.g., Molecular Cloning: A Laboratory Manual, 3rd ed. (Sambrook et al. eds., Cold Spring Harbor Press 2001); and Current Protocols in Molecular Biology (Ausubel et al. eds., John Wiley & Sons 1995). Methods for RNA extraction from paraffin-embedded tissues also are well known in the art. See, e.g., Rupp & Locker (1987) Lab Invest. 56:A67; and De Andres et al. (1995) Biotechniques 18:42-44. Moreover, isolation/purification kits are commercially available for isolating polynucleotides such as RNA (Qiagen; Valencia, Calif.). For example, total RNA from cells in culture can be isolated using Qiagen RNeasy® Mini-Columns. Other commercially available RNA isolation/purification kits include MasterPure™ Complete DNA and RNA Purification Kit (Epicentre; Madison, Wis.) and Paraffin Block RNA Isolation Kit (Ambion; Austin, Tex.). Total RNA from tissue samples can be isolated, for example, using RNA Stat-60 (Tel-Test; Friendswood, Tex.). RNA prepared from a tumor can be isolated, for example, by cesium chloride density gradient centrifugation. Additionally, large numbers of tissue samples readily can be processed using techniques well known to those of skill in the art, such as, for example, the single-step RNA isolation process of Chomczynski (U.S. Pat. No. 4,843,155).

Once isolated, the polynucleotide, such as mRNA, can be used in hybridization or amplification assays including, but not limited to, Southern or Northern blotting, PCR and probe arrays. One method of detecting polynucleotide levels involves contacting the isolated polynucleotides with a nucleic acid molecule (probe) that can hybridize to the desired polynucleotide target. The nucleic acid probe can be, for example, a full-length DNA, or a portion thereof, such as an oligonucleotide of at least about 10, 15, 20, 30, 40, 50, 75, 100, 125, 150, 175, 200, 225, 250, 275, 300, 400 or 500 nucleotides or more in length and sufficient to specifically hybridize under stringent conditions to a polynucleotide such as an mRNA or genomic DNA encoding a therapeutic or drug target or gene of interest. Hybridization of a polynucleotide encoding the therapeutic or drug target or gene of interest with the probe indicates that the therapeutic or drug target or gene in question is being expressed.

Stringent hybridization conditions are defined as hybridizing at 68° C. in 5×SSC/5×Denhardt's solution/1.0% SDS, and washing in 0.2×SSC/0.1% SDS+/−100 μg/ml denatured salmon sperm DNA at room temperature (RT), and moderately stringent hybridization conditions are defined as washing in the same buffer at 42° C. Additional guidance regarding such conditions is readily available in the art, for example, in Molecular Cloning: A Laboratory Manual, 3rd ed. (Sambrook et al. eds., Cold Spring Harbor Press 2001); and Current Protocols in Molecular Biology (Ausubel et al. eds., John Wiley & Sons 1995).

Another method of detecting polynucleotide expression levels involves immobilized polynucleotides on a solid surface and contacting the immobilized polynucleotides with a probe, for example by running isolated mRNA on an agarose gel and transferring the mRNA from the gel to a membrane, such as nitrocellulose. Alternatively, the probes can be immobilized on a solid surface and isolated mRNA is contacted with the probes, for example, in an Agilent Gene Chip Array.

For example, microarrays can be used to detect polynucleotide expression. Microarrays are particularly well suited because of the reproducibility between different experiments. DNA microarrays provide one method for the simultaneous measurement of the expression levels of large numbers of polynucleotides. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, e.g., U.S. Pat. Nos. 6,040,138; 5,800,992; 6,020,135; 6,033,860 and 6,344,316. High-density oligonucleotide arrays are particularly useful for determining expression profiles for a large number of polynucleotides in a sample.

Methods of synthesizing these arrays using mechanical synthesis methods are described in, for example, U.S. Pat. No. 5,384,261. Although a planar array surface generally is used, the array can be fabricated on a surface of virtually any shape or even a multiplicity of surfaces. Arrays can be nucleic acids (or peptides) on beads, gels, polymeric surfaces, fibers (such as fiber optics), glass or any other appropriate substrate. See, e.g., U.S. Pat. Nos. 5,770,358; 5,789,162; 5,708,153; 6,040,193 and 5,800,992.

As such, PCR-amplified inserts of cDNA clones can be applied to a substrate in a dense array. For example, at least about 10,000 nucleotide sequences can be applied to the substrate. The microarrayed genes, immobilized on the microchip at 10,000 elements each, are suitable for hybridization under stringent conditions. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of polynucleotide can be hybridized pairwise to the array. The relative abundance of the transcripts from the two sources corresponding to each specified molecule is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels. See, Schena et al. (1996) Proc. Natl. Acad Sci. USA 93:106-149. Advantageously, microarray analysis can be performed by commercially available equipment, following manufacturer's protocols, such as by using the Affymetrix® GenChip Technology, or Agilent® Ink-Jet Microarray Technology. The development of microarray methods for large-scale analysis of gene expression makes it possible to search systematically for molecular markers of cancer classification and outcome prediction in a variety of tumor types.

Another method of detecting polynucleotide expression levels involves a digital technology developed by NanoString® Technologies (Seattle, Wash.) and based on direct multiplexed measurement of gene expression, which offers high levels of precision and sensitivity (<1 copy per cell). The method uses molecular “barcodes” and single molecule imaging to detect and count hundreds of unique transcripts in a single reaction. Each color-coded barcode is attached to a single target-specific probe corresponding to a gene of interest. Mixed together with controls, they form a multiplexed CodeSet. Two ˜50 base probes per mRNA can be included for hybridization. The reporter probe carries the signal, and the capture probe allows the complex to be immobilized for data collection. After hybridization, the excess probes are removed and the probe/target complexes aligned and immobilized in an nCounter® Cartridge. Sample cartridges are placed in a digital analyzer for data collection. Color codes on the surface of the cartridge are counted and tabulated for each target molecule.

Another method of detecting polynucleotide expression levels involves nucleic acid amplification, for example, by RT-PCR (U.S. Pat. No. 4,683,202), ligase chain reaction (Barany (1991) Proc. Natl. Acad Sci. USA 88:189-193), self-sustained sequence replication (Guatelli et al. (1990) Proc. Natl. Acad Sci. USA 87:1874-1878), transcriptional amplification system (Kwoh et al. (1989) Proc. Natl. Acad Sci. USA 86:1173-1177), Q-Beta Replicase (Lizardi et al., (1988) Bio/Technology 6:1197), rolling circle replication (U.S. Pat. No. 5,854,033), or any other nucleic acid amplification method, followed by the detection of the amplified molecules using techniques well known in the art. Likewise, therapeutic or drug target or gene expression can be assessed by quantitative fluorogenic RT-PCR (i.e., the TaqMan® System). For PCR analysis, methods and software are available to determine primer sequences for use in the analysis. These methods are particularly useful for detecting polynucleotides present in very low numbers.

Additional methods of detecting polynucleotide expression levels of RNA may be monitored using a membrane blot (such as used in hybridization analysis such as Northern or Southern blotting, dot, and the like), or microwells, sample tubes, gels, beads or fibers (or any solid support comprising bound nucleic acids). See, e.g., U.S. Pat. Nos. 5,770,722; 5,874,219; 5,744,305; 5,677,195 and 5,445,934. Polynucleotide therapeutic or drug target or gene expression also can include using nucleic acid probes in solution.

Another method of detecting polynucleotide expression levels involves SAGE, which is a method that allows the simultaneous and quantitative analysis of a large number of polynucleotides without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules that can be sequenced, revealing the identity of the multiple tags simultaneously. The expression pattern of any population of transcripts can be quantitatively evaluated by determining the abundance of individual tags and identifying the gene corresponding to each tag. See, Velculescu et al. (1995), supra.

Another method of detecting polynucleotide expression levels involves massively parallel signature sequencing (“MPSS”). See, Brenner et al. (2000) Nat. Biotech. 18:630-634. This sequencing combines non-gel-based signature sequencing with in vitro cloning of millions of templates on separate diameter microbeads. First, a microbead library of DNA templates can be constructed by in vitro cloning. This is followed by assembling a planar array of the template-containing microbeads in a flow cell at a high density (typically greater than 3.0×106 microbeads/cm2). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast DNA library.

Likewise, methods of detecting and quantifying polypeptides in a sample are well known in the art and include, but are not limited to, immunohistochemistry and proteomics-based methods.

For example, a tissue sample can be collected by, for example, biopsy techniques known in the art. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

Some samples may need to be subjected to antigen retrieval or antigen unmasking to make the therapeutic or drug target or gene polypeptides accessible to, for example, antibody binding. As used herein, “antigen retrieval” or “antigen unmasking” means methods for increasing antigen accessibility or recovering antigenicity in, for example, formalin-fixed, paraffin-embedded tissue samples. Formalin fixation of tissue samples results in extensive cross-linking of proteins that can lead to the masking or destruction of antigen sites and, subsequently, poor antibody staining. Any method of making antigens more accessible for antibody binding may be used in the practice of the invention, including those antigen retrieval methods known in the art. See, e.g., Tumor Marker Protocols (Hanausek & Walaszek, eds., Humana Press, Inc. 1988); and Shi et al., Antigen Retrieval Techniques: Immunohistochemistry and Molecular Morphology (Eaton Publishing 2000).

Methods of antigen retrieval are well known in the art. Examples of such methods include, but are not limited to, treatment with proteolytic enzymes (e.g., trypsin, chymotrypsin, pepsin, pronase and the like) or antigen retrieval solutions. Antigen retrieval solutions can include citrate buffer, pH 6.0, Tris buffer, pH 9.5, EDTA, pH 8.0, L.A.B. (“Liberate Antibody Binding Solution”; Polysciences; Warrington, Pa.), antigen retrieval Glyca solution (Biogenex; San Ramon, Calif.), citrate buffer solution, pH 4.0, Dawn® detergent (Proctor & Gamble; Cincinnati, Ohio), deionized water and 2% glacial acetic acid. Such an antigen retrieval solutions can be applied to a formalin-fixed tissue sample and then heated in an oven (e.g., at 60° C.), steamed (e.g., at 95° C.) or pressure cooked (e.g., at 120° C.) for a pre-determined time periods. Alternatively, antigen retrieval can be performed at room temperature. As such, incubation times will vary with the particular antigen retrieval solution selected and with the incubation temperature. For example, an antigen retrieval solution can be applied to a sample for as little as about 5, 10, 20 or 30 minutes or up to overnight. The design of assays to determine the appropriate antigen retrieval solution and optimal incubation times and temperatures is standard and well within the routine capabilities of one of skill in the art.

Following antigen retrieval, samples are blocked using an appropriate blocking agent (e.g., hydrogen peroxide). An antibody directed to a therapeutic or drug target or gene of interest then is incubated with the sample for a time sufficient to permit antigen-antibody binding. As described elsewhere, at least five antibodies directed to five distinct therapeutic or drug targets or genes can be used to detect cancer. Where more than one antibody may be used, these antibodies can be added to a single sample sequentially as individual antibody reagents, or simultaneously as an antibody cocktail. Alternatively, each individual antibody can be added to a separate tissue section from a single patient sample, and the resulting data pooled.

Methods of detecting antibody binding are well known in the art. Antibody binding to a therapeutic or drug target or gene of interest can be detected through the use of chemical reagents that generate a detectable signal that corresponds to the level of antibody binding, and, accordingly, to the level of therapeutic or drug target or gene protein expression. For example, antibody binding can be detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell or tissue staining that corresponds to expression level of the therapeutic or drug target or gene of interest. Enzymes of particular interest include horseradish peroxidase (HRP) and alkaline phosphatase (AP). Commercially antibody detection systems include, for example, the Dako Envision+system (Glostrup; Denmark) and Biocare Medical's Mach 3 System (Concord, Calif.), and can be used herein.

Detecting antibody binding can be facilitated by coupling the antibody to a detectable moiety. Examples of detectable moieties include various enzymes, prosthetic groups, fluorescent materials, luminescent materials, bioluminescent materials, and radioactive materials. Examples of suitable enzymes include horseradish peroxidase, alkaline phosphatase, galactosidase and acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriaziny-lamine fluorescein, dansyl chloride and phycoerythrin. An example of a luminescent material is luminol. Examples of bioluminescent materials include luciferase, luciferin and aequorin. Examples of radioactive materials include 125I, 131I, 35S and 3H.

In regard to additional antibody detection methods, there also exists video microscopy and software methods for quantitatively determining an amount of multiple molecular species (e.g., therapeutic or drug target or gene proteins) in a biological sample, where each molecular species present is indicated by a representative dye marker having a specific color. Such methods are known in the art as a colorimetric analysis method. In these methods, video-microscopy is used to provide an image of the biological sample after it has been stained to visually indicate the presence of a particular therapeutic or drug target or gene of interest. See, e.g., U.S. Pat. Nos. 7,065,236 and 7,133,547, which disclose the use of an imaging system and associated software to determine the relative amounts of each molecular species present based on the presence of representative color dye markers as indicated by those color dye markers' optical density or transmittance value, respectively, as determined by an imaging system and associated software. These methods provide quantitative determinations of the relative amounts of each molecular species in a stained biological sample using a single video image that is “deconstructed” into its component color parts.

Once expression levels of the plurality of therapeutic or drug targets or genes are determined, the expression data is processed according to the methods, systems, algorithms, programs, and codes described above. Such processing generates a plurality of genes which have enhanced, enriched, increased, decreased, or reduced expression levels. The plurality of genes are once processed are compared to the genes listed in Appendix A, Appendix B, Appendix C, Appendix D, Appendix E, Appendix F, Appendix G, Appendix H, Appendix I, Appendix J, Appendix K, Appendix L, Appendix M, Appendix N, Table B, Table C, Table D, Table E, Table F, Table G, Table H, Table I, Table J, Table K, Table L, Table M, Table N, Table O, Table AP, Table AQ, Table AR, Table AS, Table AT, Table AU, Table AV, Table AX, Table AY, Table AZ, Table AAA, Table AAB, Table AAC, Table AAD, Table AAF, Table AAG, Table AAH, Table AAJ, Table AAK, Table AAL, Table AAM, Table AAN, or Table AAO, or combinations thereof.

In some embodiments, based on the comparison, the presence of the genes listed in Appendix A, Appendix B, Table B, Table C, Table AT, Table AU, Table AV, or combination thereof, is an indication that the subject is likely to be afflicted with BRCA.

In some embodiments, based on the comparison, the presence of the genes listed in Appendix G, Appendix H, Table H, Table I, Table AAB, Table AAC, Table AAD, or combination thereof, is an indication that the subject is likely to be afflicted with LUAD or LUSC.

In some embodiments, based on the comparison, the presence of the genes listed in Appendix I, Appendix J, Table J, Table K, Table AAF, Table AAG, Table AAH, or combination thereof, is an indication that the subject is likely to be afflicted with Luminal A or Luminal B.

In some embodiments, based on the comparison, the presence of the genes listed in Appendix C, Appendix D, Table D, Table E, Table AX, Table AY, Table AZ, Table AAA, or combination thereof, is an indication that the subject is likely to be afflicted with ER positive or ER negative.

In some embodiments, based on the comparison, the presence of the genes listed in Appendix E, Appendix F, Table F, Table G, Table AP, Table AQ, Table AR, Table AS, or combination thereof, is an indication that the subject is likely to be afflicted with KIRP or KIRC.

In some embodiments, based on the comparison, the presence of the genes listed in Appendix K, Table L, Table M, Table AAJ, Table AAK, or combination thereof, is an indication that the subject is likely to be afflicted with cancer.

In some embodiments, based on the comparison, the presence of the genes listed in Appendix M, Appendix N, Table N, Table O, Table AAL, AAM, AAN, AAO, or combination thereof, is an indication that the subject is likely to not be afflicted with cancer, or likely to survive cancer.

Provided herein are diagnostic systems (i.e., kits and panels) comprising the therapeutic or drug targets or genes listed in Appendix A, Appendix B, Appendix C, Appendix D, Appendix E, Appendix F, Appendix G, Appendix H, Appendix I, Appendix J, Appendix K, Appendix L, Appendix M, Appendix N, Table B, Table C, Table D, Table E, Table F, Table G, Table H, Table I, Table J, Table K, Table L, Table M, Table N, Table O, Table AP, Table AQ, Table AR, Table AS, Table AT, Table AU, Table AV, Table AX, Table AY, Table AZ, Table AAA, Table AAB, Table AAC, Table AAD, Table AAF, Table AAG, Table AAH, Table AAJ, Table AAK, Table AAL, Table AAM, Table AAN, or Table AAO, or combinations thereof.

In some embodiments, the diagnostic systems (i.e., kits and panels) comprise reagents for detecting, diagnosing, or prognosing an individual having or suspected of having cancer (e.g., any of the cancers listed in Table A). As used herein, “kit” or “kits” means any manufacture (e.g., a package or a container) including at least one reagent, such as a nucleic acid probe, an antibody or the like, for specifically detecting the expression of the any of the genes described herein. In some embodiments, a plurality of reagents may be used.

As used herein, “probe” means any molecule that is capable of selectively binding to a specifically intended target biomolecule, for example, a nucleotide transcript or a protein encoded by or corresponding to a therapeutic or drug target. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies and organic molecules.

In other embodiments, primer (e.g., oligonucleotide) sequences are useful for detecting or analyzing gene expression of therapeutic or drug targets. In other embodiments, the invention provides oligonucleotides which are able to amplify a therapeutic or drug target, for example, including at least one forward and one reverse primer, which together can be used for amplification and/or sequencing of an intended therapeutic or drug target, can be suitably packaged in a kit. In one embodiment, nested pairs of amplification and sequencing primers are provided. In still another embodiment, the kit comprises a set of primers. The primers in such kits can be labeled or unlabeled. The kit can also include additional reagents such as reagents for performing an amplification (e.g., PCR) reaction, a reverse transcriptase for conversion of RNA to cDNA for amplification, DNA polymerases, dNTP and ddNTP feedstocks. Kits of the present invention can also include instructions for use.

The kits can be promoted, distributed or sold as units for performing any of the methods described herein. Additionally, the kits can contain a package insert describing the kit and methods for its use. For example, the insert can include instructions for correlating the level of therapeutic or drug target expression measured with a subject's likelihood of having developed cancer or the likely prognosis of a subject already diagnosed with cancer.

The kits therefore can be for detecting, diagnosing and prognosing a cancer (e.g., any of the cancers listed in Table A) with therapeutic or drug targets at the nucleic acid level. Such kits are compatible with both manual and automated nucleic acid detection techniques (e.g., gene arrays, Northern blotting or Southern blotting. Likewise, the kits can be for detecting, diagnosing and prognosing a cancer with therapeutic or drug targets at the amino acid level. Such kits are compatible with both manual and automated immunohistochemistry techniques (e.g., cell staining, ELISA or Western blotting).

Any or all of the kit reagents can be provided within containers that protect them from the external environment, such as in sealed containers. Positive and/or negative controls can be included in the kits to validate the activity and correct usage of reagents employed in accordance with the invention. Controls can include samples, such as tissue sections, cells fixed on glass slides, RNA preparations from tissues or cell lines, and the like, known to be either positive or negative for any of the therapeutic or drug targets set forth in Table B, Table C, Table D, Table E, Table F, Table G, Table H, Table I, Table J, Table K, Table L, Table M, Table N, Table O, Table AP, Table AQ, Table AR, Table AS, Table AT, Table AU, Table AV, Table AX, Table AY, Table AZ, Table AAA, Table AAB, Table AAC, Table AAD, Table AAF, Table AAG, Table AAH, Table AAJ, Table AAK, Table AAL, Table AAM, Table AAN, or Table AAO. The design and use of controls is standard and well within the routine capabilities of one of skill in the art.

Methods of Prognosing Cancers

Methods of the invention include prognosing the likelihood of metastasis in an individual having a cancer (e.g., any of the cancers listed in Table A). The methods include detecting the expression of therapeutic or drug targets or genes in a biological sample from a subject having a cancer at a first point in time prior to treatment with an anti-cancer therapy or therapeutic regimen, and then at least one subsequent point in time after the subject has undergone treatment, completed treatment, and/or is in remission for the cancer.

In some embodiments, the subject has undergone chemotherapy, radiation therapy, or surgical removal of tumor. In some embodiments, the subject has been treated or administered any of the therapeutic agents or drugs set forth in Tables P-AO.

Absence, presence, or altered expression levels of a therapeutic or drug target or gene or combination of therapeutic or drug targets or genes can be used to indicate cancer prognosis (i.e., poor or good prognosis). As such, presence, absence, or altered expression of a particular therapeutic or drug target or gene or combination of therapeutic or drug targets or genes permits the differentiation of subjects having a cancer that are likely to experience disease recurrence and/or metastasis (i.e., poor prognosis) from those who are more likely to remain cancer free (i.e., good prognosis).

In some embodiments, the absence of the genes listed in Appendix A, Appendix B, Table B, Table C, Table AT, Table AU, Table AV, or combination thereof, is an indication that the subject is likely to progress, or that the therapeutic agent or drug treats BRCA in the subject.

In some embodiments, the absence of the genes listed in Appendix G, Appendix H, Table H, Table I, Table AAB, Table AAC, Table AAD, or combination thereof, is an indication that the subject is likely to progress, or that the therapeutic agent or drug treats LUAD or LUSC in the subject.

In some embodiments, the absence of the genes listed in Appendix I, Appendix J, Table J, Table K, Table AAF, Table AAG, Table AAH, or combination thereof, is an indication that the subject is likely to progress, or that the therapeutic agent or drug treats Luminal A or Luminal B in the subject.

In some embodiments, the absence of the genes listed in Appendix C, Appendix D, Table D, Table E, Table AX, Table AY, Table AZ, Table AAA, or combination thereof, is an indication that the subject is likely to progress, or that the therapeutic agent or drug treats ER positive or ER negative in the subject.

In some embodiments, the absence of the genes listed in Appendix E, Appendix F, Table F, Table G, Table AP, Table AQ, Table AR, Table AS, or combination thereof, is an indication that the subject is likely to progress, or that the therapeutic agent or drug treats KIRP or KIRC in the subject.

In some embodiments, the absence of the genes listed in Appendix K, Table L, Table M, Table AAJ, Table AAK, or combination thereof, is an indication that the subject is likely to progress, or that the therapeutic agent or drug treats cancer in the subject.

In some embodiments, the presence of the genes listed in Appendix M, Appendix N, Table N, Table O, Table AAL, AAM, AAN, AAO, or combination thereof, is an indication that the subject is likely to progress, or that the therapeutic agent or drug treats cancer in the subject.

As used herein, “prognose,” “prognoses,” “prognosis” and “prognosing” means predictions about or predicting a likely course or outcome of a disease or disease progression, particularly with respect to a likelihood of, for example, disease remission, disease relapse, tumor recurrence, metastasis and death (i.e., the outlook for chances of survival). As used herein, “good prognosis” or “favorable prognosis” means a likelihood that an individual having cancer will remain disease-free (i.e., cancer-free). As used herein, “poor prognosis” means a likelihood of a relapse or recurrence of the underlying cancer or tumor, metastasis or death. Individuals classified as having a good prognosis remain free of the underlying cancer or tumor. Conversely, individuals classified as having a bad prognosis experience disease relapse, tumor recurrence, metastasis or death.

Additional criteria for evaluating the response to anti-cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

One of skill in the art is familiar with the time frame(s) for assessing prognosis and outcome. Examples of such time frames include, but are not limited to, less than one year, about one, two, three, four, five, six, seven, eight, nine, ten, fifteen, twenty or more years. With respect to cancer, the relevant time for assessing prognosis or disease-free survival time often begins with the surgical removal of the tumor or suppression, mitigation or inhibition of tumor growth. Thus, for example, a good prognosis can be a likelihood that the individual having cancer will remain free of the underlying cancer or tumor for a period of at least about five, more particularly, a period of at least about ten years. In contrast, for example, a bad prognosis can be a likelihood that the individual having cancer experiences disease relapse, tumor recurrence, metastasis or death within a period of less than about five years, more particularly a period of less than about ten years.

Methods of prognosing cancer are well known in the art. One method to evaluate the prognostic performance of the therapeutic or drug targets or genes and/or other clinical parameters utilizes PAM. PAM is a statistical technique for class prediction from gene expression data using nearest shrunken centroids. See, Tibshirani et al. (2002) Proc. Natl. Acad. Sci. 99:6567-6572.

Another method is the nearest shrunken centroids, which identifies subsets of genes that best characterize each class. This method is general and can be used in many other classification problems. It can also be applied to survival analysis problems. The method computes a standardized centroid for each class, which is the average gene expression for each gene in each class divided by the within-class standard deviation for that gene. Nearest centroid classification takes the gene expression profile of a new sample, and compares it to each of these class centroids. The class whose centroid that it is closest to, in squared distance, is the predicted class for that new sample. Nearest shrunken centroid classification makes one important modification to standard nearest centroid classification. It “shrinks” each of the class centroids toward the overall centroid for all classes by an amount we call the threshold. This shrinkage consists of moving the centroid towards zero by threshold, setting it equal to zero if it hits zero. For example if threshold was 2.0, a centroid of 3.2 would be shrunk to 1.2, a centroid of −3.4 would be shrunk to −1.4, and a centroid of 1.2 would be shrunk to zero. After shrinking the centroids, the new sample is classified by the usual nearest centroid rule, but using the shrunken class centroids. This shrinkage has two advantages: 1) it can make the classifier more accurate by reducing the effect of noisy genes; and 2) it does automatic gene selection. The user decides on the value to use for threshold. Typically one examines a number of different choices.

Alternatively, prognostic performance of the therapeutic or drug targets or genes and/or other clinical parameters can be assessed by Cox Proportional Hazards Model Analysis, which is a regression method for survival data that provides an estimate of the hazard ratio and its confidence interval. The Cox model is a well-recognized statistical method for exploring the relationship between the survival of a patient and particular variables. This statistical method permits estimation of the hazard (i.e., risk) of individuals given their prognostic variables (e.g., overexpression of particular therapeutic or drug targets or genes, as described herein). Cox model data are commonly presented as Kaplan-Meier curves or plots. The “hazard ratio” is the risk of death at any given time point for patients displaying particular prognostic variables. See generally, Spruance et al. (2004) Antimicrob. Agents & Chemo. 48:2787-2792.

The therapeutic or drug targets or genes of interest can be statistically significant for assessment of the likelihood of cancer recurrence or death due to the underlying cancer. Methods for assessing statistical significance are well known in the art and include, for example, using a log-rank test, Cox analysis and Kaplan-Meier curves. A p-value of less than 0.05 can be used to constitute statistical significance.

The expression levels of at least one therapeutic or drug target or gene in a tumor sample can be indicative of a poor cancer prognosis and thereby used to identify individuals who are more likely to suffer a recurrence of the underlying cancer. The therefore methods involve detecting the expression levels of at least one therapeutic or drug target or gene in a tumor sample that is indicative of early stage disease.

In some embodiments, overexpression of a therapeutic or drug target or gene or combination of therapeutic or drug targets or genes of interest in a sample can be indicative of a poor cancer prognosis. As used herein, “indicative of a poor prognosis” is intended that altered expression of particular therapeutic or drug target or gene or combination of therapeutic or drug targets or genes is associated with an increased likelihood of relapse or recurrence of the underlying cancer or tumor, metastasis or death. For example, “indicative of a poor prognosis” may refer to an increased likelihood of relapse or recurrence of the underlying cancer or tumor, metastasis, or death within ten years, such as five years. In other aspects of the invention, the absence of overexpression of a therapeutic or drug target or gene or combination of therapeutic or drug targets or genes of interest is indicative of a good prognosis. As used herein, “indicative of a good prognosis” refers to an increased likelihood that the patient will remain cancer free. In some embodiments, “indicative of a good prognosis” refers to an increased likelihood that the patient will remain cancer-free for ten years, such as five years.

Methods of Treating Cancers

The therapeutic or drug targets or genes, and detection, diagnosing and prognosing methods described above can be used to assist in selecting appropriate treatment regimen and to identify individuals that would benefit from more aggressive therapy.

Approaches to the treating cancers include surgery, immunotherapy, chemotherapy, radiation therapy, a combination of chemotherapy and radiation therapy, or biological therapy. Additional approaches to treating cancer include administering or prescribing to the subject having cancer with any of the therapeutic agents set forth in Tables P-AO. In some embodiments, the subject is administered a therapeutically effective amount of any of the therapeutic agents set forth in Tables P-AO to mediate a therapeutic. In some embodiments, the subject is administered a defined treatment based upon the diagnosis.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds set forth in Tables P-AO may be administered in a sufficient amount to produce a reasonable benefit/risk ratio applicable to such treatment.

The terms “therapeutically-effective amount” and “effective amount” as used herein means that amount of a compound, material, or composition comprising a compound set forth in Tables P-AO which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95% or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

In some embodiments, the subject is determined to have ER positive or ER negative cancer, and therefore is administered or prescribed any of the therapeutic agents, drugs, or treatment is defined in Table R, Table S, Table AE, or Table AF.

In some embodiments, the subject is determined to have BRCA cancer, and therefore is administered or prescribed any of the therapeutic agent or treatment is defined in Table P, Table Q, Table AC, or Table AD.

In some embodiments, the subject is determined to have KIRP or KIRC cancer, and therefore is administered or prescribed any of the therapeutic agent or treatment is defined in Table T, Table U, Table AG, or Table AH.

In some embodiments, the subject is determined to have LUAD or LUSC cancer, and therefore is administered or prescribed any of the therapeutic agent or treatment is defined in Table V, Table W, Table AI, or Table AJ.

In some embodiments, the subject is determined to have Luminal A or Luminal B cancer, and therefore is administered or prescribed any of the therapeutic agent or treatment is defined in Table X, Table Y, Table AK, or Table AL.

Clinical efficacy can be measured by any method known in the art. For example, the response to a therapy, such as to any of the therapeutic agents or treatments set forth in Tables P-AO, relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Cin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular therapeutic agent set forth in Table P to AO is at least 25%, 30%, 35%, 40%, 45%, 50%, 55%, 60%, 65%, 70%, 75%, 80%, 85%, or more.

For example, in order to determine appropriate threshold values, a particular therapeutic agent as set forth in Tables P-AO can be administered to a population of subjects and the outcome can be correlated to therapeutic or drug target measurements that were determined prior to administration of any of the therapeutic agents set forth in Tables P-AO. The outcome measurement may be pathologic response to therapy given in the neoadjuvant setting. Alternatively, outcome measures, such as overall survival and disease-free survival can be monitored over a period of time for subjects following administering any of the therapeutic agents set forth in Tables P-AO for whom therapeutic or drug target measurement values are known. In certain embodiments, the same doses of any of the therapeutic agents set forth in Tables P-AO are administered to each subject. In related embodiments, the doses administered are standard doses known in the art for any of the therapeutic agents set forth in Tables P-AO. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

The methods described above therefore find particular use in selecting appropriate treatment for early- or late-stage cancer patients. The majority of individuals having cancer diagnosed at an early-stage of the disease enjoy long-term survival following surgery and/or radiation therapy without further adjuvant therapy. However, a significant percentage of these individuals will suffer disease recurrence or death, leading to clinical recommendations that some or all early-stage cancer patients should receive adjuvant therapy (e.g., chemotherapy). The methods of the present invention can identify this high-risk, poor prognosis population of individuals having early-stage cancer and thereby can be used to determine which ones would benefit from continued and/or more aggressive therapy and close monitoring following treatment. For example, individuals having early-stage cancer and assessed as having a poor prognosis by the methods disclosed herein may be selected for more aggressive adjuvant therapy, such as chemotherapy, following surgery and/or radiation treatment. In the situation where the subject has late-stage cancer, the methods of the present invention can identify appropriate therapeutic drugs or agents that a doctor, physician, or health provider can prescribed having short treatment regimens or quicker efficacy time frames. The methods of the present invention may be used in conjunction with standard procedures and treatments to permit physicians to make more informed cancer treatment decisions.

Exemplary Results

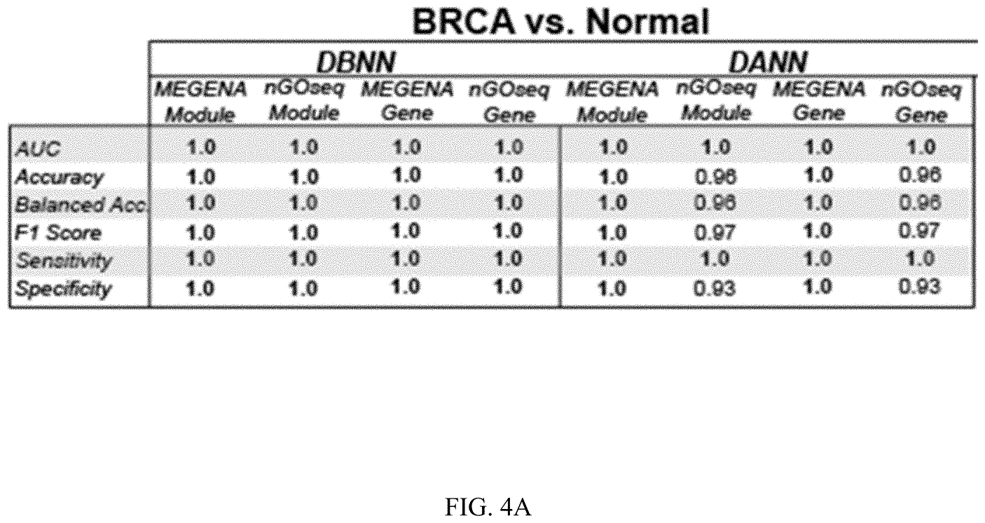

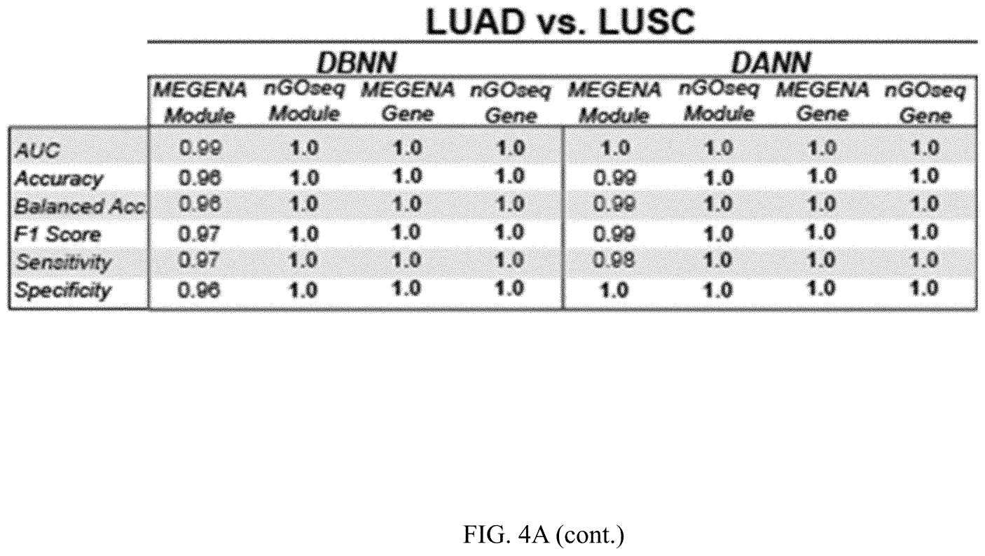

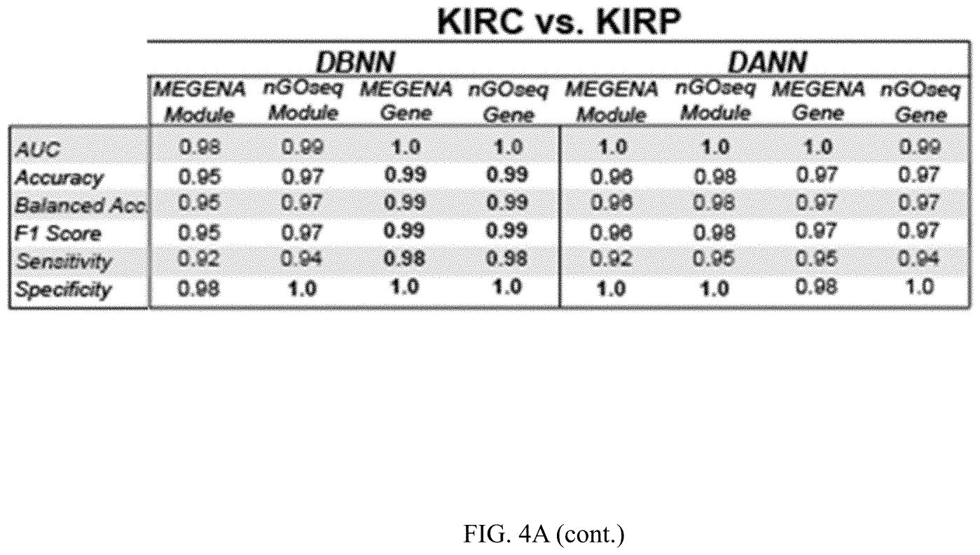

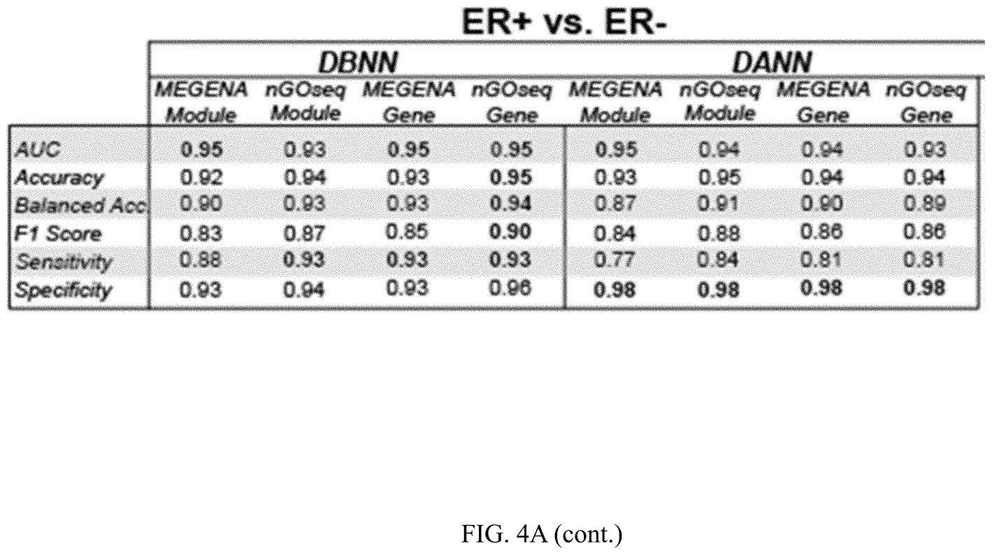

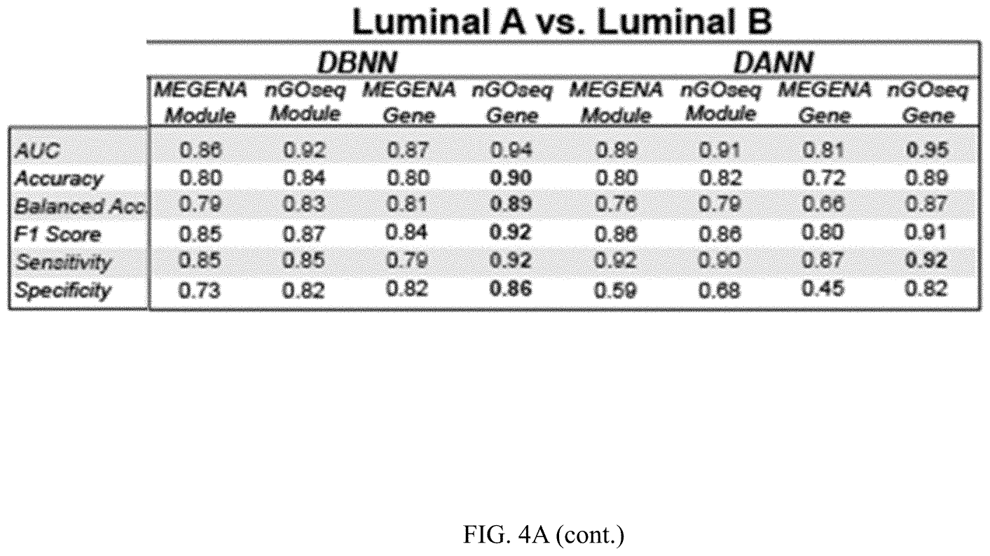

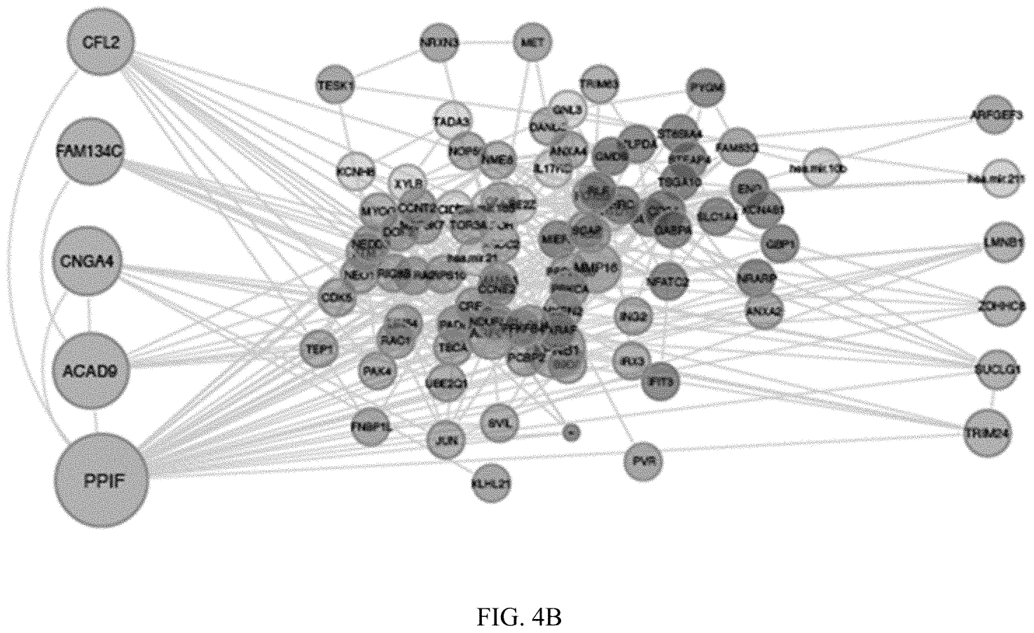

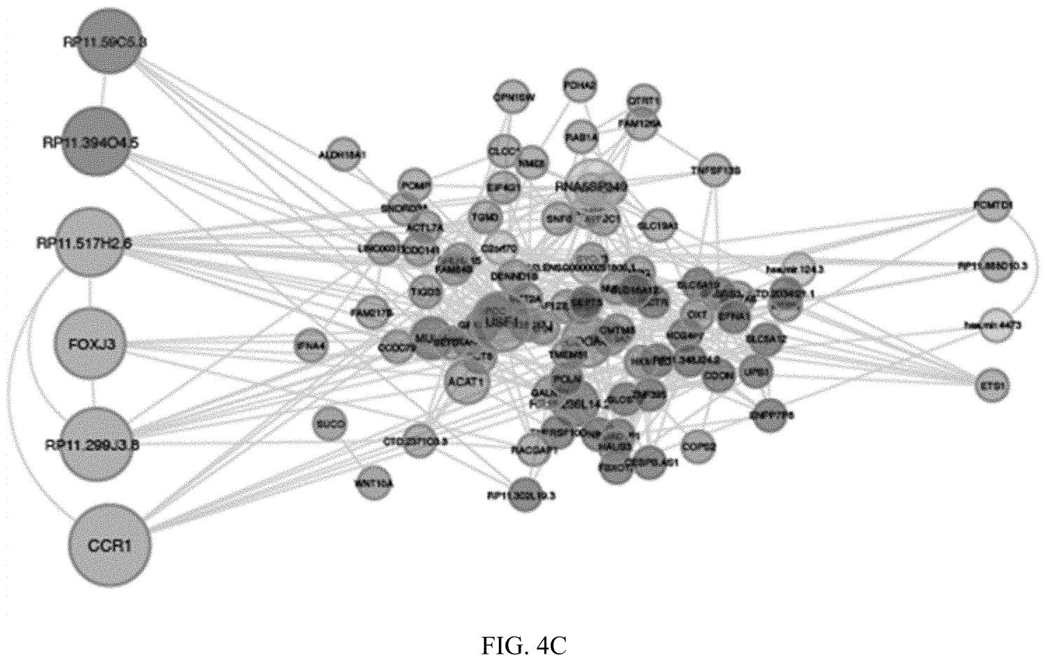

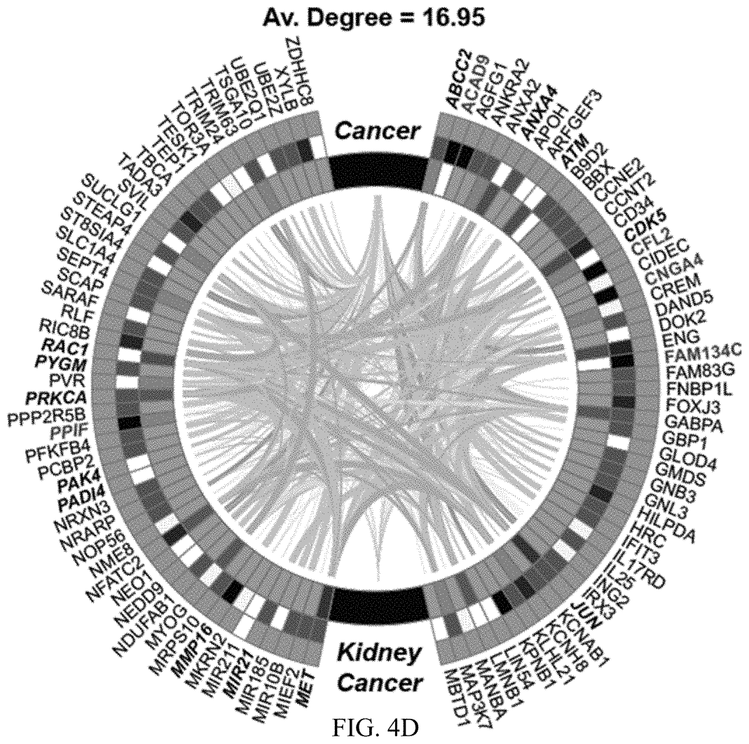

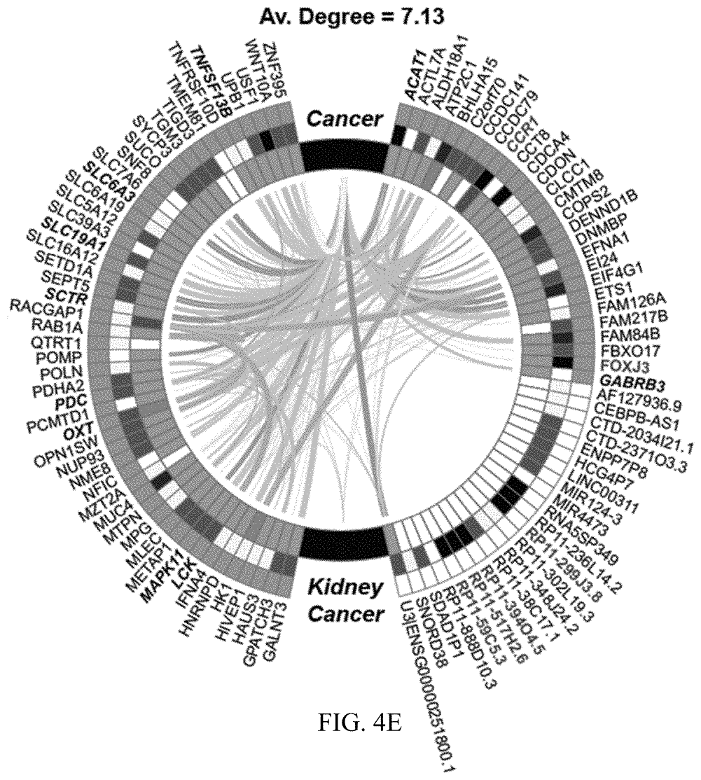

Referring now to FIGS. 4-7, exemplary results of a system according to the present disclosure are presented.

In FIG. 4, binomial model comparisons at both the module and gene level specifically highlighting kidney renal papillary cell carcinoma (KIRP) versus kidney renal clear cell carcinoma (KIRC) are shown. FIG. 4A is a table showing various test data set model statistics (area under curve (AUC), accuracy, balanced accuracy, F1 score, sensitivity, and specificity) for each of the five binomial comparisons at the module level (MEGENA Module and nGOseq Module) and gene level (MEGENA Gene and nGOseq Gene). Bolded values indicate the highest value of each statistic. FIGS. 4B-C show nGOseq (b) and MEGENA (c) derived directed acyclic graphs (DAG) from the training data set showing causal drivers of the 100 most informative genes for KIRP vs. KIRC. Genes on the left side of the DAG are the most likely upstream causal drivers while the genes on the right are the most likely downstream targets (both determined based on incoming and outgoing edges in the DAG). Data types: METH (blue), mRNA (red), miRNA (orange), STV (Pink), CNV (green). FIGS. 4D-E show nGOseq (d) and MEGENA (e) natural language processing diagrams showing known literature connections between the 100 most informative genes and cancer and/or kidney cancer (using MESH terms as detailed in Methods) as well as known literature gene to gene connections. For each gene, the outer ring indicates the presence (blue) or absence (white) of functional annotation, the middle ring displays the difference in outgoing to incoming edges from the respective DAG (colored with 10 bins where white—lowest and black—highest, see Methods), and the inner ring indicates the total number of edges (colored with 6 bins where white—lowest and dark purple—highest, see Methods). Inner chord colors for gene to cancer relationships (gene to cancer and/or kidney cancer): red—inhibitory, grey—neither inhibitory or stimulatory, green—stimulatory, yellow—both inhibitory and stimulatory. Inner chord colors for gene to gene relationships: pink—inhibitory, purple—neither inhibitory or stimulatory, orange—stimulatory, blue—both inhibitory and stimulatory. Genes highlighted in red are those that appear on the left side of the DAGs and those that are bold and italicized are known drug targets. Average degree of gene connections to both cancer and/or kidney cancer and other genes is displayed above the diagram.

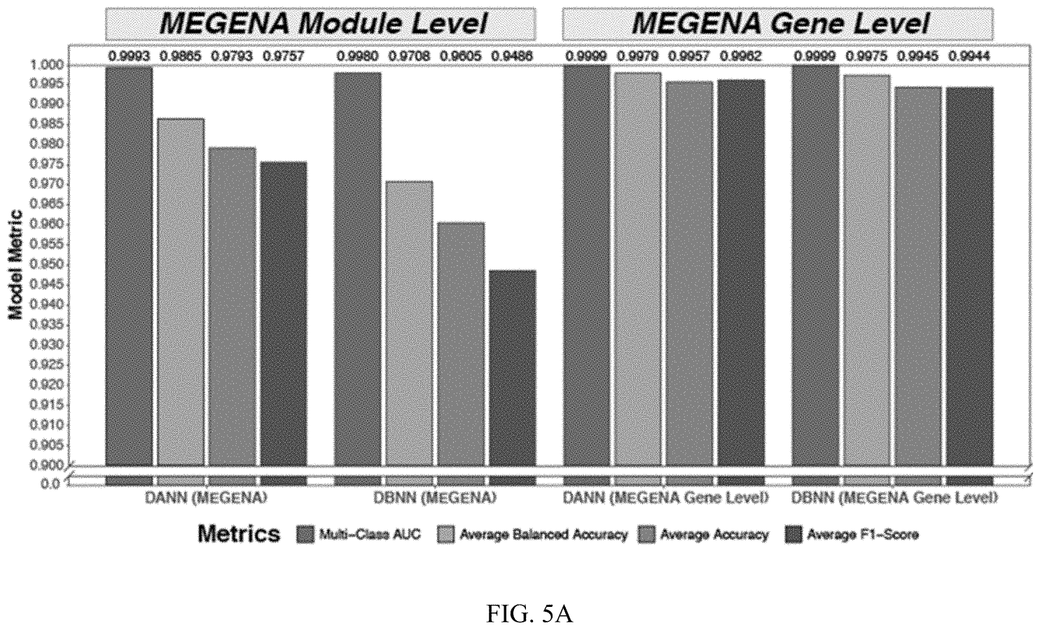

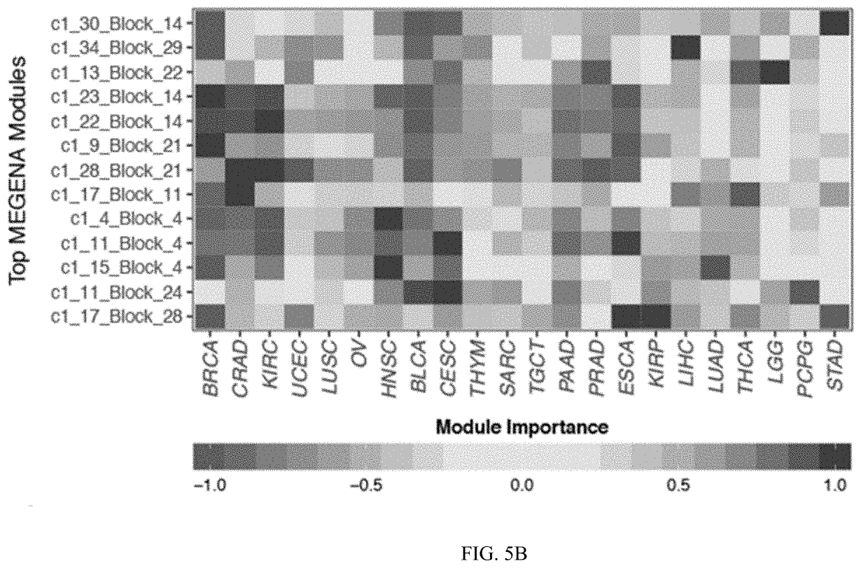

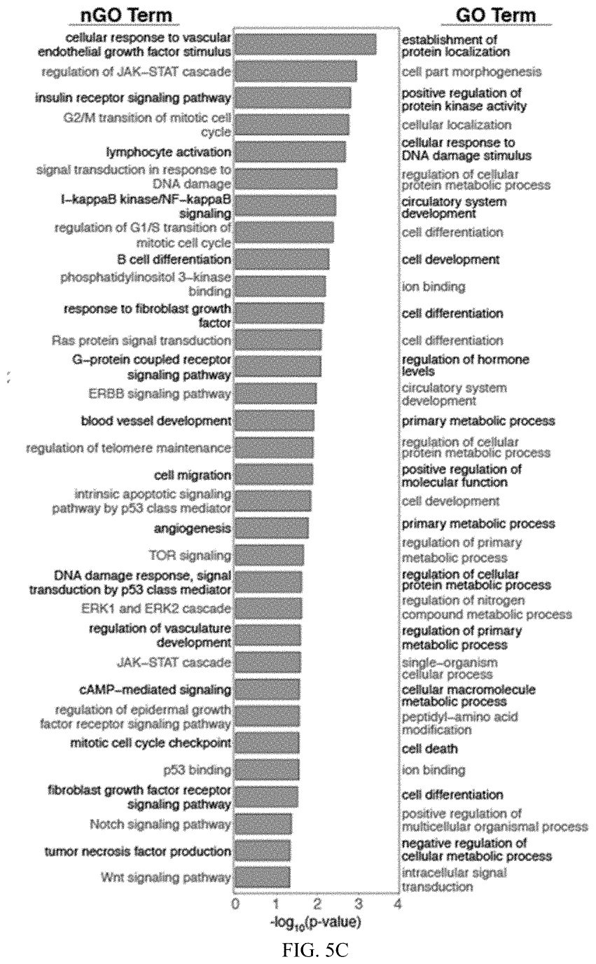

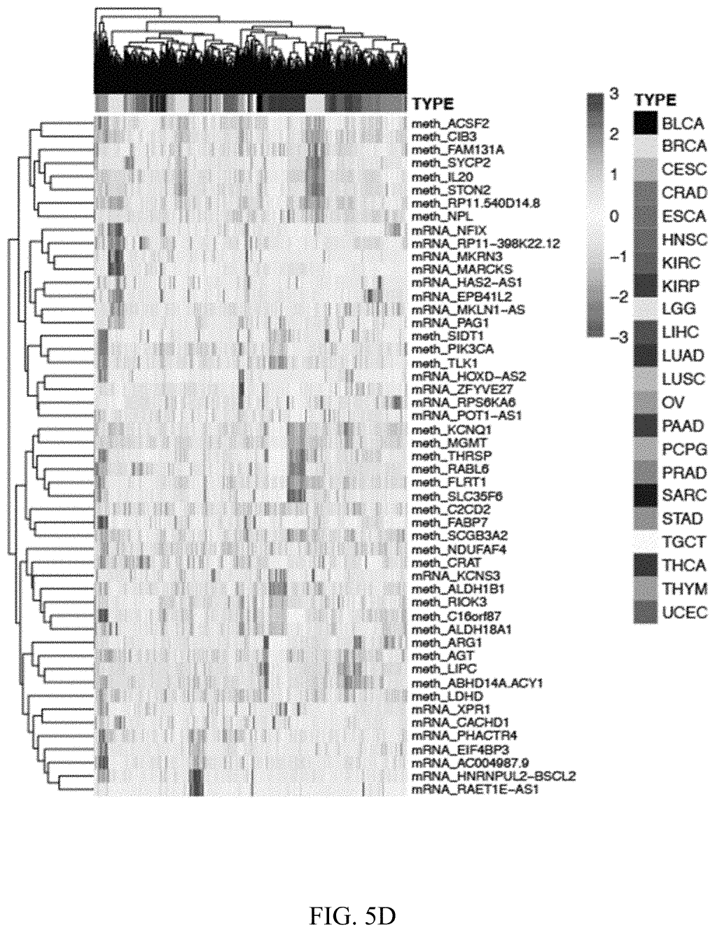

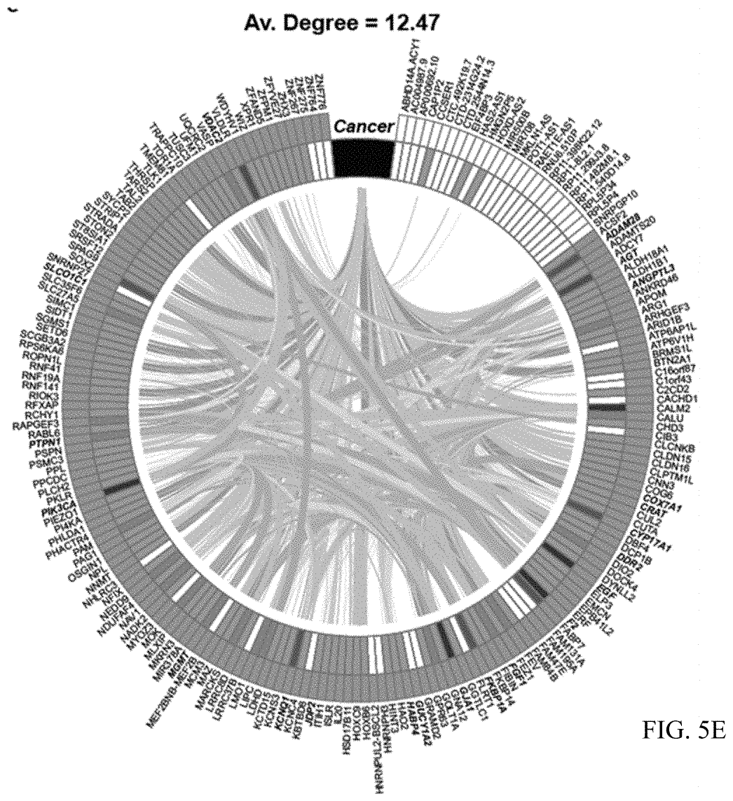

FIG. 5 illustrates multinomial models at the module and gene level comparing 22 cancer types from the TCGA database. FIG. 5A shows test data set model statistics (area under curve (AUC), accuracy, balanced accuracy, F1 score) at the module level (MEGENA Module) and gene level (MEGENA Gene). FIG. 5B is a clustergram showing the similarities between all 22 cancers for the training data set of the 13 most informative MEGENA modules. The rankings were derived based on the ensemble rankings of DANN and DBNN models at the module level for each cancer type (see Methods). Signed module importance is normalized between −1 (blue) and 1 (red) where 0 (beige-white) represents a non-important module. FIG. 5C shows selected nGOseq enrichment terms for the gene level data matrix. The gene level data matrix was derived from each of the important MEGENA modules by breaking out the genes from each summary statistic of clusters. The left column indicates the nested GO terms while the right column indicates which GO terms the nested GO terms were nested inside of. FIG. 5D is a clustergram showing 51 genes with an informative rank at the gene level in 5 or more cancer types across all 8,272 samples (training and testing data sets) and 22 cancer types. Data is z-scored between ≤−3 (blue) and ≥3 (red). FIG. 5E is a natural language processing diagram showing known literature connections between the 200 most informative genes (based on informative rank in 4 or more cancer types) and cancer (using MESH terms as detailed in Methods) as well as known literature gene to gene connections. For each gene, the outer ring indicates the presence (blue) or absence (white) of functional annotation, the middle ring displays the difference in outgoing to incoming edges from the respective DAG (colored with 10 bins where white—lowest and black—highest), and the inner ring indicates the total number of edges (colored with 6 bins where white—lowest and dark purple—highest). Inner chord colors for gene to cancer relationships (gene to cancer and/or kidney cancer): red—inhibitory, grey—neither inhibitory or stimulatory, green—stimulatory, yellow—both inhibitory and stimulatory. Inner chord colors for gene to gene relationships: pink—inhibitory, purple—neither inhibitory or stimulatory, orange—stimulatory, blue—both inhibitory and stimulatory. Average degree of gene connections to both cancer and other genes is displayed above the diagram.

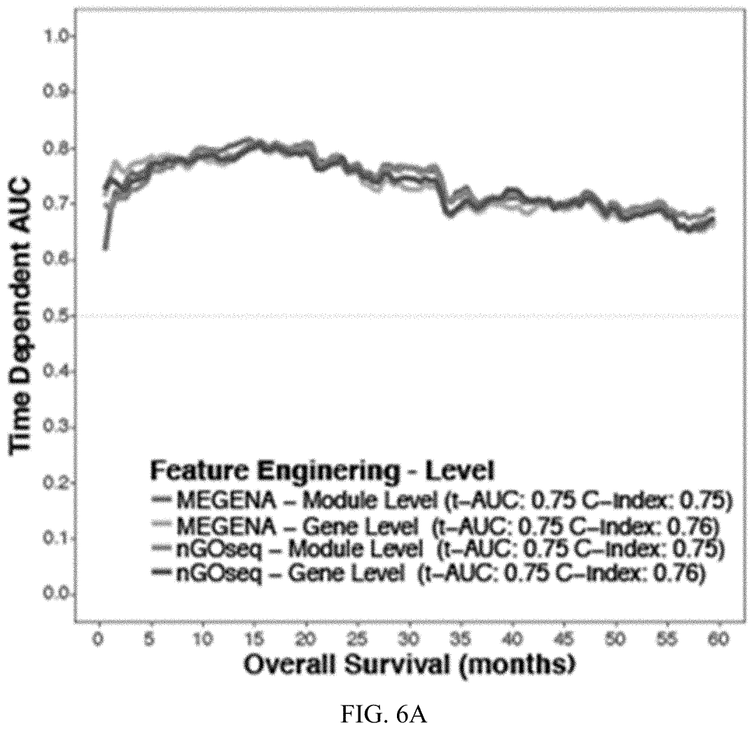

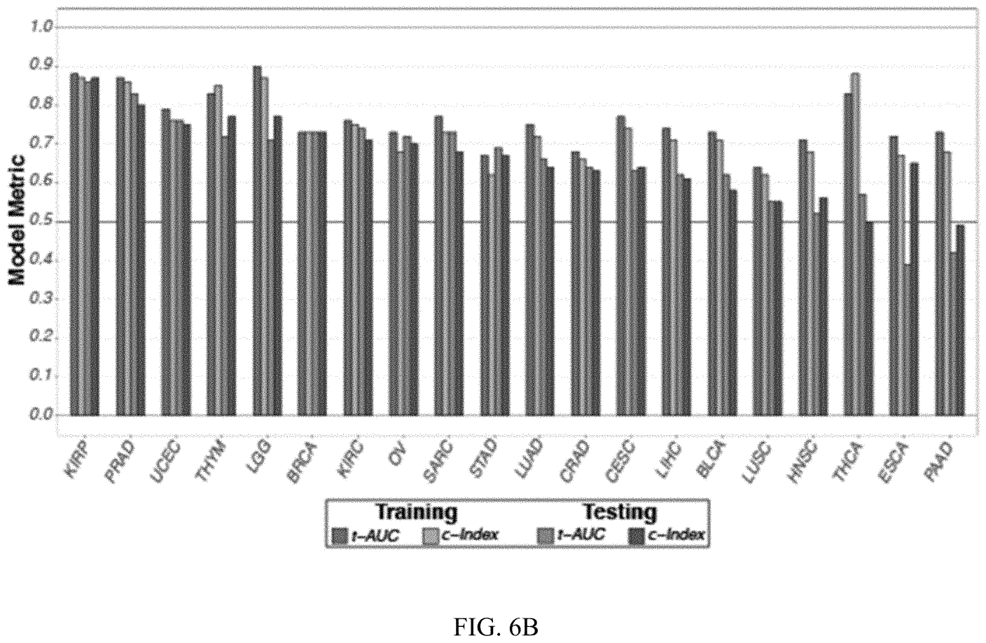

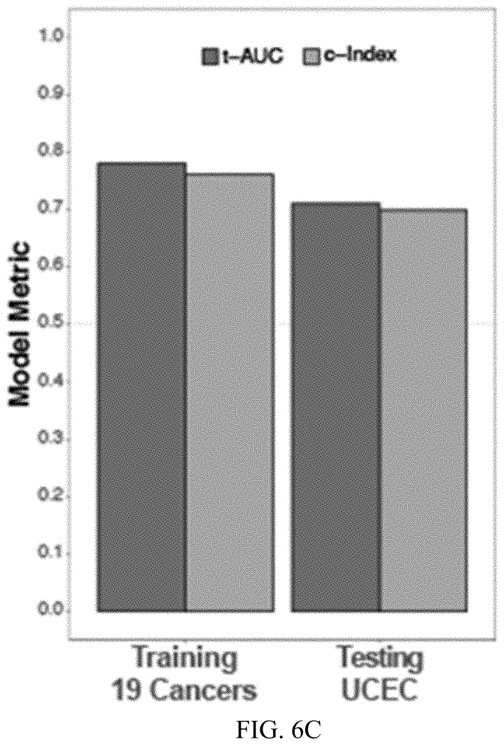

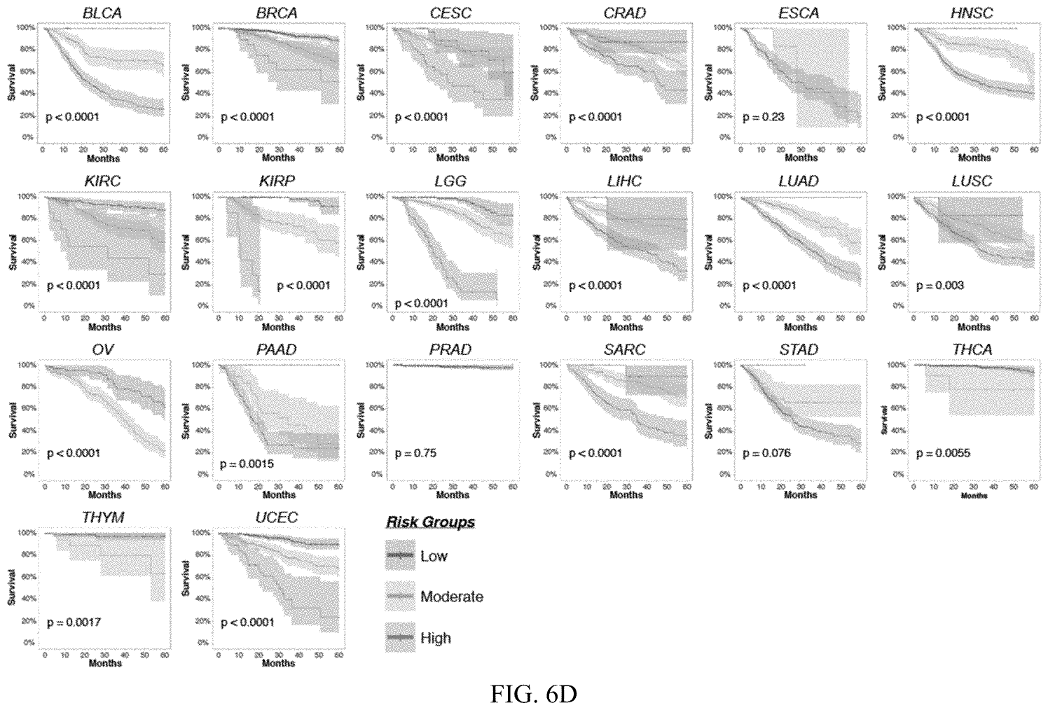

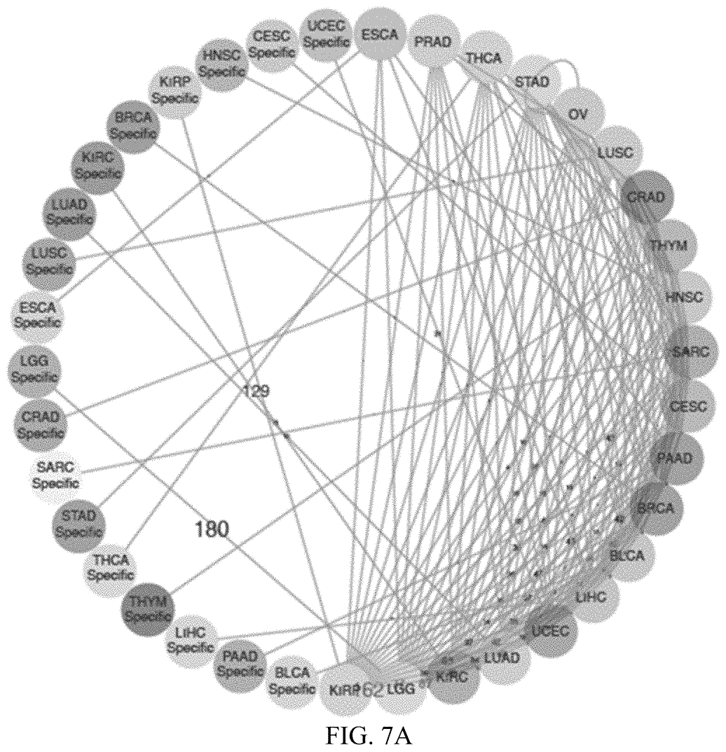

FIG. 6 illustrates survival models at the module and gene level comparing 20 cancer types from the TCGA database. FIG. 6A shows test data set survival model statistics (temporal area under curve (t-AUC) and Harrel's C-Index) at the module level (MEGENA Module—red and nGOseq Module—green) and gene level (MEGENA Gene—light blue and nGOseq Gene—dark blue). FIG. 6B shows survival model statistics at the MEGENA module level (for both training and testing data sets) broken down by each of the 20 cancer types. 9 of 20 cancers have a test data set model statistic above 0.70. FIG. 6C shows Statistics for a survival model built at the MEGENA module level and trained on 19 cancers and tested on a left-out cancer type, UCEC. FIG. 6D shows Kaplan-Meier plots for each of the 20 cancer types stratified into 3 risk groups (Low—red, Moderate—blue, and High—green). Risk stratification was determined by grouping the predicted risks from the survival model at the MEGENA module level into 3 quantiles for all 7,822 samples. P values were calculated via uncorrected log-rank tests for each pairwise risk group comparison (3 per cancer type) for each individual cancer type (20 cancer types).

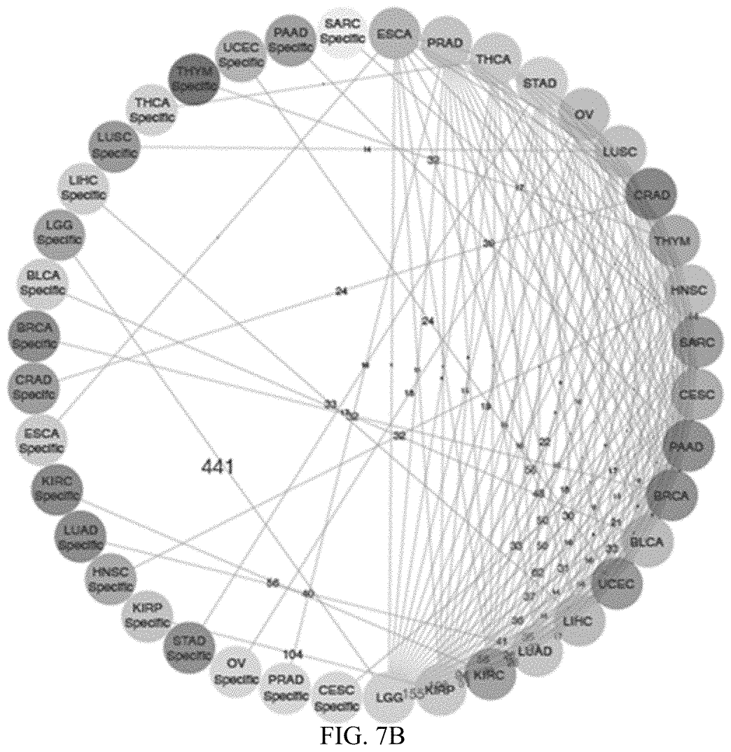

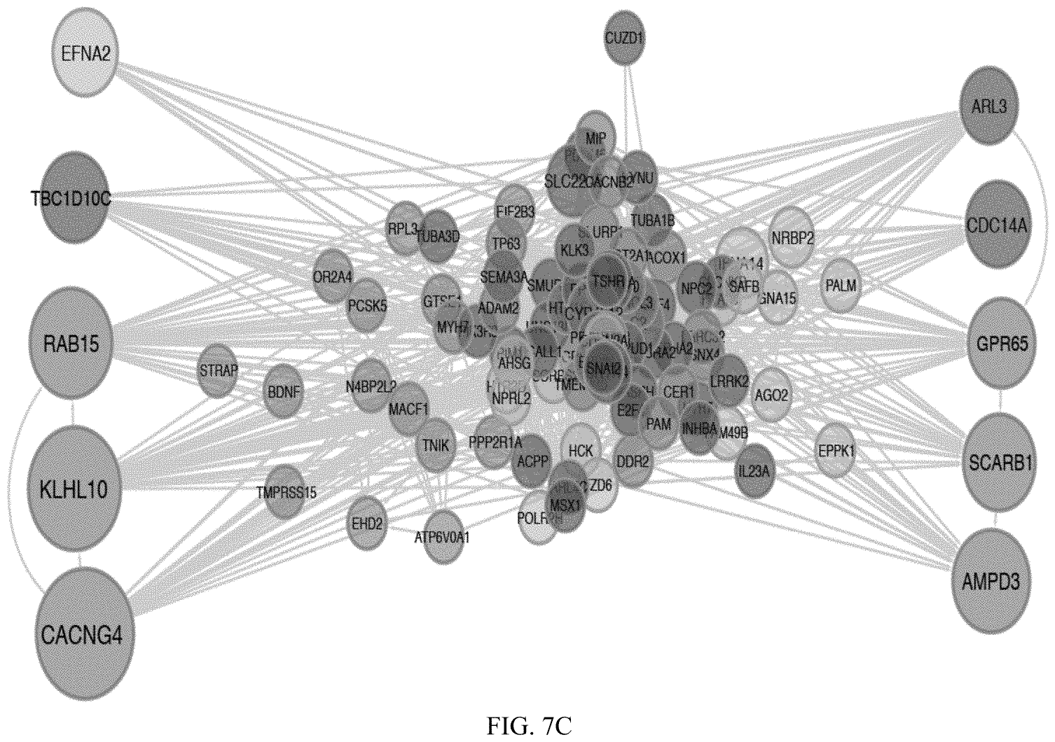

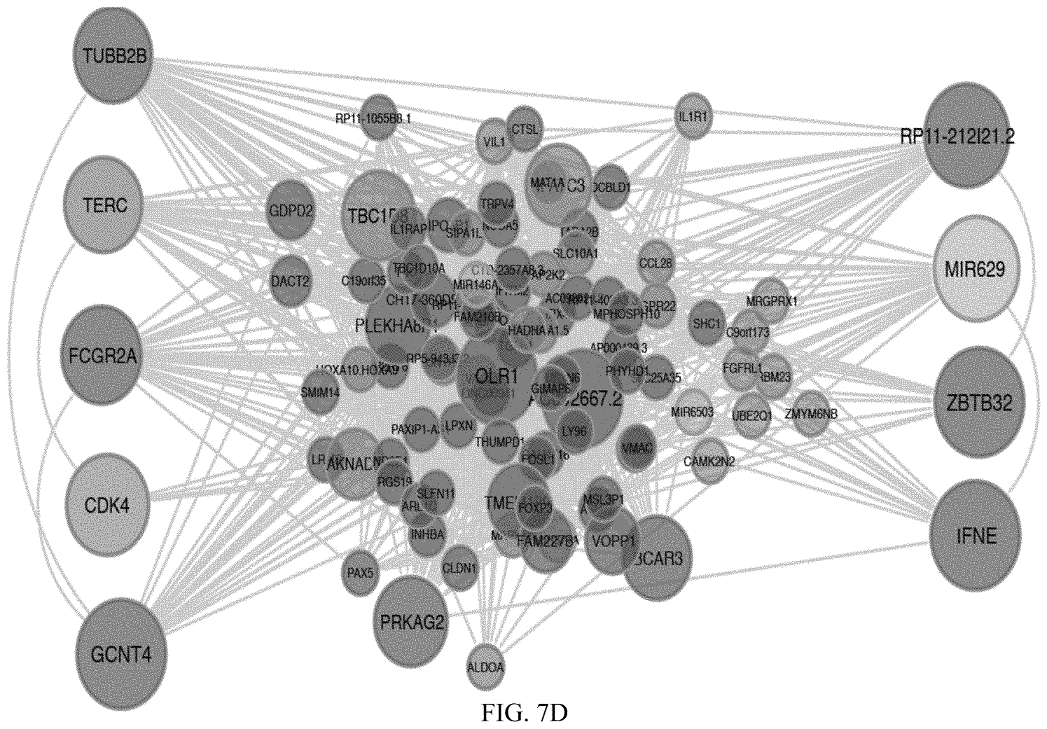

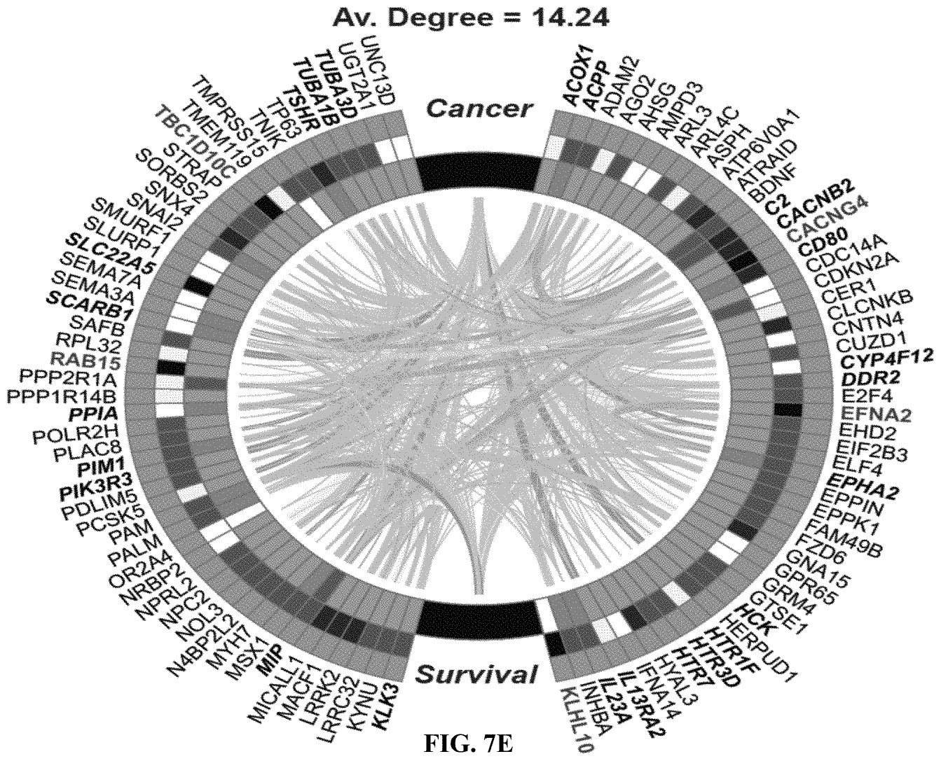

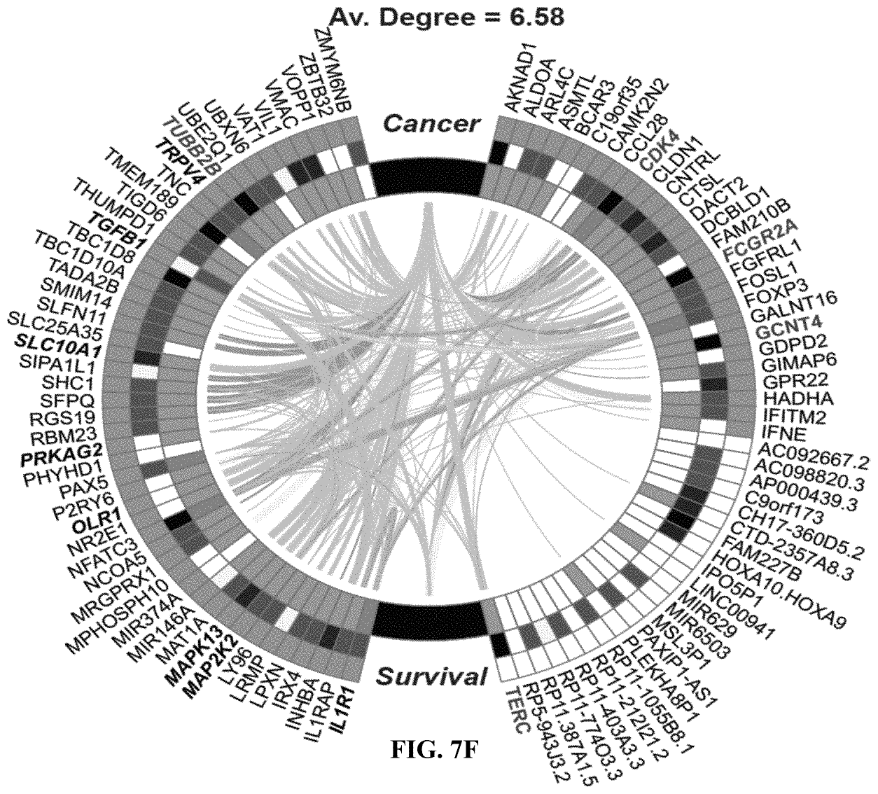

FIG. 7 illustrates an analysis of the most informative survival genes. FIGS. 7A-B show nGOseq (a) and MEGENA (b) networks showing the shared significant hazard ratios (calculated by univariate cox-proportional hazards models and correcting for false discovery with the Benjamini-Hochberg procedure) between different cancer types for the full gene level inputs. Edges connecting cancer types are labeled with the number of significant hazard ratios shared between the cancer types. Also shown are significant hazard ratios that are specific to a single cancer type (i.e. LGG Specific). FIGS. 7C-D show nGOseq (c) and MEGENA (d) derived directed acyclic graphs (DAG) from the training data set showing causal drivers of the 100 most informative genes for survival. Genes on the left side of the DAG are the most likely upstream causal drivers while the genes on the right are the most likely downstream targets (both determined based on incoming and outgoing edges in the DAG). Data types: METH (blue), mRNA (red), miRNA (orange), STV (Pink), CNV (green). FIGS. 7E-F shows nGOseq (e) and MEGENA (f) natural language processing diagrams showing known literature connections between the 100 most informative genes cancer, and survival (using MESH terms as detailed in Methods) as well as known literature gene to gene connections. For each gene, the outer ring indicates the presence (blue) or absence (white) of functional annotation, the middle ring displays the difference in outgoing to incoming edges from the respective DAG (colored with 10 bins where white—lowest and black—highest), and the inner ring indicates the total number of edges (colored with 6 bins where white—lowest and dark purple—highest). Inner chord colors for gene to cancer relationships (gene to cancer and/or kidney cancer): red—inhibitory, grey—neither inhibitory or stimulatory, green—stimulatory, yellow—both inhibitory and stimulatory. Inner chord colors for gene to gene relationships: pink—inhibitory, purple—neither inhibitory or stimulatory, orange—stimulatory, blue—both inhibitory and stimulatory. Genes highlighted in red are those that appear on the left side of the DAGs and those that are bold and italicized are known drug targets. Average degree of gene connections to cancer, survival, and other genes is displayed above the diagram.

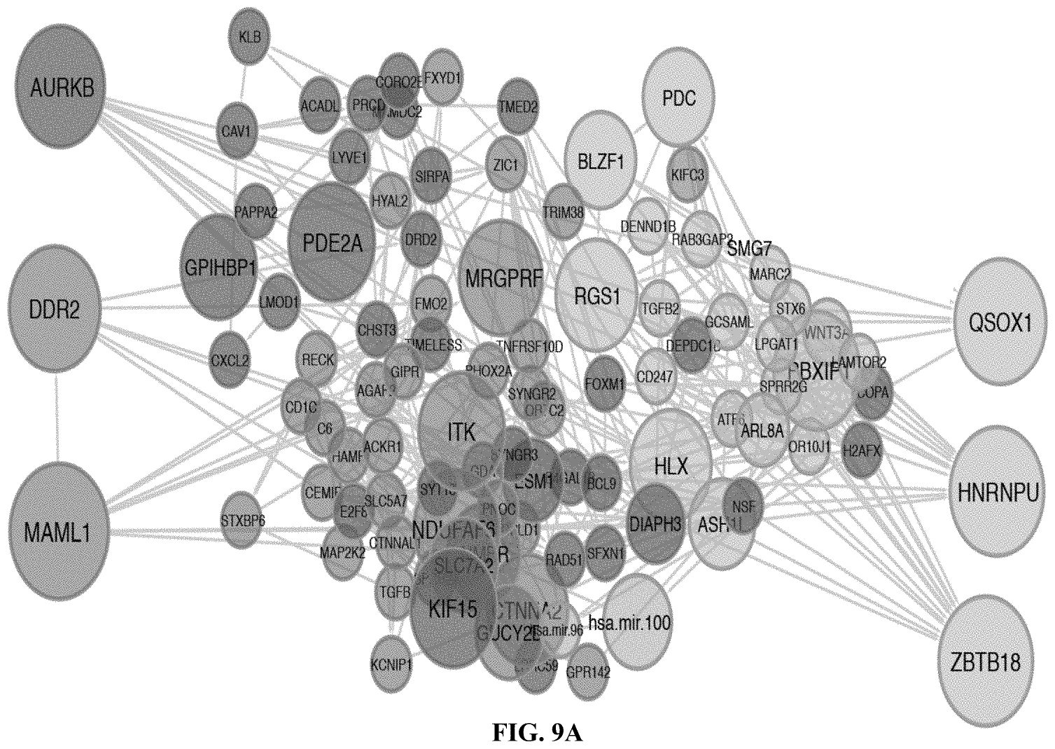

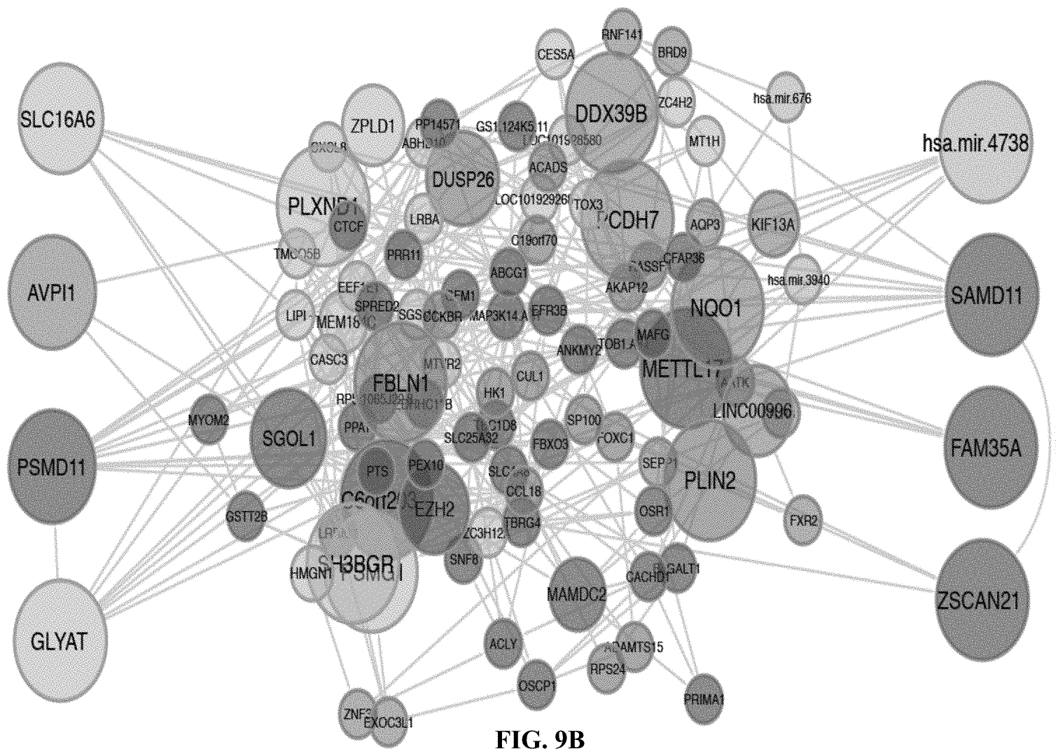

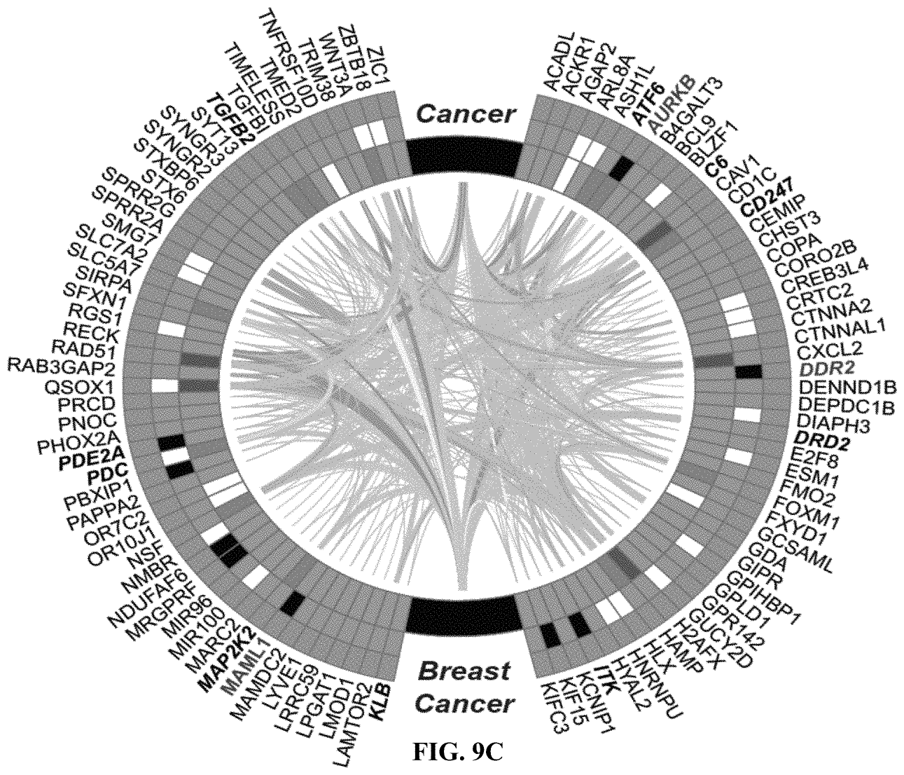

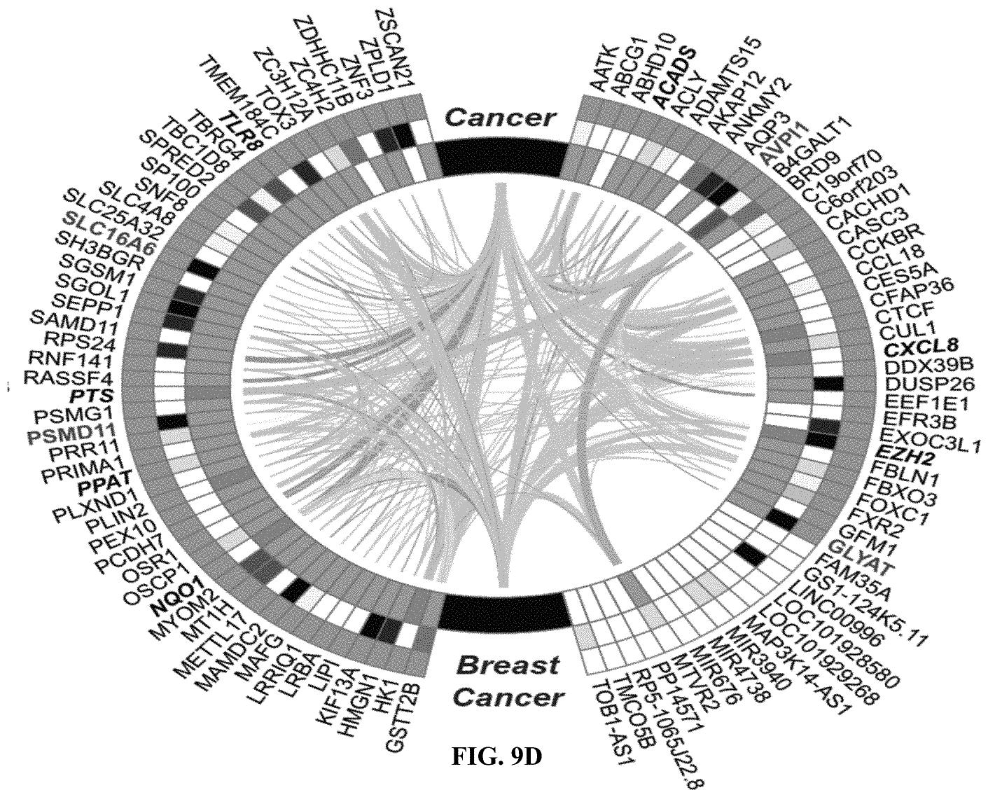

FIG. 9A-FIG. 9D depict binomial model comparisons at both the module and gene level specifically highlighting breast cancer (BRCA) versus normal tissue. FIG. 9A and FIG. 9B show nGOseq (FIG. 9A) and MEGENA (FIG. 9B) derived directed acyclic graphs (DAG) from the training data set showing causal drivers of the 100 most informative genes for BRCA vs. Normal. Genes on the left side of the DAG are the most likely upstream causal drivers while the genes on the right are the most likely downstream targets (both determined based on incoming and outgoing edges in the DAG). Data types: METH (blue), mRNA (red), miRNA (orange), STV (Pink), CNV (green) FIG. 9C and FIG. 9D show nGOseq (FIG. 9C) and MEGENA (FIG. 9D) natural language processing diagrams showing known literature connections between the 100 most informative genes cancer and/or breast cancer (using MESH terms as detailed in Methods) as well as known literature gene to gene connections. For each gene, the outer ring indicates the presence (blue) or absence (white) of functional annotation, the middle ring displays the difference in outgoing to incoming edges from the respective DAG (colored with 10 bins where white—lowest and black—highest, see Methods), and the inner ring indicates the total number of edges (colored with 6 bins where white—lowest and dark purple—highest, see Methods). Inner chord colors for gene to cancer relationships (gene to cancer and/or kidney cancer): red—inhibitory, grey—neither inhibitory or stimulatory, green—stimulatory, yellow—both inhibitory and stimulatory. Inner chord colors for gene to gene relationships: pink—inhibitory, purple—neither inhibitory or stimulatory, orange—stimulatory, blue—both inhibitory and stimulatory. Genes highlighted in red are those that appear on the left side of the DAGs and those that are bold and italicized are known drug targets. Average degree of gene connections to both cancer and/or breast cancer and other genes is displayed above the diagram.

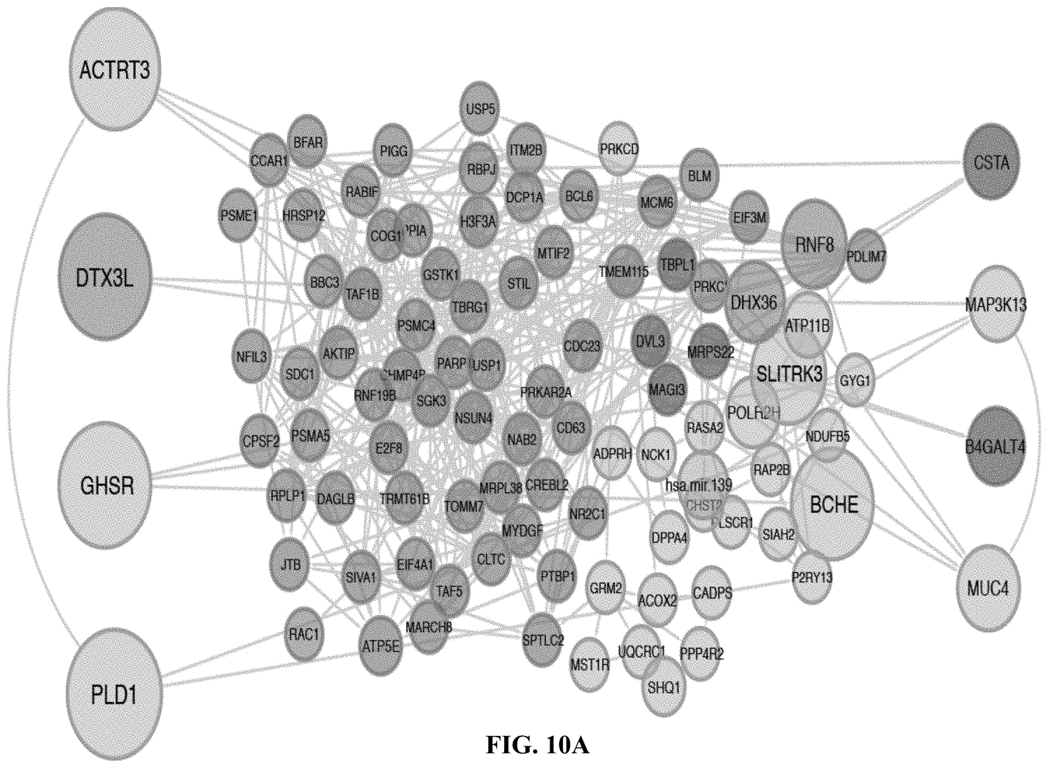

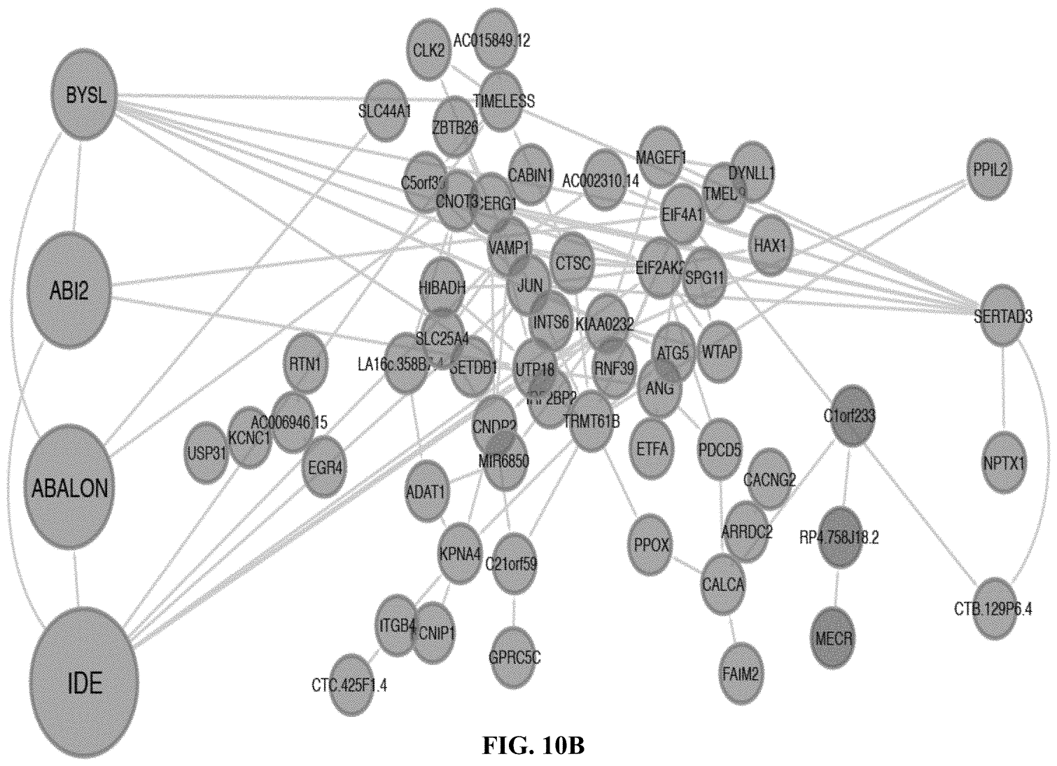

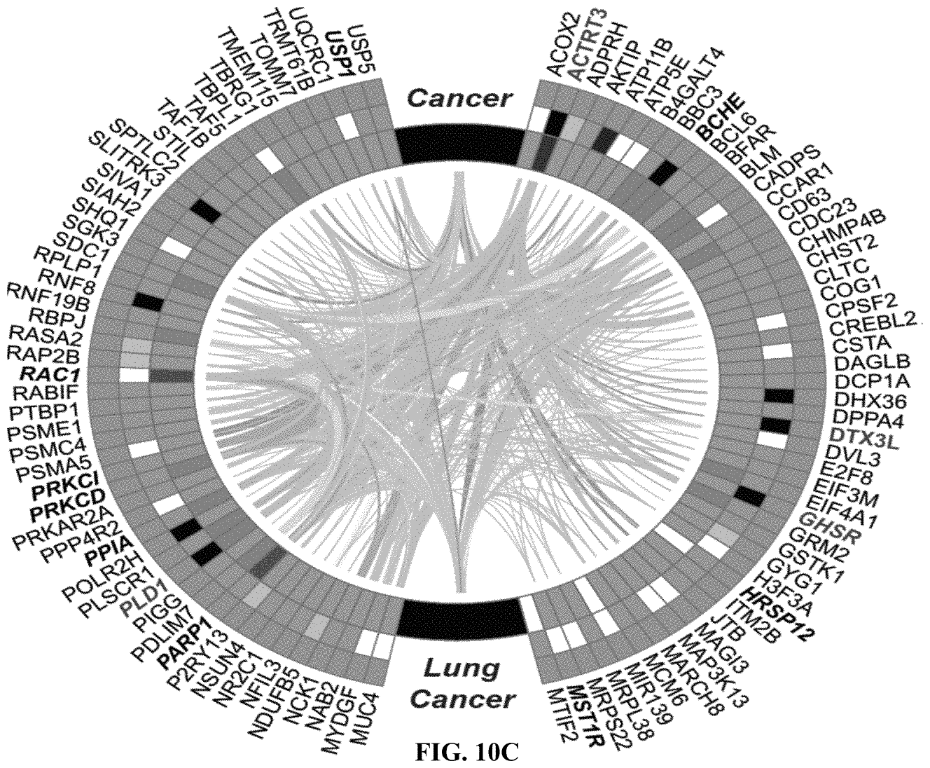

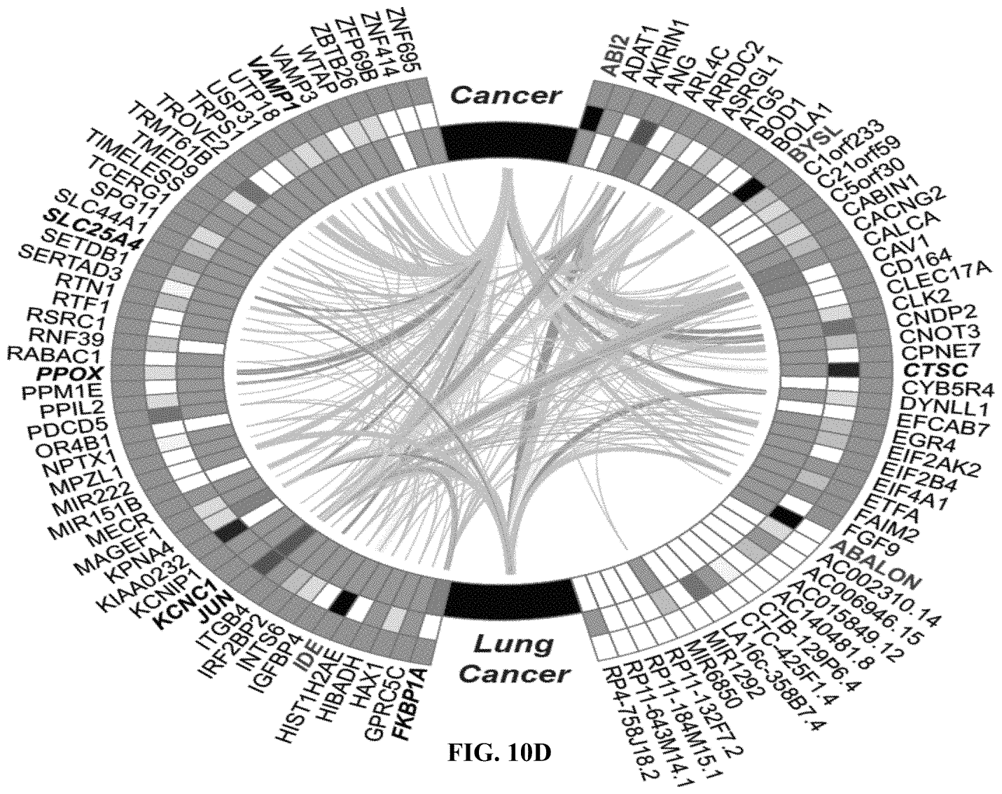

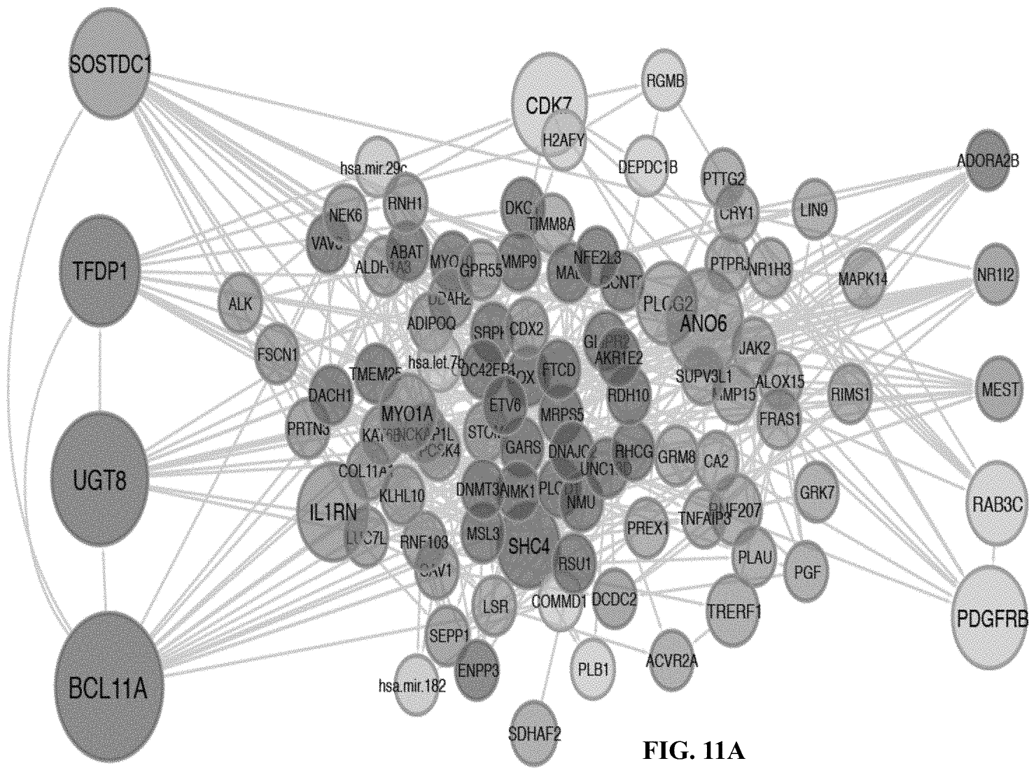

FIG. 10A-FIG. 10D depict binomial model comparisons at both the module and gene level specifically highlighting LUAD versus LUSC lung cancer subtypes. FIG. 10A and FIG. 10B show nGOseq (FIG. 10A) and MEGENA (FIG. 10B) derived directed acyclic graphs (DAG) from the training data set showing causal drivers of the 100 most informative genes for LUAD versus LUSC. Genes on the left side of the DAG are the most likely upstream causal drivers while the genes on the right are the most likely downstream targets (both determined based on incoming and outgoing edges in the DAG). Data types: METH (blue), mRNA (red), miRNA (orange), STV (Pink), CNV (green). FIG. 10C and FIG. 10D show nGOseq (FIG. 10C) and MEGENA (FIG. 10D) natural language processing diagrams showing known literature connections between the 100 most informative genes and cancer (using MESH terms as detailed in Methods) as well as known literature gene to gene connections. For each gene, the outer ring indicates the presence (blue) or absence (white) of functional annotation, the middle ring displays the difference in outgoing to incoming edges from the respective DAG (colored with 10 bins where white—lowest and black—highest, see Methods), and the inner ring indicates the total number of edges (colored with 6 bins where white—lowest and dark purple—highest, see Methods). Inner chord colors for gene to cancer relationships (gene to cancer and/or kidney cancer): red—inhibitory, grey—neither inhibitory or stimulatory, green—stimulatory, yellow—both inhibitory and stimulatory. Inner chord colors for gene to gene relationships: pink—inhibitory, purple—neither inhibitory or stimulatory, orange—stimulatory, blue—both inhibitory and stimulatory. Genes highlighted in red are those that appear on the left side of the DAGs. Average degree of gene connections to both cancer and/or lung cancer and other genes is displayed above the diagram.

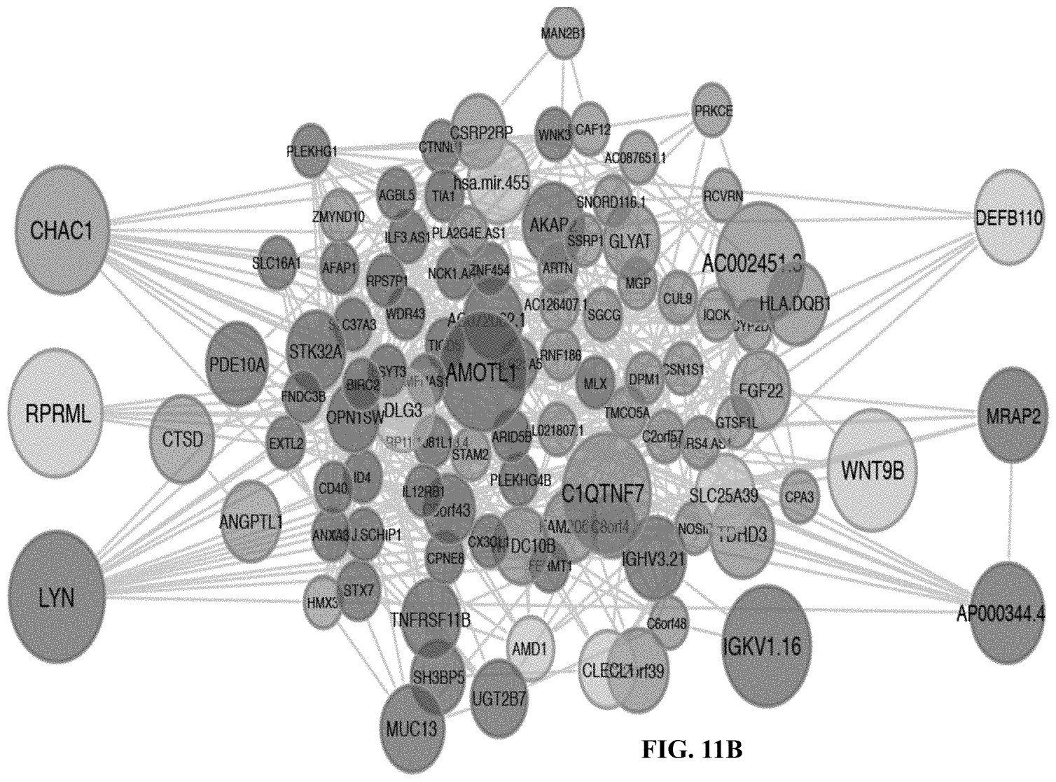

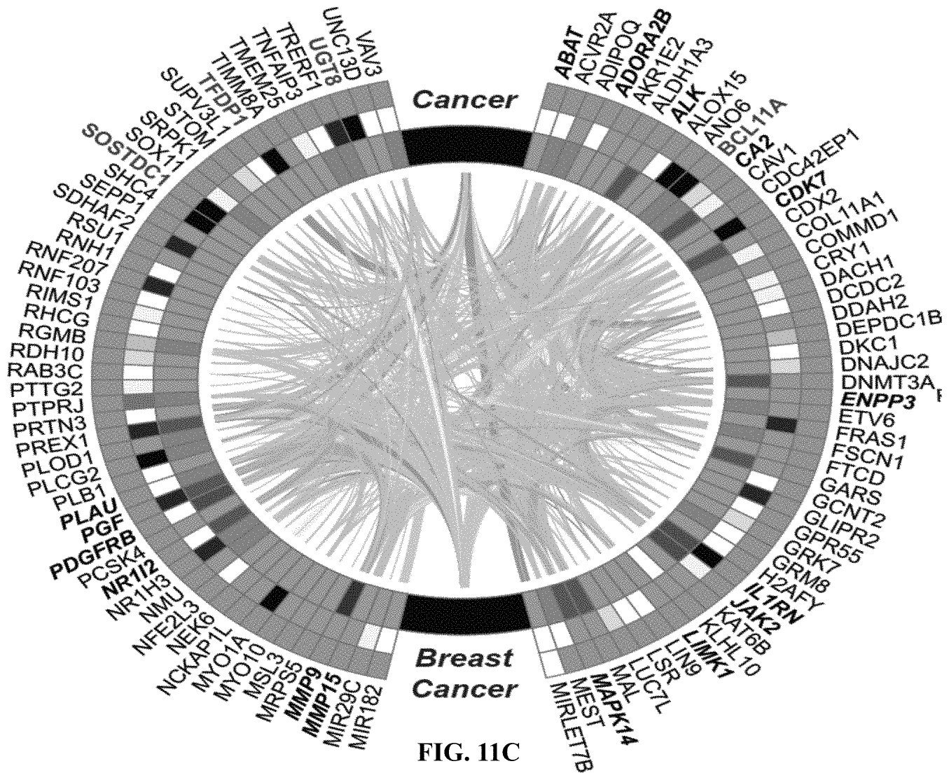

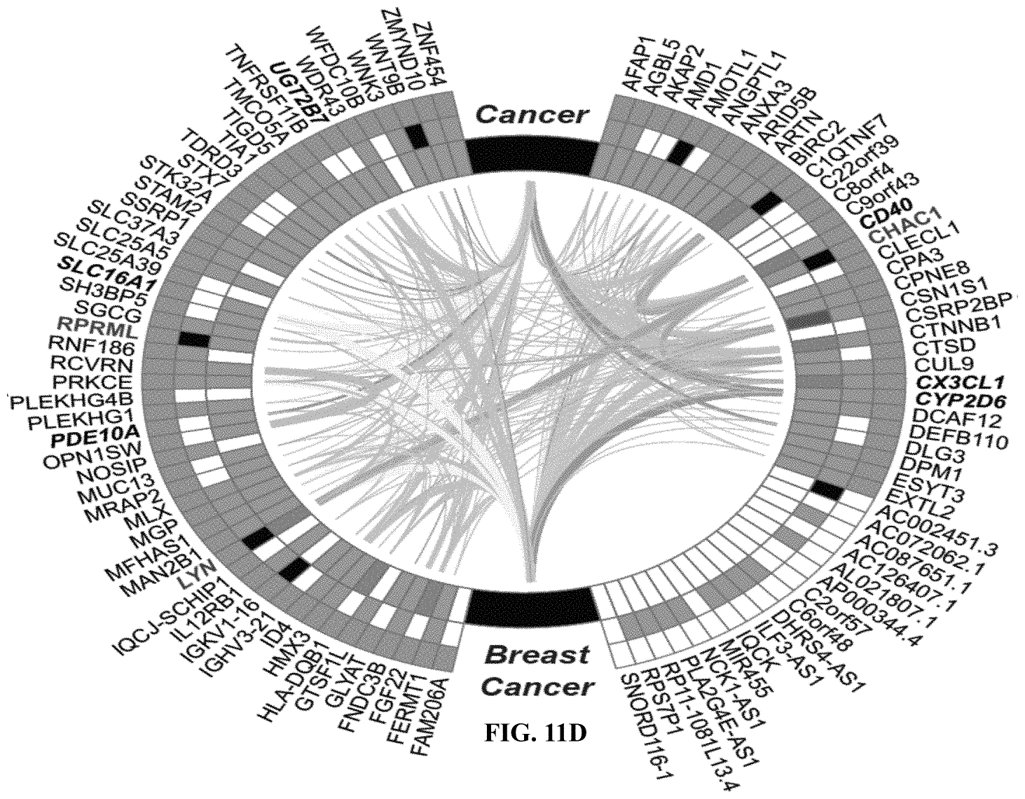

FIG. 11A-FIG. 11D depict binomial model comparisons at both the module and gene level specifically highlighting ER+ versus ER− breast cancer subtypes. FIG. 11A and FIG. 11B show nGOseq (FIG. 11A) and MEGENA (FIG. 11B) derived directed acyclic graphs (DAG) from the training data set showing causal drivers of the 100 most informative genes for ER positive versus ER negative. Genes on the left side of the DAG are the most likely upstream causal drivers while the genes on the right are the most likely downstream targets (both determined based on incoming and outgoing edges in the DAG). Data types: METH (blue), mRNA (red), miRNA (orange), STV (Pink), CNV (green). FIG. 11C and FIG. 11D show nGOseq (FIG. 11C) and MEGENA (FIG. 11D) natural language processing diagrams showing known literature connections between the 100 most informative genes and cancer (using MESH terms as detailed in Methods) as well as known literature gene to gene connections. For each gene, the outer ring indicates the presence (blue) or absence (white) of functional annotation, the middle ring displays the difference in outgoing to incoming edges from the respective DAG (colored with 10 bins where white—lowest and black—highest, see Methods), and the inner ring indicates the total number of edges (colored with 6 bins where white—lowest and dark purple—highest, see Methods). Inner chord colors for gene to cancer relationships (gene to cancer and/or kidney cancer): red—inhibitory, grey—neither inhibitory or stimulatory, green—stimulatory, yellow—both inhibitory and stimulatory. Inner chord colors for gene to gene relationships: pink—inhibitory, purple—neither inhibitory or stimulatory, orange—stimulatory, blue—both inhibitory and stimulatory. Genes highlighted in red are those that appear on the left side of the DAGs and those that are bold and italicized are known drug targets. Average degree of gene connections to both cancer and/or breast cancer and other genes is displayed above the diagram.

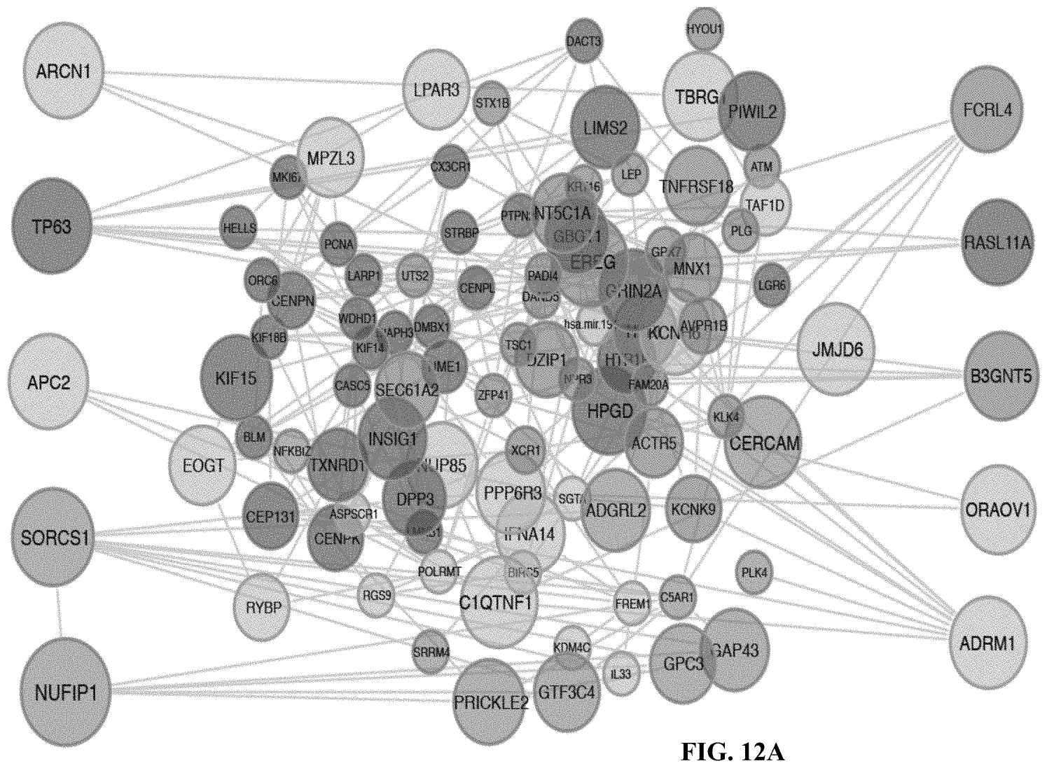

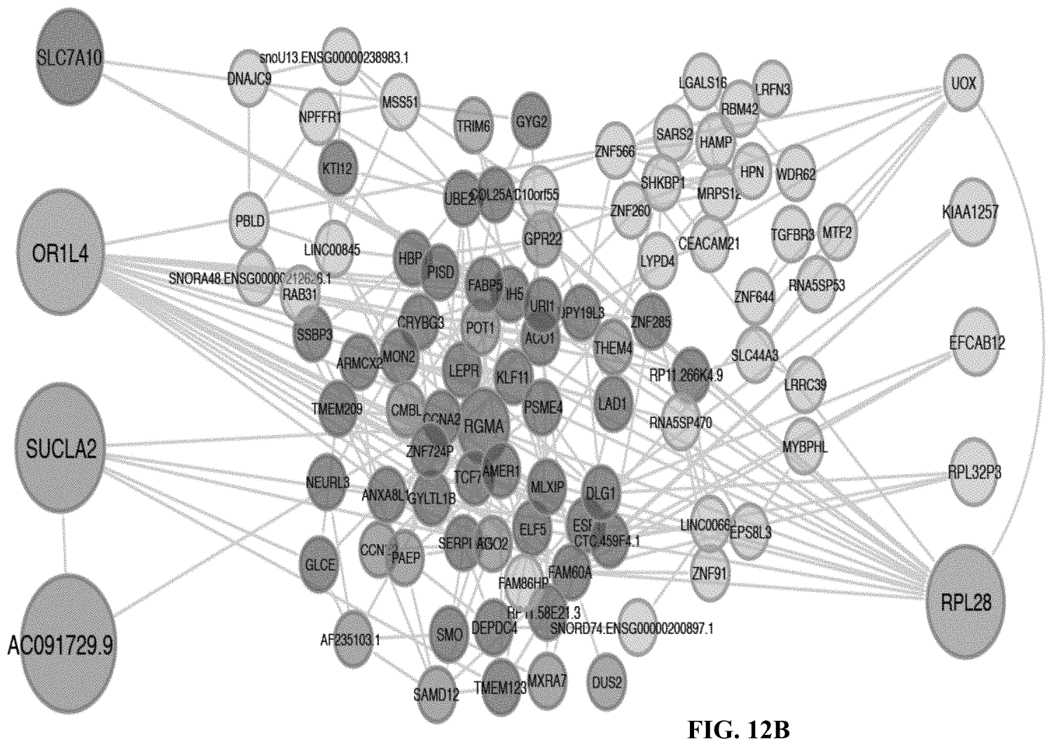

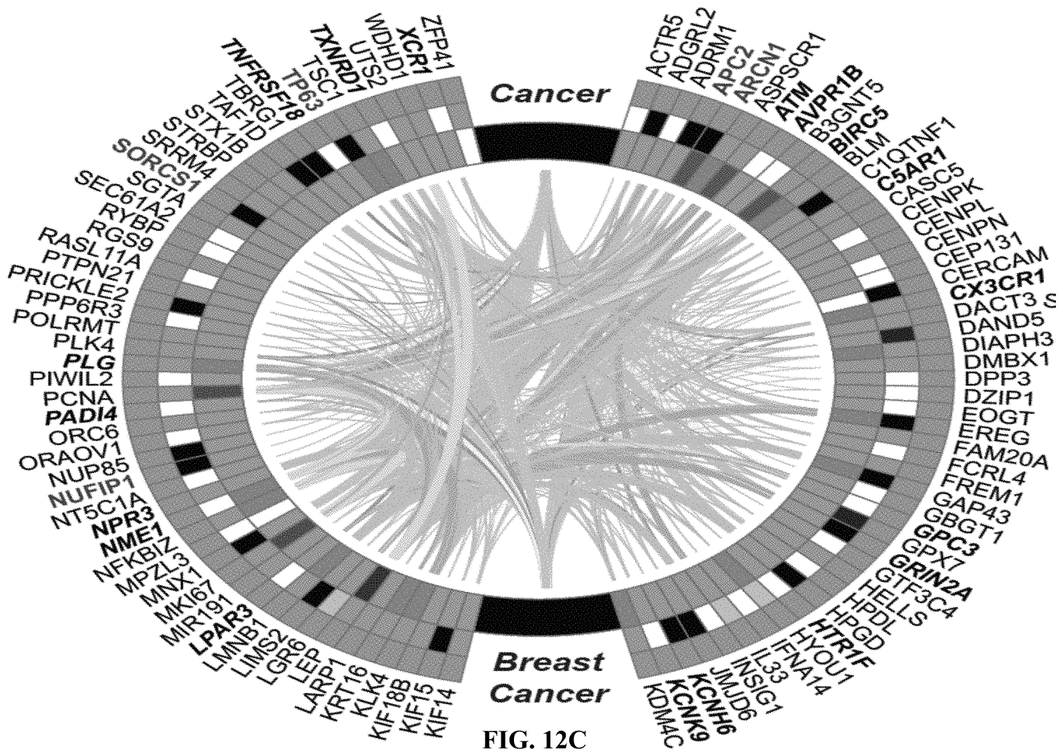

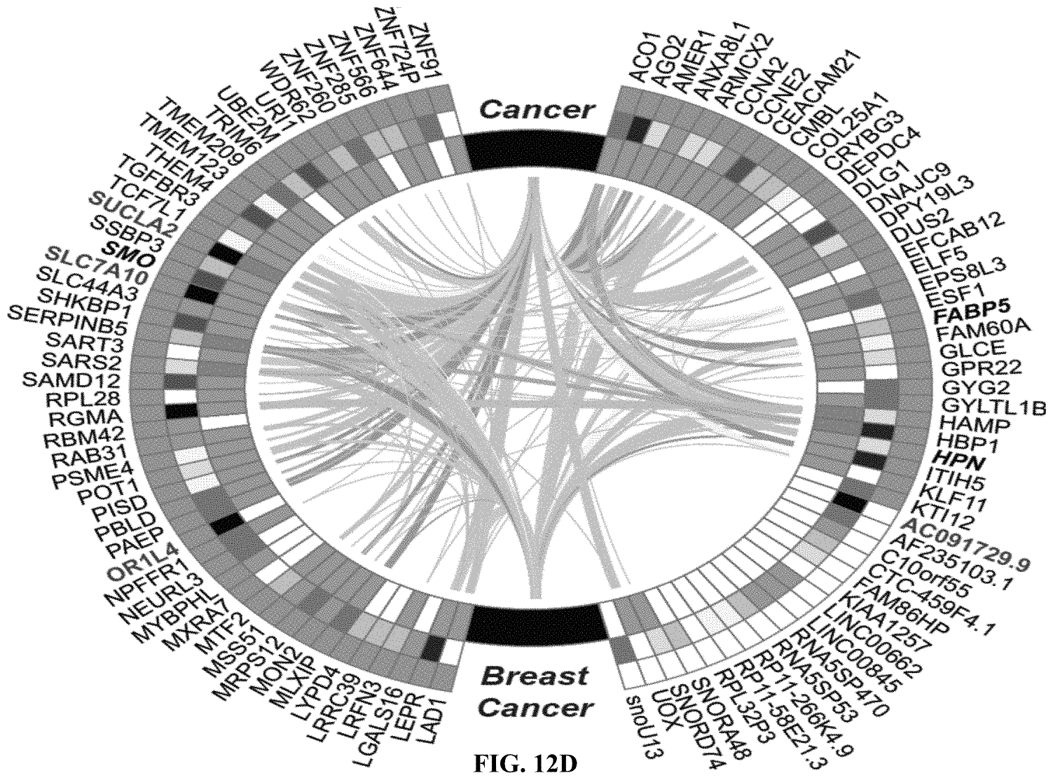

FIG. 12A-FIG. 12D depict binomial model comparisons at both the module and gene level specifically highlighting Luminal A versus Luminal B breast cancer subtypes. FIG. 12A and FIG. 12B show nGOseq (FIG. 12A) and MEGENA (FIG. 12B) derived directed acyclic graphs (DAG) from the training data set showing causal drivers of the 100 most informative genes for Luminal A versus Luminal B. Genes on the left side of the DAG are the most likely upstream causal drivers while the genes on the right are the most likely downstream targets (both determined based on incoming and outgoing edges in the DAG). Data types: METH (blue), mRNA (red), miRNA (orange), STV (Pink), CNV (green). FIG. 12C and FIG. 12D show nGOseq (FIG. 12C) and MEGENA (FIG. 12D) natural language processing diagrams showing known literature connections between the 100 most informative genes and cancer (using MESH terms as detailed in Methods) as well as known literature gene to gene connections. For each gene, the outer ring indicates the presence (blue) or absence (white) of functional annotation, the middle ring displays the difference in outgoing to incoming edges from the respective DAG (colored with 10 bins where white—lowest and black—highest, see Methods), and the inner ring indicates the total number of edges (colored with 6 bins where white—lowest and dark purple—highest, see Methods). Inner chord colors for gene to cancer relationships (gene to cancer and/or kidney cancer): red—inhibitory, grey—neither inhibitory or stimulatory, green—stimulatory, yellow—both inhibitory and stimulatory. Inner chord colors for gene to gene relationships: pink—inhibitory, purple—neither inhibitory or stimulatory, orange—stimulatory, blue—both inhibitory and stimulatory. Genes highlighted in red are those that appear on the left side of the DAGs and those that are bold and italicized are known drug targets. Average degree of gene connections to both cancer and/or breast cancer and other genes is displayed above the diagram.

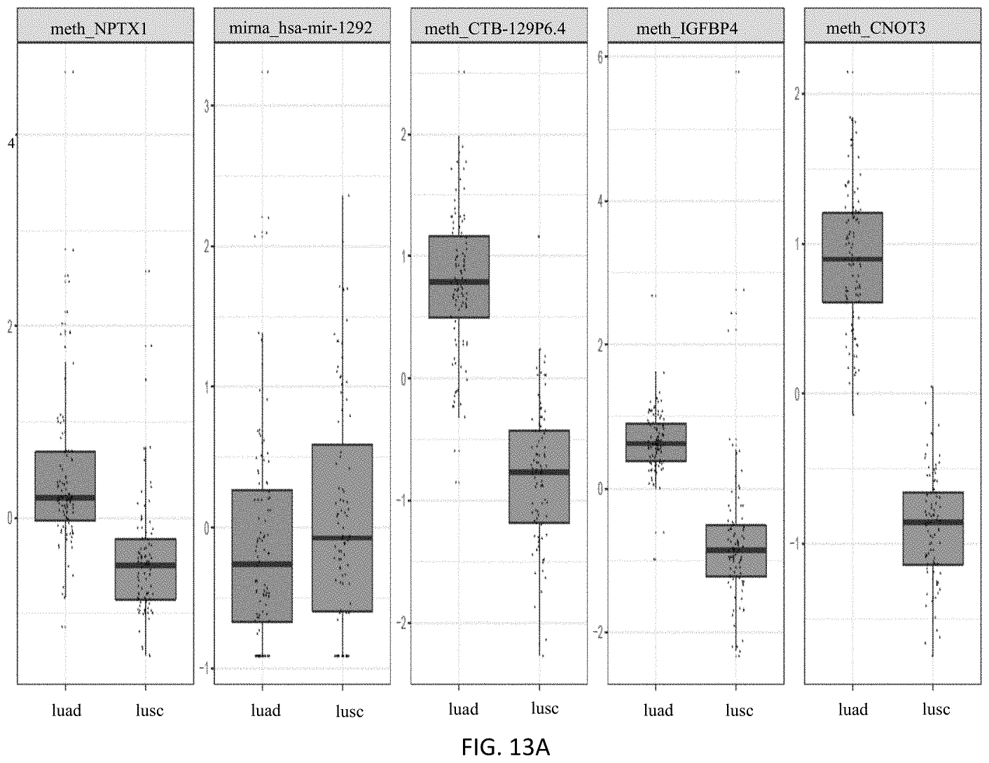

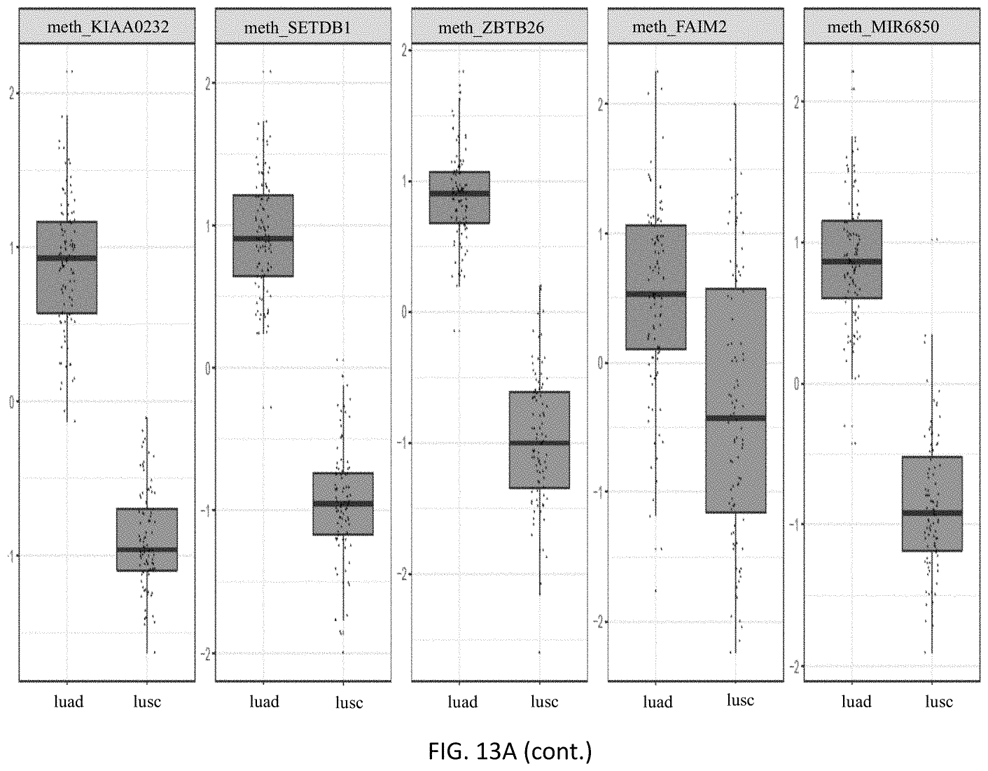

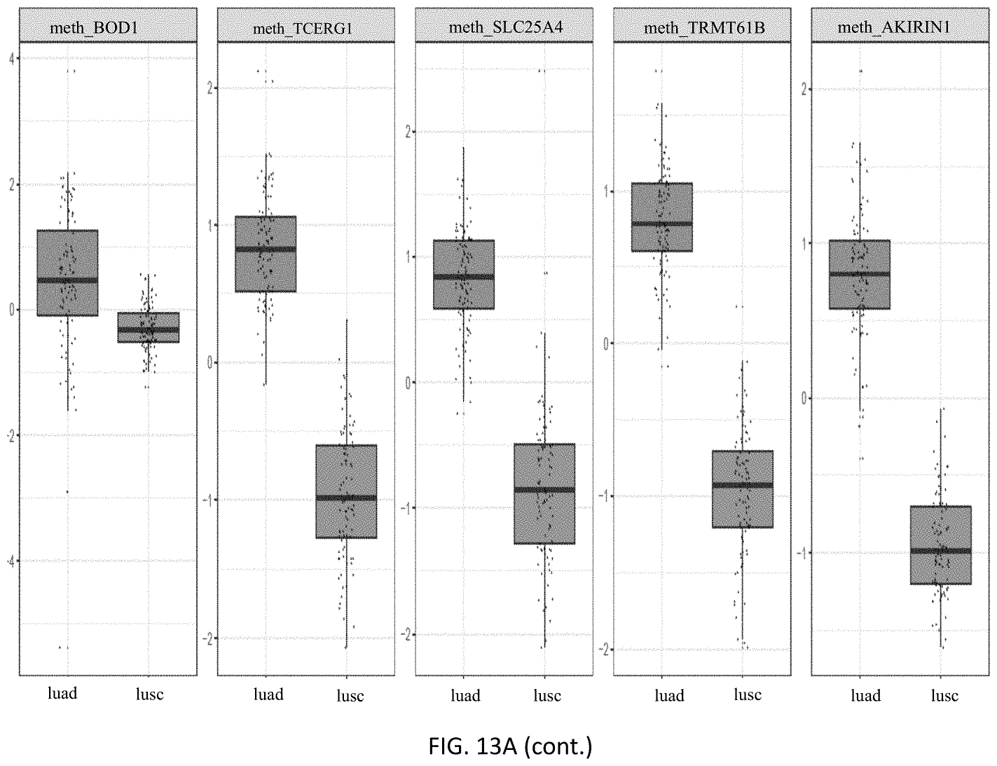

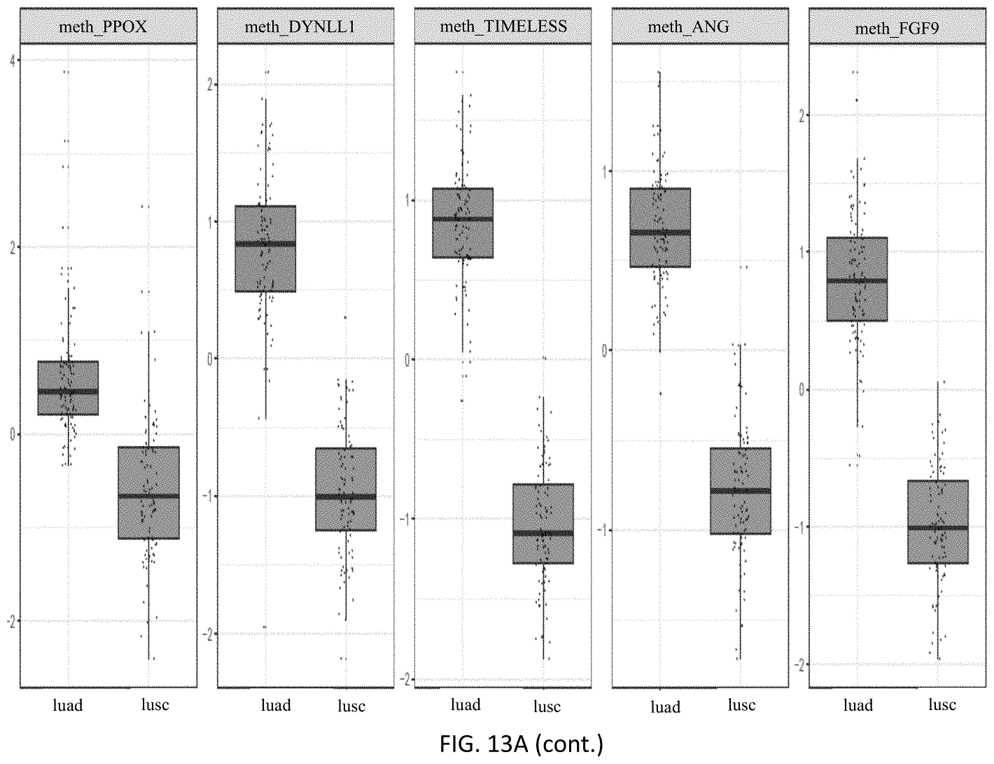

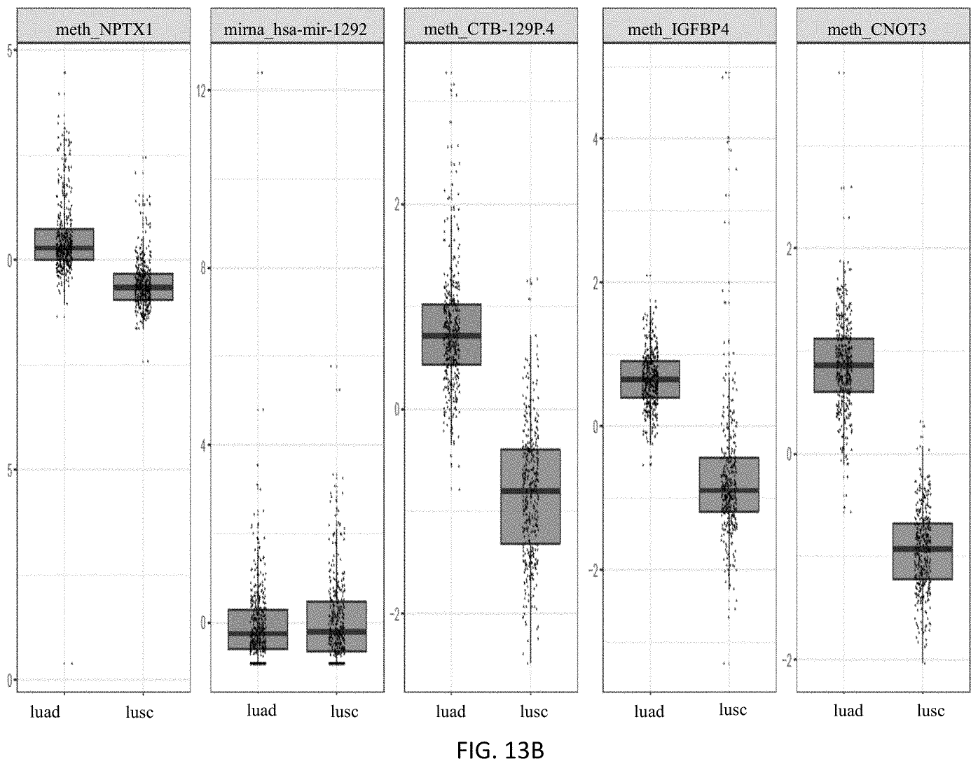

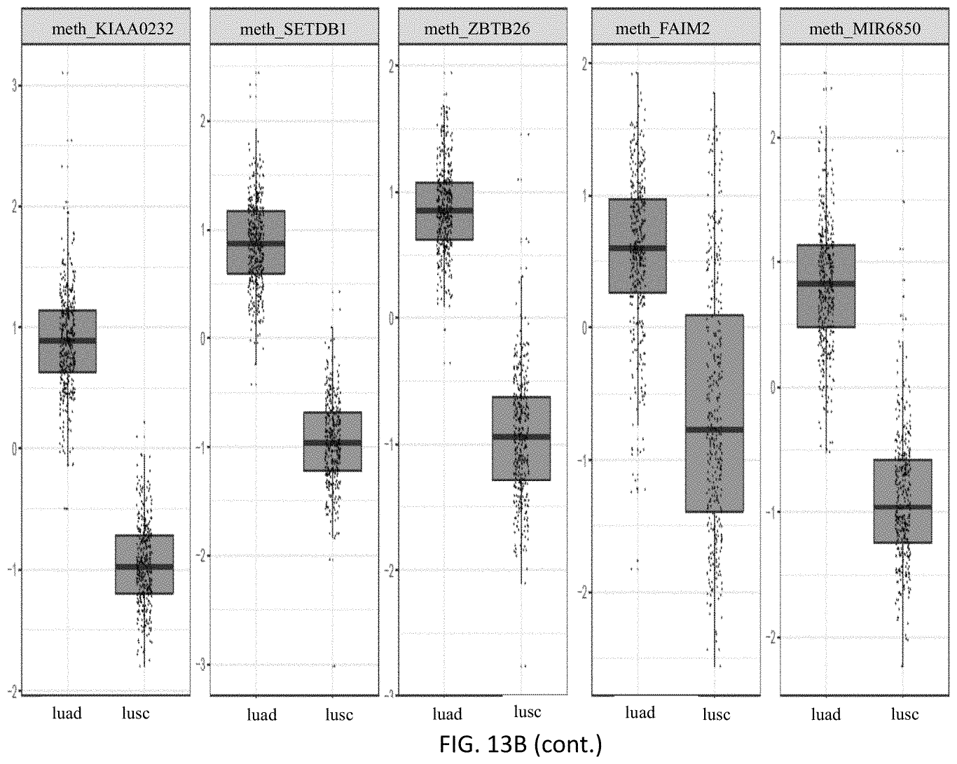

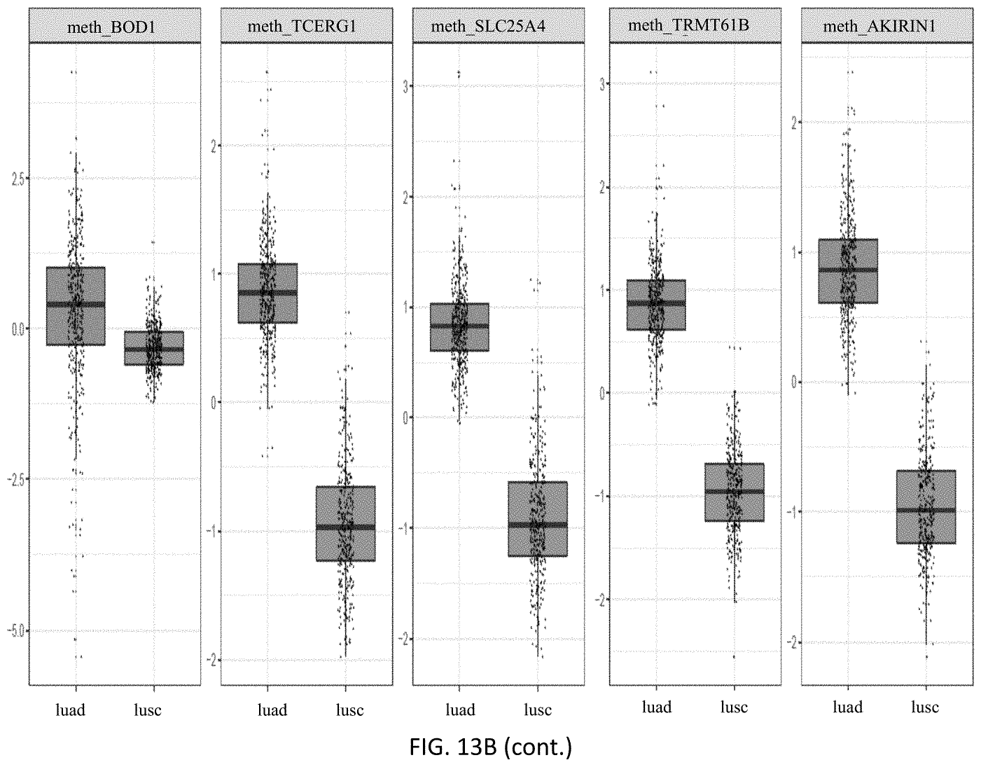

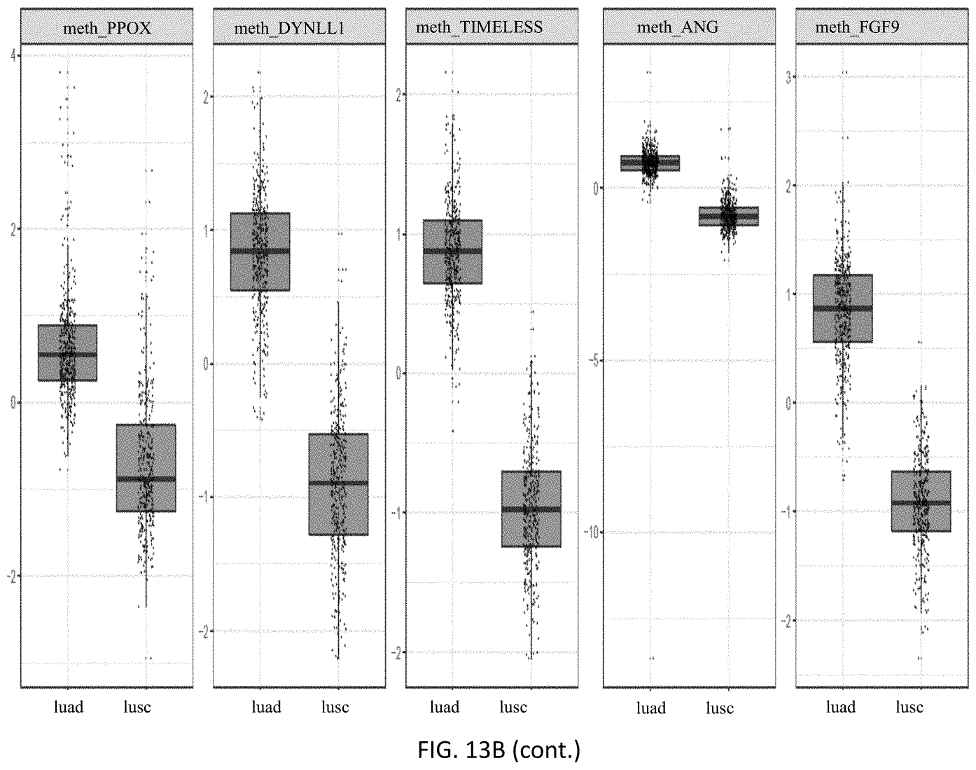

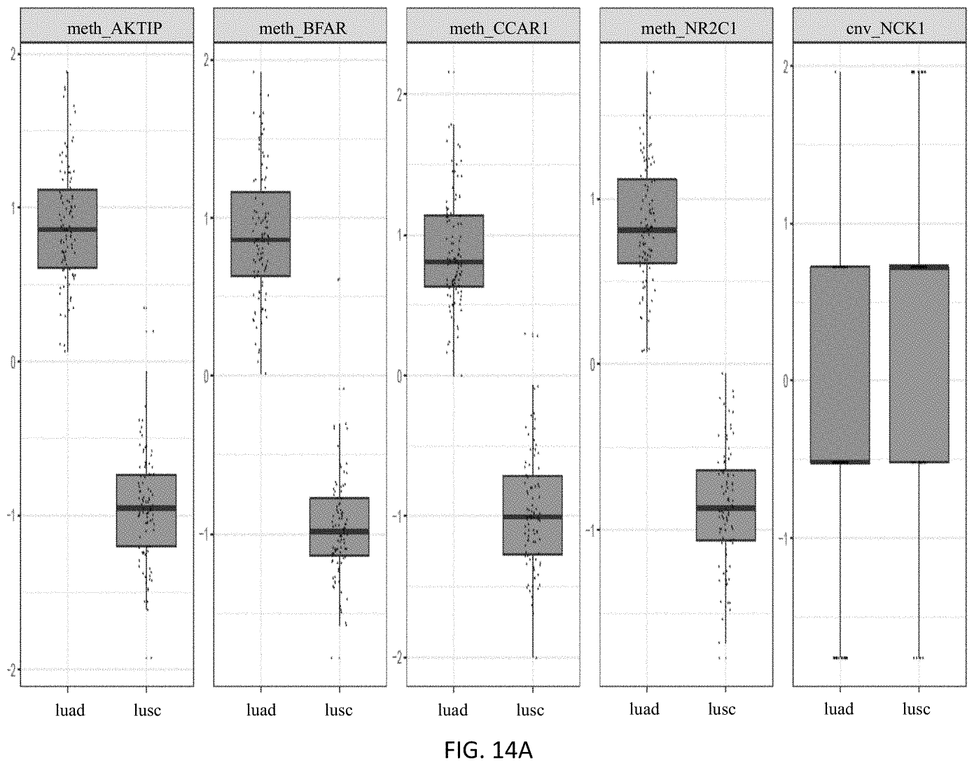

FIG. 13A and FIG. 13B depict the top 20 most informative MEGENA genes at the gene level for Lung Adenocarcinoma (LUAD) versus Lung Squamous Cell (LUSC) lung cancer subtypes (for both training (FIG. 13B) and testing data sets (13A)).

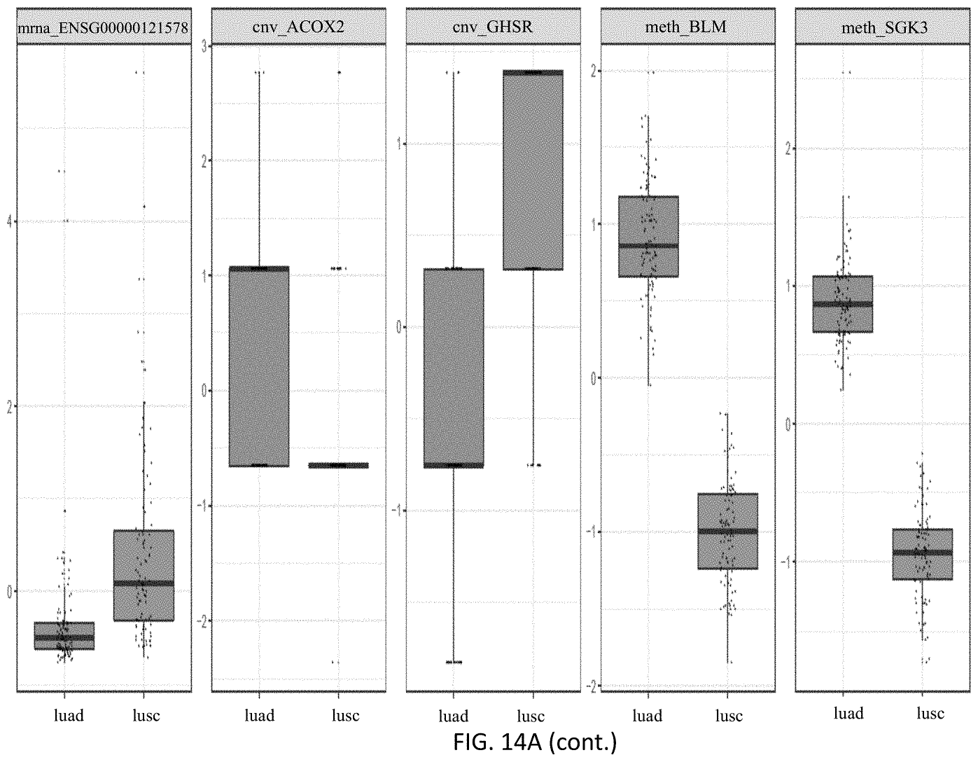

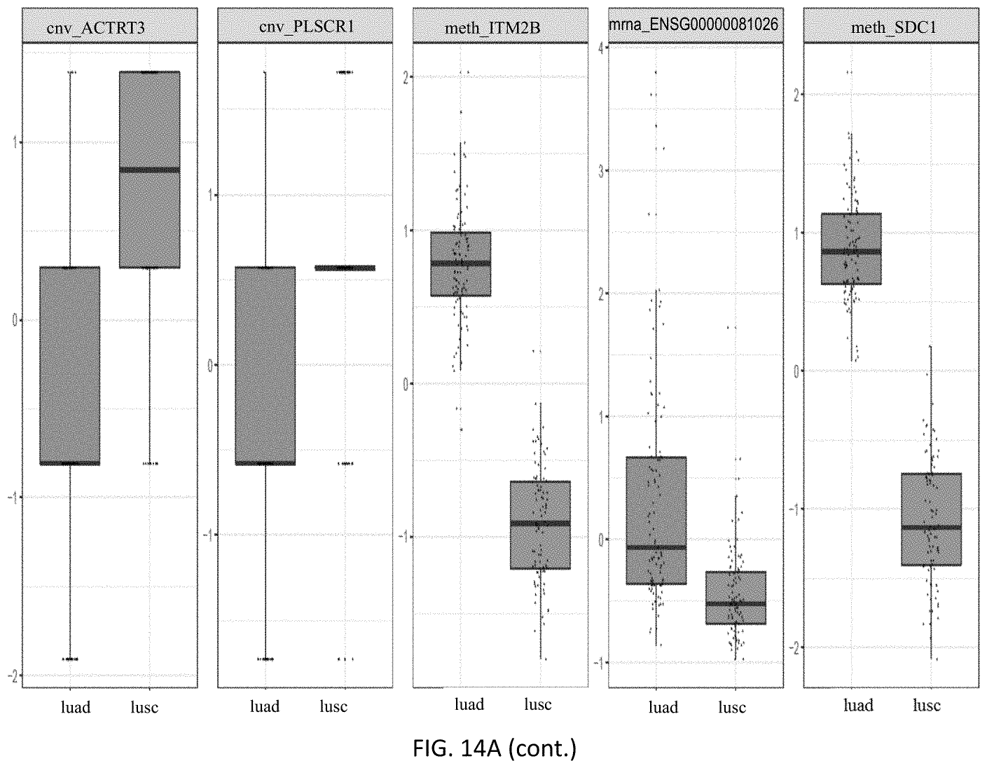

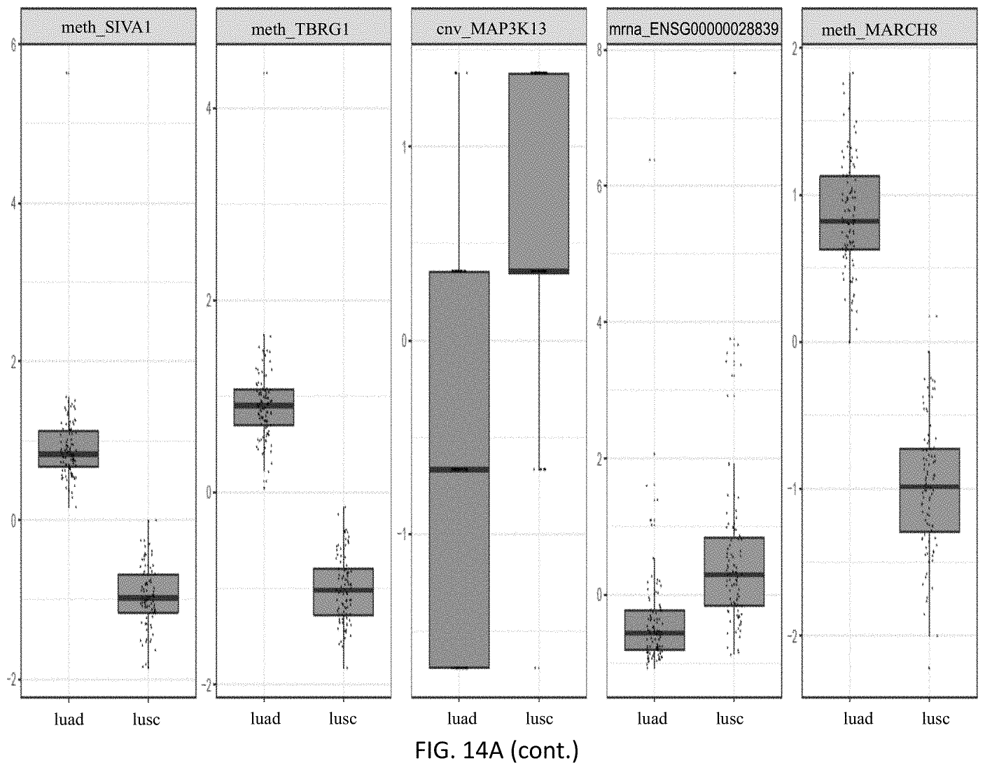

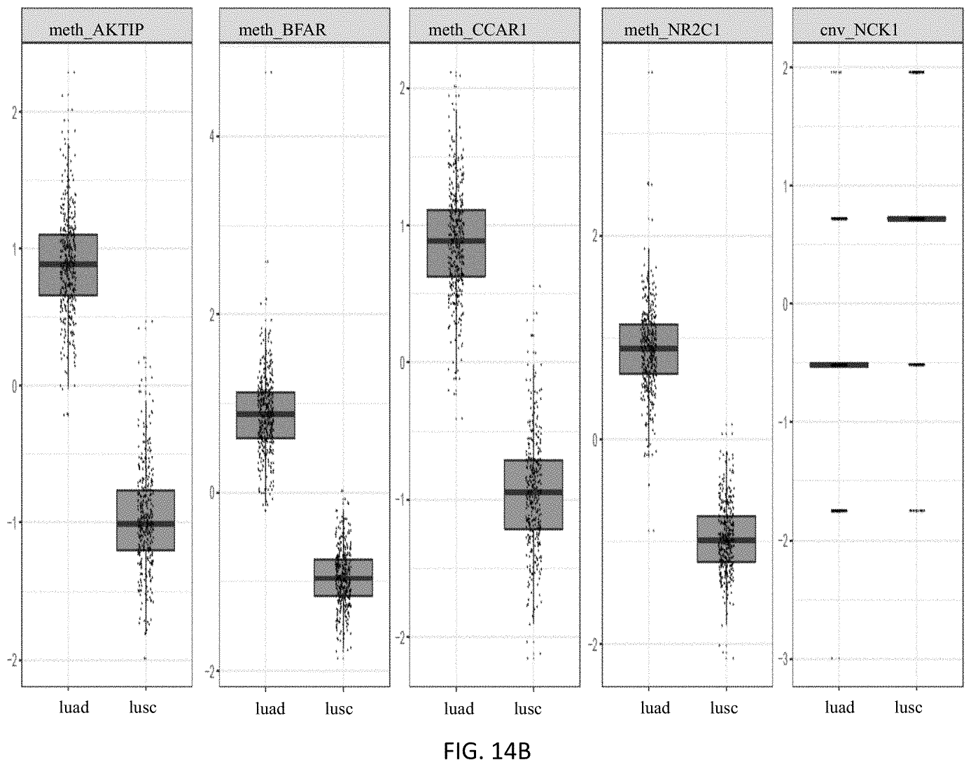

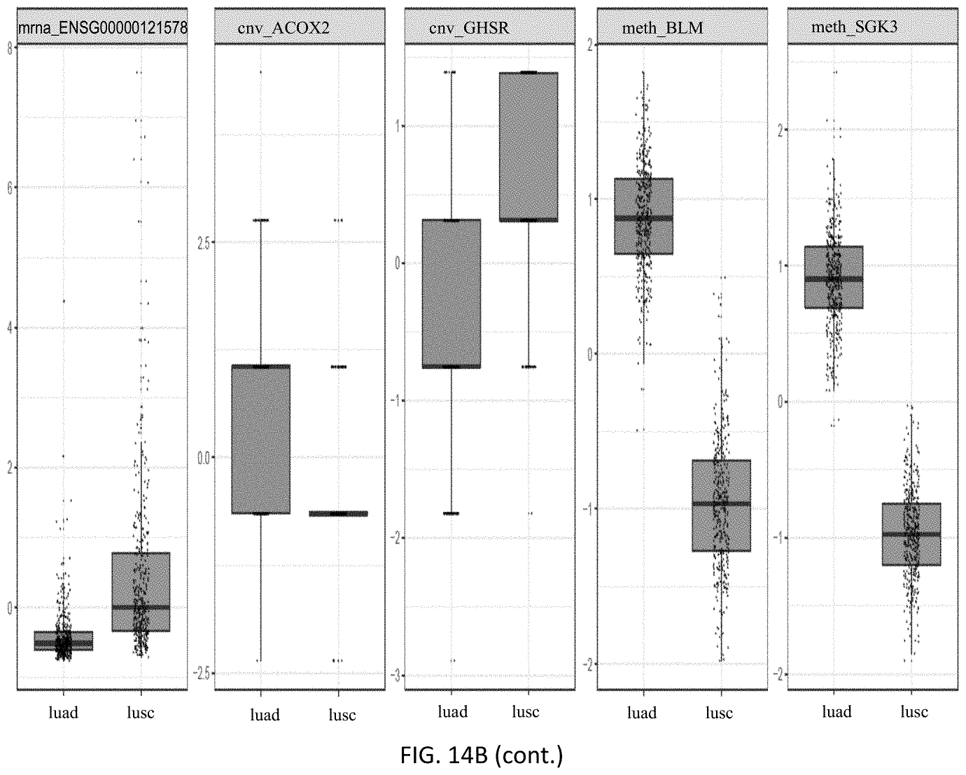

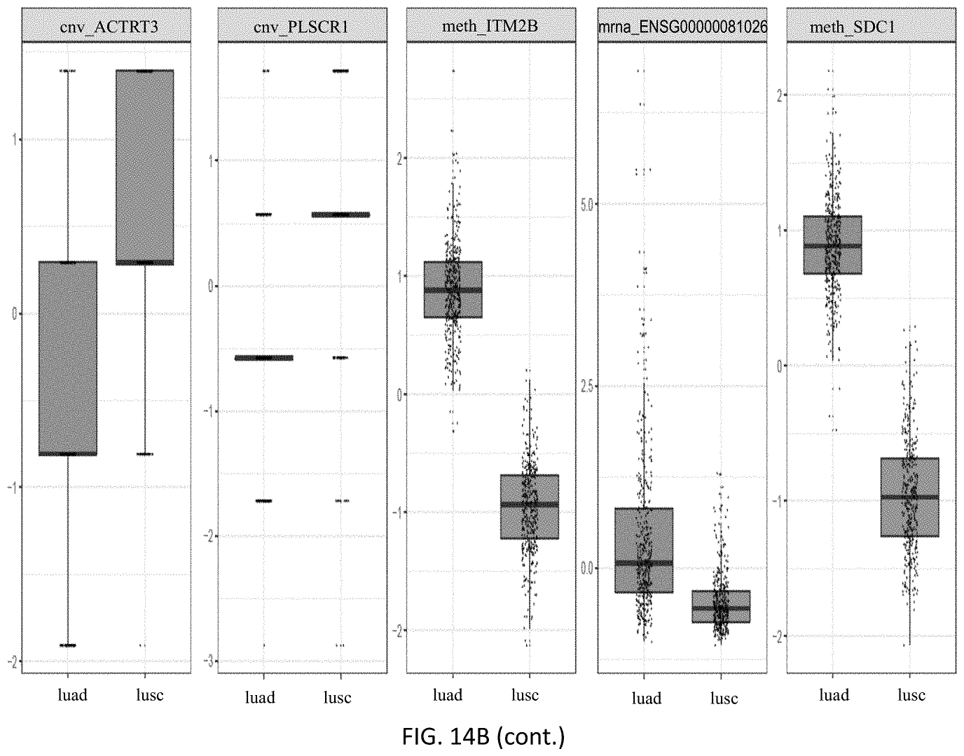

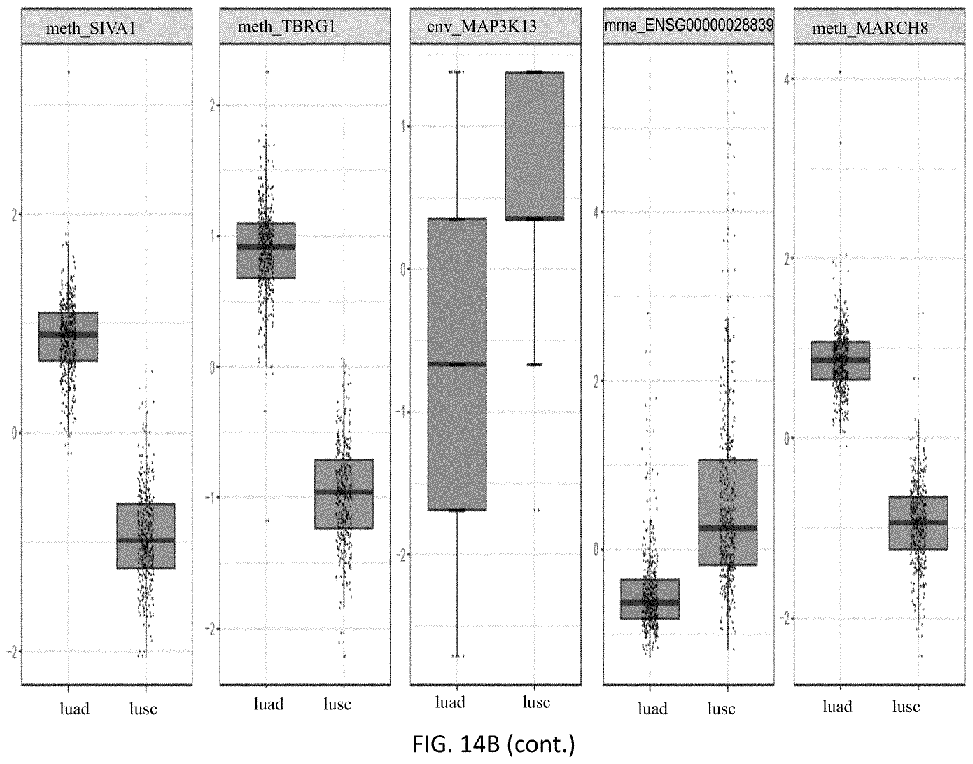

FIG. 14A and FIG. 14B depict the top 20 most informative nGOseq genes at the gene level for Lung Adenocarcinoma (LUAD) versus Lung Squamous Cell (LUSC) lung cancer subtypes (for both training (FIG. 14B) and testing data sets (14A)).

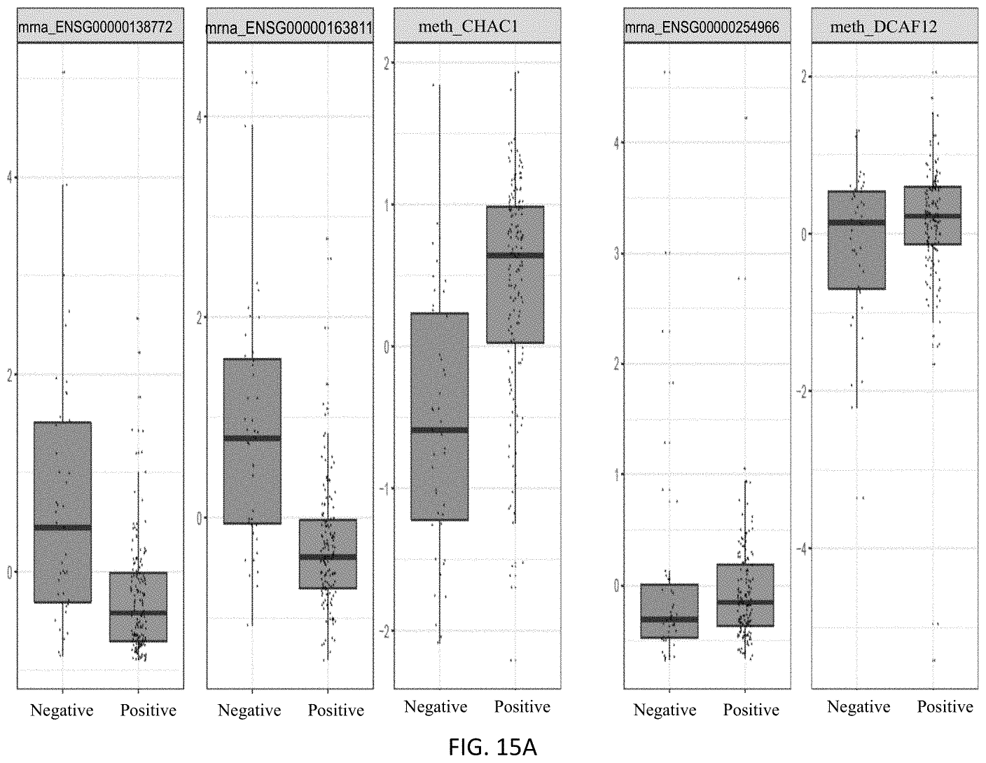

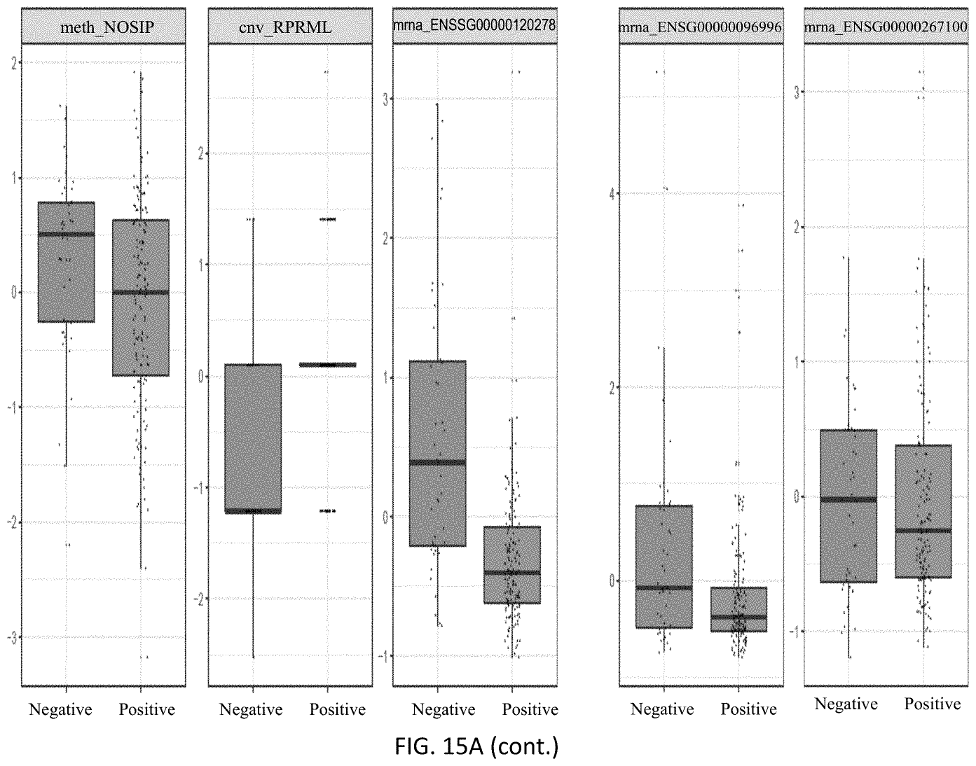

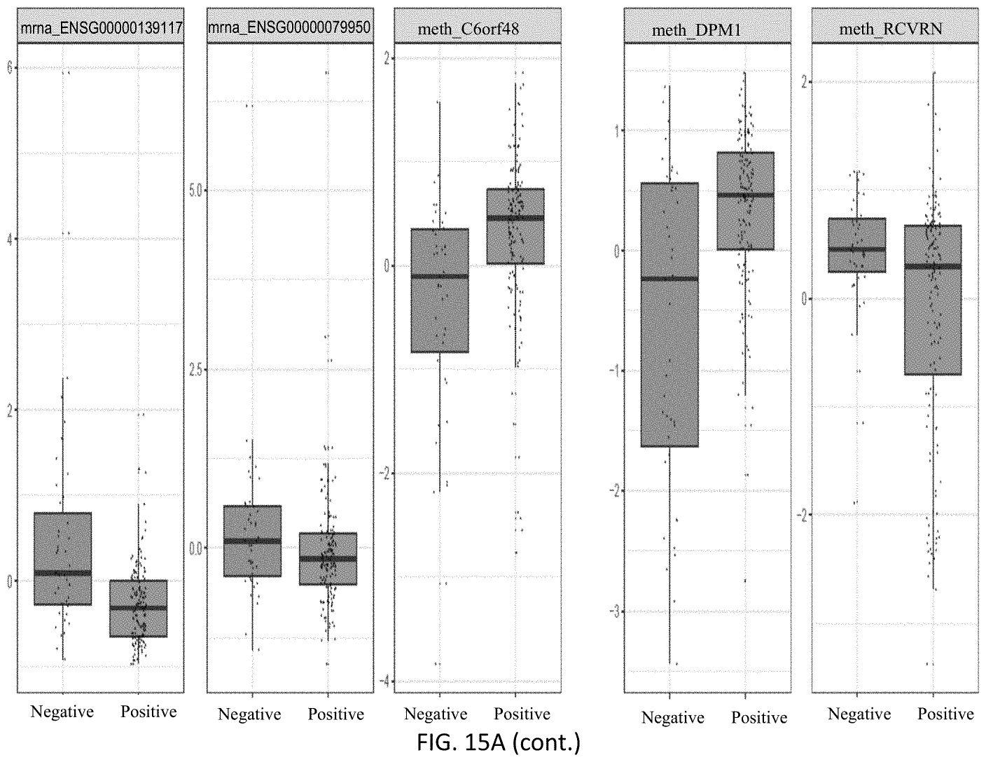

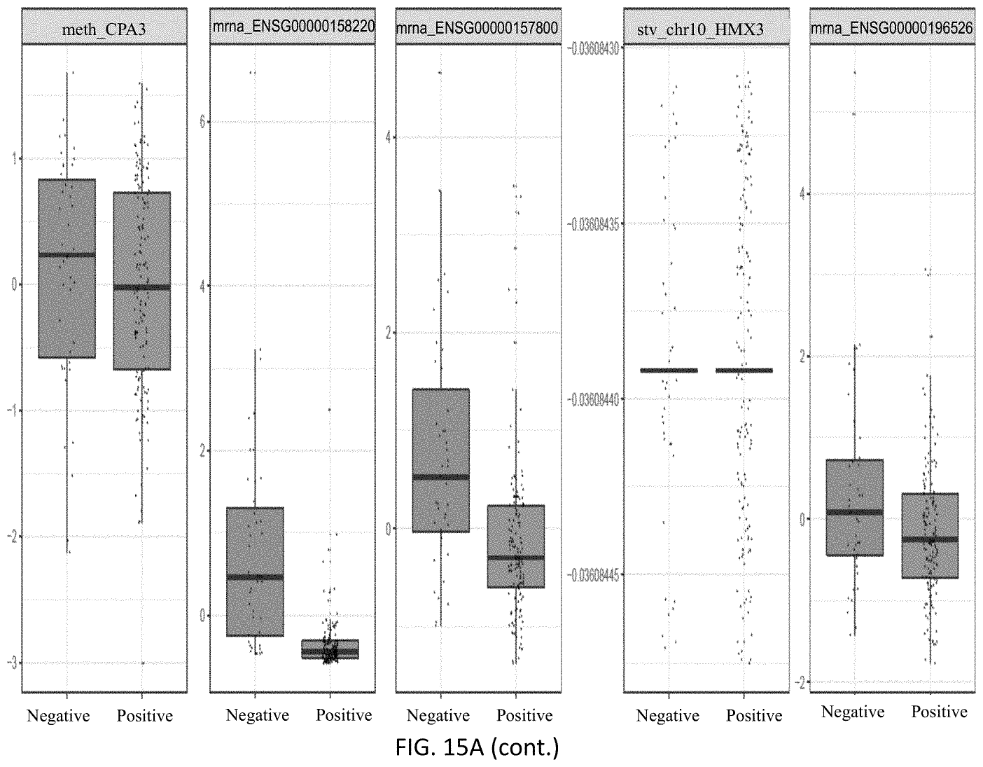

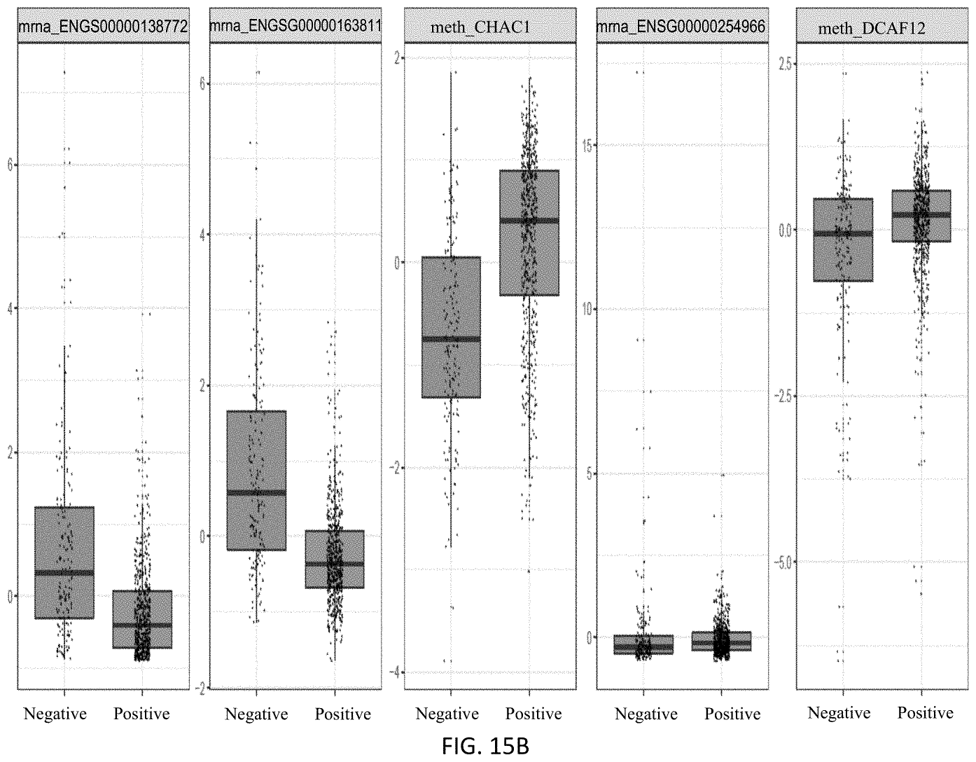

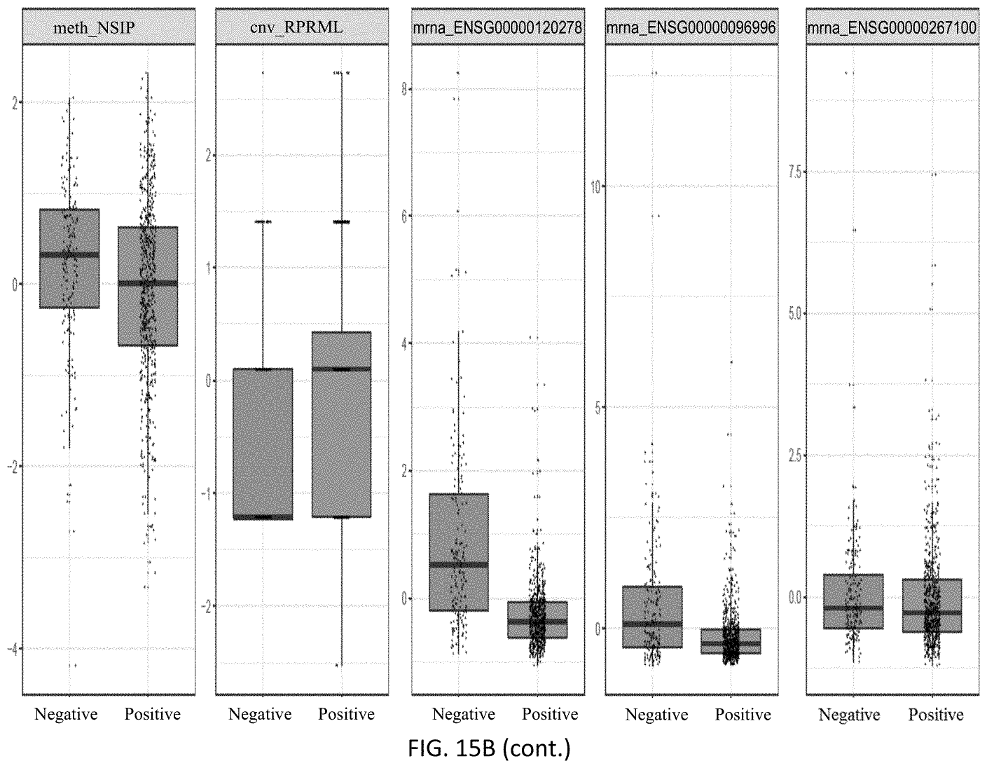

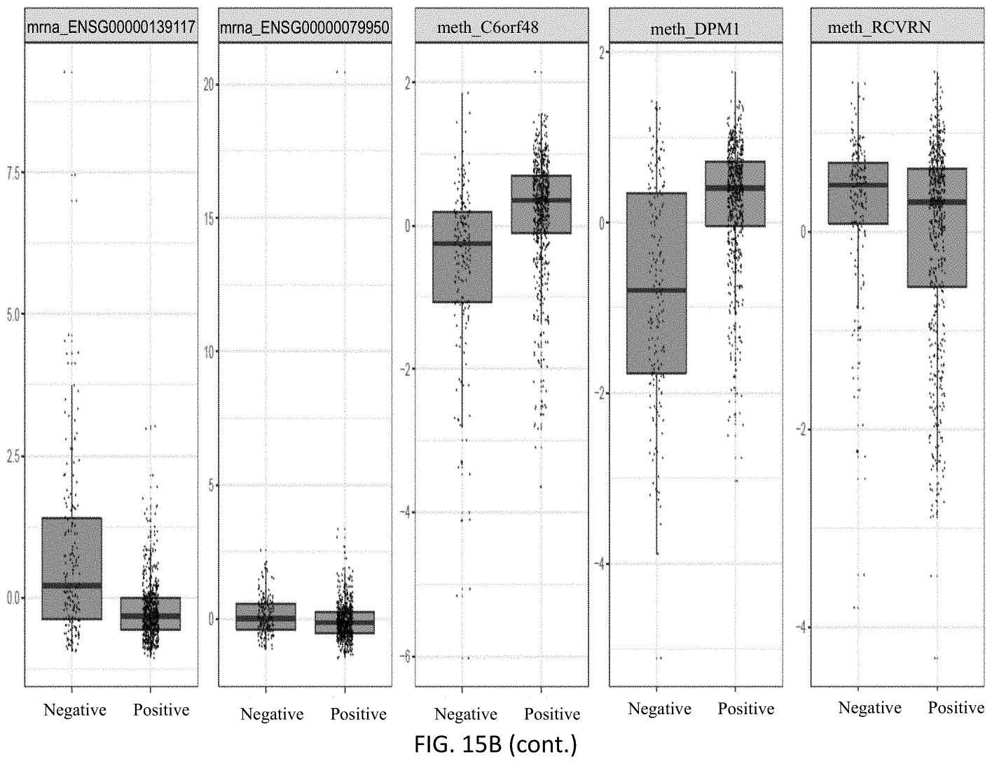

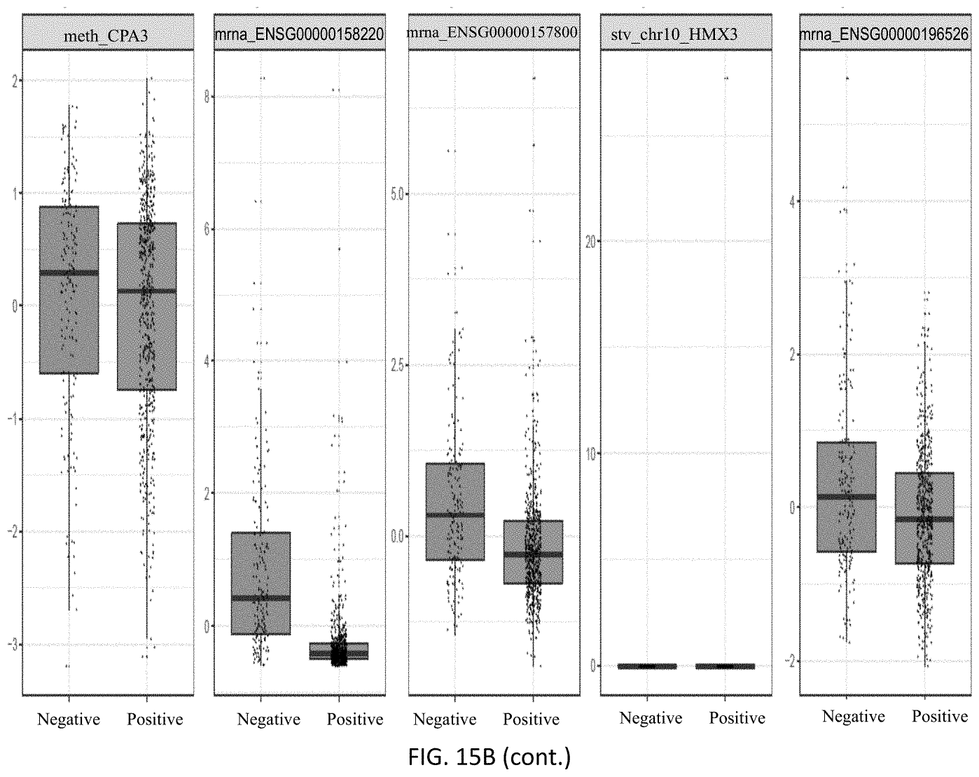

FIG. 15A and FIG. 15B depicts the top 20 most informative MEGENA genes at the gene level for ER+ versus ER− breast cancer subtypes (for both training (FIG. 15B) and testing data sets (15A)).

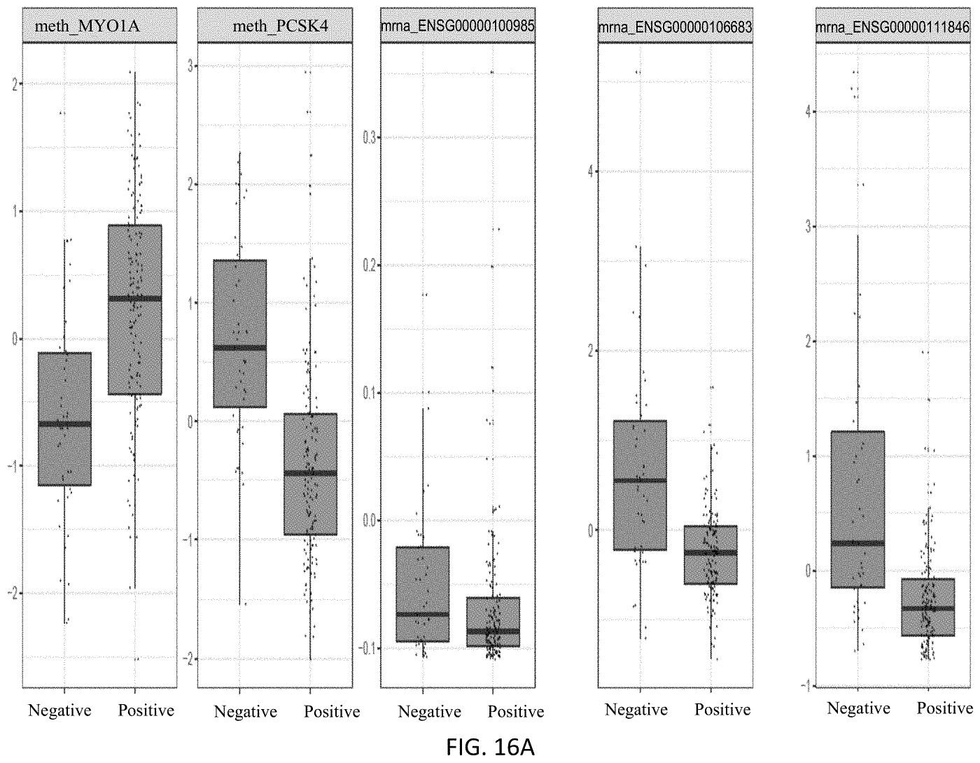

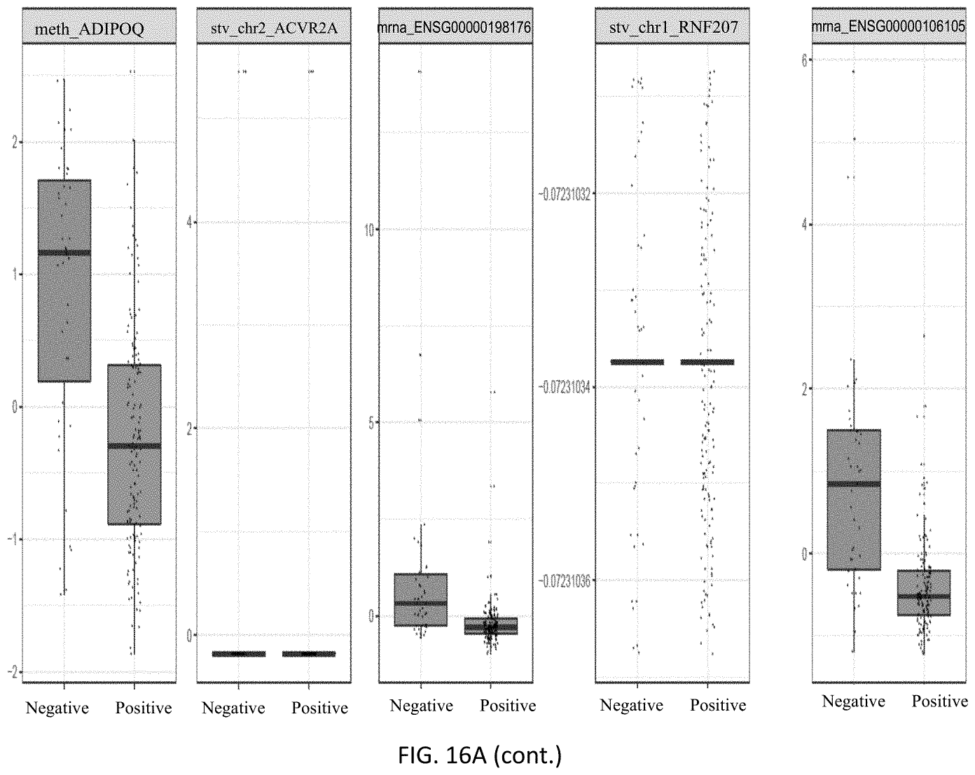

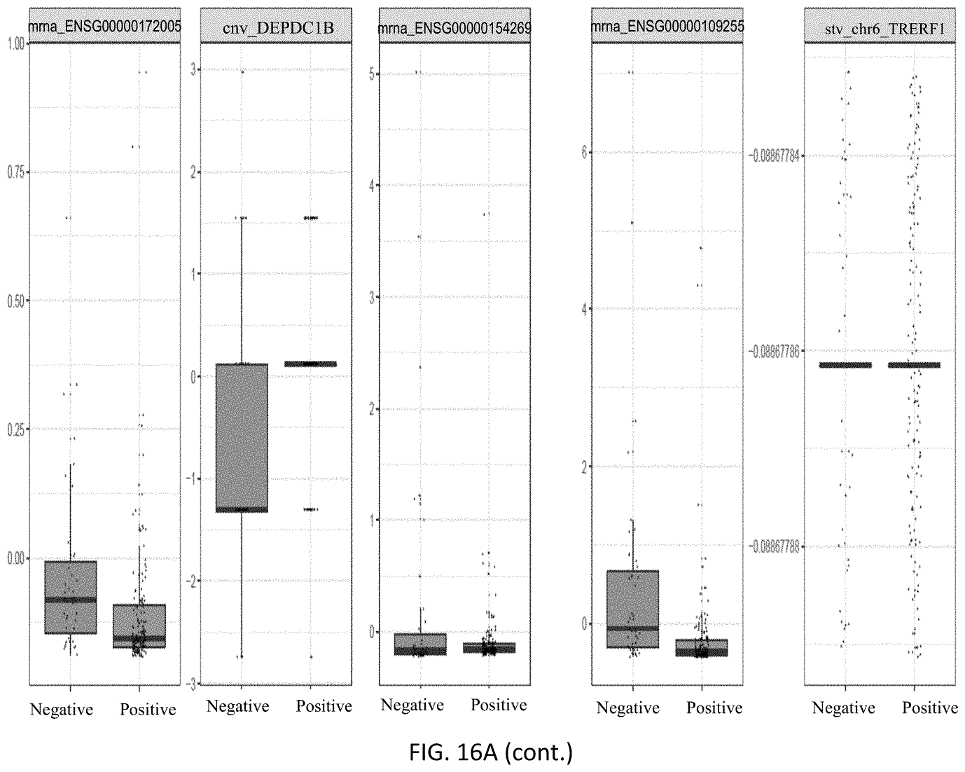

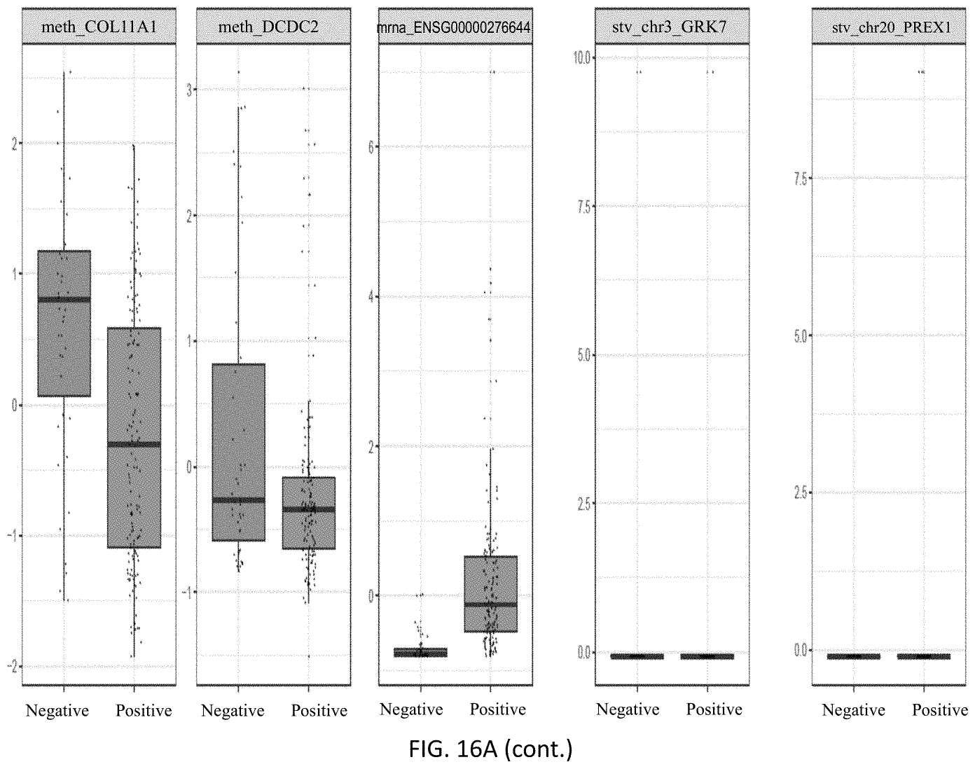

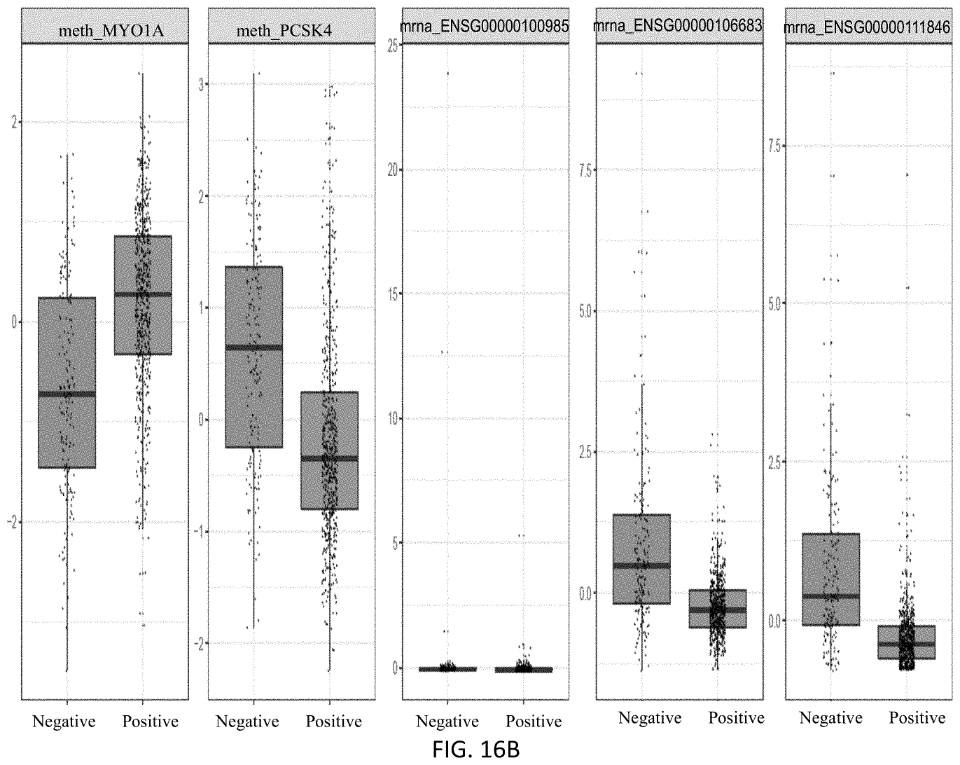

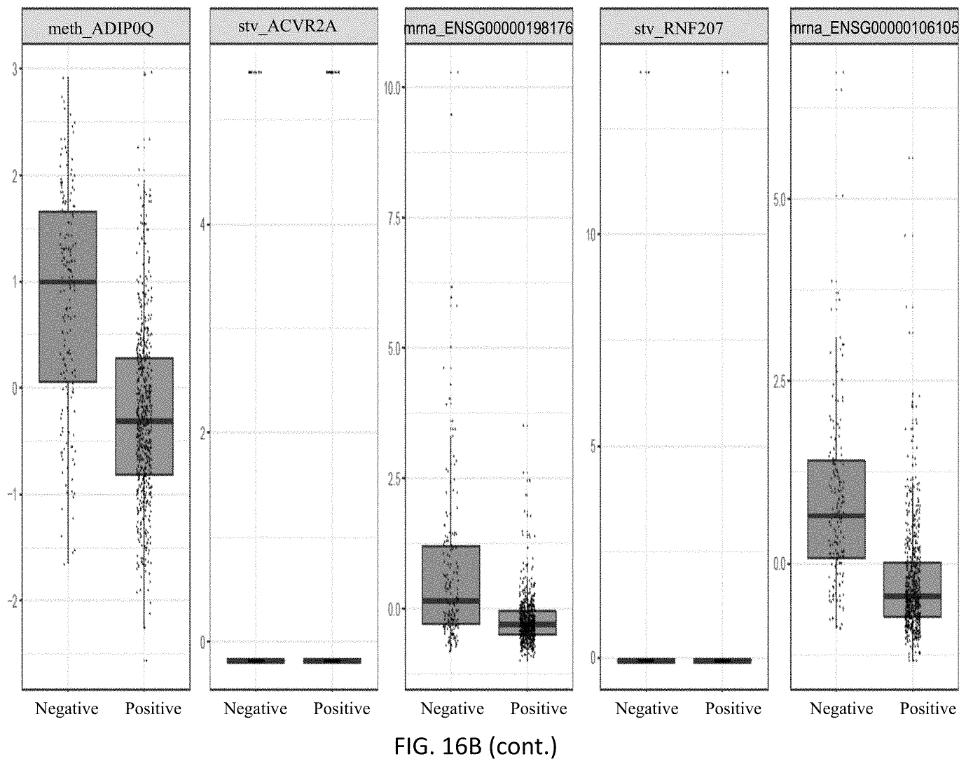

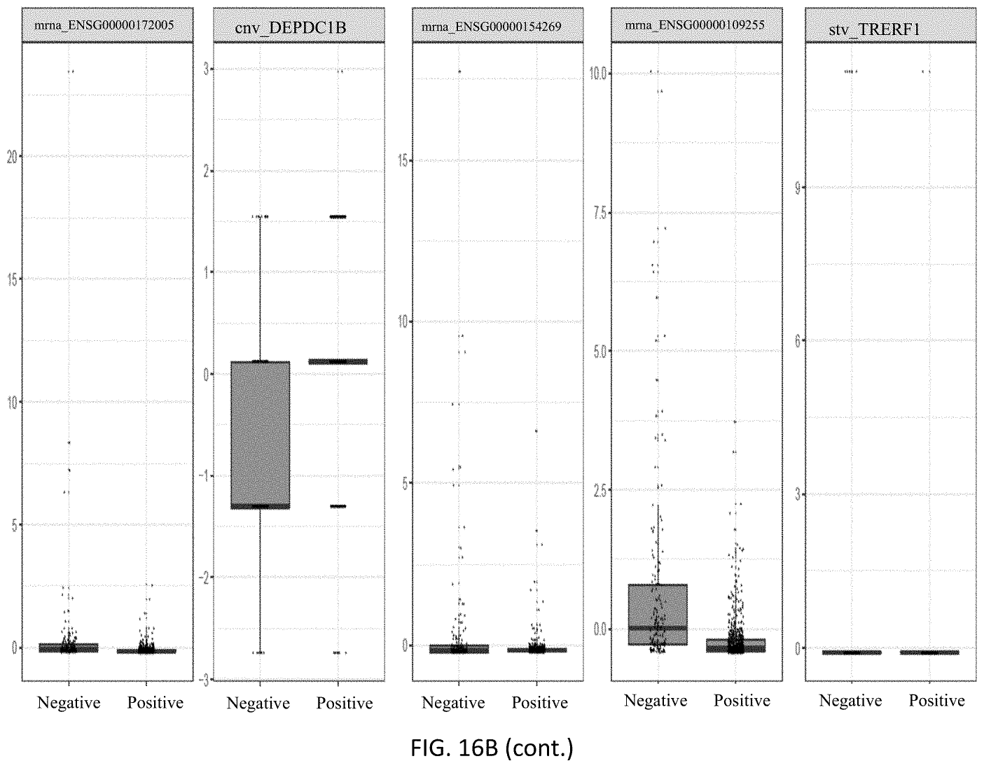

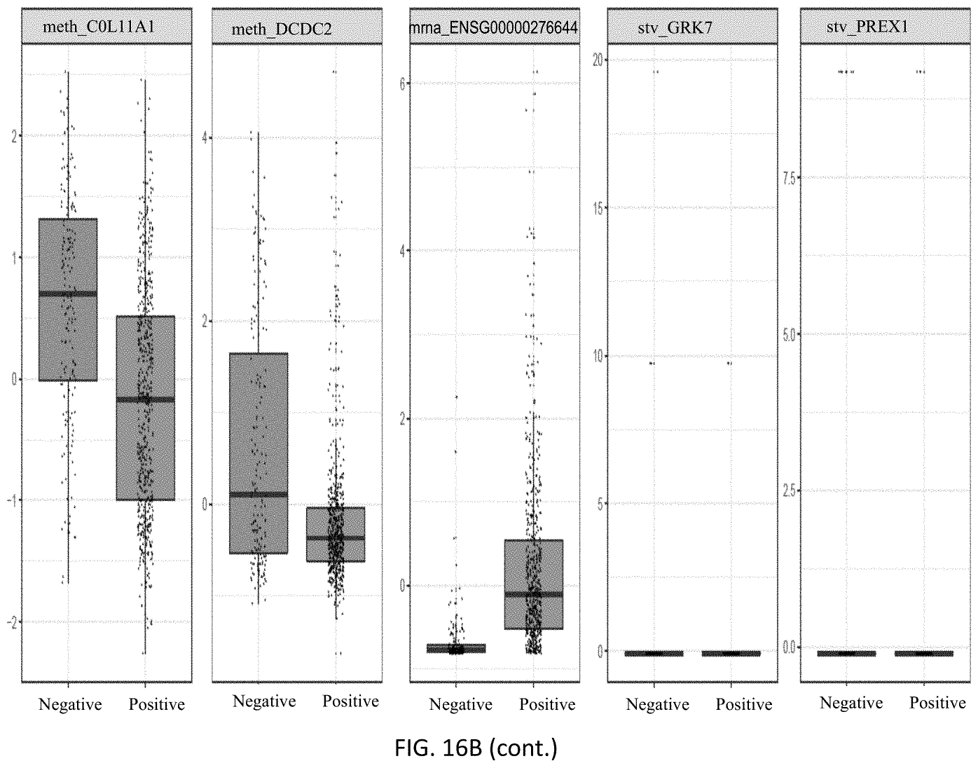

FIG. 16A and FIG. 16B depicts the top 20 most informative nGOseq genes at the gene level for ER+ versus ER− breast cancer subtypes (for both training (FIG. 16B) and testing data sets (16A)).

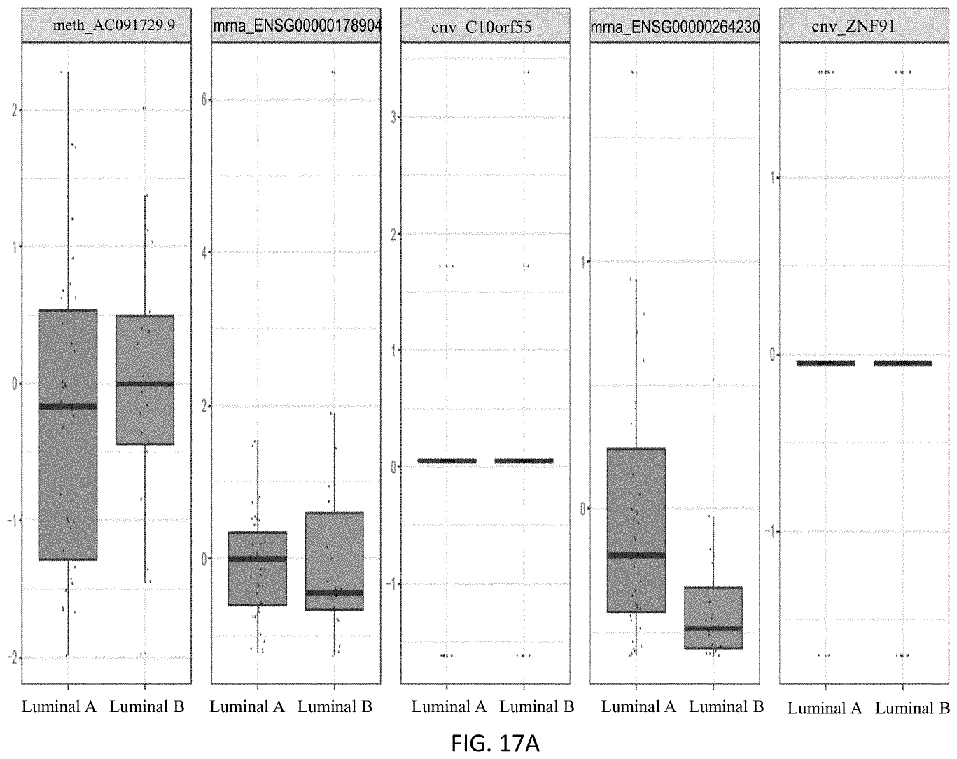

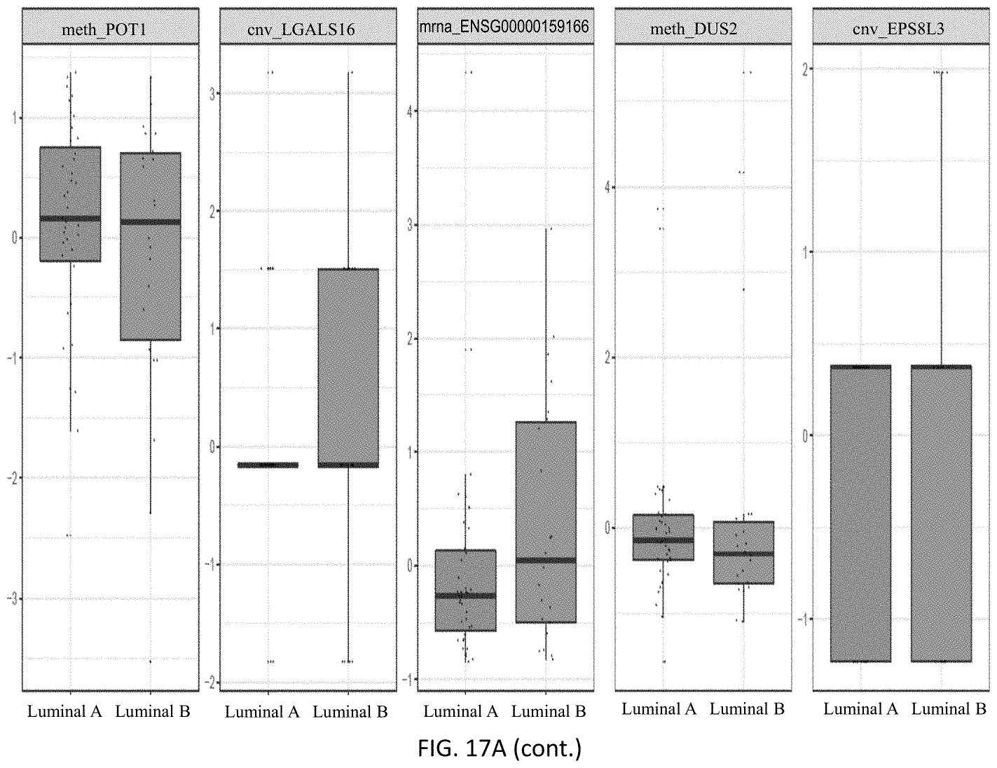

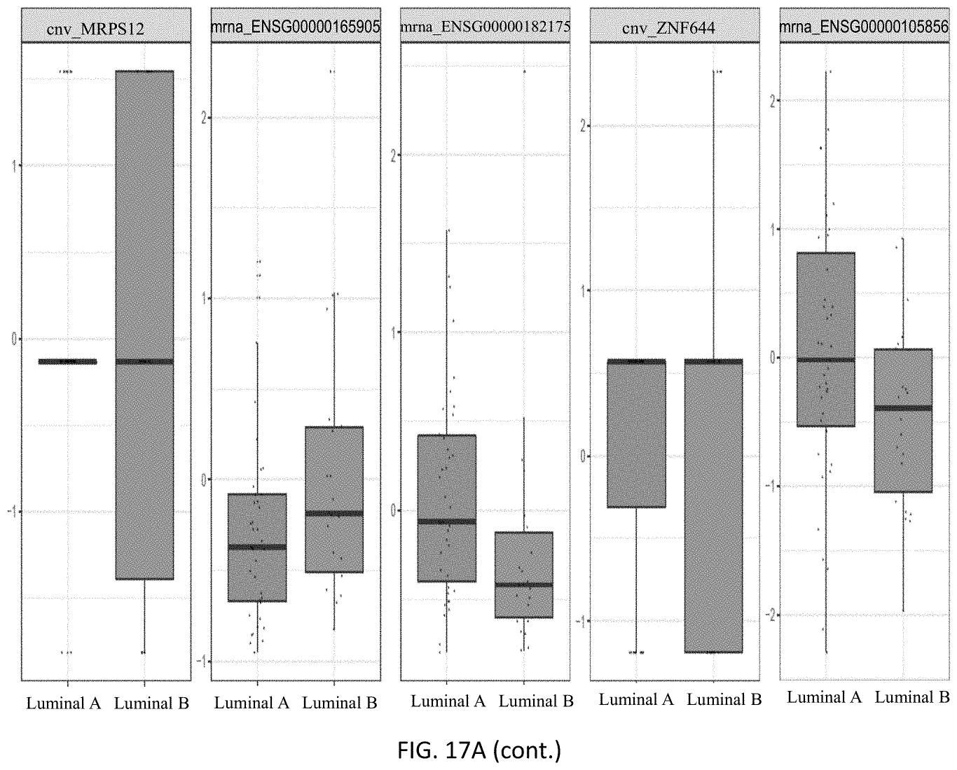

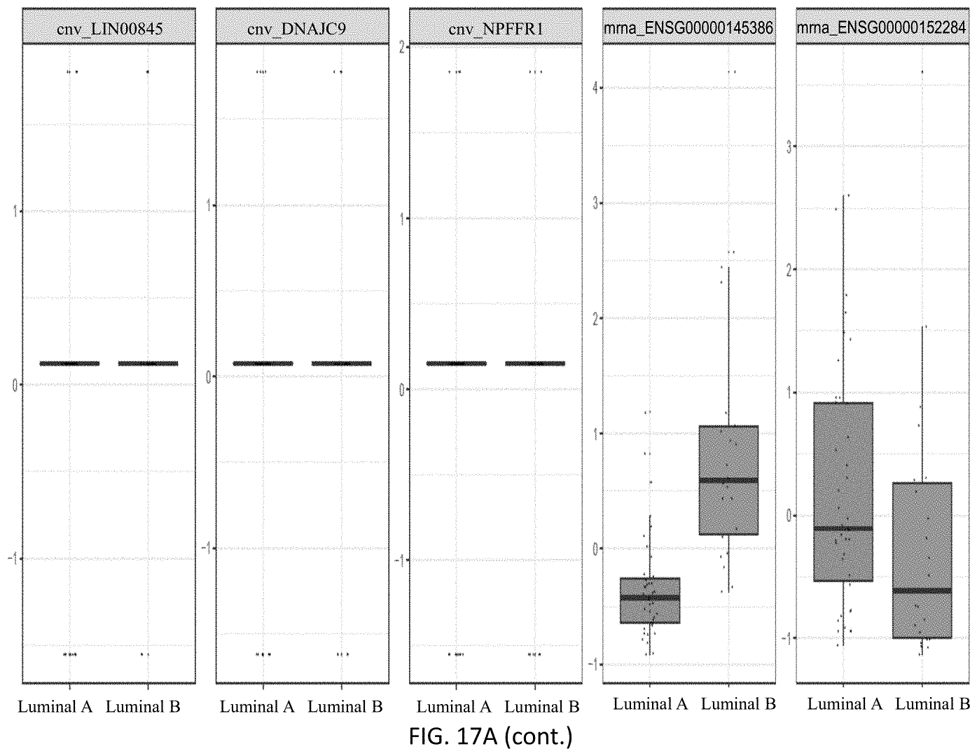

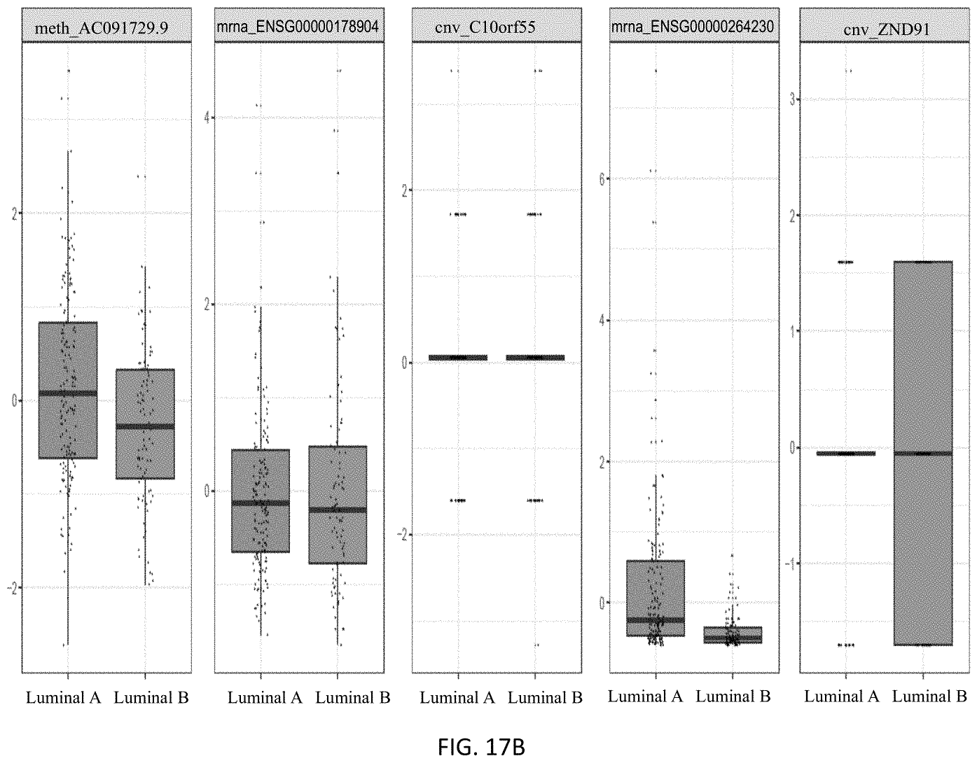

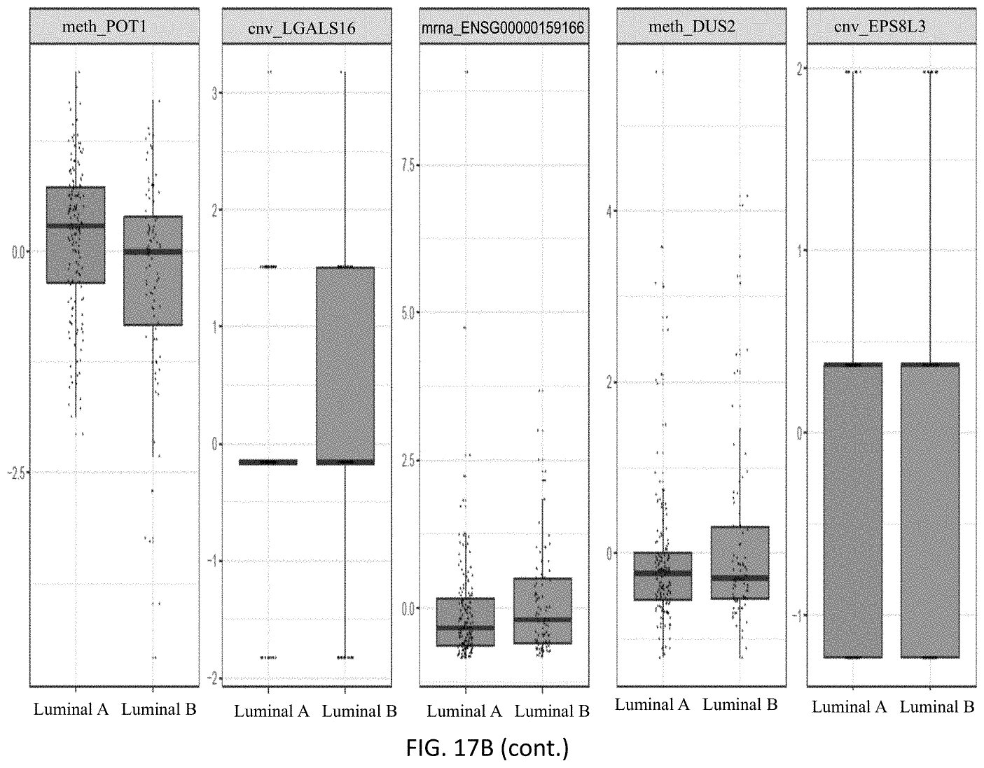

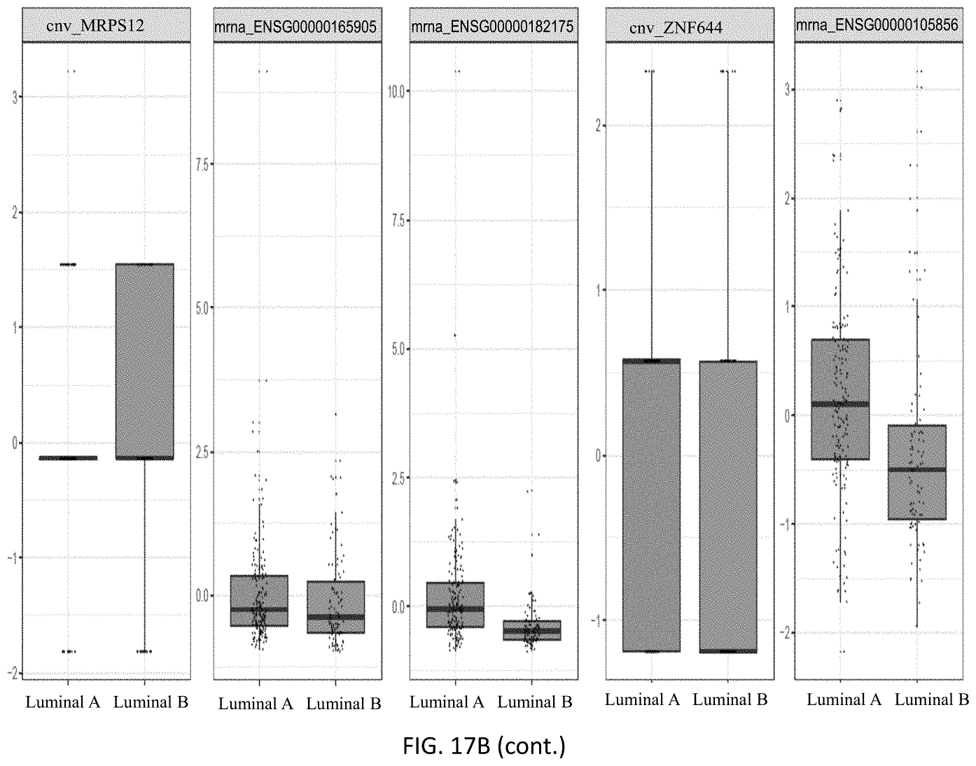

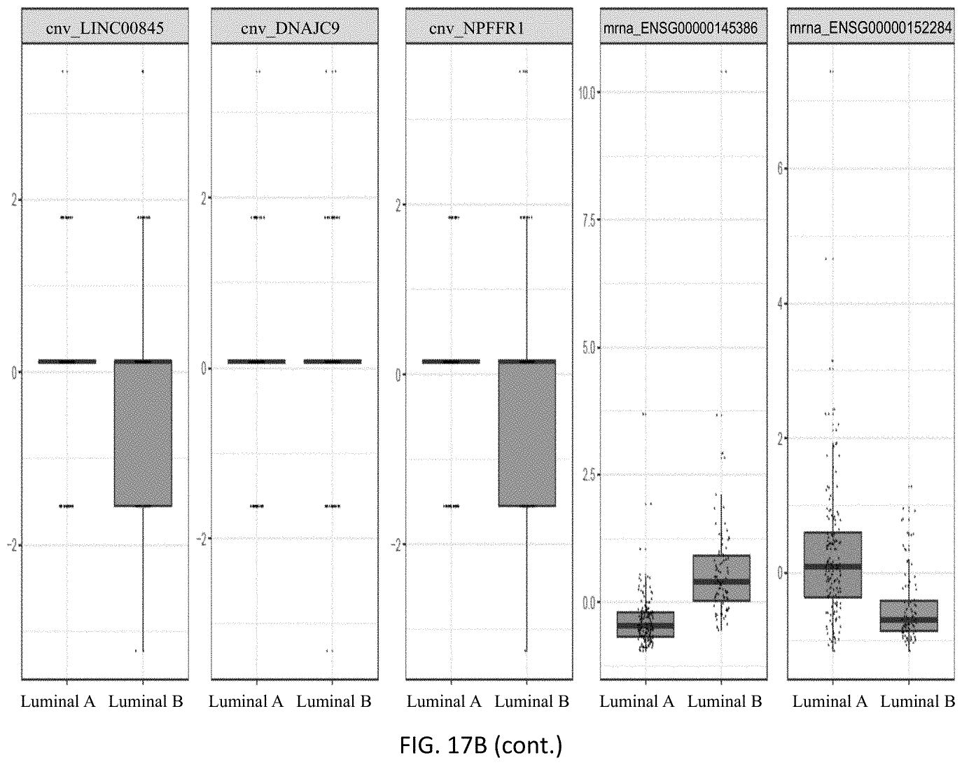

FIG. 17A and FIG. 17B depicts the top 20 most informative MEGENA genes at the gene level for Luminal A versus Luminal B breast cancer subtypes (for both training (FIG. 17B) and testing data sets (17A)).

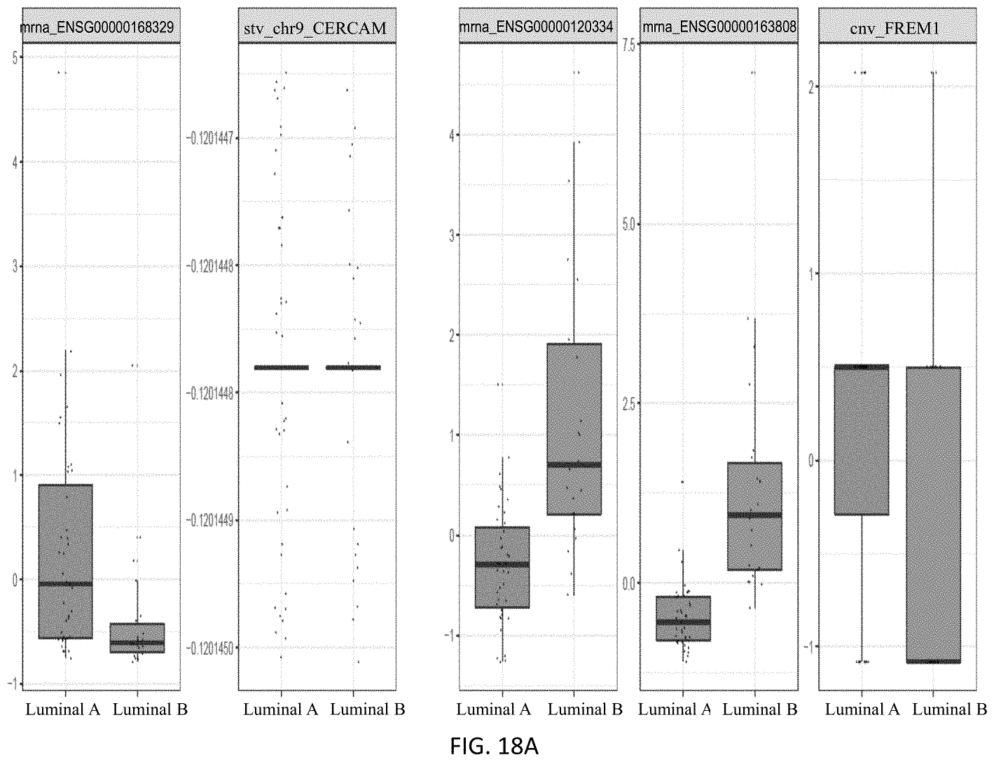

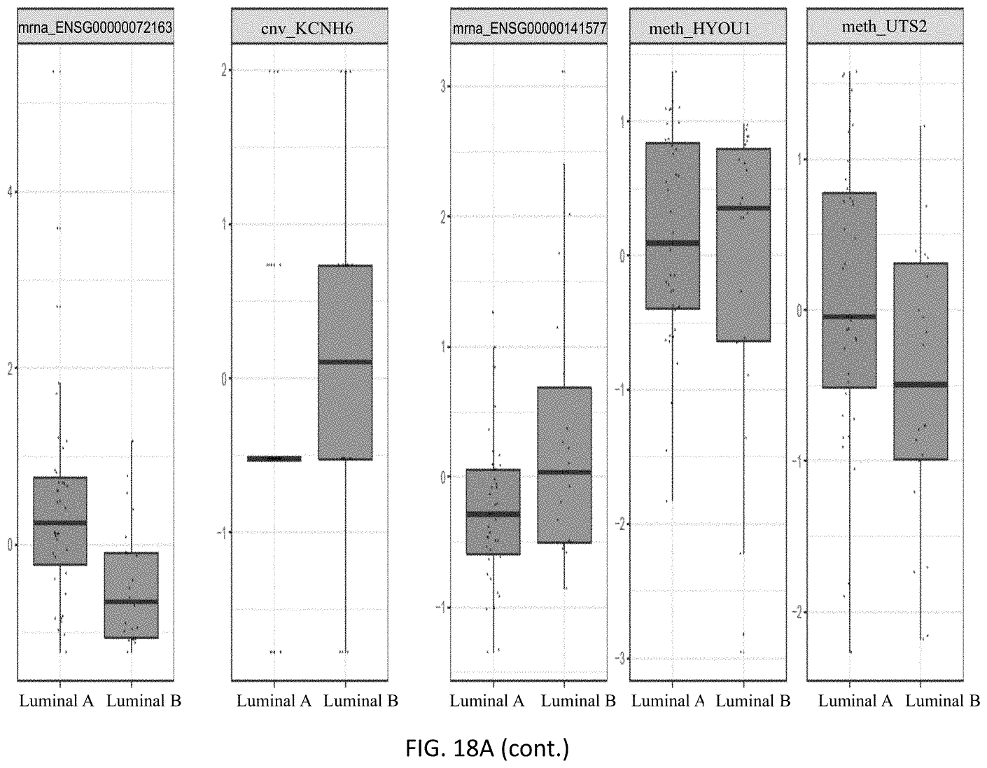

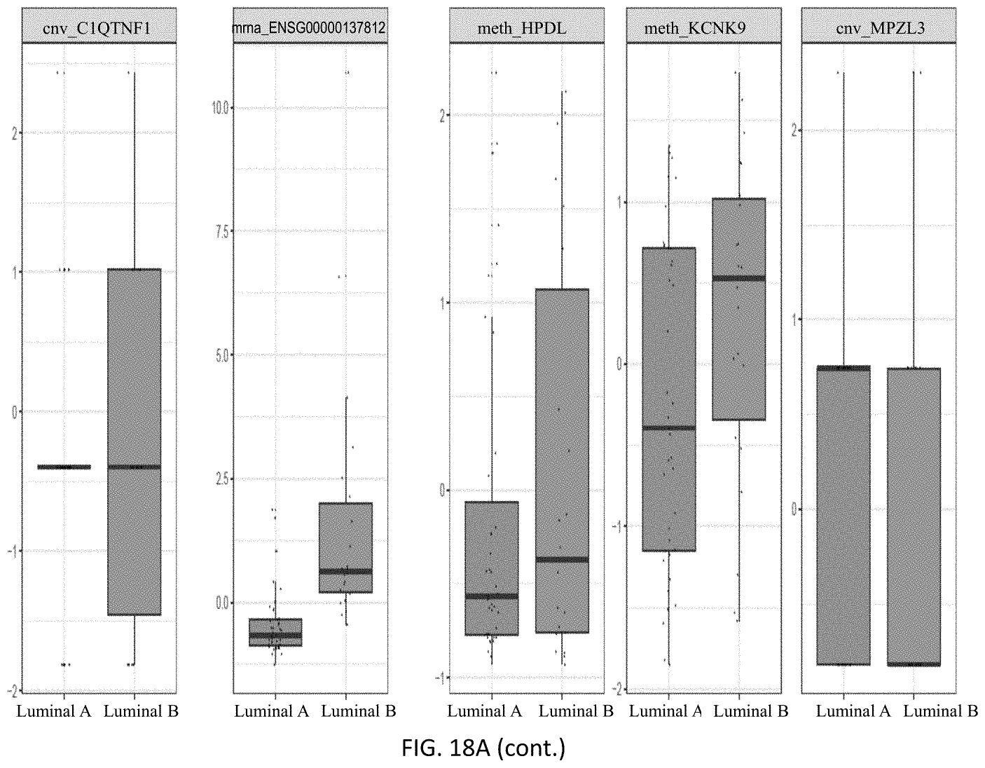

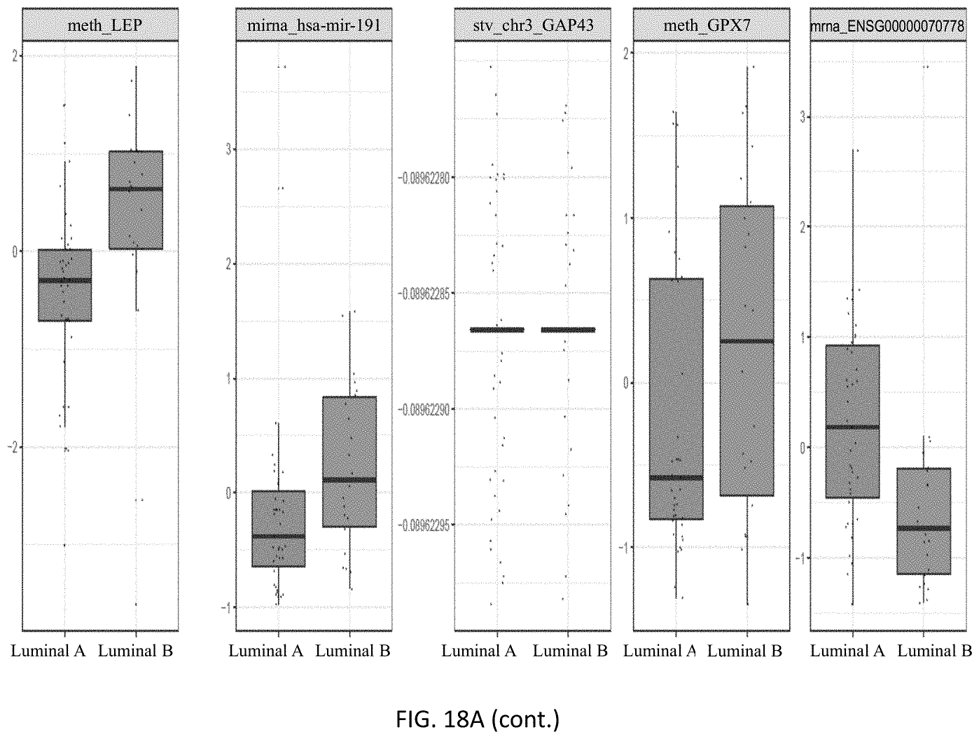

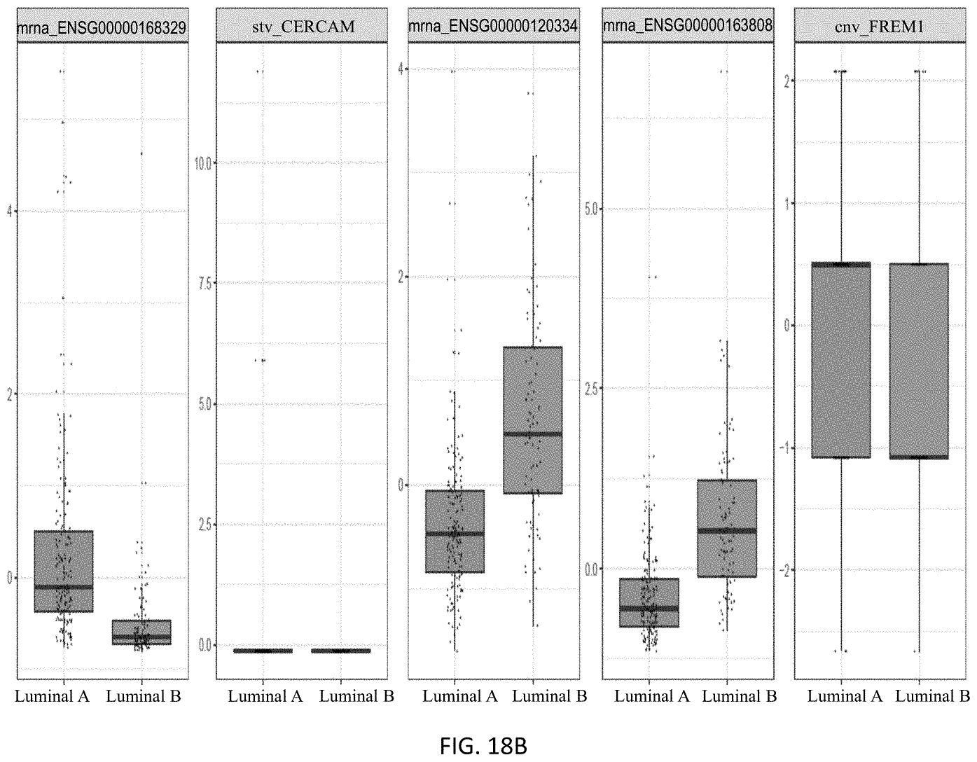

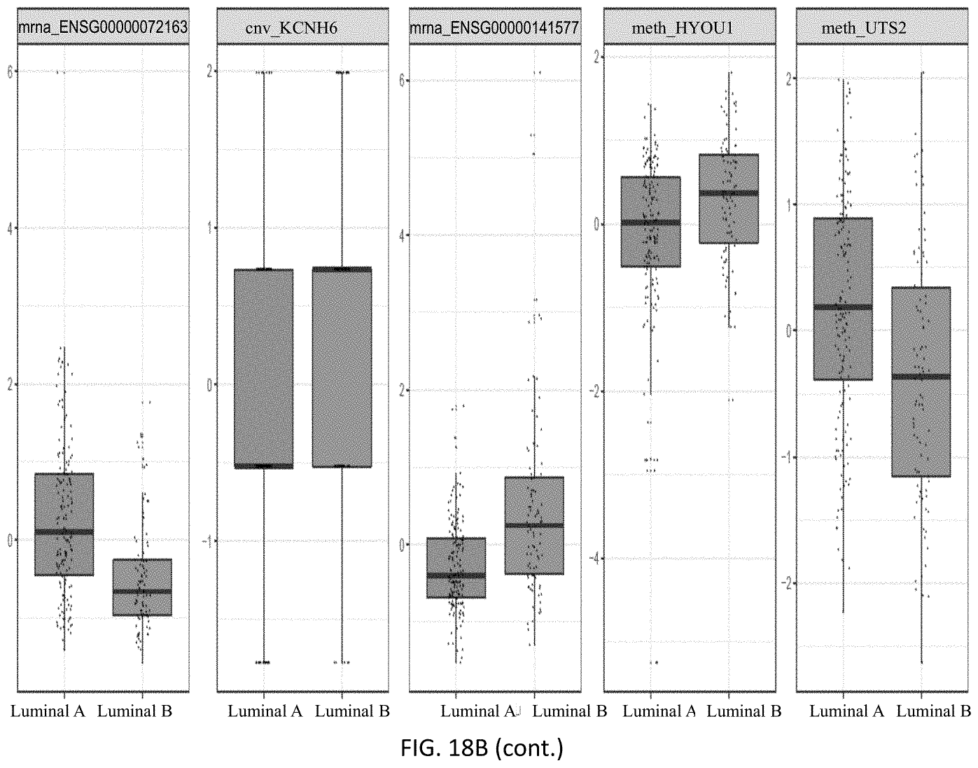

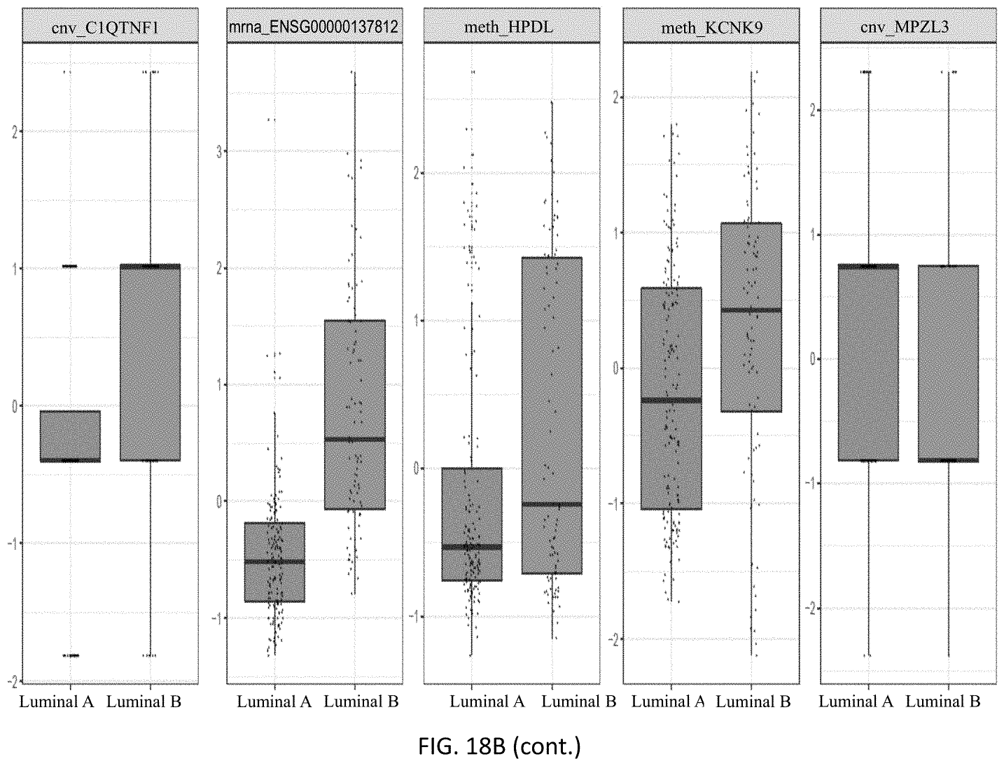

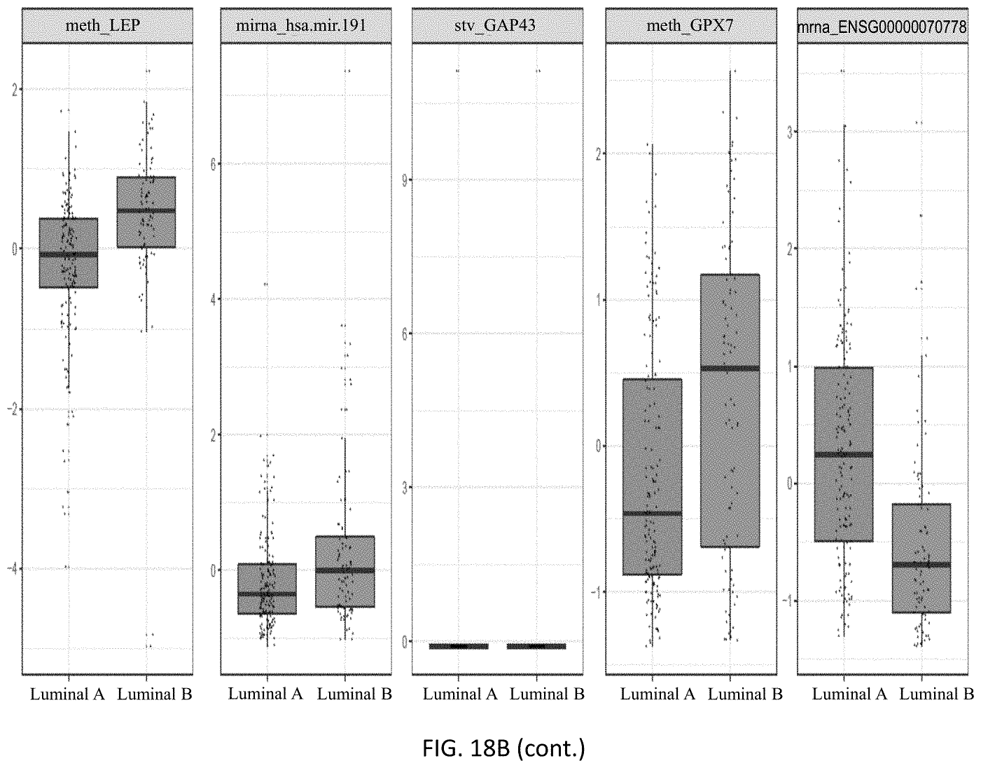

FIG. 18A and FIG. 18B depicts the top 20 most informative nGOseq genes at the gene level for Luminal A versus Luminal B breast cancer subtypes (for both training (FIG. 18A) and testing data sets (18B)).

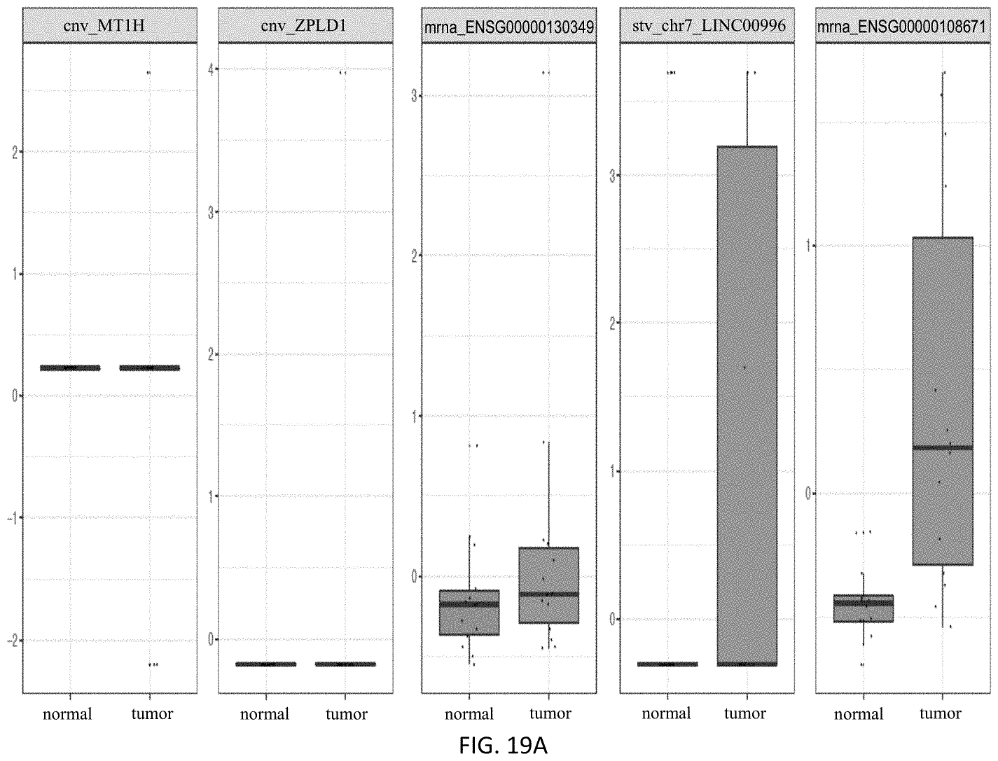

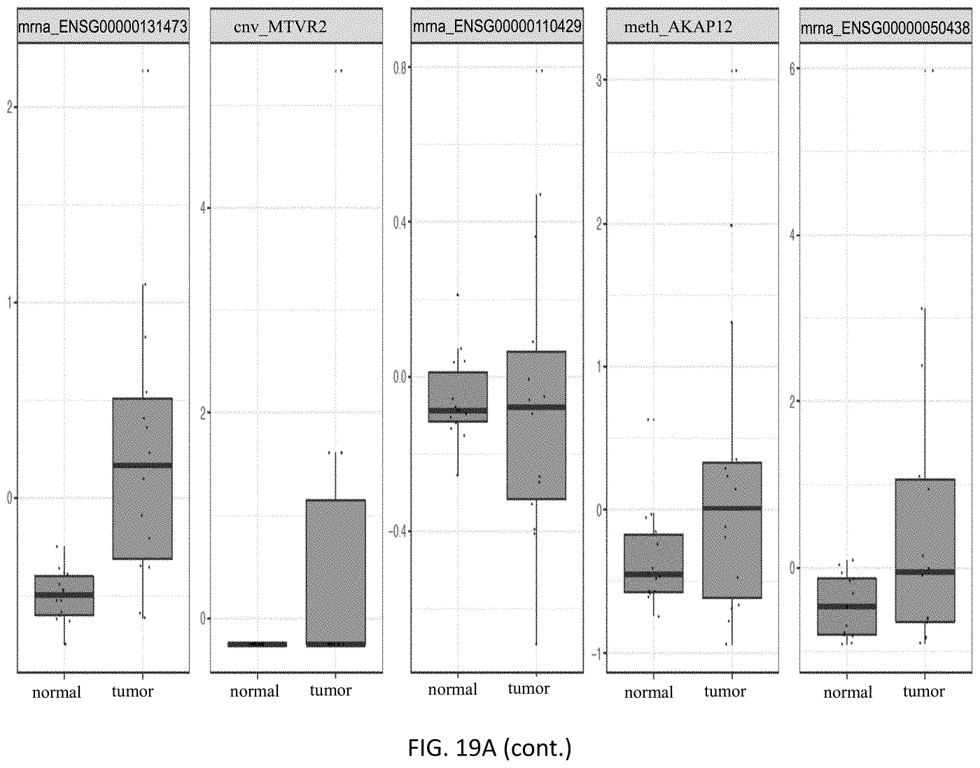

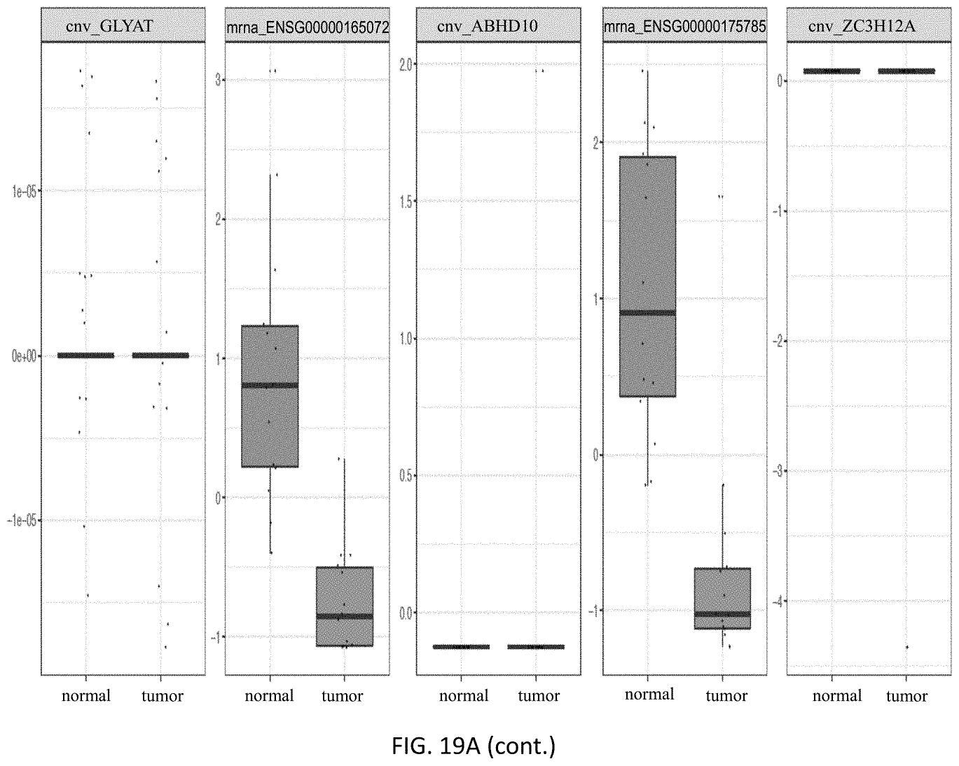

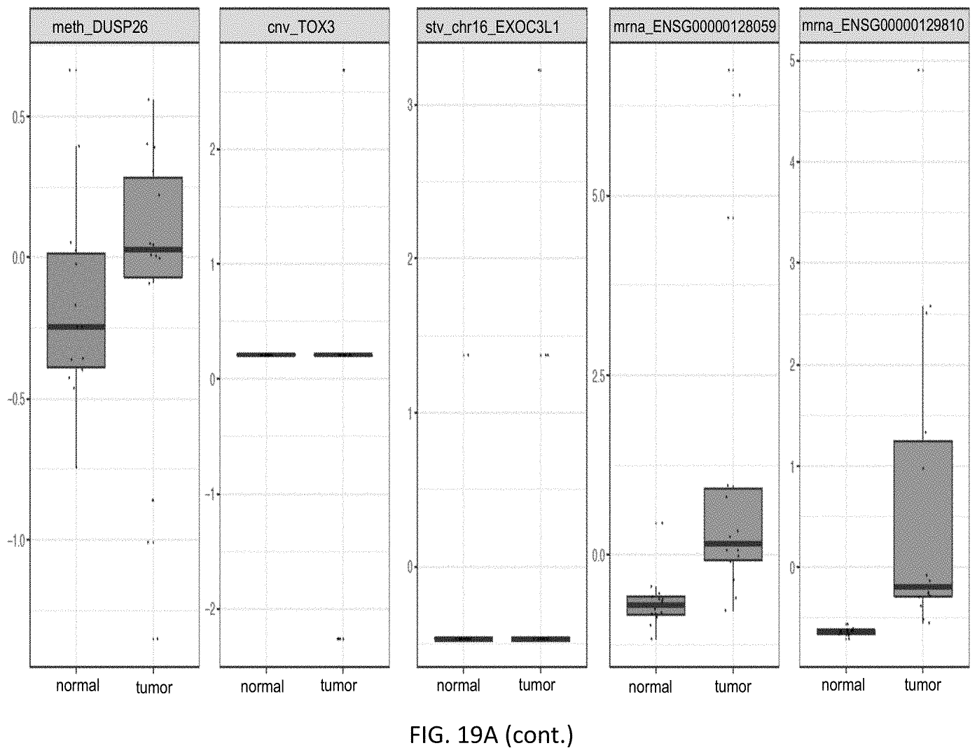

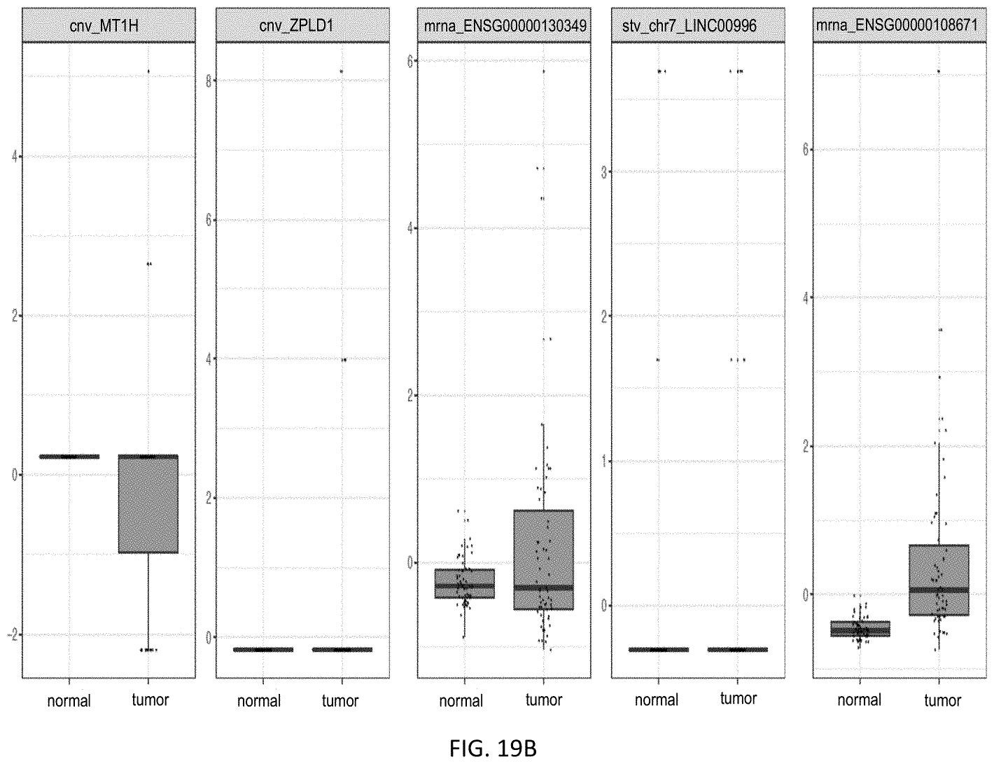

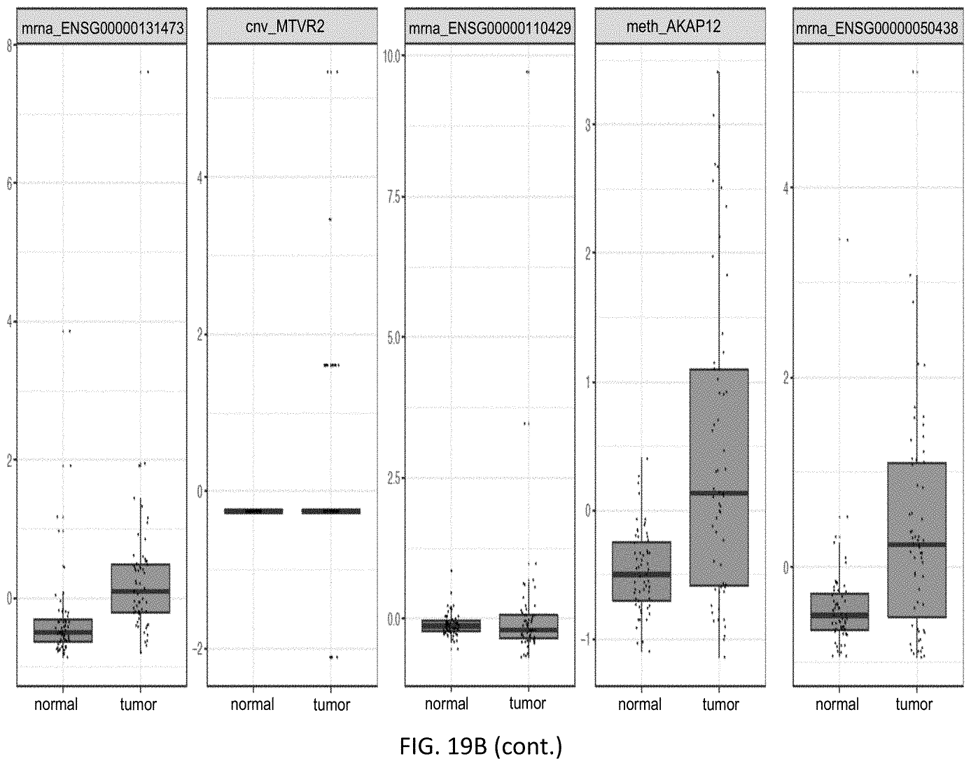

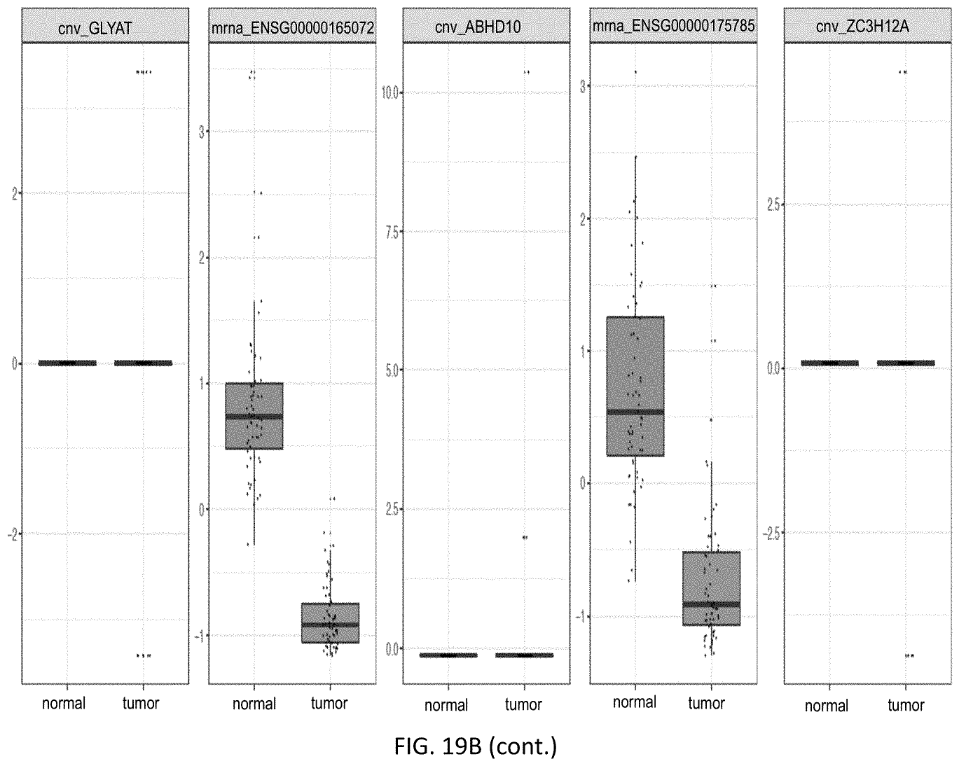

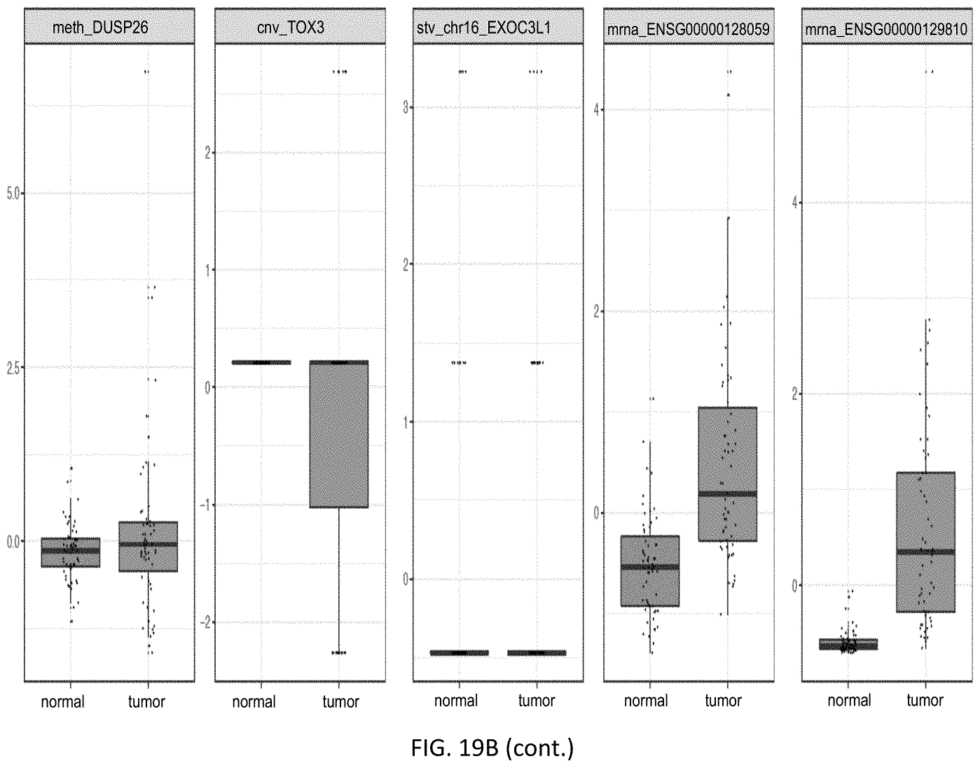

FIG. 19A and FIG. 19B depicts the top 20 most informative MEGENA genes at the gene level for breast cancer (BRCA) versus normal tissue (for both training (FIG. 19B) and testing data sets (19A)).

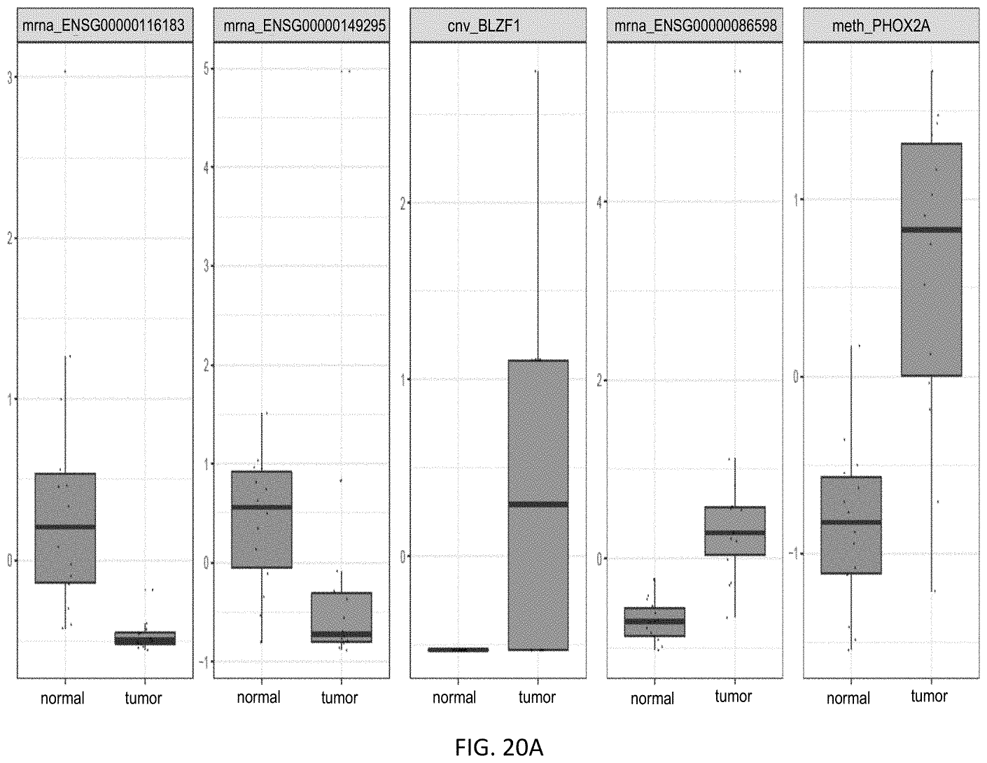

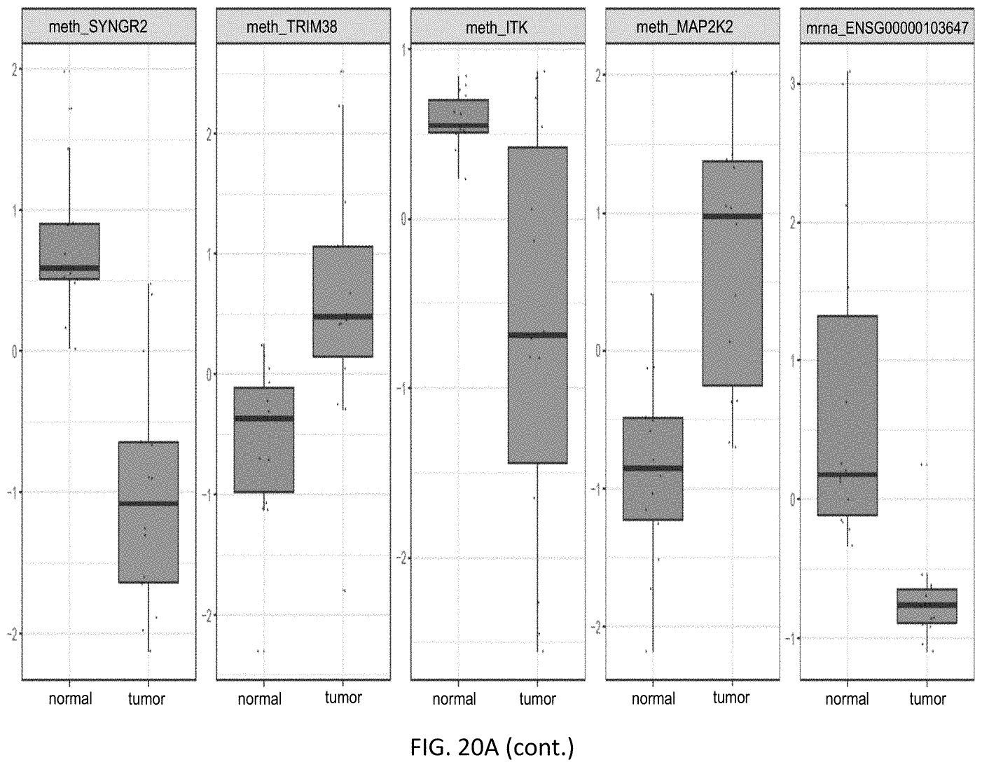

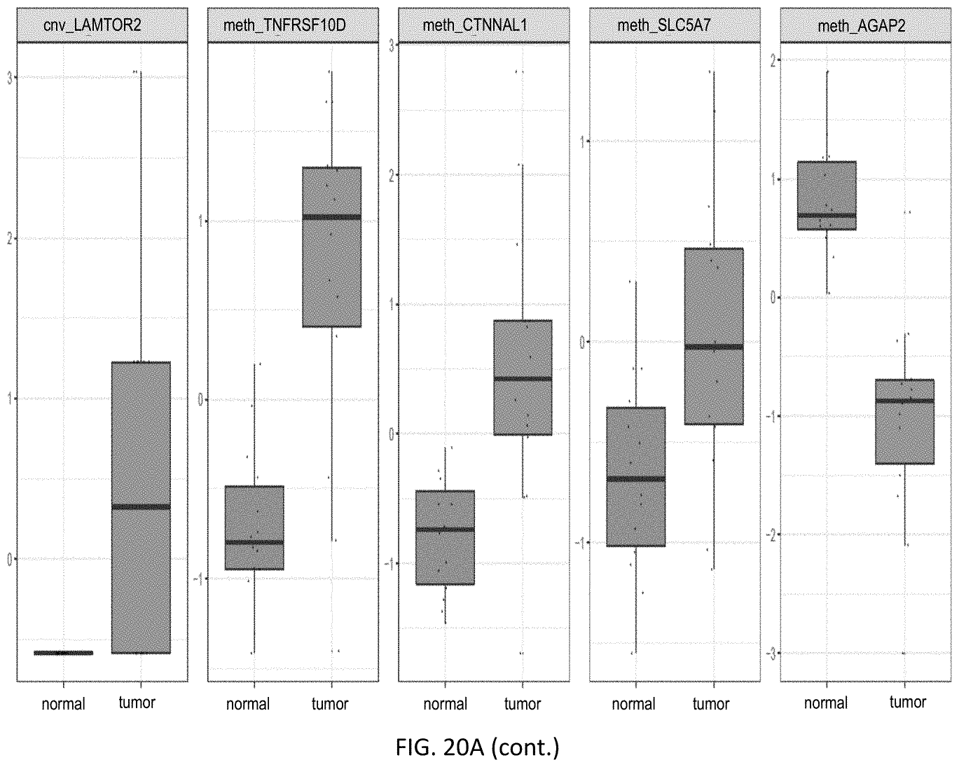

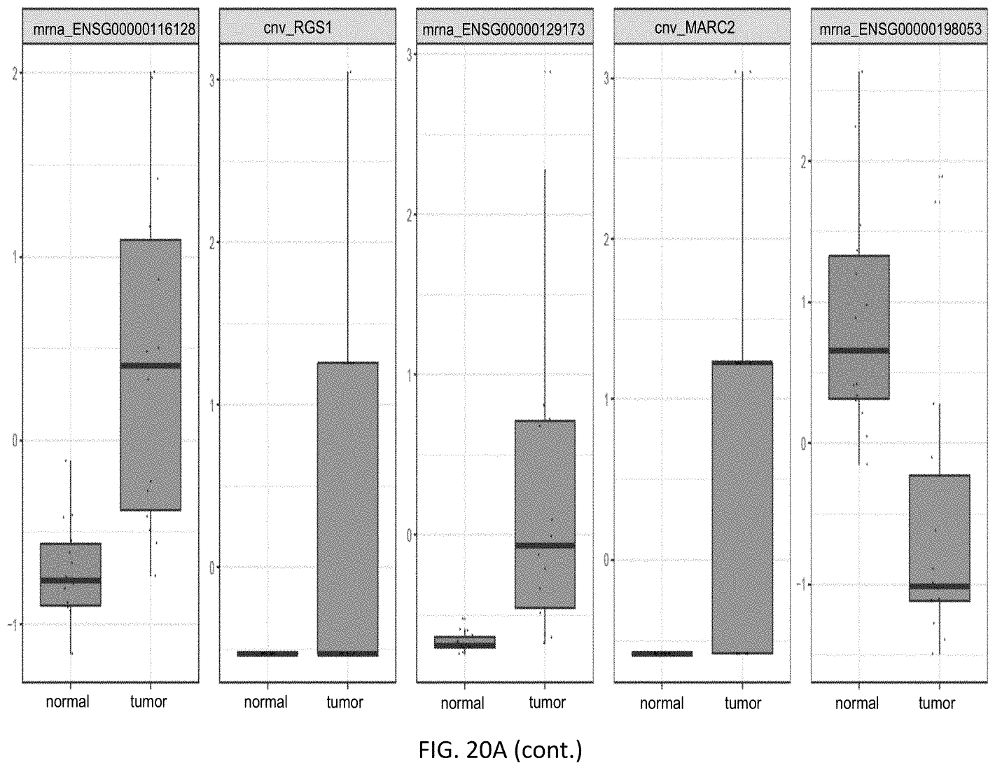

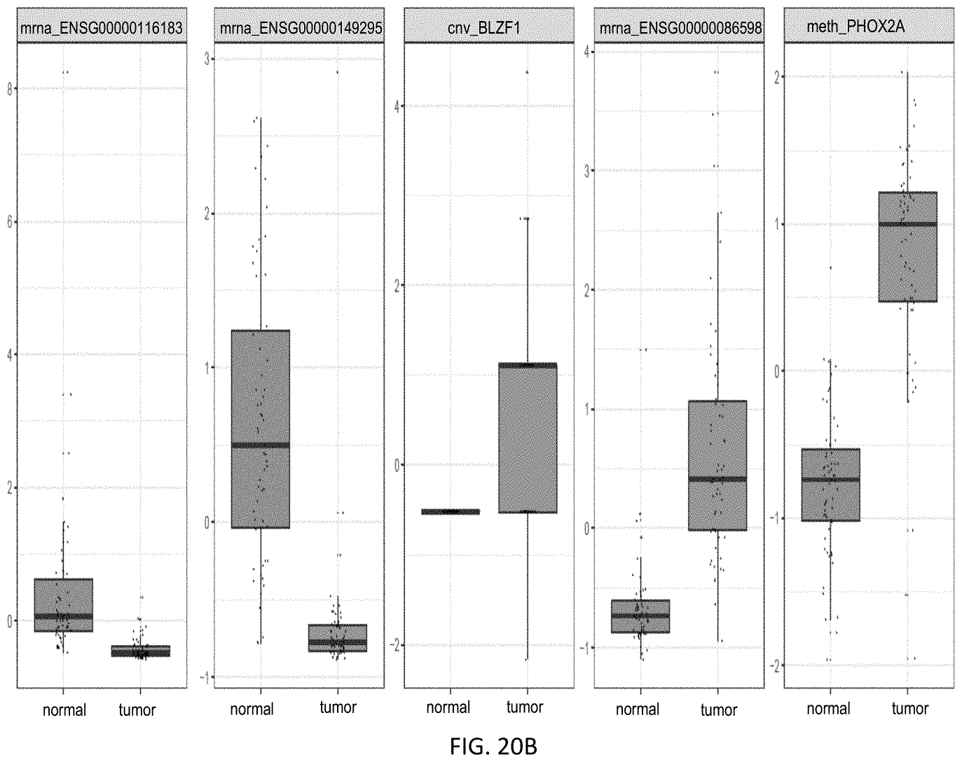

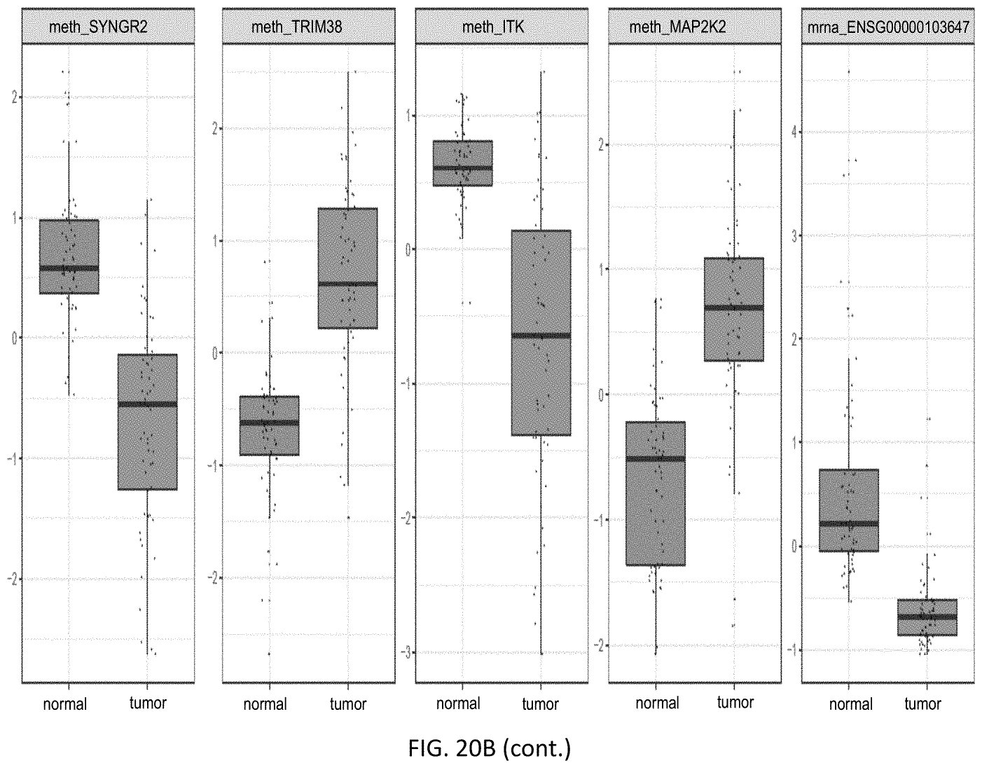

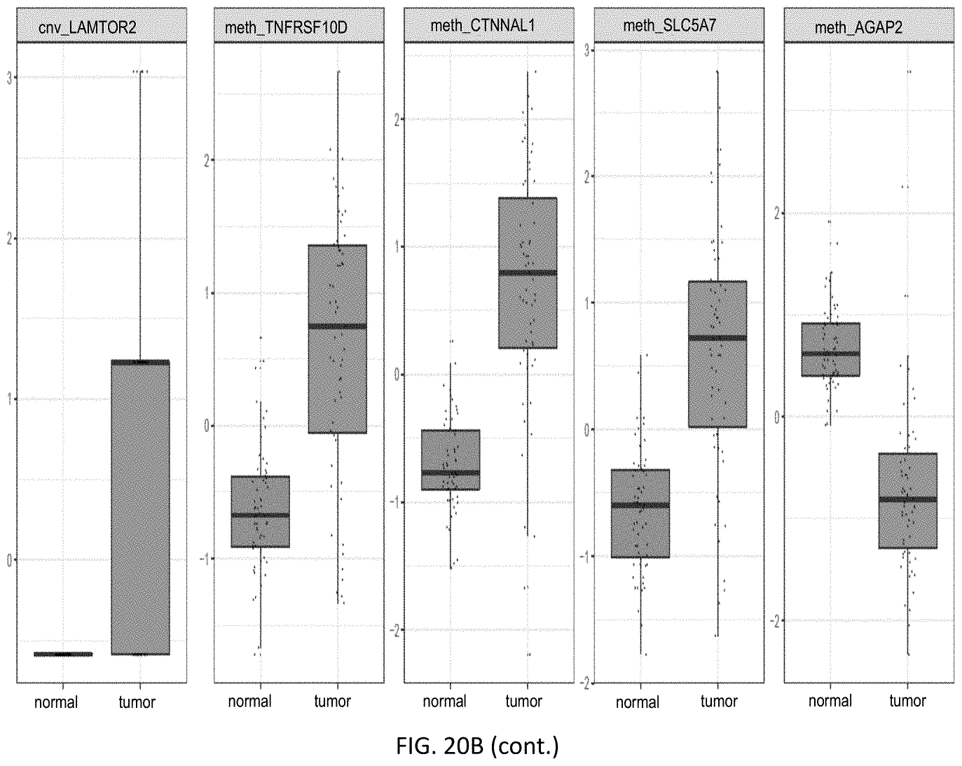

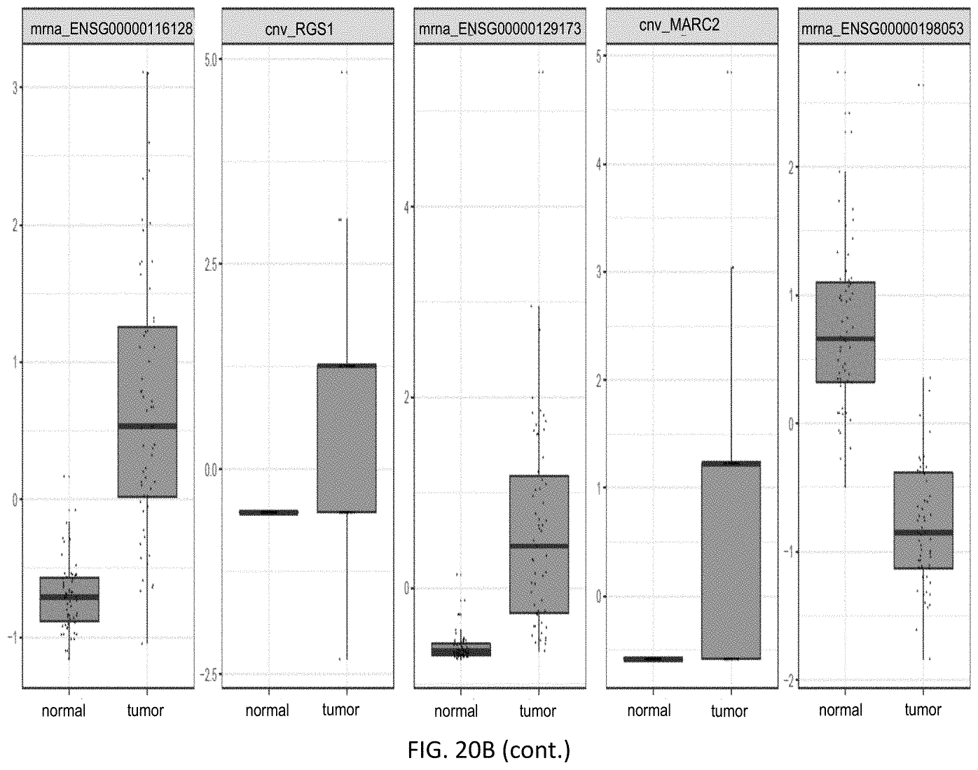

FIG. 20A and FIG. 20B depicts the top 20 most informative nGOseq genes at the gene level for breast cancer (BRCA) versus normal tissue (for both training (FIG. 20B) and testing data sets (20A)).

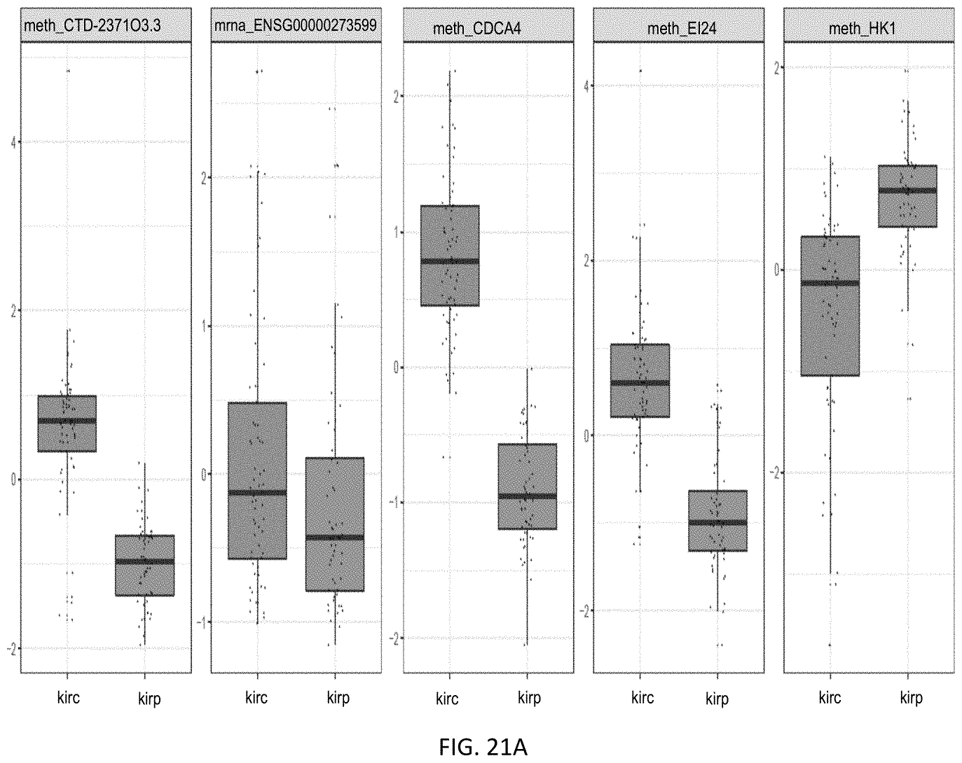

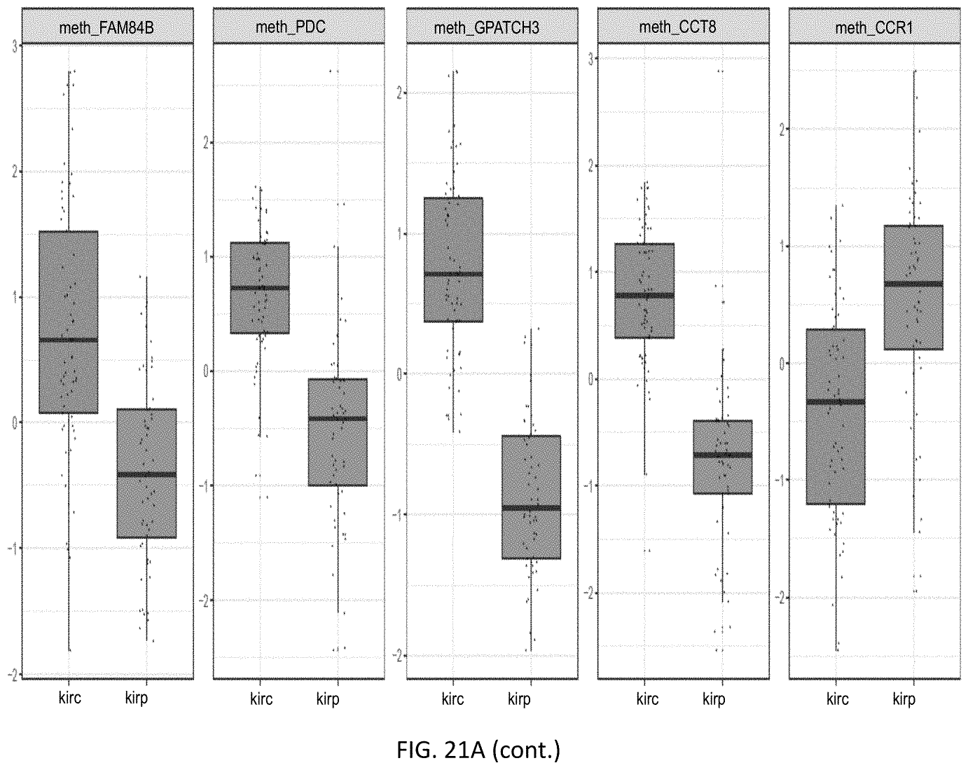

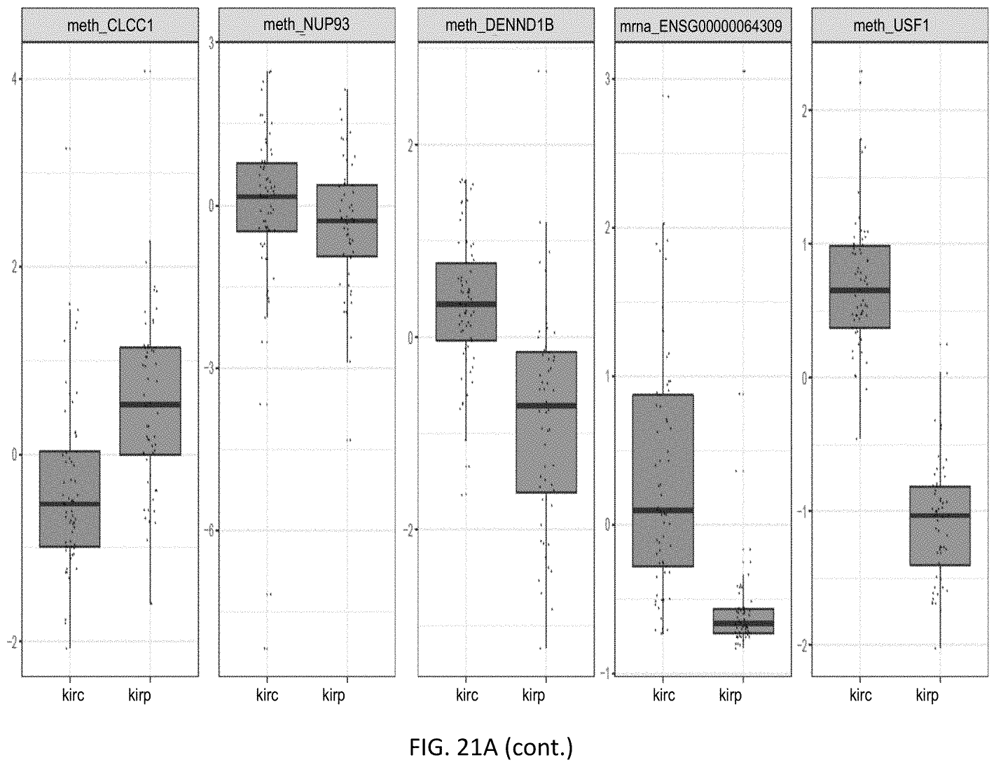

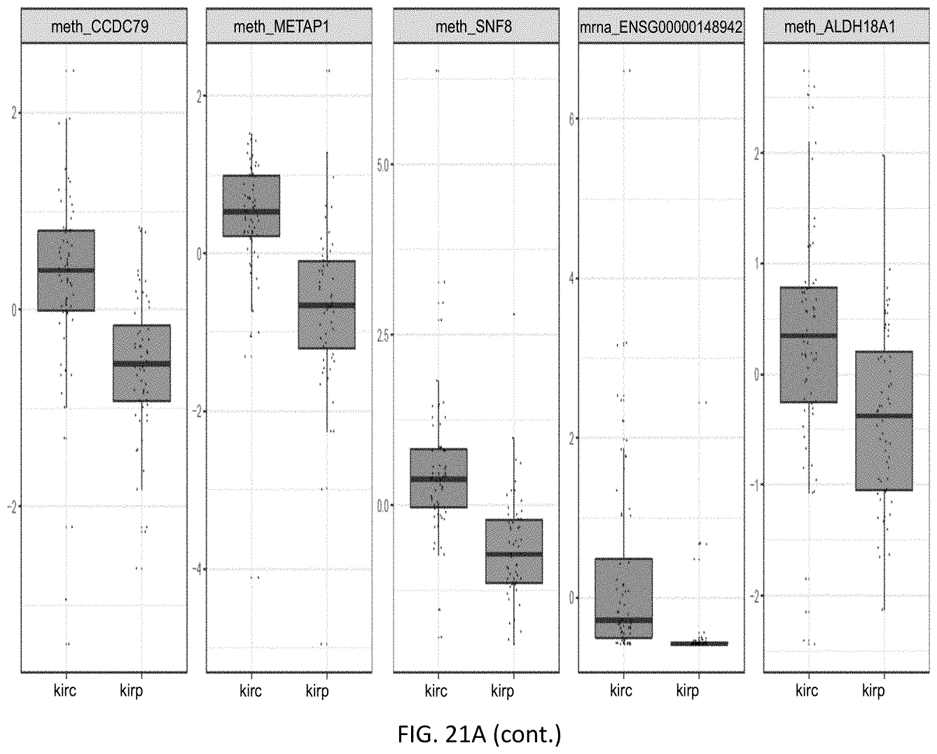

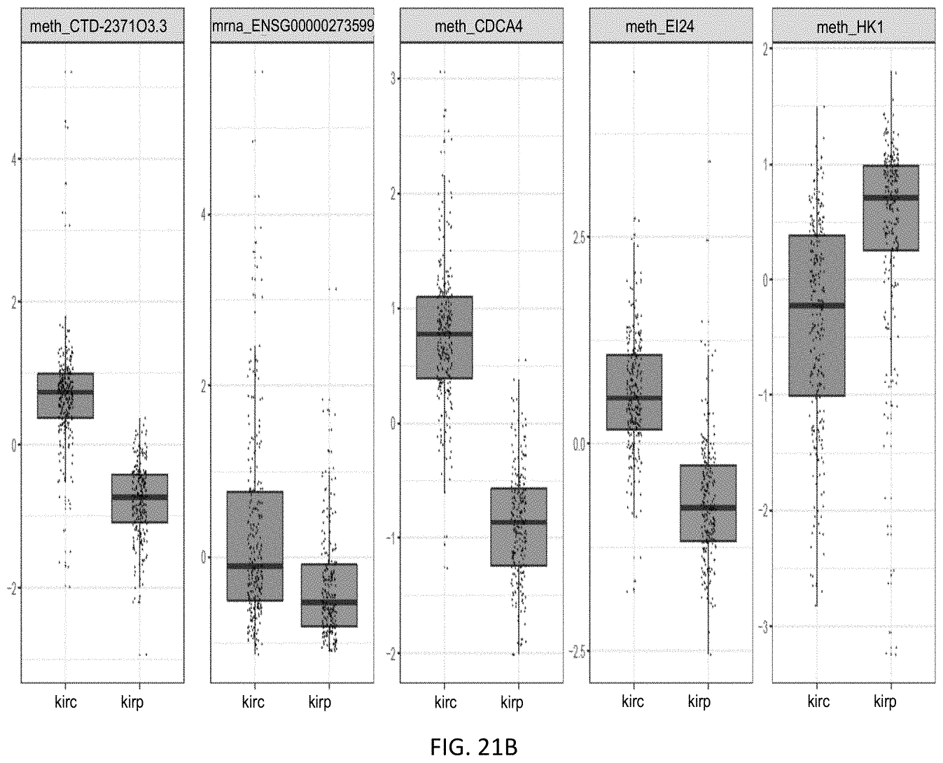

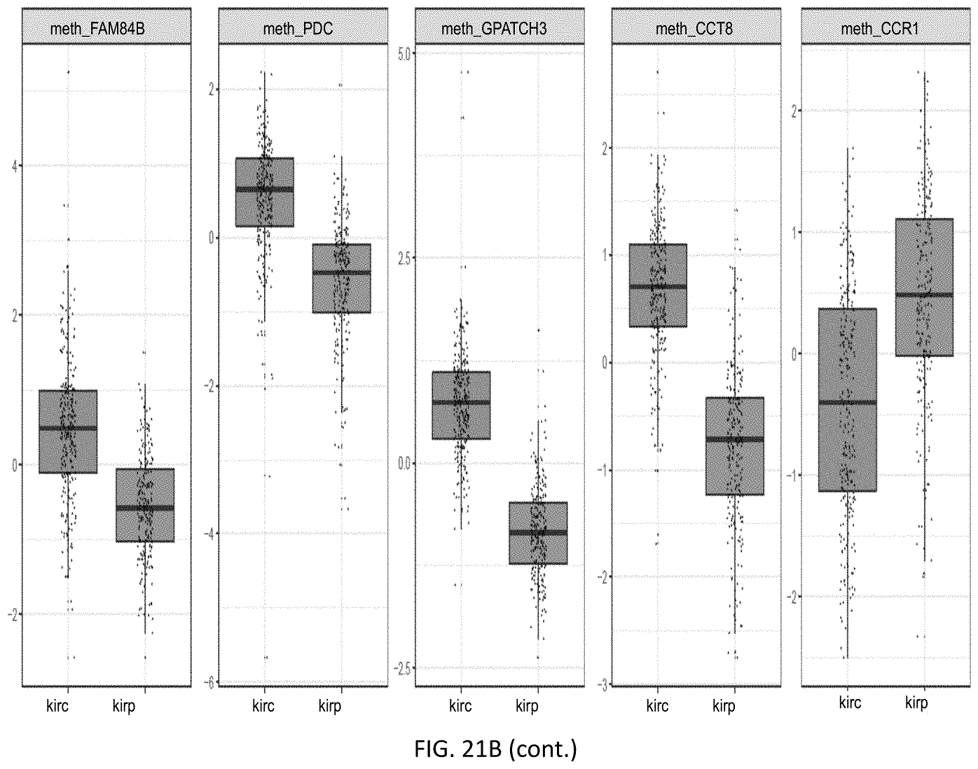

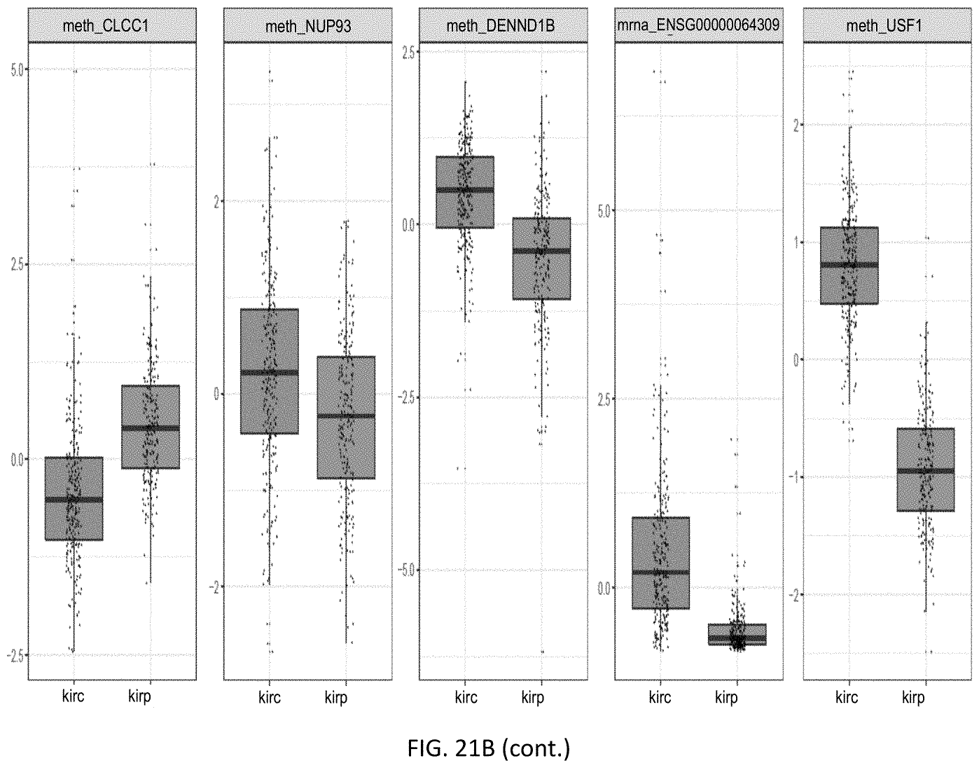

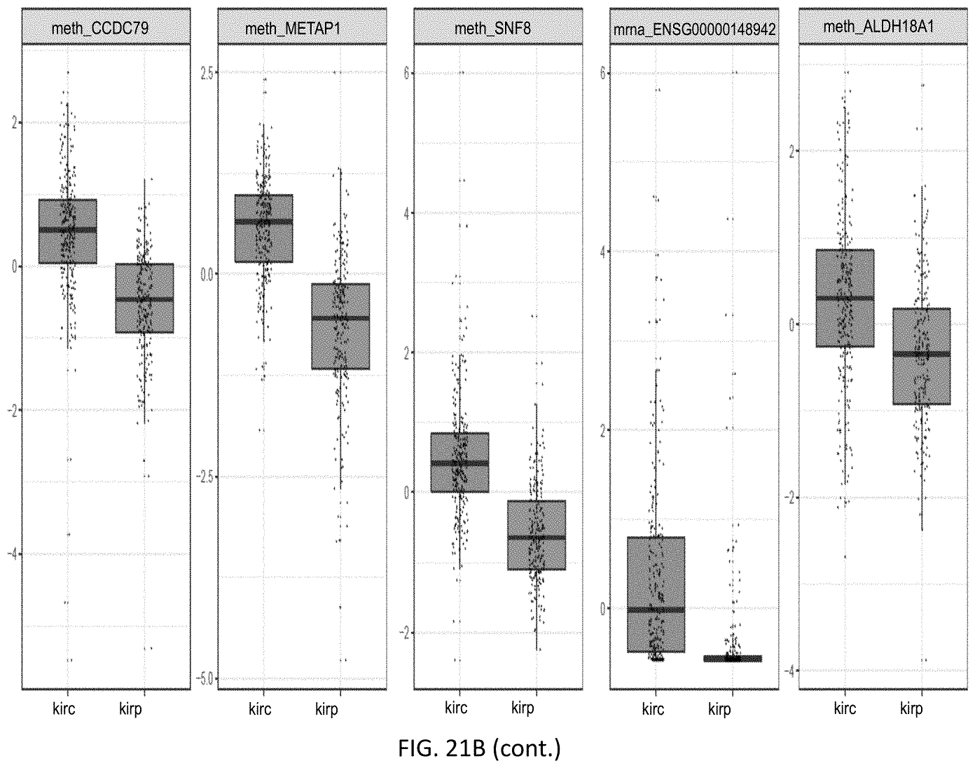

FIG. 21A and FIG. 21B depicts the top 20 most informative MEGENA genes at the gene level for kidney renal papillary cell carcinoma (KIRP) versus kidney renal clear cell carcinoma (KIRC) (for both training (FIG. 21B) and testing data sets (21A)).

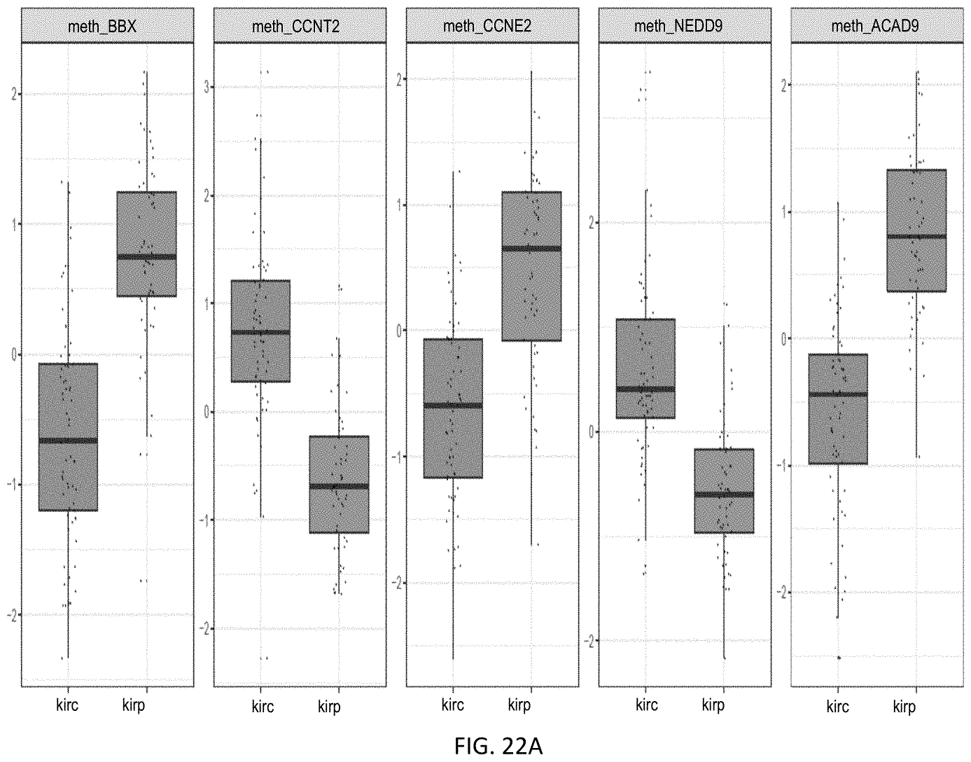

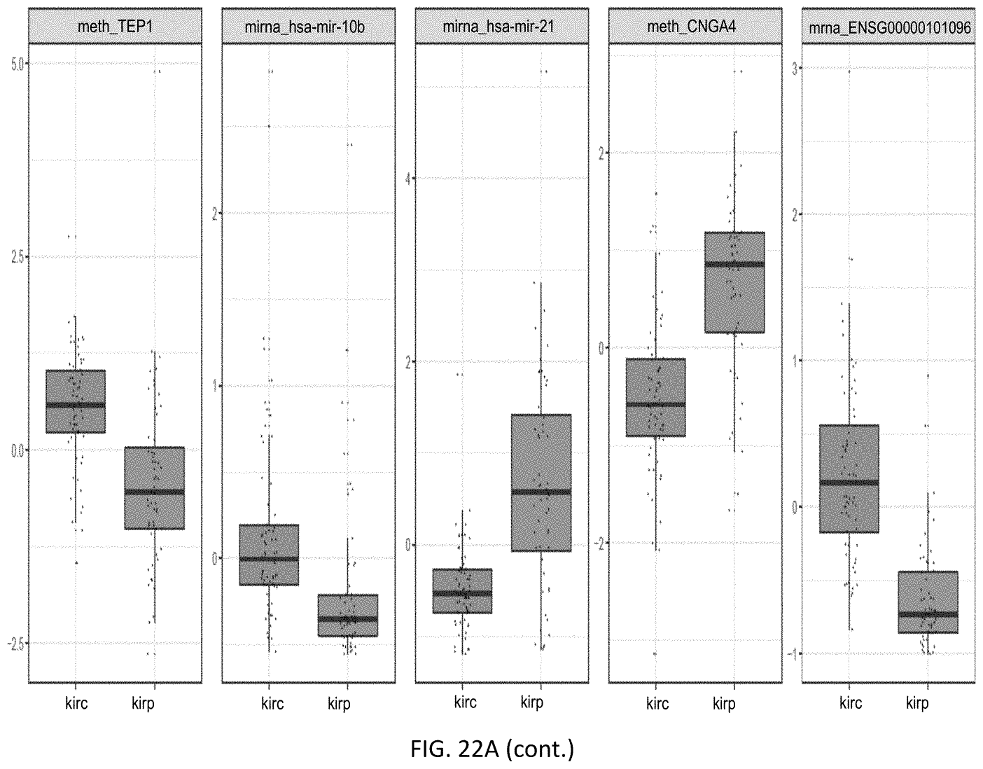

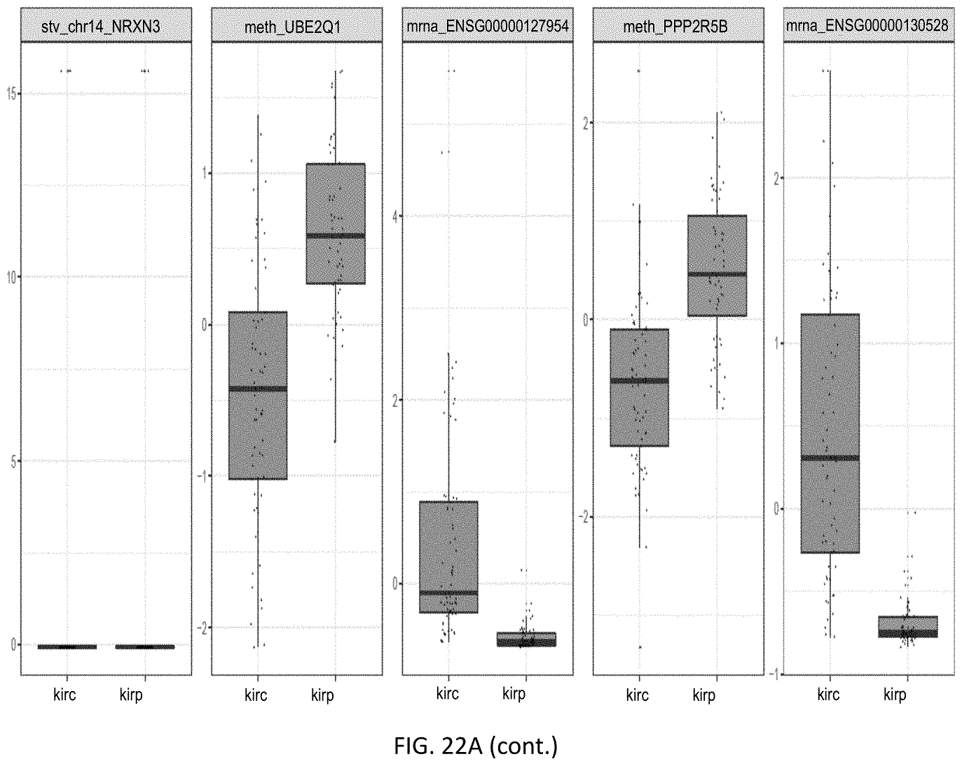

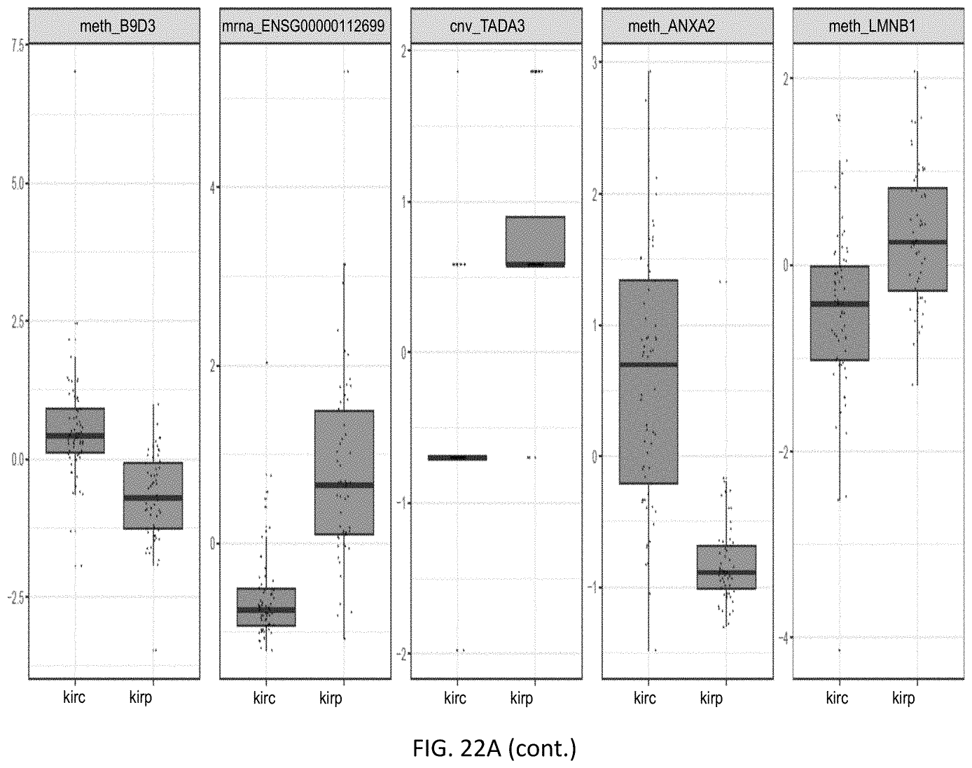

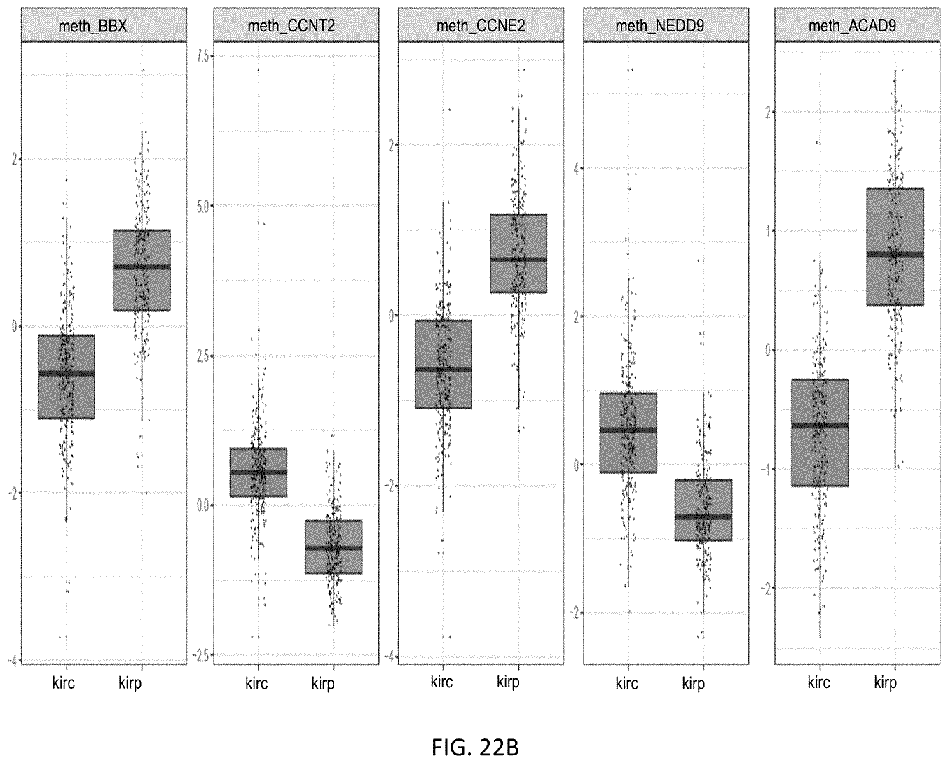

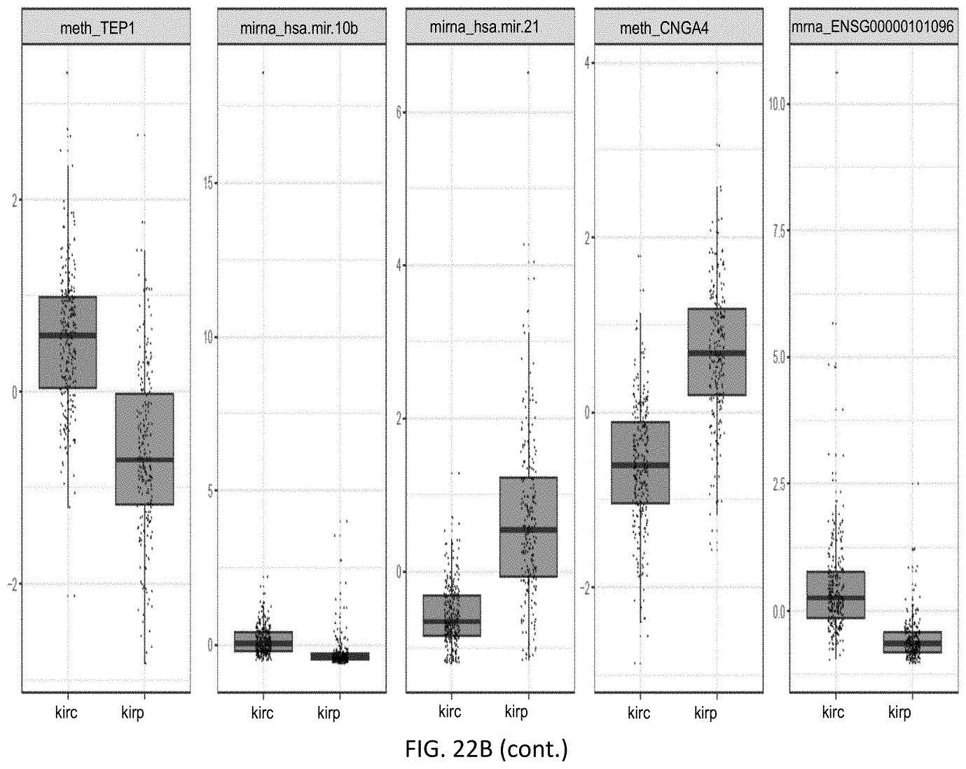

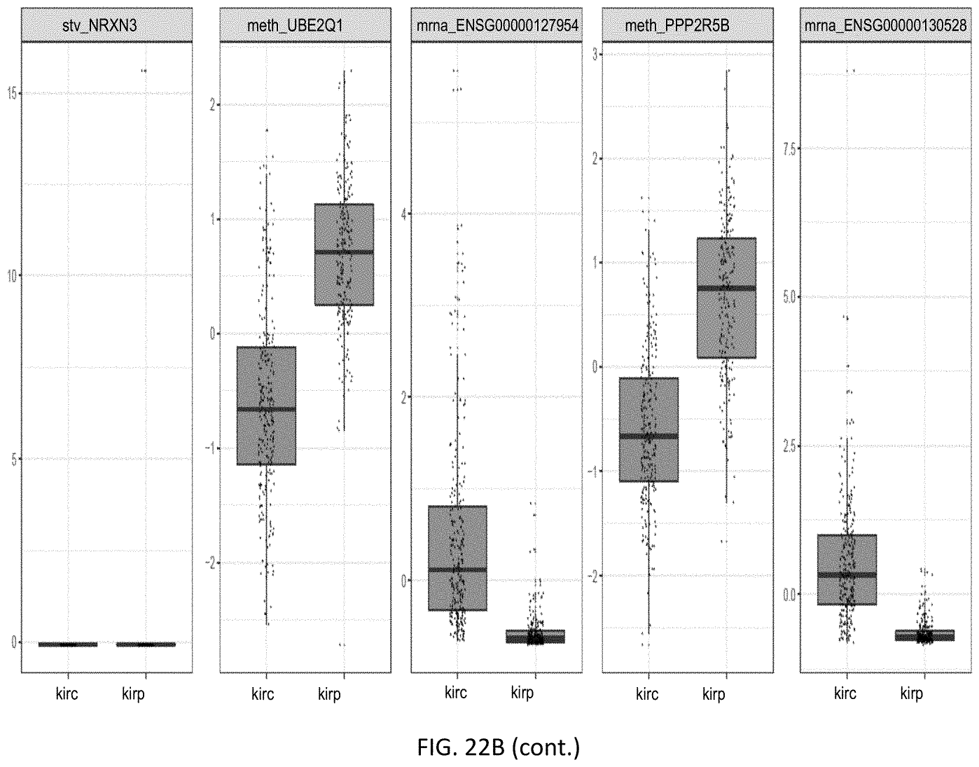

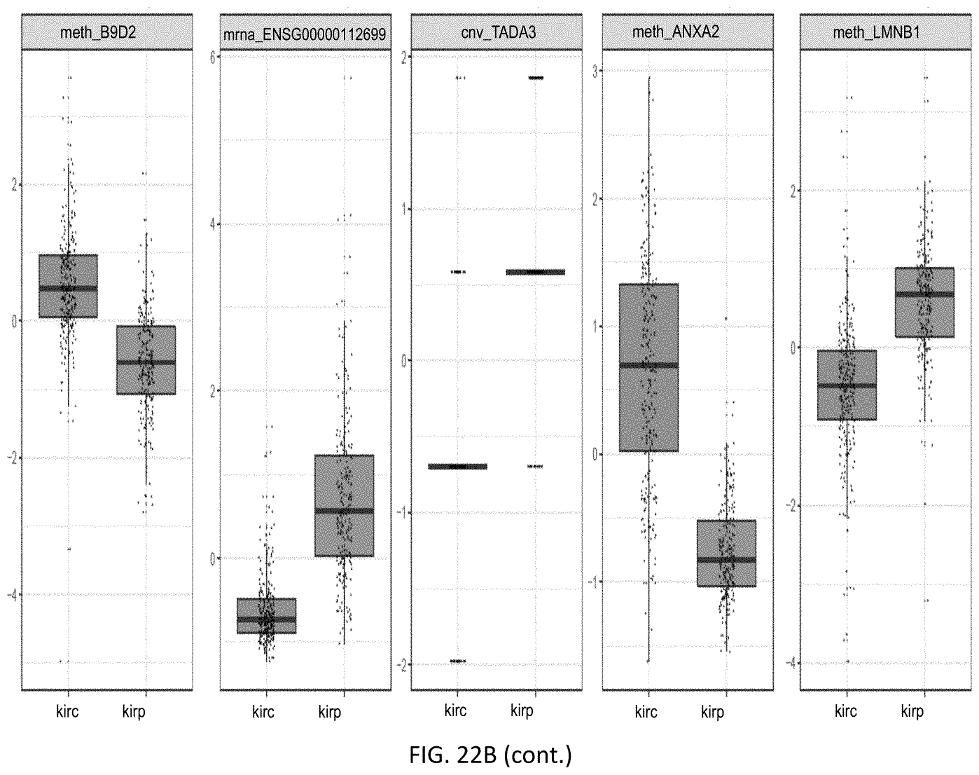

FIG. 21A and FIG. 21B depicts the top 20 most informative nGOseq genes at the gene level for kidney renal papillary cell carcinoma (KIRP) versus kidney renal clear cell carcinoma (KIRC) (for both training (FIG. 22B) and testing data sets (22A)).

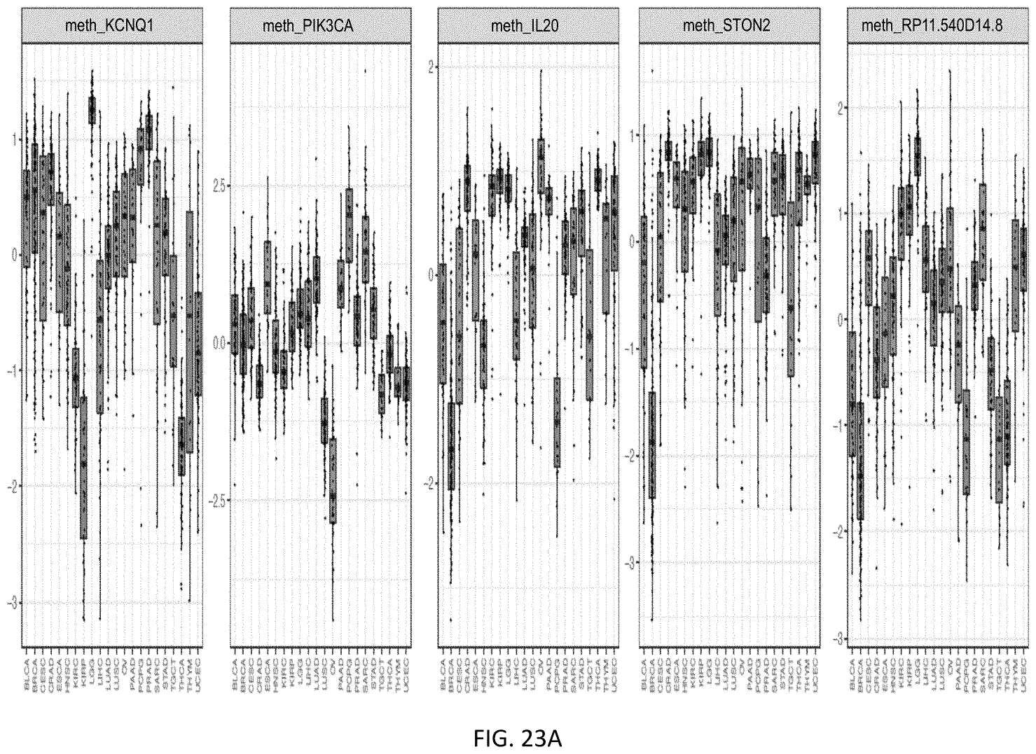

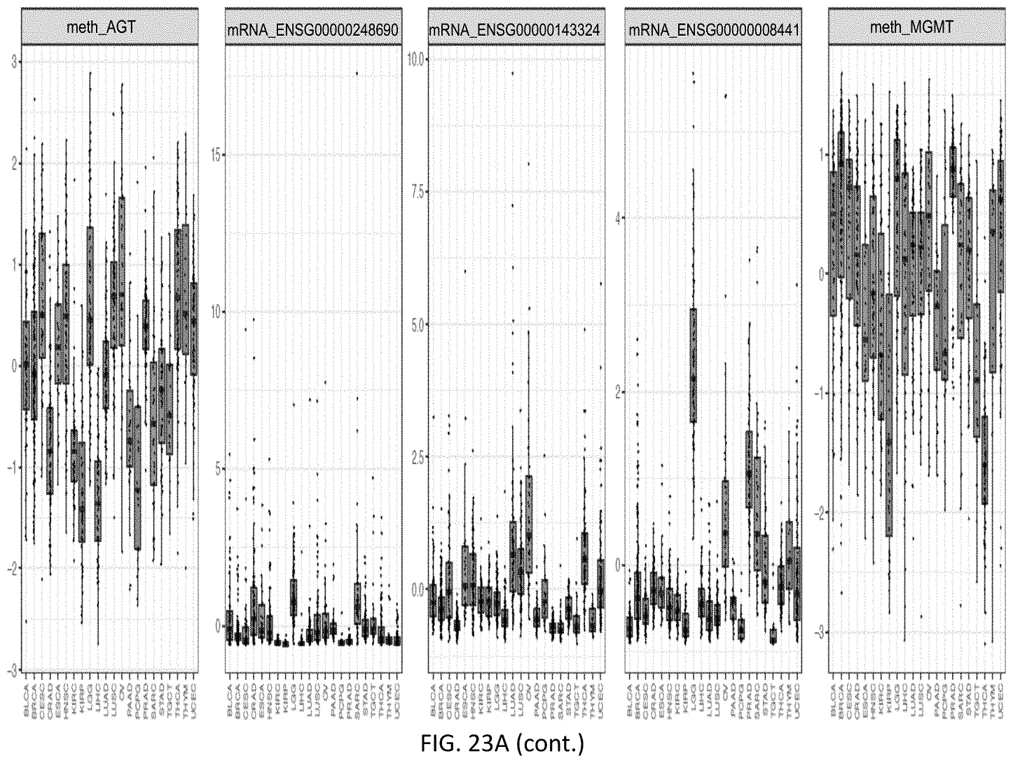

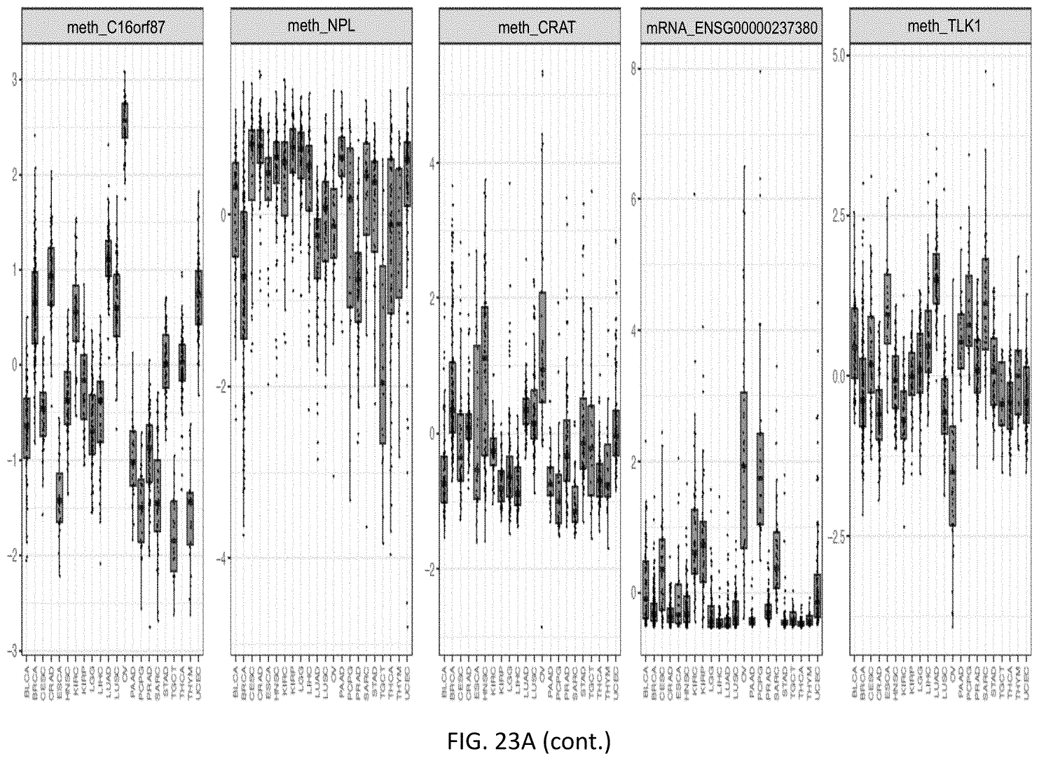

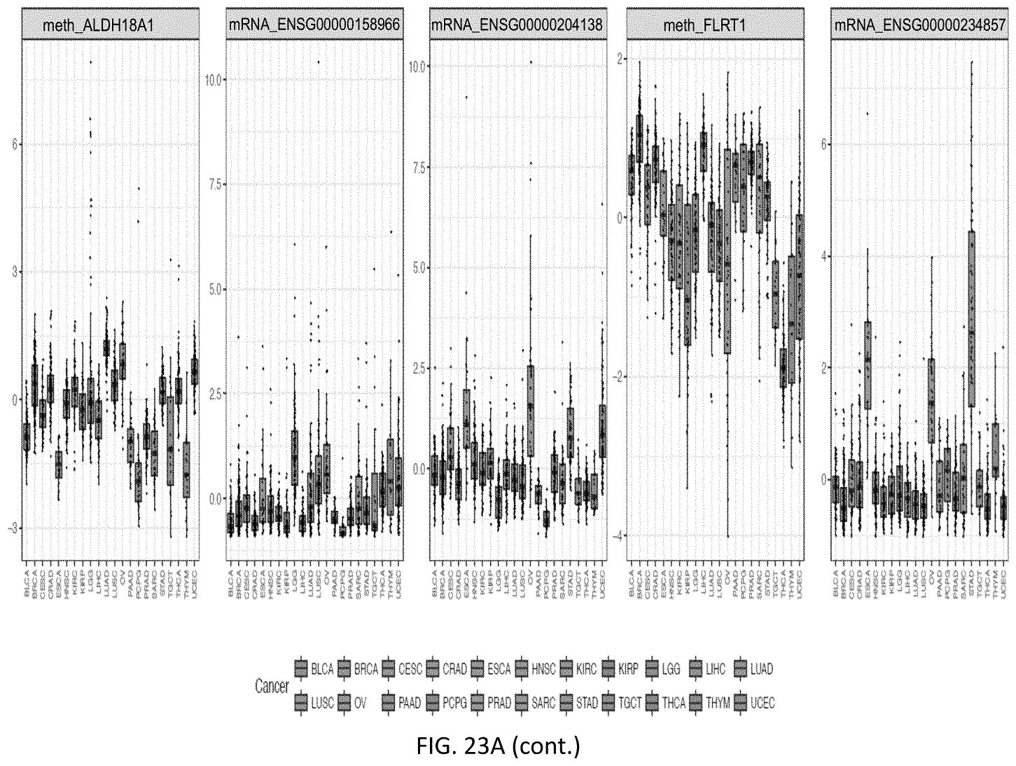

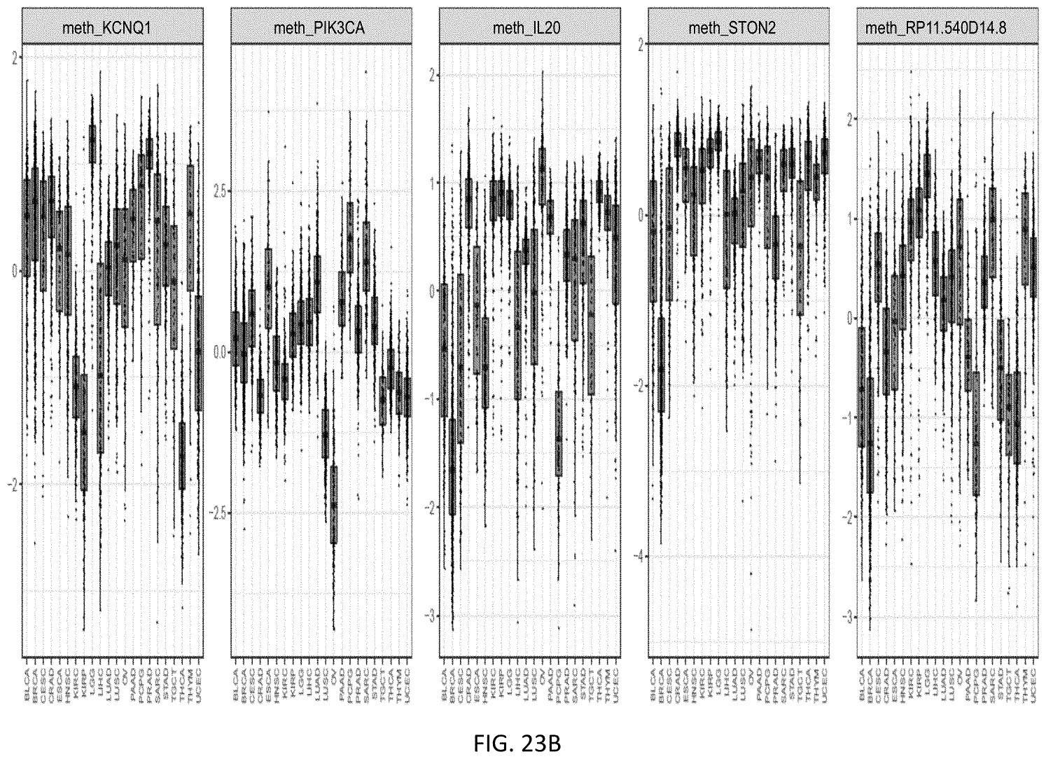

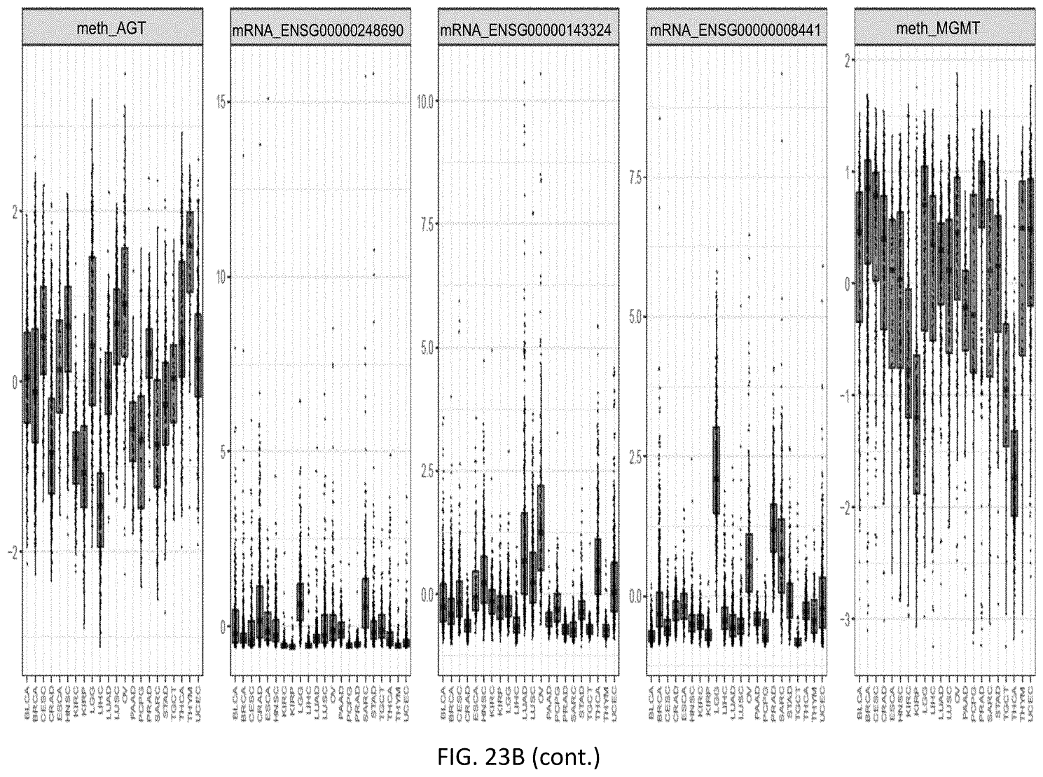

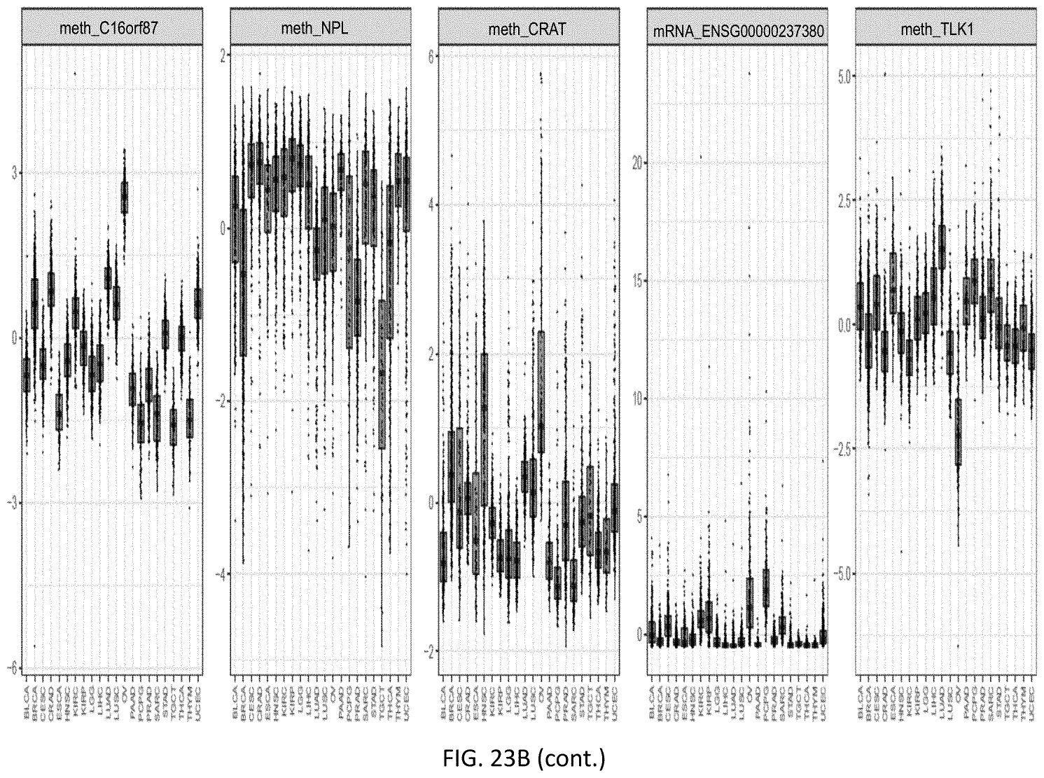

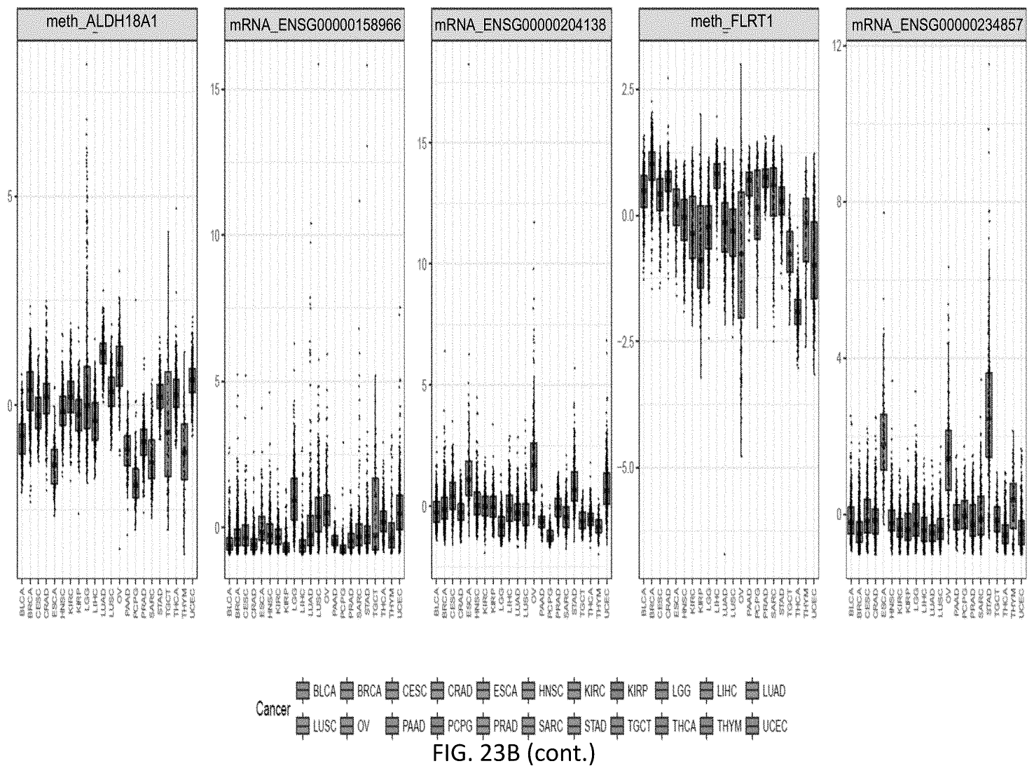

FIG. 23A and FIG. 23B depicts the top 20 most informative MEGENA genes at the gene level for the pan 22 cancer comparison (for both training (FIG. 23B) and testing data sets (23A))

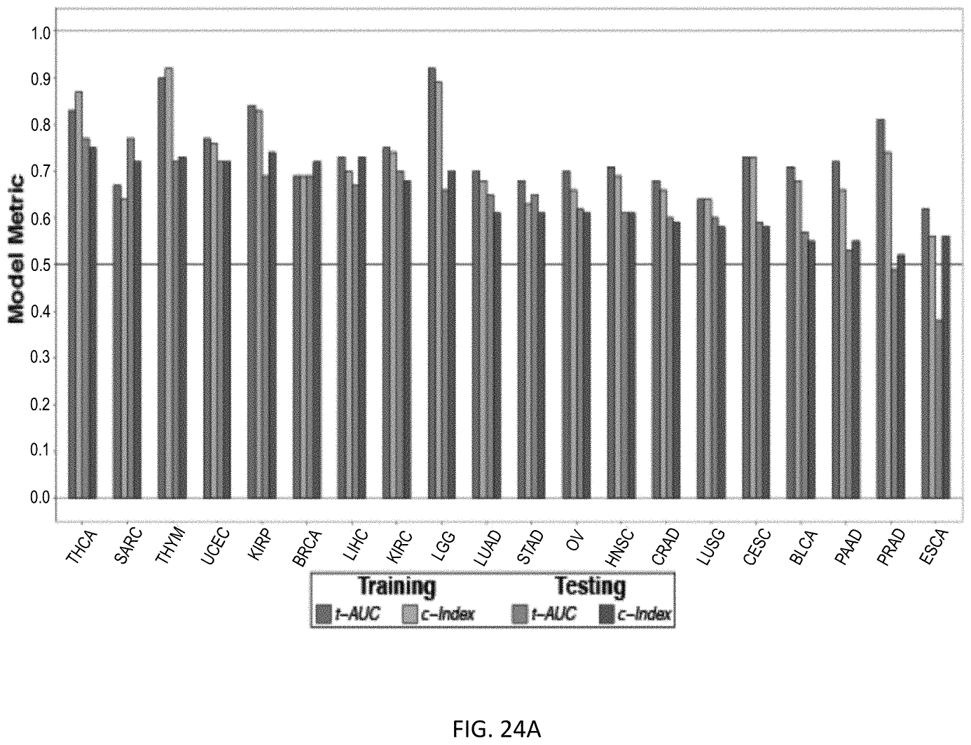

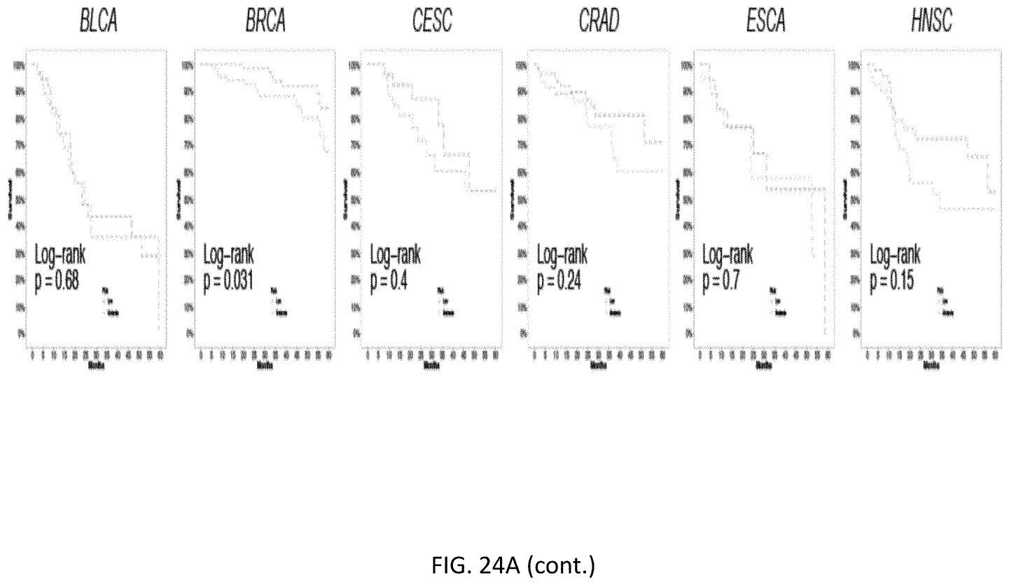

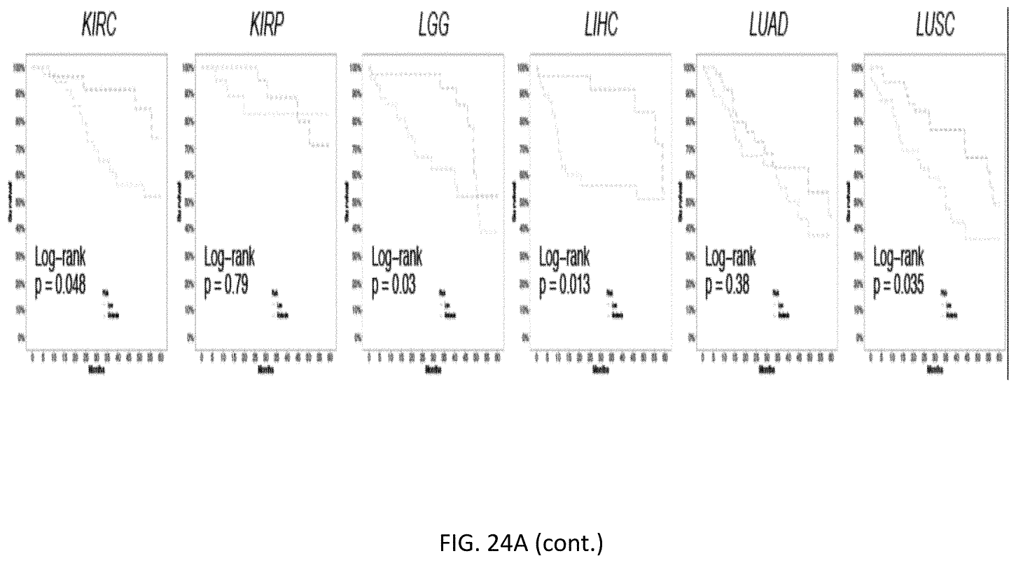

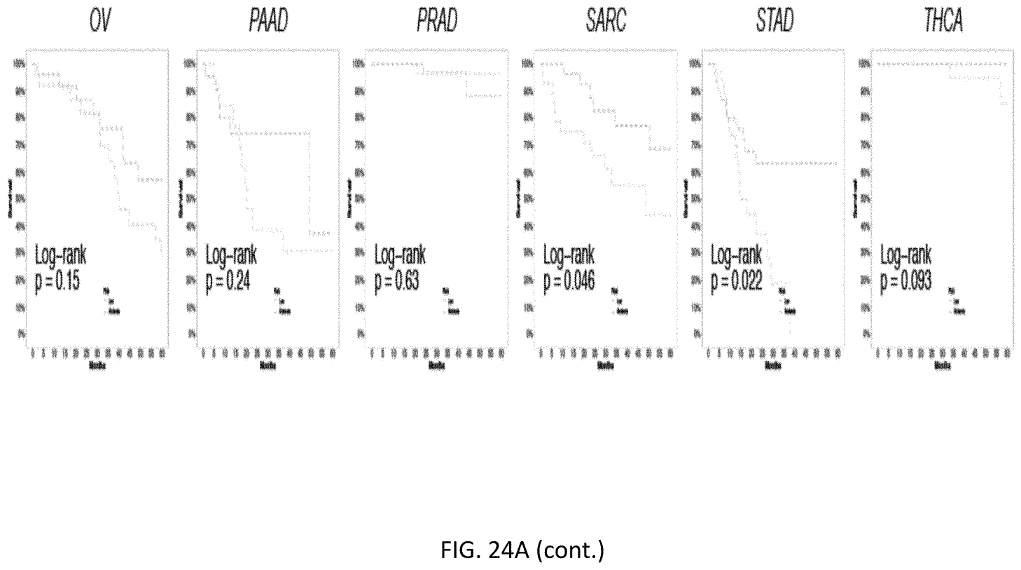

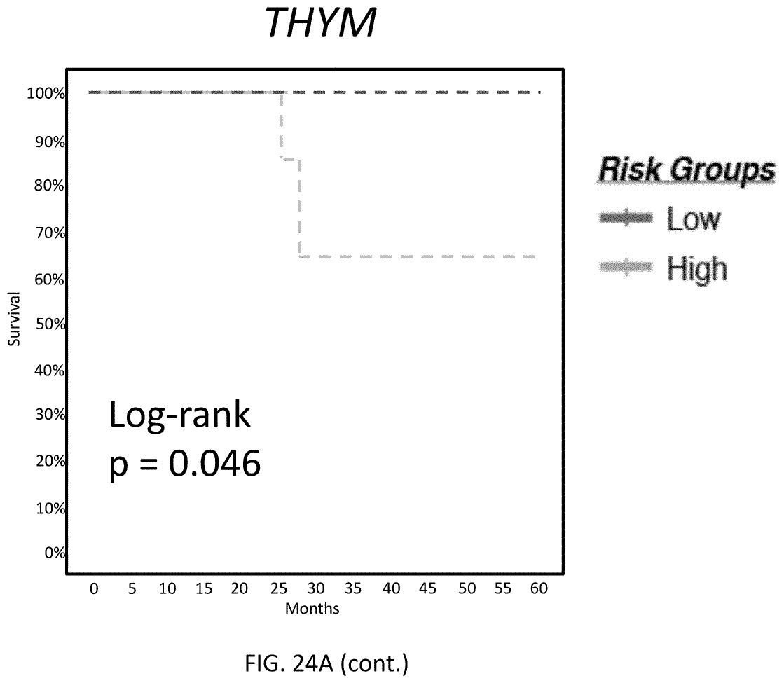

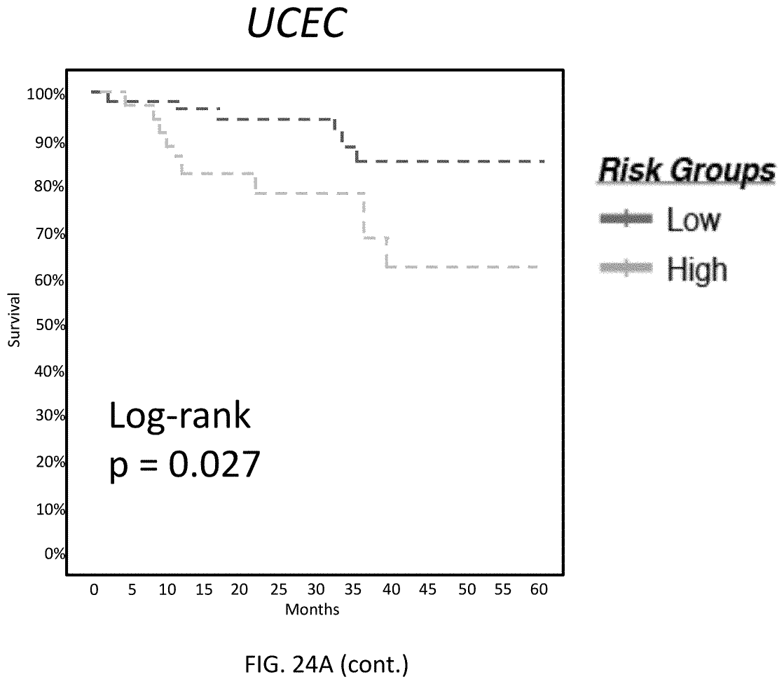

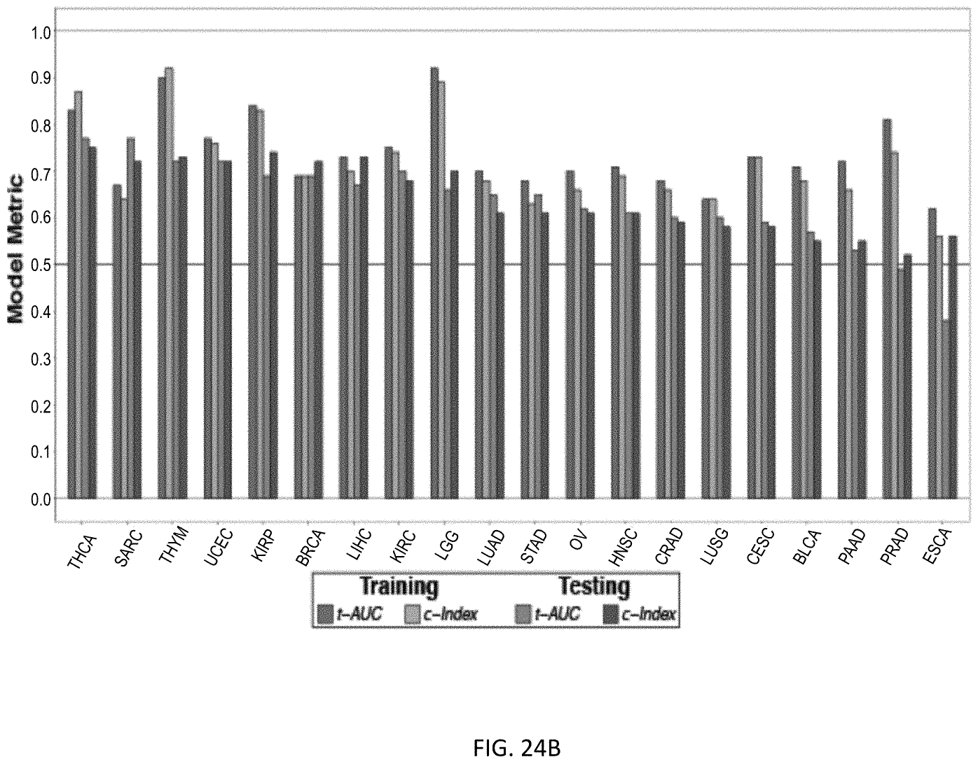

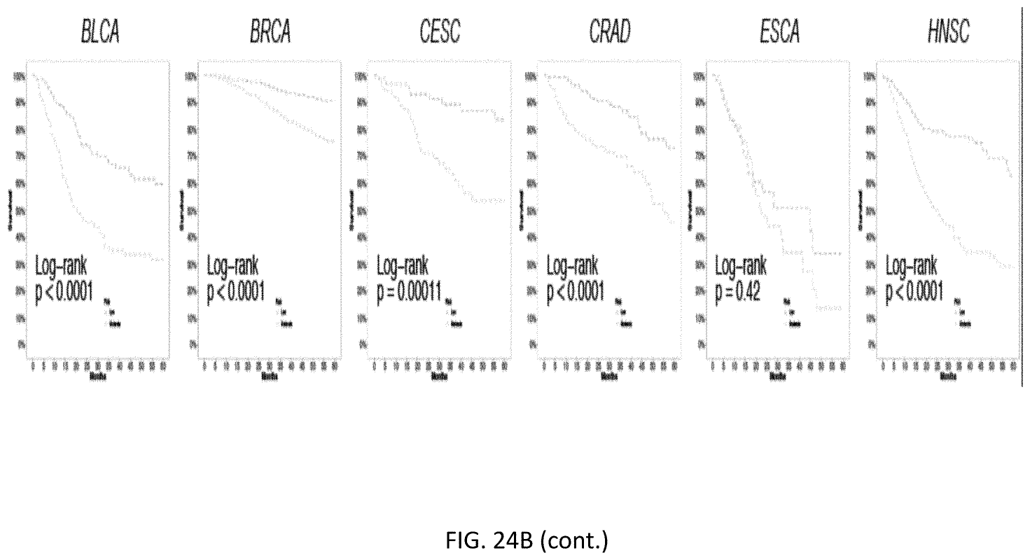

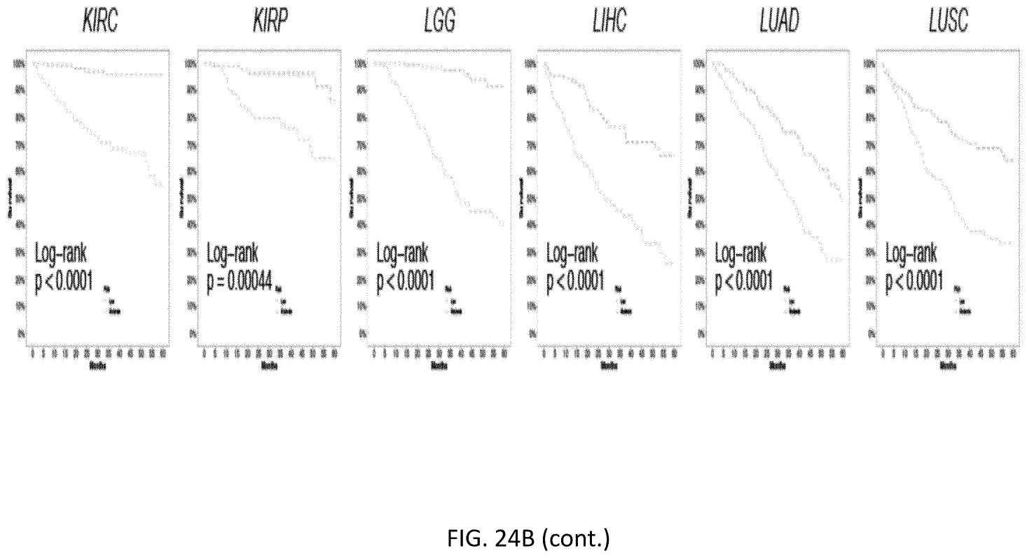

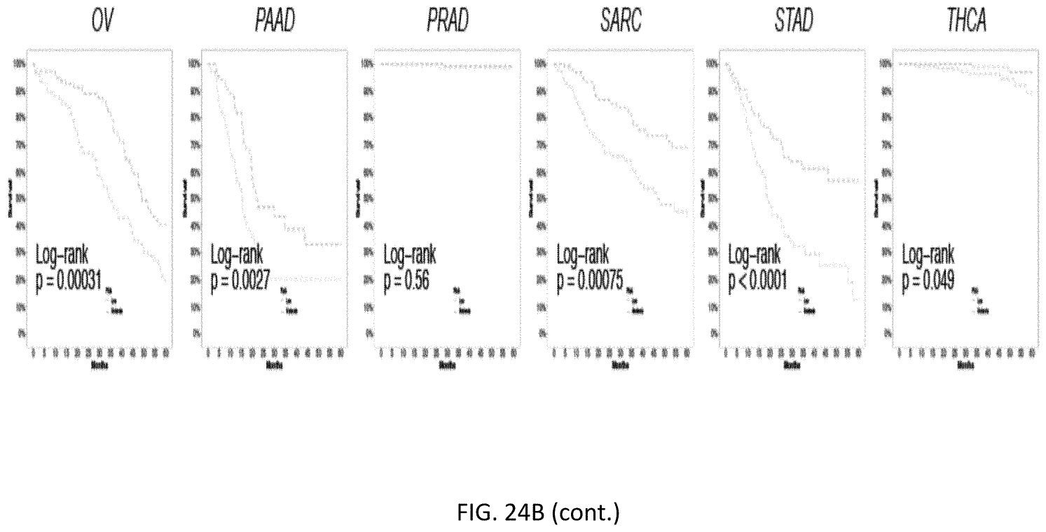

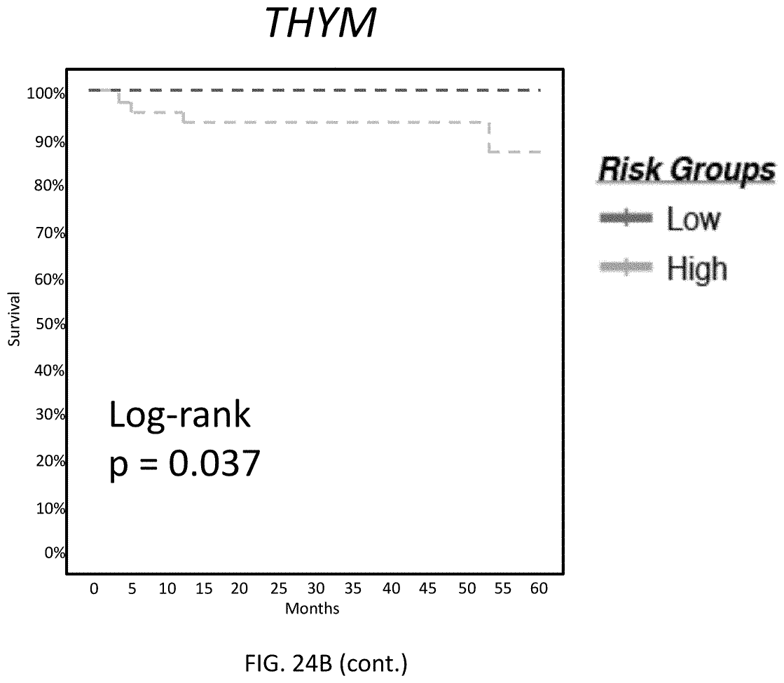

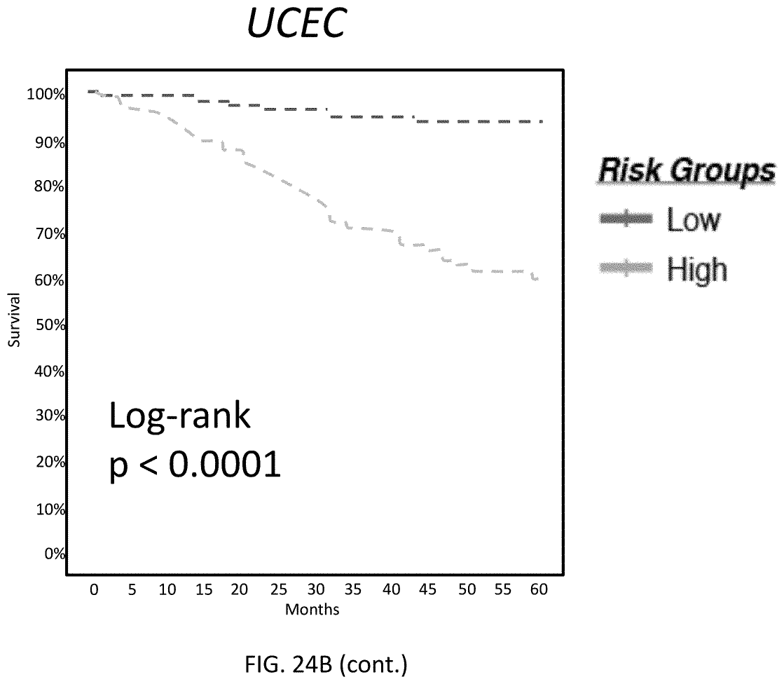

FIG. 24A and FIG. 24B depicts survival models at the nGOseq module level comparing 20 cancer types from the TCGA database. (top) Survival model statistics (for both training (FIG. 24B) and testing (FIG. 24A) data sets) broken down by each of the 20 cancer types. (bottom) Kaplan-Meier plots for each of the 20 cancer types stratified into 2 risk groups (low risk—red, high risk—blue, solid—testing data, dashed—training data). Risk stratification was determined by grouping the predicted risks for each cancer type from the survival model at the MEGENA module level into 2 quantiles for all training samples and using the same median value to stratify the testing samples. P values were calculated via log-rank tests.

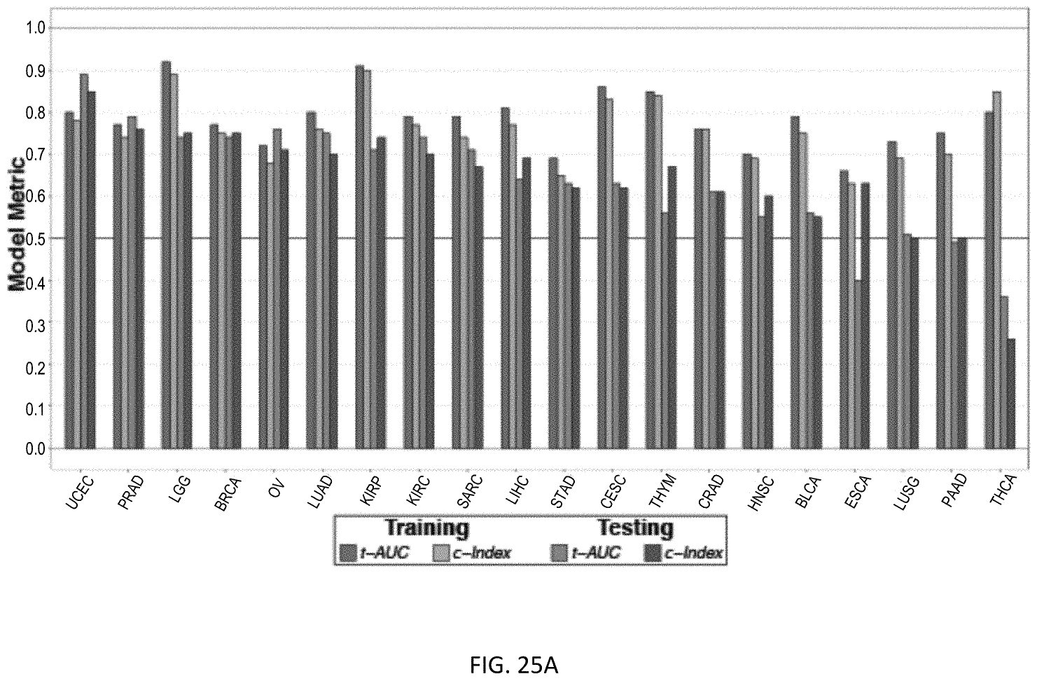

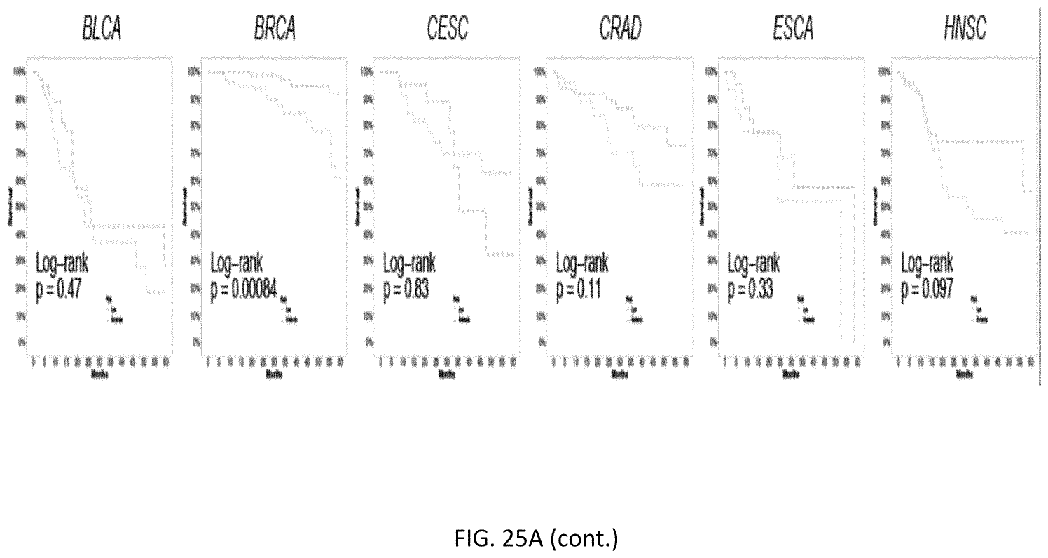

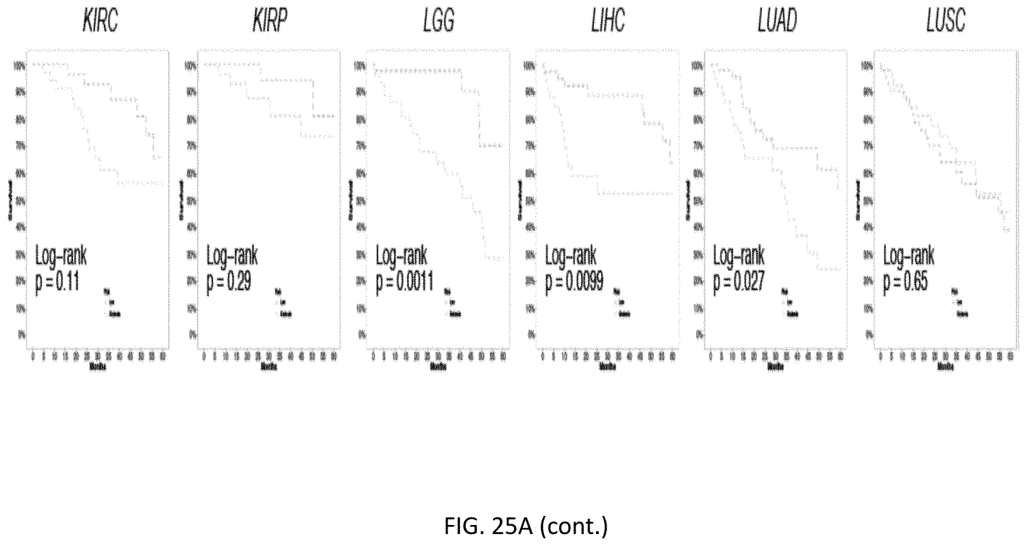

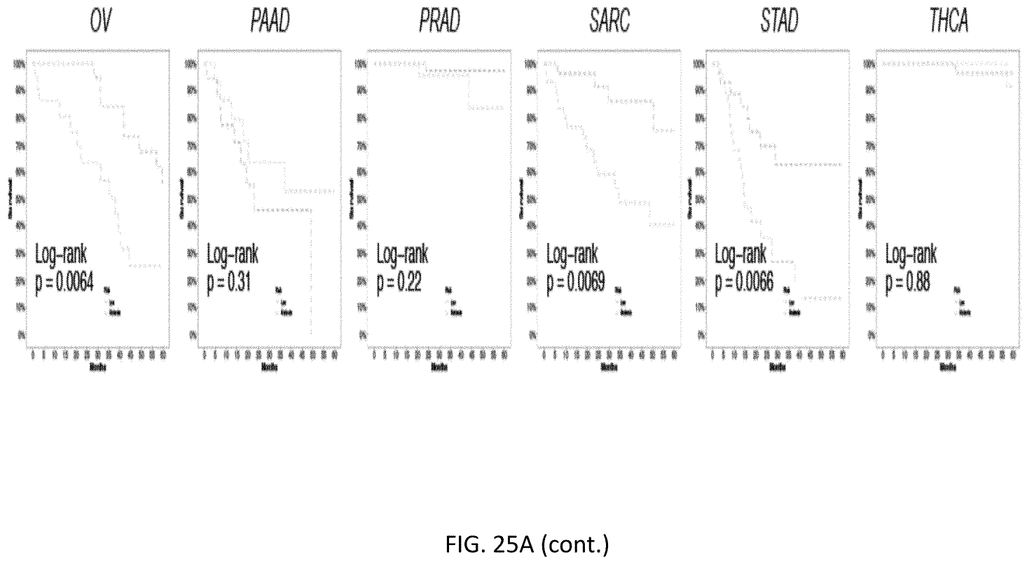

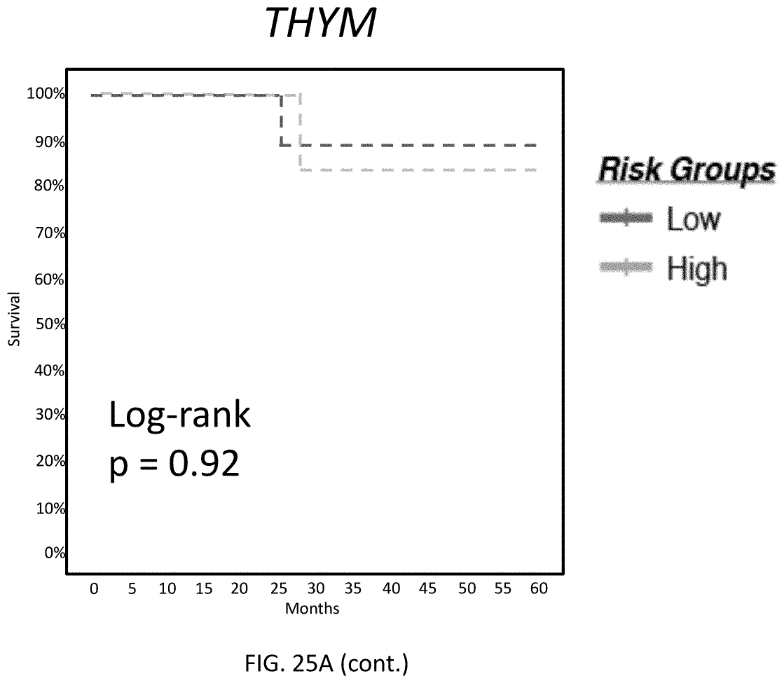

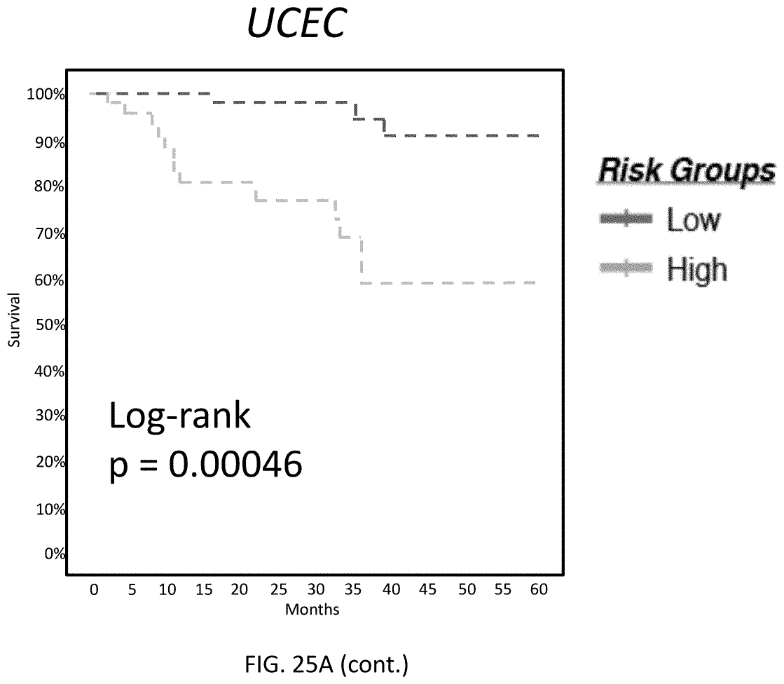

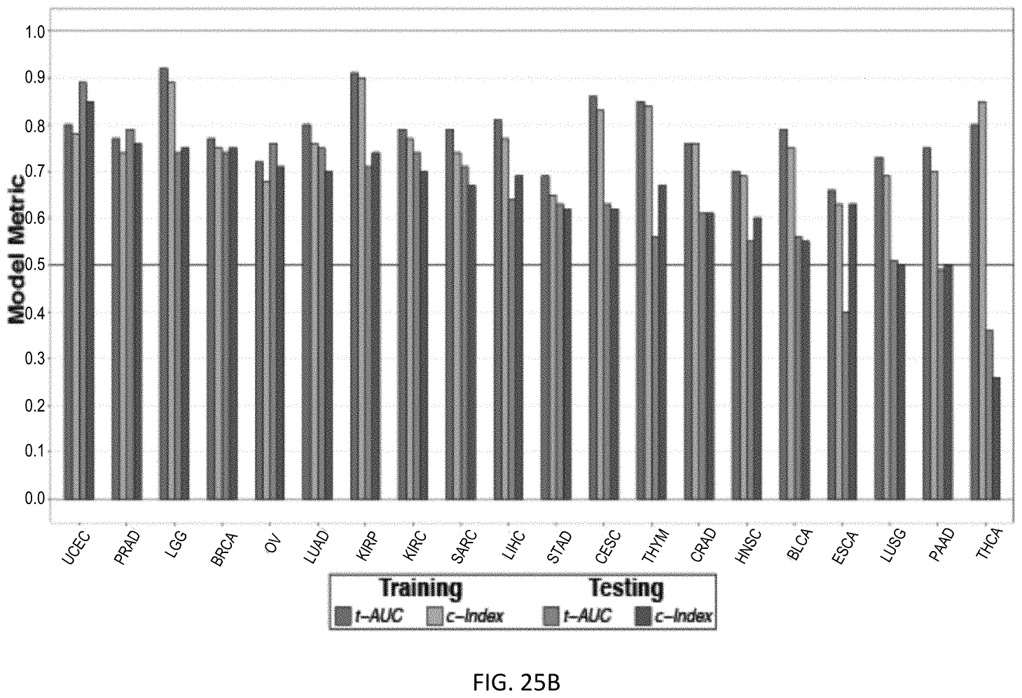

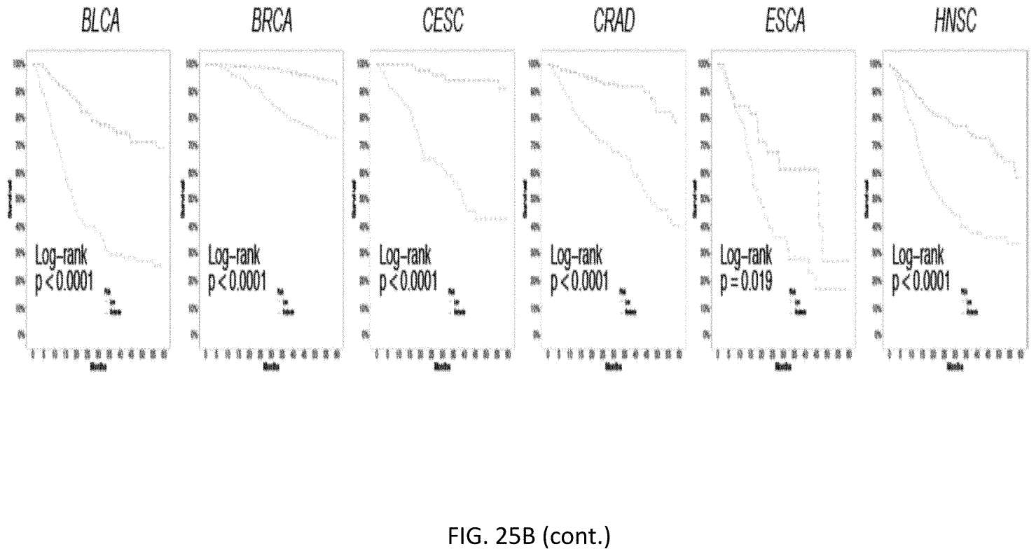

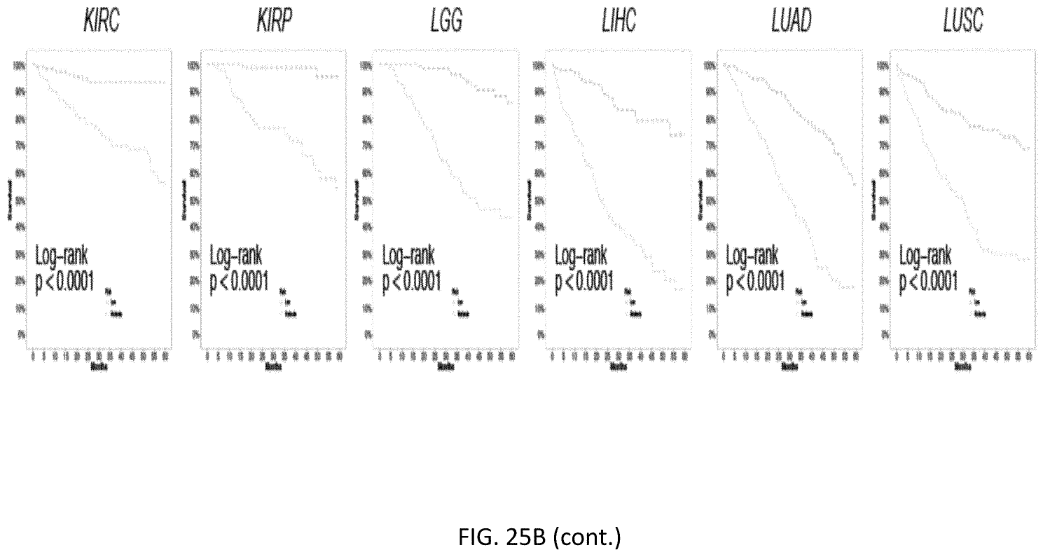

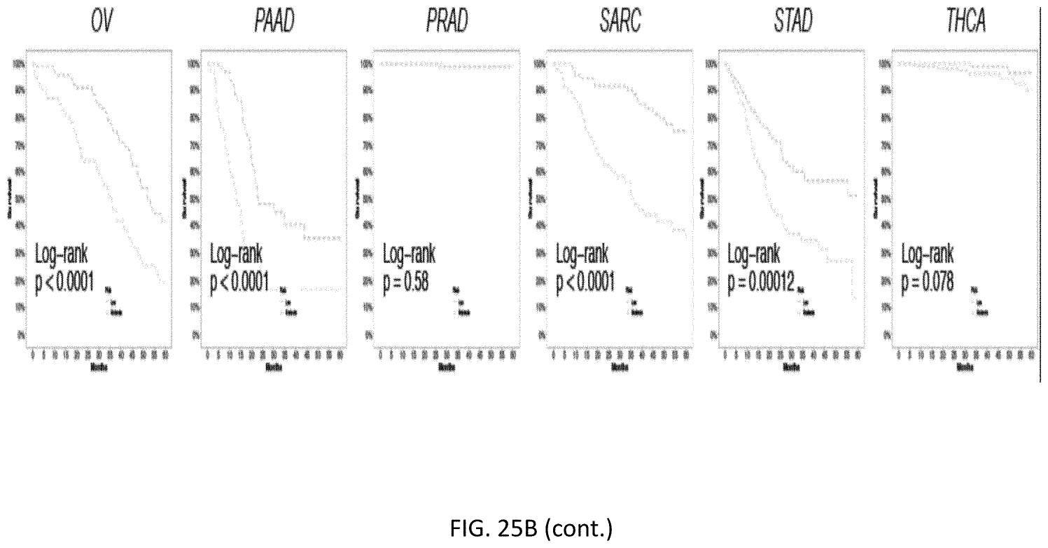

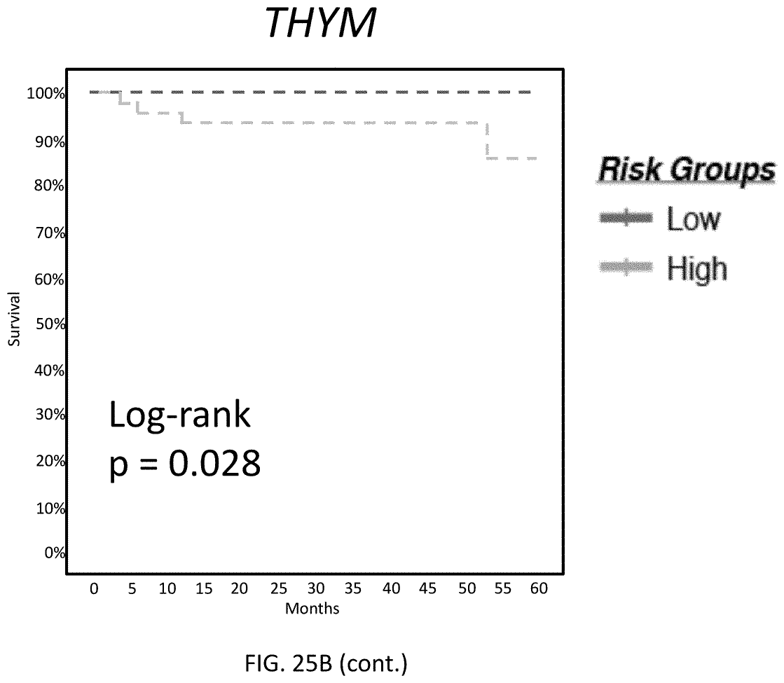

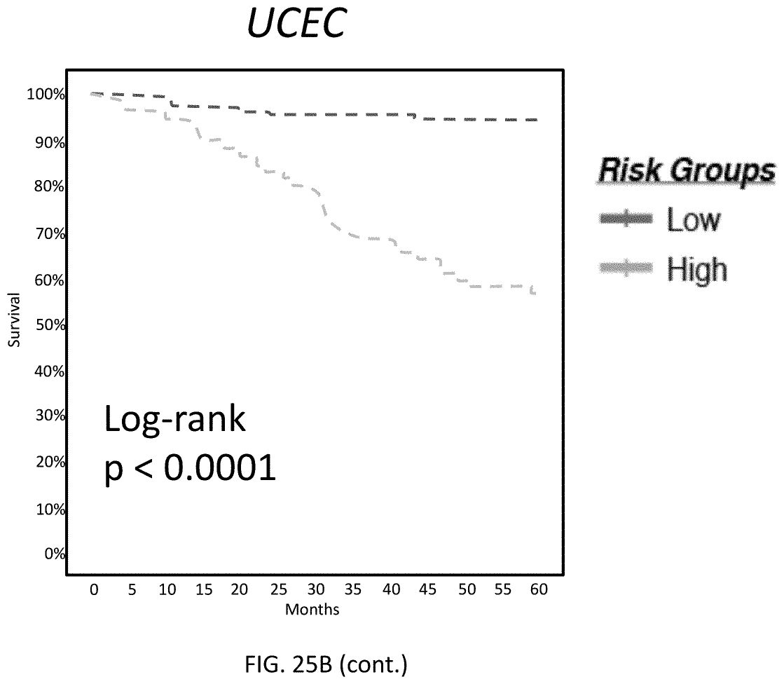

FIG. 25A and FIG. 25B depicts survival models at the MEGENA gene level comparing 20 cancer types from the TCGA database. (top) Survival model statistics (for both training (FIG. 24B) and testing (FIG. 24A) data sets) broken down by each of the 20 cancer types. (bottom) Kaplan-Meier plots for each of the 20 cancer types stratified into 2 risk groups (low risk—red, high risk—blue, solid—testing data, dashed—training data). Risk stratification was determined by grouping the predicted risks for each cancer type from the survival model at the MEGENA module level into 2 quantiles for all training samples and using the same median value to stratify the testing samples. P values were calculated via log-rank tests.

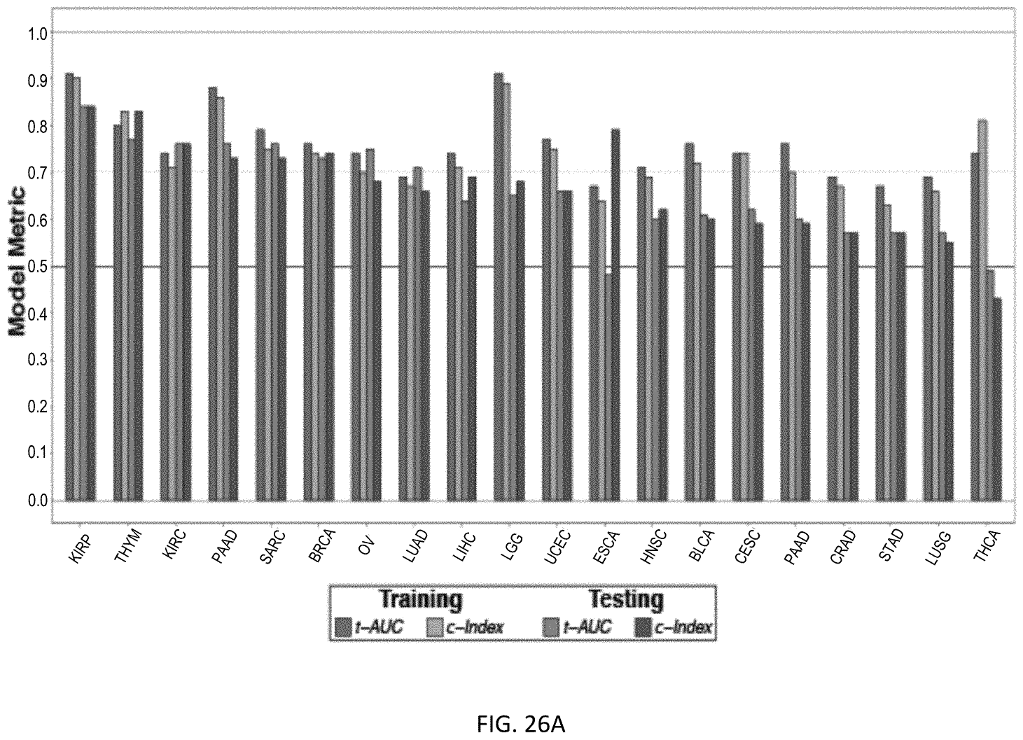

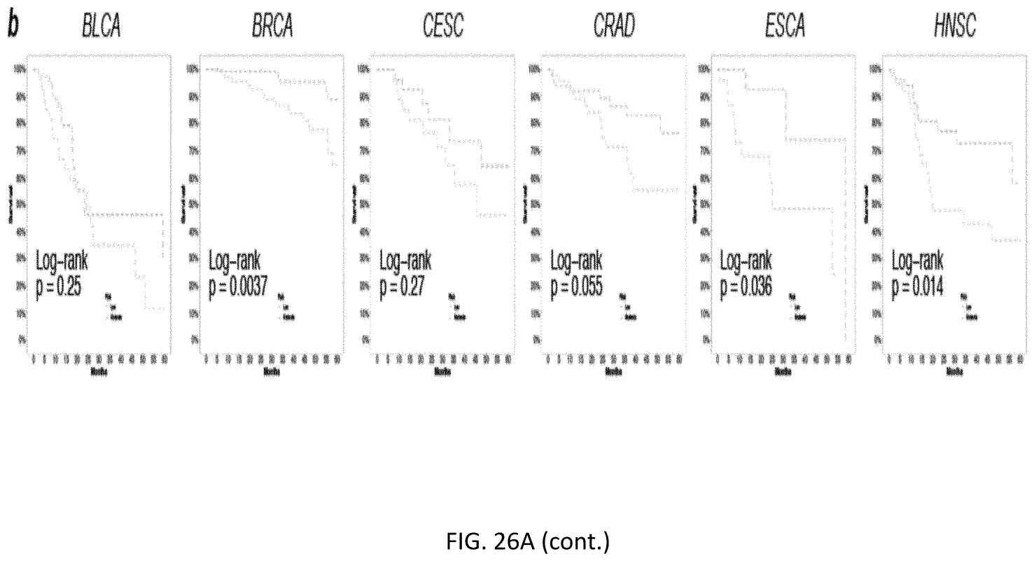

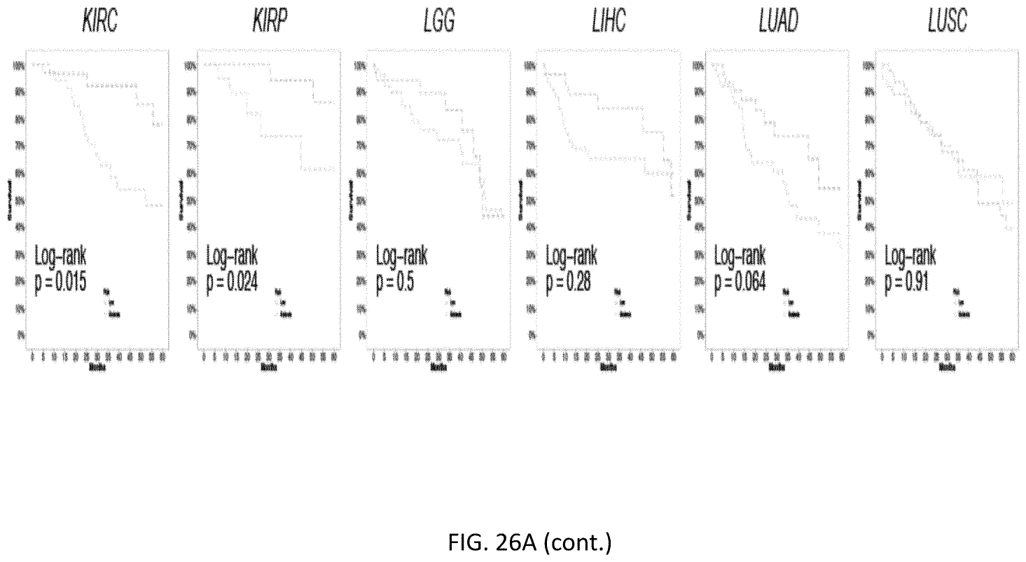

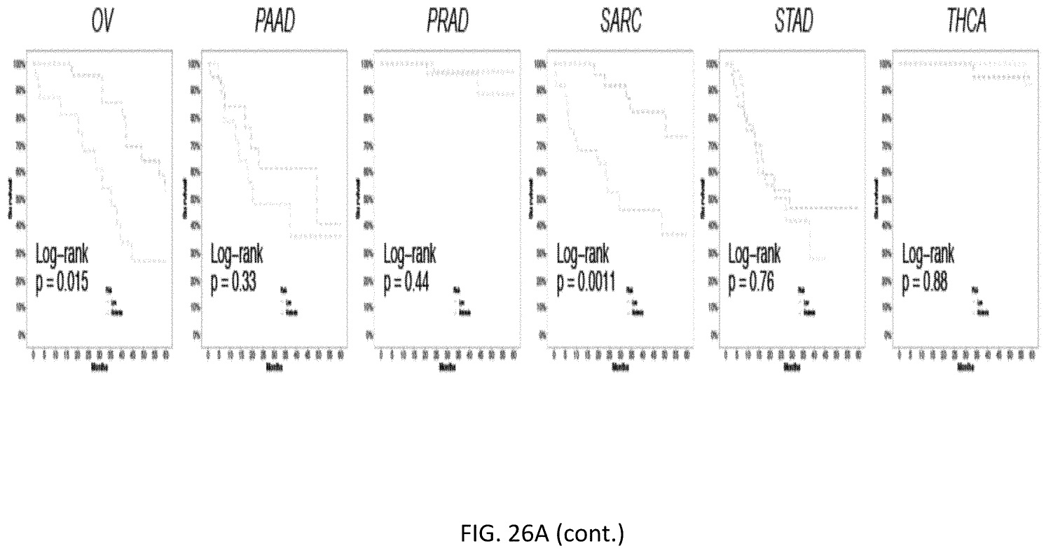

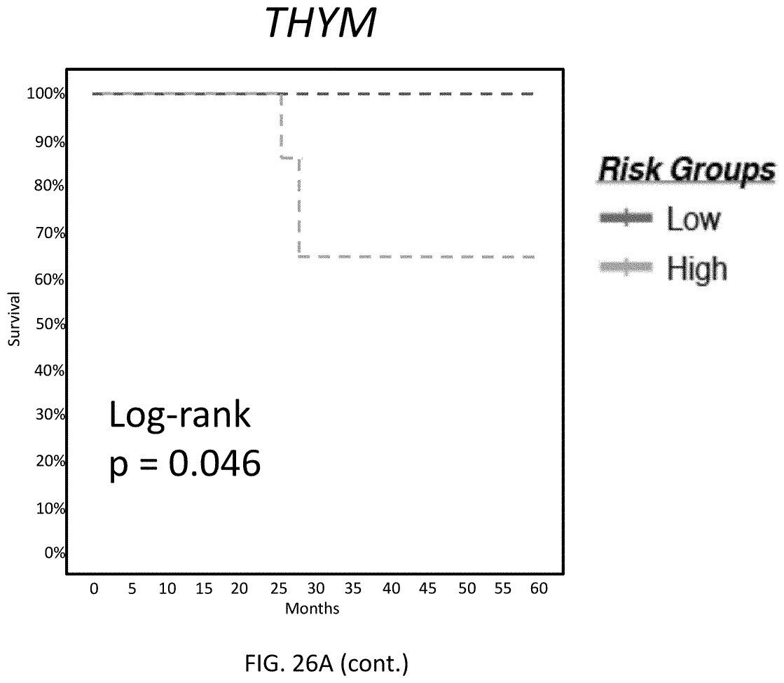

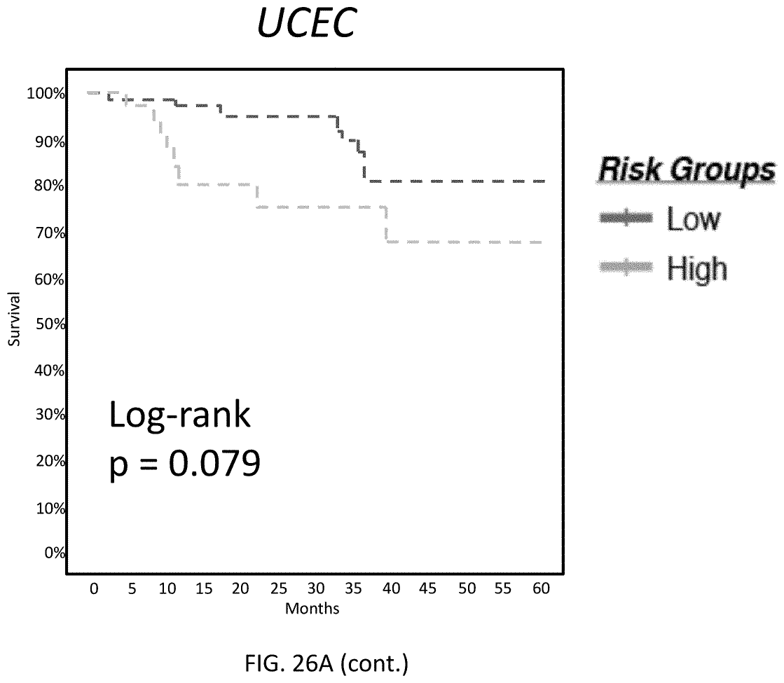

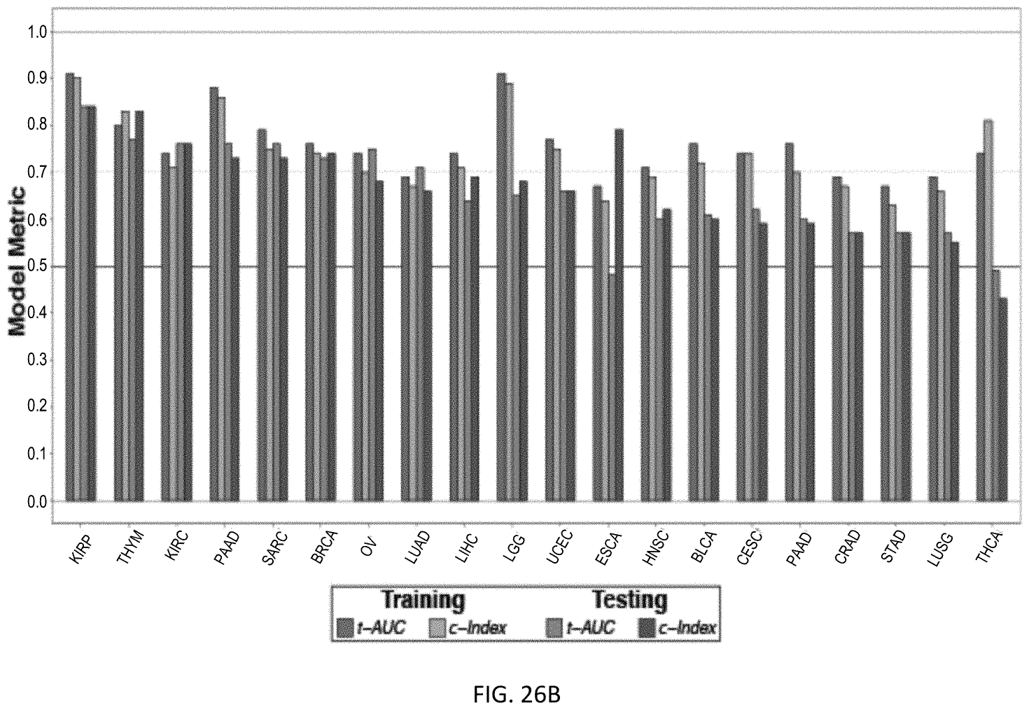

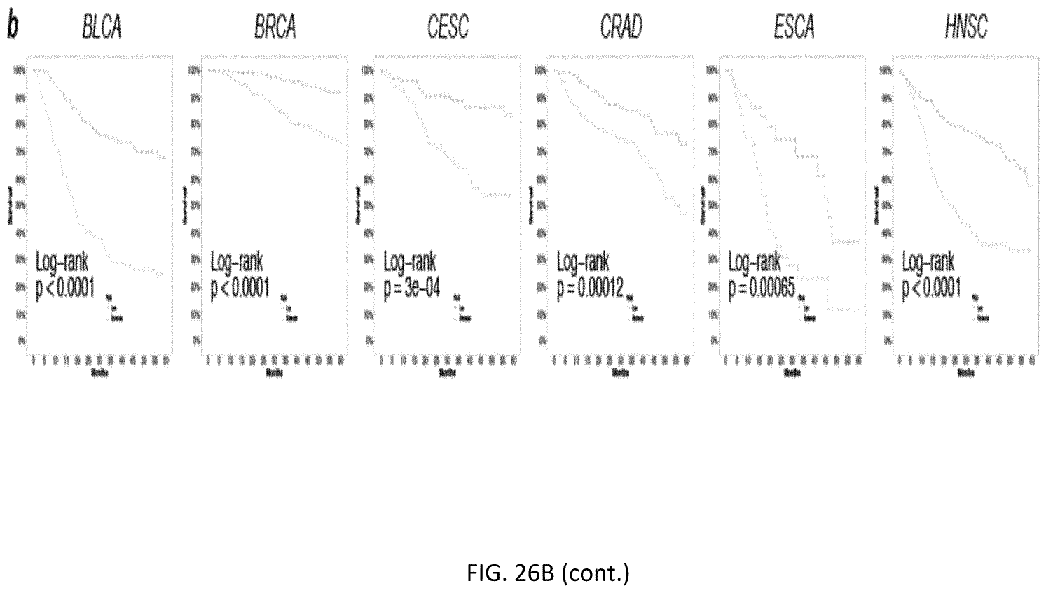

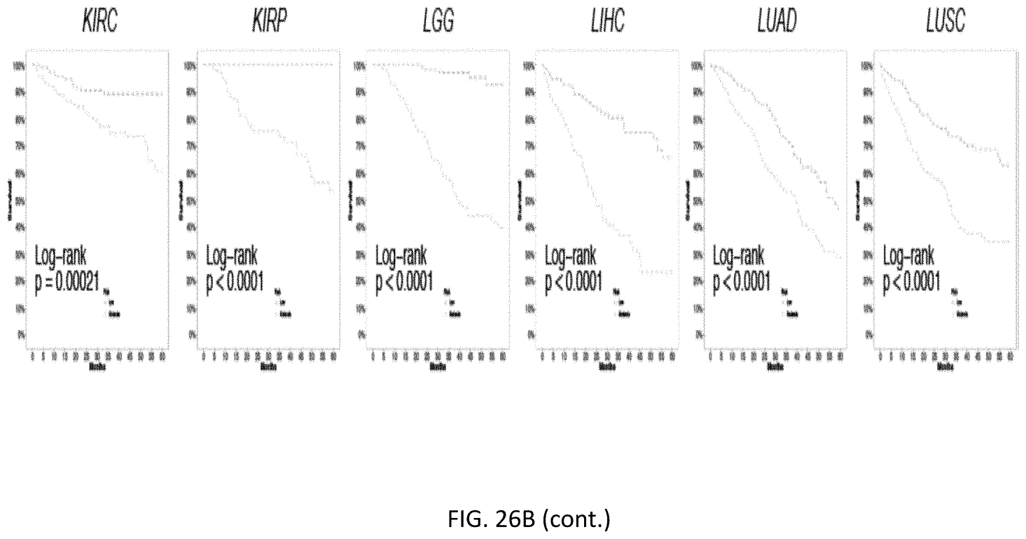

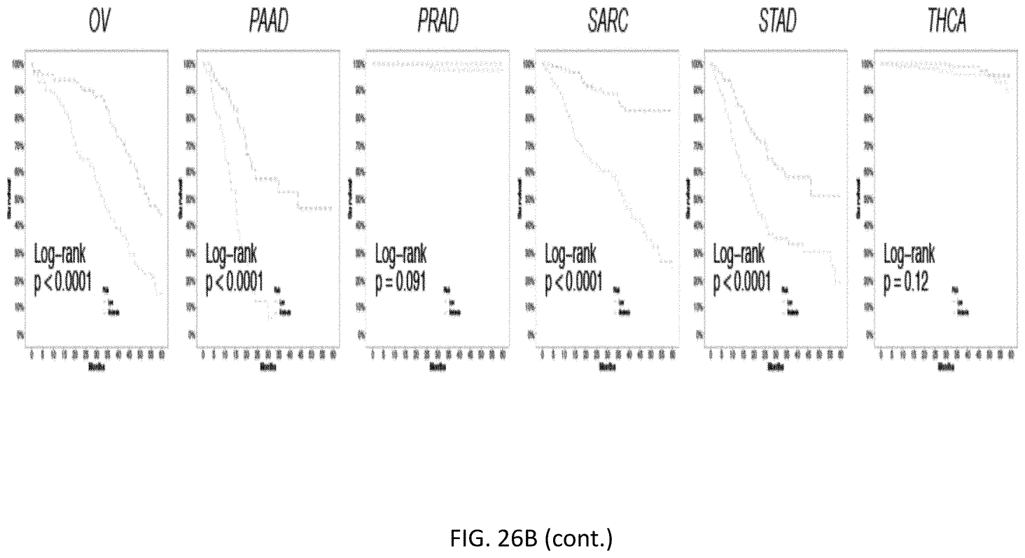

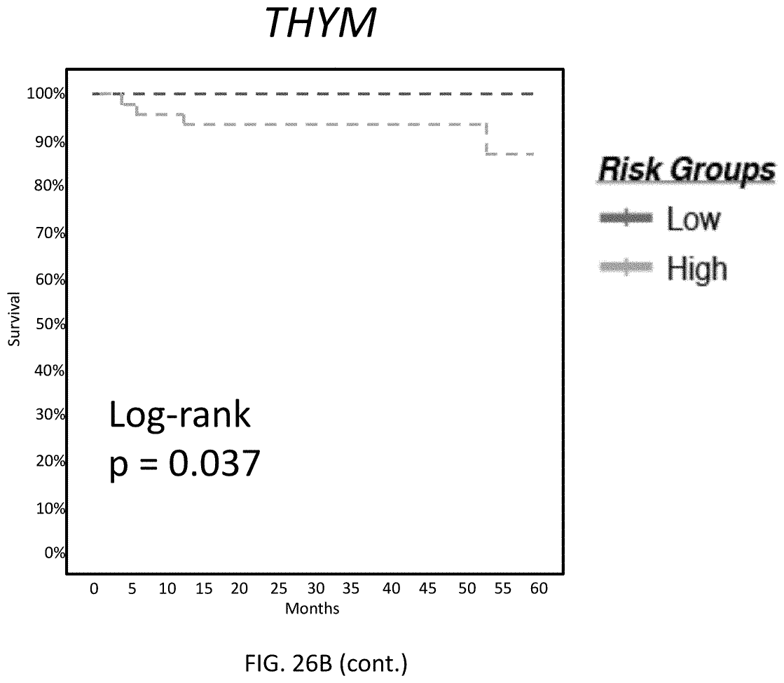

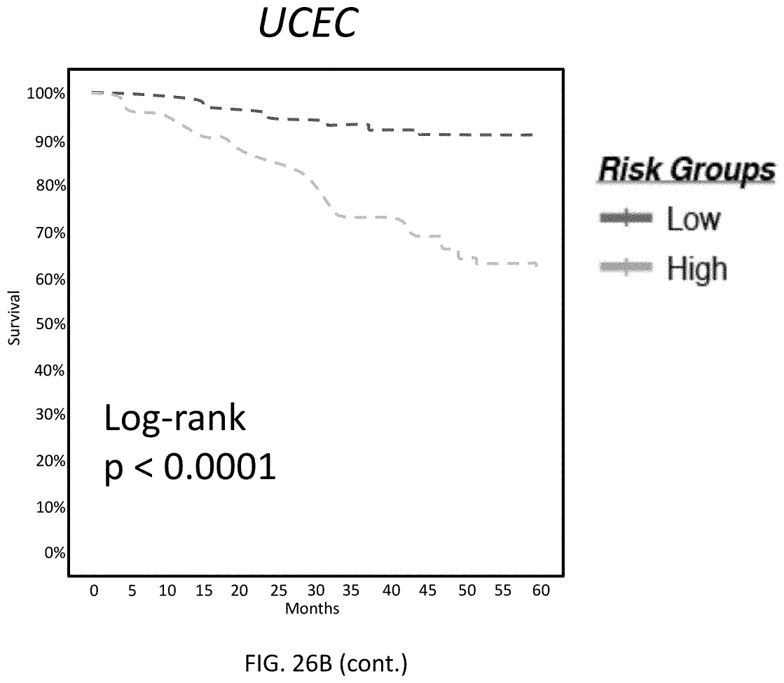

FIG. 26A and FIG. 26B depicts survival models at the nGOseq gene level comparing 20 cancer types from the TCGA database. (top) Survival model statistics (for both training (FIG. 25B) and testing (FIG. 25A) data sets) broken down by each of the 20 cancer types. (bottom) Kaplan-Meier plots for each of the 20 cancer types stratified into 2 risk groups (low risk—red, high risk—blue, solid—testing data, dashed—training data). Risk stratification was determined by grouping the predicted risks for each cancer type from the survival model at the MEGENA module level into 2 quantiles for all training samples and using the same median value to stratify the testing samples. P values were calculated via log-rank tests.

We sought to understand and evaluate the use of deep learning methodologies in classifying tumor sub-types from the same tissue of origin. This allowed us to focus on underlying differences in tumor biology rather than possible confounding tissue of origin biology. Consequently, we focused on 4 binomial comparisons (FIG. 4A) using tumor types from lung, kidney, and breast tissues with sufficient sample size and molecular measurements from all 5 data types; LUAD vs. LUSC (n=500 and n=462), KIRC vs. KIRP (n=284 and n=327), ER+vs. ER− BRCA subtypes (n=740 and n=219), and Luminal A vs. Luminal B BRCA subtypes (n=199 and n=112). Data from each platform (mRNA, miRNA, CNV, methylation, and SNP) was pre-processed and normalized and then merged into a single data matrix containing ˜70,000 molecular measurements for each binomial comparison. For single nucleotide polymorphism data, we built a deep artificial neural network (DANN) model (and a standard machine learning LASSO model) to assess pathogenicity of missense genomic variants. Both high-scoring loss of function variants and somatic missense variants with a pathogenic probability of ≥0.51 were retained. Each variant was mapped to a gene and the counts of all variants for a given gene were added together into a single count value, thus translating sparse binomial data into a continuous value.

We applied two distinct feature learning and dimensionality reduction techniques to create an overall integrated data matrix of all 5 data types for our computational intelligence methodology. MEGENA followed by principal component analysis (PCA) is a data driven clustering methodology that combines various molecular signals into integrated modules which are then represented by their first principal components (PC), commonly known as metagenes. Integrative nGOseq followed by PCA uses differential genes (across all 5 platforms) and apriori biological knowledge (gene ontology) to find functionally enriched biological pathways which are then represented by their first PCs. For example, MEGENA feature learning collapsed the original 70,005 molecular measurements, consisting of all 5 data types, from the KIRC vs. KIRP comparison into 604 modules, while nGOseq feature learning found 1,915 unique enriched GO terms. Thus, these smaller data matrices at the module/gene-set level were used as the input for the initial deep learning models.

We applied two distinct deep learning methodologies to these training datasets at the module/gene-set level; deep artificial neural networks (DANNs) and deep Bayesian neural networks (DBNNs). Model hyper-parameters were automatically tuned (such as learning rate, layer size, dropout rate, etc.) for optimal performance. Classification performance (FIG. 4a) of both deep learning techniques using each of the feature learning methodologies on the held-out test dataset at the module/gene-set level was perfect (AUC 1.0—LUAD vs. LUSC) or near perfect (AUC>0.90—KIRC vs. KIRP, ER+vs. ER−) for 3 of the 4 binomial comparisons while Luminal A vs. B showed reasonable classification performance (AUC>0.85). To further assess robustness of our feature learning approaches, independent of classification scheme and experimental platform, LASSO classifiers were trained using the nGOseq feature learning methodology with RNA-seq data only (mRNA) for the ER+vs. ER−, Luminal A vs. B, and LUAD vs. LUSC comparisons. These classifiers were then validated on independently available microarray datasets (Network, C. G. A. Nature 490, 61-70, (2012); Gyorffy, B. et al. PLoS One 8, e82241, (2013))_ENREF_45. The models achieved near perfect (AUC>0.90) classification performance on the validation microarray mRNA expression profiles for all comparisons. These cross-platform results indicate that the nGOseq feature learning strategy robustly captures a significant degree of biological signal within each experimental comparison. Interestingly, the LUAD vs. LUSC comparison uncovered an informative nGOseq term, containing 16 genes (DVL3, GRHL3, GJB6, USHIG, SLC9A3R1, WNT5A, FZD6, DLX5, NRPI, HPN, WNT3A, FGFR2, GLI2, CLICS, VANGL2, TFAP2A), annotated for the GO term ear morphogenesis. These findings suggest that our feature learning approaches are capable of identifying informative genes annotated for seemingly unrelated biological processes, thus affording novel hypothesis testing of disease etiology.

Although the classification performance at the module/gene-set level is remarkable, it is difficult to interpret underlying biological factors driving class separation due to the aggregation of multiple genes across integrated data types. Therefore, we developed a novel strategy to transition from the module/gene-set level to the gene level for both feature learning methodologies. We utilized an ensemble strategy, applied to each feature learning methodology independently, by taking the intersection of the most important modules/gene-sets identified through saliency mapping of both DANN and DBNN models. The most informative modules/gene-sets were determined and all molecular measurements within these modules/gene-sets were aggregated into a gene level matrix. For example, the KIRC vs. KIRP matrices consisted of 2,880 genes for nGOseq (592 CNVs, 663 METH, 36 miRNA, 612 mRNA, and 977 STVs) and 1,046 genes for MEGENA (177 CNVs, 340 METH, 35 miRNA, 382 mRNA, and 112 STVs).

We then re-trained DANNs and DBNNs on these gene level training datasets and automatically tuned model hyper-parameters (such as learning rate, layer size, dropout rate, etc.) for optimal performance. Classification performance at the gene level (FIG. 4a) of both deep learning techniques and both feature learning methodologies on the held-out test dataset, now at the gene level, remained high for LUAD vs. LUSC (AUC=1.0) and increased for KIRC vs. KIRP (increased accuracy, balanced accuracy, F1 score, and sensitivity), ER+vs. ER− (increased balanced accuracy and F1 score), and Luminal A vs. B (increased AUC, accuracy, balanced accuracy, F1 score, sensitivity, and specificity). Therefore, when moving from module/gene-set level to gene-level we retain and in 3 of 4 cases gain class separability with the added benefit of increased biological interpretability discussed below.

We next identified and examined important molecular markers for each feature learning methodology that contributed most to class separability between each of the 4 binomial comparisons. These molecular markers help give insights into the biology driving disease and can lead to novel hypotheses of pathways and genes implicated in cancer. Herein, we focus our discussion on the KIRC vs. KIRP comparison, however all methodology described was applied to the other comparisons (LUAD vs. LUSC, ER+vs. ER−, and Luminal A vs. Luminal B) and is discussed briefly below.

We first applied our ensemble saliency mapping methodology to our deep learning models at the gene level in order to calculate a ranked list of the most informative genes for each feature learning methodology. We then used the top 100 most informative genes (in some cases 99 genes if ties were present in rankings) to build Bayesian Belief Networks (BBNs) for each feature learning methodology to better understand the causal dependencies between informative genes (FIG. 4B-C). Genes that end up closer to the top of the directed acyclic graph (DAG) are more likely to have causal influence over those lower in the DAG. Changes in these upstream genes are more likely to lead to state changes of the downstream genes, thus affecting genes that are informative in class separability. We hypothesize that upstream genes in the BBNs would be useful molecular markers for class discrimination (diagnostics) or novel therapeutic targets. For the integrative nGOseq feature learning, we identified multiple methylated genes, CFPL2, FAM134C, CNGA4, ACAD9, and PPIF (FIG. 4B), that lie upstream in the BBN, while for MEGENA feature learning we identified 2 expression genes and 3 methylated genes, RP11.59C5.3, RP11.39404.5, RP11.517H2.6, FOXJ3, RP11.299J3.8 9 (FIG. 4C), and CCRI, that lie upstream in the BBN. Most striking is the MEGENA feature learning derived BBN has 4 of 6 non-functionally annotated upstream genes. In addition, several other genes had upstream qualities in the BBNs for both feature learning methodologies (FIG. 4D-E—black band), thus also being hypothetical candidates as molecular markers or therapeutic targets. Selected upstream genes for the other 3 binomial comparisons include; LUAD vs. LUSC—nGOseq: DTX3L and PLD1, MEGENA: ABI2, ABALON, and IDE, ER+vs. ER−—nGOseq: TFDP1, BCL11A, and SOSTDC1, MEGENA: LYN, RPRML, and CHAC1, Luminal A vs. Luminal B—nGOseq: TP63, SORCS1, and APC2, MEGENA: OR1L4, SLC7A10, and SUCLA2.

We mined available literature using natural language processing (NLP) to determine the connectivity of the top 100 genes to cancer, tissue specific cancer, and to other genes⁴⁶. Unsurprisingly, we found that informative genes from nGOseq feature learning were more significantly connected to cancer, survival, and between themselves in comparison to MEGENA feature learning with an average degree (edges per node) of 16.95 compared to 7.13 (FIG. 4D-E). This trend is consistent across the other 3 binomial comparisons. Moreover, 22 of the most informative MEGENA genes for KIRC vs. KIRP are functionally un-annotated (FIG. 4E—blue band) with 6 being considered upstream genes in the BBN. This demonstrates that a significant amount of biological information exists in functionally un-annotated genes that would not have be discovered with apriori knowledge approaches (e.g. nGOseq). However, both approaches also identified many known cancer and immune related genes (FIG. 4D-E—purple band) including; nGOseq: ATM, CD34, CDK5, JUN, MET, NFATC2, PRKCA, RAC1 and MEGENA: CCR1, HK1, RACGAP1.

We then examined if the top 100 genes for each feature learning methodology were associated with any known drug targets by mining DrugBank and Pharmacodia for existence of clinical trials in any indication. We found 14 genes from nGOseq and 11 genes from MEGENA, for the KIRC vs. KIRP comparison, that have existing therapeutics in which the gene is linked to the mechanism of action, some specifically in cancers such as CDK5, LCK, MAPK11, MET, and MMP16. This indicates that a portion of the identified genes are already therapeutic targets, but also that a substantial amount of the discovered gene space is still unexplored including many functionally un-annotated genes.

Given our methodologies success in classifying various tumor subtypes, we sought to understand the genetic similarities and differences driving a diverse set of tumors across multiple tissues of origin. We extended the applicability of our deep learning approach to a multinomial comparison of 22 cancer types across the TCGA database, following a similar strategy as described above for the binomial models. We focused on TCGA cancer types (Table A) with sufficient sample size (>100) and molecular measurements from all 5 data types. Thus, a total of 8,272 samples representing 22 cancer types (Table A) were used for further analysis. Due to the difficulty in establishing viable multinomial statistical models to calculate differential genes within the 5 data types, we only applied our data-driven MEGENA feature learning approach for this analysis. The multinomial deep learning models served as a benchmark of the scalability of our methodology and provided further insights into the applicability of our approach in understanding molecular cues underlying diverse cancer types.

MEGENA feature learning collapsed the original 78,915 molecular measurements from the 5 data types into 743 modules and this data matrix at the module level was used as the input for the two initial deep learning models. In short, we again trained both DANNs and DBNNs (using training data) and automatically tuned model hyper-parameters. Classification performance (FIG. 5A) of both deep learning techniques consisted of multiclass AUCs of 0.999, model accuracies greater than 0.95, and F1 scores greater than 0.90. These statistics indicated that our deep learning models performed exceptionally well in multinomial classification similar to our binomial models (FIG. 4A). Next, we calculated the relative importance, based on saliency maps derived from our ensemble DANN and DBNN deep learning models, of the most informative MEGENA modules for each cancer type (FIG. 5B). For each cancer type, there was a unique set of modules important for classification that differed among these cancer types. However, to our surprise, we also found important modules that are shared among different cancer types (e.g., c1_22_Block_14) which suggests a high degree of shared biology across cancers, despite their differences. This supports the notion that there are overlapping molecular factors underlying cancer biology.

One possible explanation for how well our models classified different tumor types is that the discovered molecular signatures simply reflect tissue of origin biology rather than specific tumor biology. Interestingly, important modules did not appear to cluster by tissue of origin as lung cancer subtypes (LUSC and LUAD) as well as kidney cancer subtypes (KIRP and KIRC) were separated from each other in the clustergram (FIG. 5B). However, to directly assess the possible confounding issues of tissue of origin signal, we employed our multinomial ensemble computational intelligence approach using only mRNA expression data (RNA-seq) to classify 19 cancer types along with sufficient matching normal tissue samples (17 tissues from GTEx and/or TCGA)(Consortium, G. T. Nat Genet 45, 580-585, (2013); Consortium, G. T. Science 348, 648-660, (2015); Consortium, G. T. et al. Nature 550, 204-213, (2017)). Our methodology led to near perfect classification (multiclass AUCs greater than 0.99, model accuracies greater than 0.95, and F1 scores greater than 0.95) at both the MEGENA module (n=236) and gene levels (n=3059) in also segregating specific tumor types from matching normal tissue samples.

In addition, we utilized our computational approach on only normal tissues (as described above) and used it to classify the 17 tissues of origin which showed perfect discriminatory capabilities. We assessed if we could use this model, trained on only normal tissues, to predict tissue of origin of the 19 cancer types. The model showed marginal ability to predict tissue of origin of tumors. This concept is further illustrated by a 5th integrated binomial comparison of BRCA vs. normal (73 matched tumor and normal samples). As with the integrated binomial LUAD vs. LUSC comparison described above, this model yielded perfect classification performance (AUC=1; model accuracy=1; F1 Score=1) with both deep learning techniques and both feature learning methodologies on the held-out test dataset at the module/gene-set and gene levels. Moreover, BNN analysis of nGOseq and MEGENA top 100 genes identified potential molecular markers or therapeutic targets, including AURKB, DDR2, MAML, AVPI1 and PSMD11 which overlap with known breast cancer related genes. Interestingly, we also discovered a gene related to the dopamine receptor pathway (DRD2) that has recently garnered attention as an anti-cancer target using thioridazine (an anti-psychotic). Taken together, these results demonstrate that the similarities and differences between the diverse cancer types identified by our computational intelligence approach are not primarily due to a tissue of origin signal.

Therefore, we assessed the biological significance of the genes in the most informative MEGENA modules from the pan 22 cancer DANNs and DBNNs with integrative nGOseq functional enrichment (selected nGO terms in FIG. 5C). We discovered that the genes making up the 13 modules showed significant enrichment (p-value 0.05) for all 10 of the hallmarks of cancer_ENREF_50 (Hanahan, D. et al. Cell 144, 646-674, doi:10.1016/j.cell.2011.02.013 (2011).). Even more notable was that we identified these enriched pathways nested in highly relevant GO terms (FIG. 5B—left column is nGO term and right column is GO term). For example, enrichment of lymphocyte activation, an immune related process, was nested in the cellular response to DNA damage stimulus GO term indicating that the immune response is tied to canonical oncogenic processes. In addition, we found more well-known process such as PI3K binding nested in ion binding, response to FGF nested in cell differentiation, and regulation of G1/S transition of mitotic cell cycle nested in cell differentiation. Taken together, these results indicate that our deep learning approach at the module level can identify relevant cancer biology shared across multiple tumor types.

As we did for the binomial models above, the most important modules were then determined and all molecular measurements that were within these modules/gene-sets were aggregated into a gene level matrix. This matrix consisted of 1316 genes made up of 445 mRNA, 20 miRNA, 22 STV, and 829 methylation measurements. CNV data was not present most likely due to the low frequency of alterations shared across cancers with similar reasoning justifying the low number of STVs in the final gene matrix. As with our binomial approach, we observed a marked increase in model performance on the test data set at the gene level compared to the module level with AUCs, accuracies, and F1 scores all greater than 0.99. We misclassified only 7 of 1645 and 9 of 1645 test samples using DANN and DBNN models respectively, with 5 overlapping misclassifications. We then calculated the top 100 most informative genes for each of the 22 cancer types, based on the intersection of saliency maps derived from our ensemble DANN and DBNN deep learning models, ordered the union set by the total number of occurrences (i.e. the number of cancers the gene is important in), and subsequently filtered the list by removing genes important in less than 5 cancers which lead to a list that consisted of 200 informative genes shared across 22 cancer types (Table M).

The top 51 genes, which are informative in 6 or more cancers, are shown in FIG. 5D for all 8,272 samples (training and testing data sets) with KCNQ1 (METH), PIK3CA (METH), IL-20 (METH), STON2 (METH), RP11.540D14.8 (METH), AGT (METH), HAS2-AS1 (mRNA), XPR1 (mRNA), NFIX(mRNA), and MGMT (METH) ranked as the top 10 genes respectively. PIK3CA is a member of the well-studied PI3K family which has been shown to significantly contribute to the development of cancer_ENREF_51 (Fruman, D. A. et al. Nature Reviews Drug Discovery 13, 140-156, (2014).), KCNQ1 is a voltage gated potassium channel that may have a potential role in GI cancer_ENREF_52 (Than, B. L. N. et al. Oncogene 33, 3861-3868, (2014).), AGT is part of the Renin-angiotensin system which plays a role in many oncogenic processes_ENREF_53 (Pinter, M. et al. 5616, (2017).), and IL-20 in an emerging pro-inflammatory cytokine that may regulate proliferation and metastasis (Lee, S. J. et al. Journal of Biological Chemistry 288, 5539-5552, (2013); Hsu, Y.-H. et al. The Journal of Immunology 188, 1981-1991, (2012)). Collectively, these results demonstrate that our computational methodology was able to discover both known and novel genomic details shared between multiple cancer types.

To assess the biological relevance of the outcome of our gene-level models in cancer, we again performed NPL on the top 200 informative genes from multinomial comparison (FIG. 3e). We identified associations between many of the top 200 genes and cancer in published literature. Notably, we discovered 46 informative genes across 22 cancer types that currently have no association with cancer or other genes in published literature (FIG. 5E—purple band) with 26 that have no associated functional annotation (FIG. 5E—blue band). Therefore, we believe that our deep learning models identified new associations between poorly characterized genes (i.e., RP11 genes) and cancer and propose that this is a highly valuable tool to identify new therapeutic targets. Importantly, our model also identified several genes that are known drug targets, including PIK3CA_ENREF_56 (Pixu Liu, H. C. et al. Nature Reviews Drug Discovery 8, 627-644, (2009).), EGF_ENREF_57 (Parthasarathy Seshacharyulu, M. P. P., et al. Expert Opinion on Therapeutic Targets 16, 15-31, (2012).) and ADAM28_ENREF_58 (Maeve Mullooly, P. M. M., et al. Cancer Biology & Therapy 17, 870-880, (2016).), (FIG. 5E—bold italicized names) which are highly associated with cancer and to other genes (FIG. 5E—dark purple in inner band). Combined, these two observations suggest that our multinomial model can generate testable hypotheses for new therapeutic targets as well as capture more un-known cancer biology.

We then investigated the prognostic utility of TCGA molecular data in predicting patient survival. We focused on 20 cancer types for survival analysis that included molecular data from all 5 data types, significant follow up data (more than 5% of follow-ups were reported as deceased), and sufficient sample size and thus a total of 7,822 samples were used in subsequent analysis. Unlike most existing work (Yuan, Y. et al. Nat Biotechnol 32, 644-652, (2014); Director's Challenge Consortium for the Molecular Classification of Lung, A. et al. Nat Med 14, 822-827, (2008); Cheng, W. Y. et al. Sci Transl Med 5, 181ra150, (2013); Ceccarelli, M. et al. Cell 164, 550-563, (2016)) where clinical information such as molecular subtype, grade, stage, etc. were used in survival analysis our analysis only included a single clinical variable, age, to help control for two well-known factors; risk of death as age increases and the use of overall survival (death from any cause) instead of disease-specific survival (death from the specific disease only). Therefore, our models were focused on assessing the prognostic utility of molecular scale information. We hypothesized that investigating survival across multiple cancer types would benefit from multiple factors: (1) increased statistical power due to increased sample size, (2) an increased incidence of death as right censored data is highly informative but notoriously difficult to model, and (3) there exist shared molecular factors between cancers that contain significant prognostic value when interrogating data across multiple cancer types.

In order to adequately assess the prognostic utility of molecular information, we determined that it was critical to balance for multiple factors when splitting the dataset into training and testing sets. We stratified the dataset based on age (collapsed into 2 year intervals), overall survival (collapsed into 2 month intervals), survival status (LIVING vs. DECEASED), and cancer type in order to preserve the overall data distribution between the training and testing datasets. We built our predictive survival models on the training data set using deep hazard neural networks (DHNNs, see Supplemental Materials and Methods) with the same workflow to move from the module/gene-set level to the gene level as used in previous models. Two different metrics were used to assess model performance, c-index and tAUC (Uno, H., et al. Stat Med 30, 1105-1117, (2011).), both of which scale between 0 and 1 where 0.5 is no better than random while 1.0 is perfect model concordance.

All DHNN models, MEGENA and nGOseq at both the module and gene level, showed substantial predictive performance (FIG. 6A) with overall model c-indices of (0.75, 0.76, 0.75, 0.76) and overall temporal AUCs of (0.75, 0.75, 0.75, 0.75). When model statistics at the MEGENA module level were broken down by individual cancer types (FIG. 6B), where models were trained on all cancer types but the predictive power was evaluated on each cancer type, 9 of 20 cancer types have a predictive test statistic (c-index or tAUC) above 0.70 and 15 of 20 cancers have a predictive test statistic (c-index or tAUC) above 0.60. Cancers with predictive statistics above 0.70 are similar (e.g. BRCA and LGG) or surpass the current state of the art predictive capabilities of survival models (Director's Challenge Consortium for the Molecular Classification of Lung, A. et al. Nat Med 14, 822-827, (2008); Cheng, W. Y. et al. Sci Transl Med 5, 181ra150, (2013); Ceccarelli, M. et al. Cell 164, 550-563, (2016); Bianchi, F. et al. J Clin Invest 117, 3436-3444, (2007); Guinney, J. et al. The Lancet Oncology 18, 132-142, (2017); Mankoo, P. K., et al. PLoS One 6, e24709, (2011)). Furthermore, these predictions are based on molecular scale features and contain no clinical information other than age, thus demonstrating that molecular scale information has significantly more prognostic power than previously suggested_ENREF_59 (Yuan, Y. et al. Nat Biotechnol 32, 644-652, (2014)). Survival models at the MEGENA gene level, nGOseq module level, and nGOseq gene level demonstrate similar trends in predictive power across multiple cancer types; however, these models have increased variability in predictive power between training and testing data sets.

In order to better understand the possible shared nature of molecular risk factors across multiple cancer types, we trained a survival model at the MEGENA module level on data from 19 of the 20 cancer types and tested on the left-out cancer type (in this case UCEC). The c-index and tAUC metrics (FIG. 6C) on the left-out UCEC samples were 0.70 and 0.71 respectively, which denoted that the survival model retained predictive capabilities on an unknown cancer type. This indicated that shared molecular scale risk factors exist between UCEC and at least a portion of the other 19 cancers.

To determine if risk groups exist in within the predictive survival models, we used the model predicted risks and stratified each cancer into 2 groups (high-risk and low-risk) based on the median predicted risk from the training data set (6,225 samples). FIG. 6D shows Kaplan-Meier plots for the training and held-out testing samples stratified by median training data set risk for each of the 20 cancer types at the MEGENA module level. 19 of 20 cancer types from the training data sets and 10 of 20 cancer types from the testing data set (FIG. 6D—bolded names) showed significant differences (by log rank test, p-value 0.05) in risk between the 2 groups, indicating the prognostic utility of molecular information in stratifying patients into risk groups. Again, survival models at the MEGENA gene level, nGOseq module level, and nGOseq gene level demonstrate similar trends in risk stratification. Most notably from the test data set, CESC (p=0.048, log-rank), KIRP (p=0.0033, log-rank), LGG (p=0.0039, log-rank), LUAD (p=0.014, log-rank), and STAD (p=0.014, log-rank) showed clearly delimited risk groups, with the high-risk groups having less than ˜60% survival by 30 months compared to greater than 85% survival in the lower risk group (STAD is slightly different with 25% and 70% respectively). In addition, we were able to stratify a high-risk population from the test data set for BRCA (p=0.0014, log-rank), CRAD (p=0.0033, log-rank), OV (p=0.037, log-rank), PRAD (p=0.021, log-rank), and UCEC (p=0.0019, log-rank) with BLCA, HNSC, and KIRC bordering on statistically significant risk groups (p=0.11, 0.16, and 0.055 respectively, log-rank). For BRCA, our patient stratification results were similar to those found by the DREAM breast cancer prognosis challenge_ENREF_67 (Cheng, W.-y., et al. Science translational medicine 5, 181ra150, (2013)). Similarly, LGG stratification was comparable to the hyper-methylation subset discovered within all glioblastoma stages_ENREF_68 (Ceccarelli, M. et al. Cell 164, 550-563, (2016)). These results show that prediction of risk groups in multiple cancer types could have significant impact on patient prognosis, biomarker development, and identification of appropriate treatment regimes.

We explored the most important molecular markers from each of the survival models at the gene level to gain mechanistic understanding of patterns of survival across multiple cancer types. We identified important molecular features using two complementary methods; univariate assessments of significant hazard ratios and saliency mapping of the gene level DHNNs to determine the most informative genes.

Univariate hazards ratios were calculated for each cancer type for both the input gene level lists from MEGENA and nGOseq feature learning using a simple cox proportional hazards model with the gene of interest as the only covariate. All p-values were corrected with Benjamini-Hochberg false-discovery and the number of shared hazards ratios between each pair of cancers were calculated (FIG. 7A-B). Both nGOseq and MEGENA feature learning methodologies showed a large number of shared significant hazards ratios (p-value 0.05, likelihood ratio test) between different cancer types with BRCA, BLCA, LGG, LUAD, LUSC, KIRP, KIRC, and UCEC specifically enriched for shared risk factors between each other and with other cancer types. However, the maximum number of shared cancers for significant hazard ratios was only 7 (LIHC, LGG, KIRC, LUAD, CESC, LUSC, and KIRP) indicating that we are more likely identifying shared risk factors between multiple cancers and not fully pan-cancer signals. These results demonstrate that our survival models are not finding only cancer-type specific prognostic molecular markers as a large portion of important molecular features at the gene level are shared across multiple cancers.

In order to assess the contribution of genes to survival predictions in a more multivariate manner we computed saliency maps for both MEGENA and nGOseq DHNN models at the gene level and determined the top 100 most informative genes associated with survival for each model. The top 100 genes for nGOseq consisted of methylation, CNV, mRNA and STV data types while those for MEGENA consisted of methylation, mRNA, STV, and miRNA data types. This indicates that all 5 types of molecular information have some prognostic utility. We then constructed Bayesian belief networks for the top 100 genes for both nGOseq and MEGENA (FIG. 7C-D) to better understand the causal drivers of survival. The most upstream genes in the network for nGOseq were EFNA2 (CNV), TBCDOC (mRNA), RAB15 (Methylation), KLHLIO (Methylation), and CACNG4 (Methylation). EFNA2 belongs to the Eph family of receptor tyrosine kinases while TBCIDIOC and RAB15 are part of the Ras oncogene pathway. The most upstream drivers in the network for MEGENA were TUBB2B (mRNA), TERC (Methylation), FCGR2A (mRNA), CDK4 (STV), and GCNT4 (mRNA). TUBB2D is an isoform of tubulin which forms the basis of microtubules, TERC maintains teleomere ends, FCGR2A is a major immune receptor found mainly on B-cells, and CDK4 is a well-known Ser/Thr protein kinase implicated in a multitude of cancers (also a target for multiple developed drugs). Taken together these results indicate that a multitude of biological pathways (from cellular senescence to cellular division to the immune response) play a role in determining patient survival across multiple cancer types.

To validate the importance of a portion of the top 100 most informative genes we identified significant hazard ratios for BRCA using the same univariate analysis as described above (only of the top 100 genes) and performed a similar analysis with the METABRIC dataset, another publically available BRCA dataset consisting of molecular measurements (mRNA and CNV data only) and survival information_ENREF_61 (Cheng, W. Y. et al. Sci Transl Med 5, 181ra150, (2013).). For nGOseq there were 24 significant hazard ratios of which 10 mRNAs and 3 CNVs are present in both datasets, while for MEGENA there were 23 significant hazard ratios of which 9 mRNAs and 0 CNVs are present in both datasets. Of the TCGA identified significant hazard ratios, 7 of 10 mRNA and 2 of 3 CNVs from the most informative nGOseq genes were also significant in the METABRIC data, while 4 of 9 mRNA from the most informative MEGENA genes were also significant in the METABRIC data. This demonstrates that our identified prognostic molecular markers are not dataset specific, however this needs to be further validated with additional patient data.

We mined available literature using natural language processing to determine the connectivity of the top 100 genes to survival and between the most informative genes (FIG. 5E-F). We found results similar to those shown above (binomial models) in which nGOseq genes are much more connected to cancer, survival, and between themselves in comparison to MEGENA genes. This indicates that MEGENA feature learning tends to bring more novel information to the survival models. In addition, 22 of the top 100 MEGENA genes are un-annotated indicating that there are significant prognostic molecular factors that we have limited understanding of (i.e. RP11-1055B8.1). Yet, saliency mapping (for both nGOseq and MEGENA) also identified many known cancer related processes and molecules which include; known oncogenes (i.e. TP63, MAP2K2, CDKN2A), kinase pathways (MAP2K2, CDK4), and immune related molecules (FCGR2A, CD80, TGFB1). This reinforces the theme that a multitude of biological processes contribute to patient survival and that no one single factor is the determinant of our model predictions; however, there exist a multitude of shared molecular factors that are prognostic across multiple cancer types.

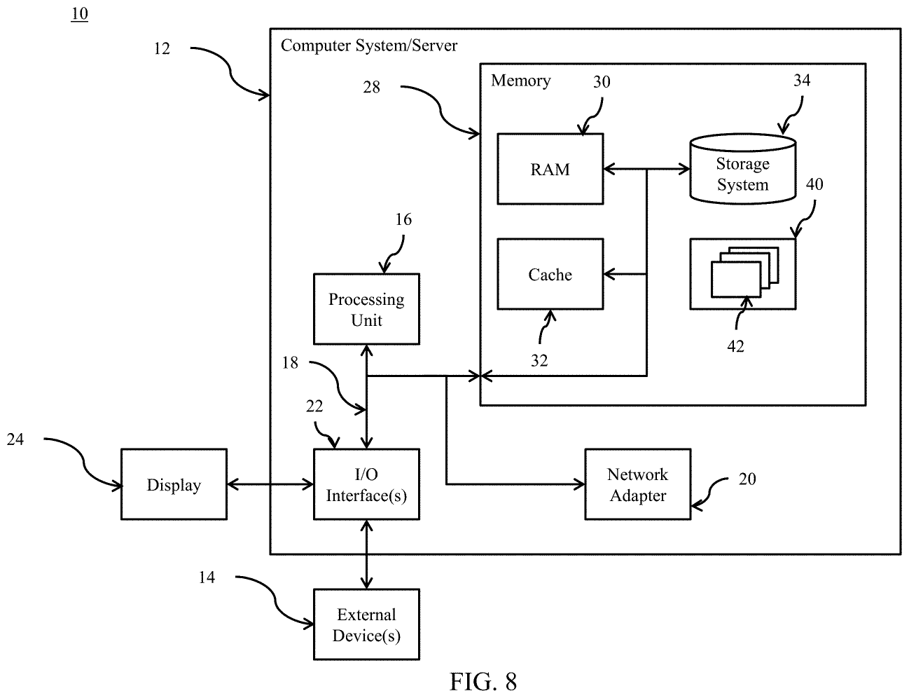

Referring now to FIG. 8, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 8, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (IO) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Lengthy table referenced here
US20200327962A1-20201015-T00001
Please refer to the end of the specification for access instructions.

Lengthy table referenced here
US20200327962A1-20201015-T00002
Please refer to the end of the specification for access instructions.

Lengthy table referenced here
US20200327962A1-20201015-T00003
Please refer to the end of the specification for access instructions.

Lengthy table referenced here
US20200327962A1-20201015-T00004
Please refer to the end of the specification for access instructions.

Lengthy table referenced here
US20200327962A1-20201015-T00005
Please refer to the end of the specification for access instructions.

Lengthy table referenced here
US20200327962A1-20201015-T00006
Please refer to the end of the specification for access instructions.

Lengthy table referenced here
US20200327962A1-20201015-T00007
Please refer to the end of the specification for access instructions.

Lengthy table referenced here
US20200327962A1-20201015-T00008
Please refer to the end of the specification for access instructions.

Lengthy table referenced here
US20200327962A1-20201015-T00009
Please refer to the end of the specification for access instructions.

Lengthy table referenced here
US20200327962A1-20201015-T00010
Please refer to the end of the specification for access instructions.

Lengthy table referenced here
US20200327962A1-20201015-T00011
Please refer to the end of the specification for access instructions.

Lengthy table referenced here
US20200327962A1-20201015-T00012
Please refer to the end of the specification for access instructions.

Lengthy table referenced here
US20200327962A1-20201015-T00013
Please refer to the end of the specification for access instructions.

Lengthy table referenced here
US20200327962A1-20201015-T00014
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20200327962A1). An electronic copy of the table will also be available from the USPTO upon request and payment of the fee set forth in 37 CFR 1.19(b)(3).

1. A method comprising:
reading biological data of a population;
extracting a plurality of features of the population from the biological data;
providing the plurality of features to a first trained classifier to determine a subset of the plurality of features distinguishing the population;
determining a plurality of genes associated with the subset of the plurality of features;
providing the plurality of genes to a second trained classifier to determine a subset of the plurality of genes distinguishing the population;
applying a dependence model to the subset of the plurality of genes to determine one or more drug target.

2. The method of claim 1, wherein the biological data comprise at least one of: molecular features of the population, phenomic data, clinical data, genomic data, proteomic data, transcriptomic data, epigenomic data, or microbiomic data.

3. (canceled)

4. (canceled)

5. (canceled)

6. The method of claim 1, wherein the extracted features comprise one or more metagene.

7. The method of claim 1, wherein the extracted features correspond to gene clusters.

8. The method of claim 1, wherein the features are extracted by clustering the biological data, wherein clustering comprises: hierarchical clustering, k-means clustering, distribution-based clustering, Gaussian mixture models, density-based clustering, or highly connected subgraphs clustering.

9. (canceled)

10. The method of claim 1, wherein the features are extracted by gene correlation, wherein gene correlation comprises: multiscale embedded gene co-expression network analysis, clustering based on measured molecular data, or clustering based on biological annotations.

11. (canceled)

12. (canceled)

13. (canceled)

14. The method of claim 1, wherein extracting the plurality of features comprises applying principle component analysis.

15. The method of claim 1, wherein extracting the plurality of features comprises applying nonlinear dimensionality reduction.

16. The method of claim 1, wherein the first trained classifier comprises an artificial neural network, the artificial neural network comprising a deep artificial neural network or a deep Baysian neural network.

17. (canceled)

18. The method of claim 1, wherein the first trained classifier comprises a support vector machine.

19. The method of claim 1, further comprising:
providing the plurality of features to a third trained classifier to determine a second subset of the plurality of features distinguishing the population; and
combining the first and second subsets of the plurality of features.

20. (canceled)

21. The method of claim 1, further comprising:
ranking the subset of the plurality of features by saliency by generating a saliency map.

22. (canceled)

23. The method of claim 1, wherein the second trained classifier comprises an artificial neural network, the artificial neural network comprising a deep artificial neural network or a deep Baysian neural network.

24. (canceled)

25. The method of claim 1, wherein the second trained classifier comprises a support vector machine.

26. The method of claim 1, further comprising:
providing the plurality of genes to a fourth trained classifier to determine a second subset of the plurality of genes distinguishing the population; and
combining the first and second subsets of the plurality of genes.

27. (canceled)

28. The method of claim 1, further comprising:
ranking the subset of the plurality of genes by saliency by generating a saliency map.

29. (canceled)

30. The method of claim 1, wherein the dependence model comprises a Bayesian belief network.

31. The method of claim 1, further comprising:
determining one or more association between the one or more drug target and a disease vocabulary term by searching existing medical literature.

32. (canceled)

33. The method of claim 31, wherein the association includes a relationship between the one or more drug target and the disease vocabulary term, wherein the relationship is stimulatory, inhibitory, neutral, or parallel.

34. (canceled)

35. The method of claim 1, further comprising:
determining one or more association between the one or more drug target and a drug vocabulary term.

36. The method of claim 35, wherein determining the one or more association comprises searching existing medical literature.

37. The method of claim 35, wherein the association includes a relationship between the one or more drug target and the drug vocabulary term, wherein the relationship is stimulatory, inhibitory, neutral, or parallel.

38. (canceled)

39. (canceled)

40. A system comprising:
a computing node comprising a computer readable storage medium having program instructions embodied therewith, the program instructions executable by a processor of the computing node to cause the processor to perform a method comprising:
reading biological data of a population;
extracting a plurality of features of the population from the biological data;
providing the plurality of features to a first trained classifier to determine a subset of the plurality of features distinguishing the population;
determining a plurality of genes associated with the subset of the plurality of features;
providing the plurality of genes to a second trained classifier to determine a subset of the plurality of genes distinguishing the population;
applying a dependence model to the subset of the plurality of genes to determine one or more drug target.

41-78. (canceled)

79. A computer program product for identifying drug targets, the computer program product comprising a computer readable storage medium having program instructions embodied therewith, the program instructions executable by a processor to cause the processor to perform a method comprising:
reading biological data of a population;
extracting a plurality of features of the population from the biological data;
providing the plurality of features to a first trained classifier to determine a subset of the plurality of features distinguishing the population;
determining a plurality of genes associated with the subset of the plurality of features;
providing the plurality of genes to a second trained classifier to determine a subset of the plurality of genes distinguishing the population;
applying a dependence model to the subset of the plurality of genes to determine one or more drug target.

80. A method of identifying at least one therapeutic or drug target for at least one cancer, the method comprising the steps of:
(a) receiving or providing at least one data set obtained from at least one cancer type; and
(b) processing the at least one data set according to the method of claim 1, to thereby identify at least one therapeutic or drug target;
wherein said at least one therapeutic or drug target is at least one gene listed in Table B, Table C, Table D, Table E, Table F, Table G, Table H, Table I, Table J, Table K, Table L, Table M, Table N, Table O, Table AP, Table AQ, Table AR, Table AS, Table AT, Table AU, Table AV, Table AX, Table AY, Table AZ, Table AAA, Table AAB, Table AAC, Table AAD, Table AAF, Table AAG, Table AAH, Table AAJ, Table AAK, Table AAL, Table AAM, Table AAN, or Table AAO.

81-163. (canceled)
